(19) United States
(12) Patent Application Publication
(10) Pub. No.: US 2024/0246920 A1
(43) Pub. Date: Jul. 25, 2024

(54) TRIAZOLE DERIVATIVES WITH ANTIFUNGAL ACTIVITY

(71) Applicants: King's College London, London (GB); Secretary of State for Health and Social Care, London (GB)

(72) Inventors: Khondaker Mirazur Rahman, London (GB); Yiyuan Chen, London (GB); John Mark Sutton, Salisbury (GB); Charlotte Hind, Salisbury (GB)

(73) Assignees: King's College London, London (GB); Secretary of State for Health and Social Care, London (GB)

(21) Appl. No.: 17/797,362

(22) PCT Filed: Feb. 5, 2021
(86) PCT No.: PCT/GB2021/050268

§ 371 Date: Jan. 30, 2023

(30) Foreign Application Priority Data
Feb. 5, 2020 (GB) 2001564.0

(51) Int. Cl. C07D 249/08; A61K 31/4196; A61K 31/438; A61K 31/454; A61K 31/506; A61K 31/5377; A61K 31/551; A61P 31/10; C07D 401/06; C07D 401/14; C07D 403/12; C07D 403/14; C07D 417/14; C07D 471/10; C07D 487/04

(52) U.S. Cl. CPC C07D 249/08; A61K 31/4196; A61K 31/438; A61K 31/454; A61K 31/506; A61K 31/5377; A61K 31/551; A61P 31/10; C07D 401/06; C07D 401/14; C07D 403/12; C07D 403/14; C07D 417/14; C07D 471/10; C07D 487/04

(57) ABSTRACT

Disclosed are compounds of the formula (I) and pharmaceutically acceptable salts thereof:

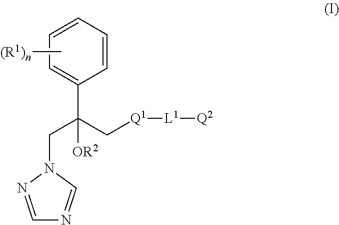

wherein R¹, R², Q¹, Q², L¹ and n are as defined herein. The compounds have antifungal properties and are useful in the treatment of fungal infections, including infections that are resistant to conventions anti-fungal agents.

This invention relates to triazole alcohol derivatives useful as antifungal agents which are active against a number of fungal species, for example Candida spp., particularly Candida spp. that are resistant to conventional anti-fungal agents such as Candida auris.

### BACKGROUND

Fungal infections are widespread and it is estimated there are more than 150 million people with serious fungal diseases (Bongomin et al., J. Fungi, 2017, 3, 57). Recent increases in immunocompromised subjects, including those with HIV, cancer and transplant recipients, has resulted in a dramatic increase of human invasive mycosis cases, particularly invasive Candidiasis, which has become a global public health problem (Limper et al., The Lancet Infectious Diseases, 2017, e334-e343; Jung et al., Emerging Infectious Diseases, 2015, 21, 1942; and Varughese et al., Clinical Infectious Diseases 2018, 67, 687-692).

Despite the prevalence of fungal infection there are a limited number of anti-fungal drugs available and many of these have limitations including low efficacy and/or undesirable toxicity. Moreover, numerous fungal strains are inherently resistant to, or have acquired resistance to conventional anti-fungal drugs, rendering numerous clinical fungal infections virtually untreatable.

Antifungal resistance is a particular problem with Candida infections. Some types of Candida are increasingly resistant to the first-line and second-line antifungal medications, such as fluconazole and the echinocandins (e.g. anidulafungin, caspofungin, and micafungin) (Perlin et al., The Lancet Infectious Diseases, 2017, 17, e383-e392; Sanguinetti et al., Mycoses, 2015, 58, 2-13; and Whaley et al., Frontiers in microbiology, 2017, 7, 2173). About 7% of all Candida bloodstream isolates tested by the U.S. Department of Health & Human Services, Centers for Disease Control and Prevention (CDC) are resistant to fluconazole. More than 70% of these resistant isolates are Candida glabrata or Candida krusei, strains which are intrinsically resistant to various azole drugs. Multidrug-resistant Candida infections (e.g. those that are resistant to both azoles and an echinocandin) have very few remaining treatment options (Perlin et al., supra).

Candida auris is a new strain of fungus first identified in Japan in 2009. C. auris infection rates have grown rapidly and despite the implementation of enhanced infection control procedures, nosocomial infection and transmission remains a problem (Jeffery-Smith et al., Clin. Microbiol. Rev. 2017 Nov. 15; 31(1):e00029-17). Reported cases of C. auris infections increased 318% in 2018 when compared to the average number of cases reported in 2015 to 2017 (CDC 2019 A R Threats Report). C. auris is associated with severe invasive infections and outbreaks with high mortality rates. Isolates of C. auris sent to CDC are almost all resistant to azoles, and up to one-third are resistant to amphotericin B, usually reserved as a last-resort treatment (Vallabhaneni et al., MMWR. Morbidity and mortality weekly report, 2016, 65). It's transmissibility and multi-drug resistance set it apart from most other Candida species and as such C. auris has been classified as an urgent threat by the CDC (CDC AR Threats Report 2019).

Emami et al., European J. Med. Chem., 2019, 173-194 is a review of certain triazole alcohol derivatives of fluconazole.

Guillon et al., ChemMedChem, 2011, 6, 1806-1815 describes certain benzylamine substituted triazole alcohols which are stated to be potent antifungal agents.

Jiang et al., European J. Med. Chem., 2014, 82 490-497, describe certain triazole derivatives containing substituted 1,2,3-triazole-piperdine side chains, which are described as antifungal agents.

WO 2011/099804 describes certain triazole derivatives which are described to be anti-fungal agents.

There remains an urgent need to identify new drugs for the treatment or prevention of fungal infections, particularly fungal infections that are resistant to current antifungal drugs.

### BRIEF SUMMARY OF THE DISCLOSURE

In accordance with the present inventions there is provided a compound of the formula (I), or a pharmaceutically acceptable salt thereof:

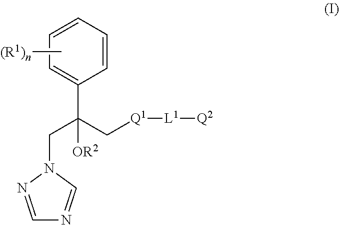

wherein:
R¹ is at each occurrence independently selected from: halo and C₁₋₄ alkyl;
n is 0, 1 or 2;
R² is selected from H and C₁₋₄ alkyl;
Q¹ is selected from:

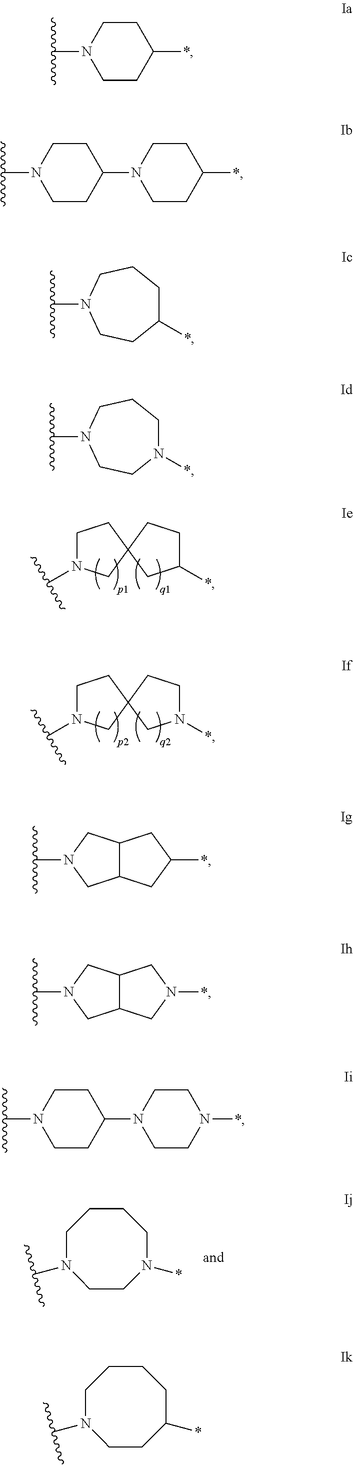

wherein * indicates the point of attachment to L¹,
p¹, q¹, p² and q² are independently an integer selected from 1 or 2,
and wherein Q¹ is optionally substituted by one or more R³;
each R³ is independently selected from: C₁₋₃ alkyl and ═O;
L¹ is a bond or is selected from —CH₂—, —O— and —NR⁴—, provided that L¹ is a bond or —CH₂-when Q¹ is bonded to L¹ via a ring nitrogen in Q¹;
R⁴ is selected from H and C₁₋₃ alkyl;
Q² is selected from phenyl or a 5- or 6-membered heteroaryl, wherein Q² is optionally substituted by one R⁵ and/or optionally one or more R⁶;
R⁵ is Q³-L³-, wherein
   L³ is a bond or is selected from: C₁₋₄ alkylene, -L⁴-O-L⁵-, -L⁴-O-L⁵¹-O-L⁵-, -L⁴-NR^{A1}-L⁵-, -L⁴-S(O)ₓ-L⁵-, -L⁴-C(═O)-L⁵, -L⁴-NR^{A1}C(═O)-L⁵-, -L⁴-C(═O)NR^{A1}-L⁵-, -L⁴-S(O)₂NR^{A1}-L⁵-, -L⁴-NR^{A1}S(O)₂-L⁵-, -L⁴-OC(═O)-L⁵ and -L⁴-C(═O)O-L⁵-,
   wherein x is 0, 1 or 2;
   L⁴ and L⁵ are independently selected from a bond and C₁₋₄ alkylene;
   L⁵¹ is C₁₋₄ alkylene;
   Q³ is selected from: phenyl, a 5- or 6-membered heteroaryl, 3- to 12-membered heterocyclyl and C₃₋₆ cycloalkyl, wherein said phenyl or 5- or 6-membered heteroaryl is optionally substituted by one or more R⁷, and said 3- to 12-membered heterocyclyl or C₃₋₆ cycloalkyl is optionally substituted by one or more R⁸:
each R⁶, R⁷ and R⁸ is independently selected from:
halo, ═O, —CN, —NO₂, C₁₋₆ alkyl, C₂₋₆ alkenyl, C₂₋₆ alkynyl, C₁₋₆ haloalkyl, C₁₋₆ cycloalkyl, —OR^{A1}, —S(O)ₓR^{A2} (wherein x is 0, 1, or 2), —NR^{A2}R^{B2}, —C(O)R^{A}, —OC(O)R^{A2}, —C(O)OR^{A2}, —NR^{B2}C(O)R^{A2}, —NR^{B2}C(O)OR^{A2}, —C(O)NR^{A2}R^{B2}, —NR^{B2}SO₂R^{A2} and —SO₂NR^{A2}R^{B2};
   wherein said C₁₋₆ alkyl, C₂₋₆ alkenyl, C₂₋₆ alkynyl are each is optionally substituted by 1 or 2 substituents independently selected from: halo, —CN, —OR^{A3}, —NR^{A3}R^{B3} and —SO₂R^{A3}; and
   wherein said C₁₋₆ cycloalkyl is optionally substituted by one or more substituents independently selected from: halo, ═O, —CN, —NO₂, C₁₋₄ alkyl and C₁₋₄ haloalkyl;
   R^{A1}, R^{A2}, R^{B2}, R^{A3} and R^{B3} are each independently selected from: H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl;
with the provisos (i) and (ii):
(i) when n is 1 or 2; R¹ is halo;
Q¹ is

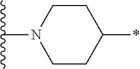 [SMILES: *C1CCN(*)CC1]

L¹ is —O—, —NH— or —N(CH₃)—; and
Q² is phenyl, pyridyl, or pyrimidyl;
   then Q² is substituted by X and optionally one or more R⁶, wherein X is selected from Q³-L³-, —O—C₁₋₆ alkyl and —O—C₁₋₄ alkyl-OR^{A4}, and R^{A4} is H or C₁₋₄ alkyl; and
(ii) when n is 1 or 2; R¹ is halo; L¹ is a bond; and
Q¹ is

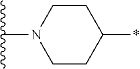 [SMILES: *C1CCN(*)CC1]

then Q² is phenyl or 6-membered heteroaryl, wherein said Q² is substituted by X and optionally one or more R⁶, wherein X is selected from Q³-L³-, —O—C₁₋₆ alkyl and —O—C₁₋₆ alkyl-OR^{A4}, and R^{A4} is H or C₁₋₄ alkyl.

In certain embodiments there is provided a compound of the formula (I), or a pharmaceutically acceptable salt thereof as defined herein, wherein:
R¹ is at each occurrence independently selected from: halo and C₁₋₄ alkyl;
n is 0, 1 or 2;
R² is selected from H and C₁₋₄ alkyl;
Q¹ is selected from:

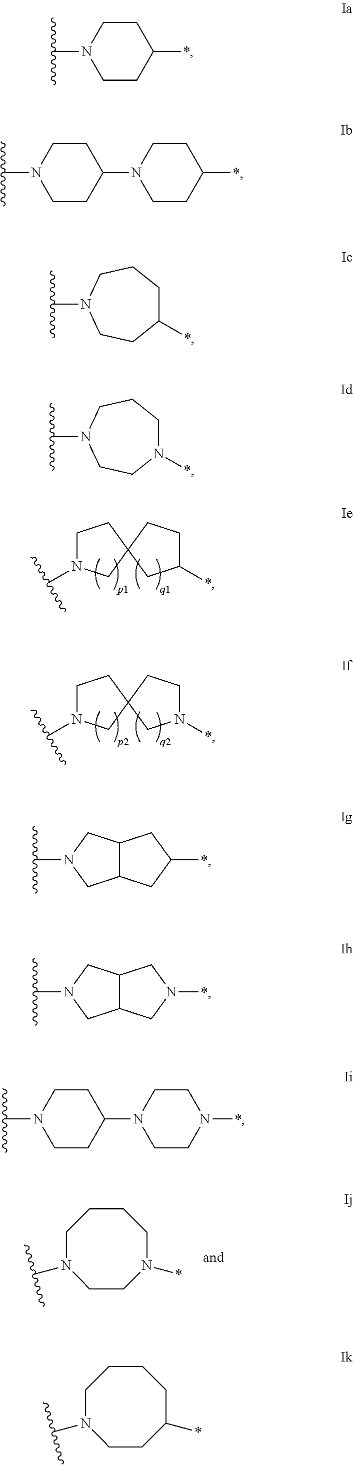

wherein * indicates the point of attachment to L¹,
p¹, q¹, p² and q² are independently an integer selected from 1 or 2,
and wherein Q¹ is optionally substituted by one or more R³;
each R³ is independently selected from: C₁₋₃ alkyl and ═O;
L¹ is a bond or is selected from —CH₂—, —O— and —NR⁴—, provided that L¹ is a bond or —CH₂-when Q¹ is bonded to L¹ via a ring nitrogen in Q¹;
R⁴ is selected from H and C₁₋₃ alkyl;
Q² is selected from phenyl or a 5- or 6-membered heteroaryl, wherein Q² is optionally substituted by one R⁵ and optionally one or more R⁶;
R⁵ is Q³-L³-, wherein
   L³ is a bond or is selected from: C₁₋₄ alkylene, -L⁴-O-L⁵-, -L⁴-NR^{A1}-L⁵-, -L⁴-S(O)ₓ-L⁵-, -L⁴-C(═O)-L⁵, -L⁴-NR^{A1}C(═O)-L⁵-, -L⁴-C(═O)NR^{A1}-L⁵-, -L⁴-S(O)₂NR^{A1}-L⁵-, -L⁴-NR^{A1}S(O)₂-L⁵-, -L⁴-OC(═O)-L⁵ and -L⁴-C(═O)O-L⁵-,
   wherein x is 0, 1 or 2;
   L⁴ and L⁵ are independently selected from a bond and C₁₋₄ alkylene;
   Q³ is selected from: phenyl, a 5- or 6-membered heteroaryl and 3- to 12-membered heterocyclyl, wherein said phenyl or 5- or 6-membered heteroaryl is optionally substituted by one or more R⁷, and said 3- to 12-membered heterocyclyl is optionally substituted by one or more R⁸;
each R⁶, R⁷ and R⁸ is independently selected from:
halo, ═O, —CN, —NO₂, C₁₋₄ alkyl, C₂₋₄ alkenyl, C₂₋₄ alkynyl, C₁₋₄ haloalkyl, C₁₋₆ cycloalkyl, —OR^{A1}, —S(O)ₓR^{A2} (wherein x is 0, 1, or 2), —NR^{A2}R^{B2}, —C(O)R², —OC(O)R^{A2}, —C(O)OR^{A2}, —NR^{B2}C(O)R^{A2}, —NR^{B2}C(O)OR^{A2}, —C(O)NR^{A2}R^{B2}, —NR^{B2}SO₂R^{A2} and —SO₂NR^{A2}R^{B2};
   wherein said C₁₋₄alkyl, C₂₋₄ alkenyl, C₂₋₄ alkynyl are each is optionally substituted by 1 or 2 substituents independently selected from: halo, —CN, —OR^{A3}, —NR^{A3}R^{B3} and —SO₂R^{A3}; and
   wherein said C₁₋₆ cycloalkyl is optionally substituted by one or more substituents independently selected from: halo, ═O, —CN, —NO₂, C₁₋₄ alkyl and C₁₋₄ haloalkyl;
   R^{A1}, R^{A2}, R^{B2}, R^{A3} and R^{B3} are each independently selected from: H, C₁₋₄ alkyl and C₁₋₄ haloalkyl;
with the provisos (i) and (ii):
(i) when n is 1 or 2; R¹ is halo;

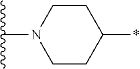 [SMILES: *C1CCN(*)CC1]

Q¹ is Ia
L¹ is —O—, —NH— or —N(CH₃)—; and
Q² is phenyl, pyridyl, or pyrimidyl;
   then Q² is substituted by Q³-L³- and optionally one or more R⁸; and
(ii) when n is 1 or 2; R¹ is halo; L¹ is a bond; and

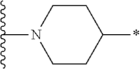 [SMILES: *C1CCN(*)CC1]

Q¹ is Ia;
   then Q² is phenyl or 6-membered heteroaryl, wherein said Q² is substituted by Q³-L³- and optionally one or more R⁶.

In certain embodiments there is provided a pharmaceutical composition comprising a compound of the invention, or a pharmaceutically acceptable salt thereof, and a pharmaceutically acceptable excipient.

In certain embodiments there is provided a compound of the invention, or a pharmaceutically acceptable salt thereof, or a pharmaceutical composition of the invention, for use as a medicament.

In certain embodiments there is provided a compound of the invention, or a pharmaceutically acceptable salt thereof, or a pharmaceutical composition of the invention, for use in the treatment or prevention of a fungal infection in a subject.

In certain embodiments there is provided a method of treating or preventing a fungal infection in a subject, comprising administering to the subject an effective amount of a compound of the invention, or a pharmaceutically acceptable salt thereof, or a pharmaceutical composition of the invention.

In certain embodiments there is provided a compound of the invention, or a pharmaceutically acceptable salt thereof, or a pharmaceutical composition of the invention, for the manufacture of a medicament for the treatment or prevention of a fungal infection in a subject.

### BRIEF DESCRIPTION OF THE DRAWINGS

Embodiments of the invention are further described hereinafter with reference to the accompanying drawings, in which:

FIG. 1 shows the % survival of Galleria mellonella over 120 hours in larvae infected with multi-drug resistant Candida auris strain TDG1912 and treated with either the compound of Example 6 or fluconazole, relative to controls. In FIG. 1, “1912” refers to the C. auris strain TDG1912; “PBS” is phosphate buffered saline used in the control arm; and “fluc” is fluconazole. FIG. 1 (a) shows the group treated with the compound of Example 6 vs. control (PBS). FIG. 1 (b) shows the group treated with fluconazole vs. control (PBS).

FIG. 2 illustrates the computer modelled interaction of a compound of the Example 2 with the lanosterol 14-alpha demethylase enzyme from Candida auris.

### DETAILED DESCRIPTION

### Definitions

Unless otherwise stated, the following terms used in the specification and claims have the following meanings set out below.

The term “a compound of the invention”, or the like refers to a compound of the Formulae (I), (II), (III), (IV), (V), (VI), (VII), (XIII), (X), (XI), (XII), (XII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), (XLII), (XLIII), (XLIV), (XLV), (XLVI), (XLVII), (XVIII), (XLIX), (L), (LI), (LII), (LIII), (LIV), (LV), (LVI), (LVII), (LVIII), (LIX), (LX), (LXI), (LXII), or (LXIII), or a pharmaceutically acceptable salt, solvate, or salt of a solvate thereof, including any of the Examples listed herein.

Reference to a “composition of the invention” refers to a pharmaceutical composition comprising a compound of the invention, or a pharmaceutically acceptable salt thereof, and a pharmaceutically acceptable excipient. For example any of the pharmaceutical compositions described herein.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical or mental well-being; or preventing or slowing the appearance of symptoms of the disease condition (i.e. prophylaxis). For example, certain methods herein describe the treatment of a fungal infection using a compound of the invention. The treatment may, for example reduce, decrease, decrease the progression of, or decrease the side effects of fungal infection. Treatment of a fungal infection includes, for example, reducing or eliminating fungal colonization, infection burden, and/or biofilm formation. It may be that the treatment reduces fungal colonization, infection burden, biofilm and/or biofilm formation by 20%, 30%, 40%, 50% 60%, 70%, 80%, 85%, 90%, 95% or 99%. It may be that the treatment substantially eliminates fungal colonization, infection burden and/or biofilm. The effectiveness of the treatment, can be assessed using well-known methods, for example, by comparing a treated subject with a subject with, or at risk of, a fungal infection who did not receive the treatment. A comparison can also be made between the treated subject and a control, a baseline, or a known level or measurement.

As used herein, the terms “prevent” or “preventing” mean decreasing the risk of contracting a fungal infection. To determine whether the prevention is effective, a comparison can be made between a subject who received a compound of the invention with a subject at risk of a fungal infection who did not receive the compound of the invention. A comparison can also be made between the subject who received the composition and a control, a baseline, or a known level or measurement.

The term “associated” or “associated with” in the context of a substance or substance activity or function associated with a disease of condition means that the disease or condition is caused by (in whole or in part), or a symptom of the disease or condition is caused by (in whole or in part) the substance (e.g. a fungus) or substance activity or function.

The terms “subject” or “patient” refer to a human or non-human animal to whom a compound of the invention is administered. In some embodiments the subject may be a human. In some embodiments, the subject may be a mammal. In some embodiments, the subject may be a non-mammalian animal. In some embodiments, the subject is a domesticated mammal, for example, a canine, equine, feline, porcine, bovine, murine, caprine or ovine. In preferred embodiments, the subject is a human. In some embodiments the subject is a human that is at least 50, 60, 70, 80, or 90 years old. In some embodiments the subject is a human child, for example a child that is Iess than 3 years old, less than 12 months old or less than 6 months old.

The term “halo” or “halogen” refers to one of the halogens, group 17 of the periodic table. In particular, the term refers to fluorine, chlorine, bromine and iodine. Preferably, the term refers to fluorine, chlorine or bromine.

The term Cₘ₋ₙ refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl, iso-pentyl, n-hexyl, or iso-hexyl. As will be realised, the term “C₁₋₄ alkyl” refers to an alkyl group containing 1, 2, 3 or 4 carbon atoms, for example methyl, ethyl, n-propyl, iso-propyl, n-butyl, sec-butyl or tert-butyl. Alkylene groups are divalent alkyl groups and may likewise be linear or branched and have two points of attachment to the remainder of the molecule. Furthermore, an alkylene group may, for example, correspond to one of those alkyl groups listed in this paragraph. The alkyl and alkylene groups may be unsubstituted or substituted by one or more substituents. Possible substituents are described below. Substituents for the alkyl group may be halogen, e.g. fluorine, chlorine, bromine and iodine, OH, C₁-C₄ alkoxy. Other substituents for the alkyl group may alternatively be used.

The term “C₁₋₆ haloalkyl”, refers to a hydrocarbon chain substituted with at least one halogen atom independently chosen at each occurrence, for example fluorine, chlorine, bromine and iodine. As will be realised, the term “C₁₋₄ haloalkyl”, refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₄ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoroethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be a fluoroalkyl group, i.e. a hydrocarbon chain substituted with at least one fluorine atom.

The term “C₂₋₆ alkenyl” includes a branched or linear hydrocarbon chain containing at least one double bond and having 2, 3, 4, 5 or 6 carbon atoms. As will be realised, the term “C₂₋₄ alkenyl” includes a branched or linear hydrocarbon chain containing at least one double bond and having 2, 3 or 4 carbon atoms. The double bond(s) may be present as the E or Z isomer. The double bond may be at any possible position of the hydrocarbon chain. For example, the “C₂₋₄ alkenyl” may be ethenyl, propenyl, butenyl, butadienyl, pentenyl, pentadienyl, hexenyl and hexadienyl.

The term “C₂₋₆ alkynyl” includes a branched or linear hydrocarbon chain containing at least one triple bond and having 2, 3, 4, 5 or 6 carbon atoms. As will be realised, the term “C₂₋₄ alkynyl” includes a branched or linear hydrocarbon chain containing at least one triple bond and having 2, 3 or 4 carbon atoms. The triple bond may be at any possible position of the hydrocarbon chain. For example, the “C₂₋₄ alkynyl” may be ethynyl, propynyl or butynyl.

The term “C₃₋₆ cycloalkyl” includes a saturated hydrocarbon ring system containing 3, 4, 5 or 6 carbon atoms. For example, the “C₃-C₆ cycloalkyl” may be cyclopropyl, cyclobutyl, cyclopentyl, cyclohexyl, bicyclo[2.1.1]hexane or bicyclo[1.1.1]pentane.

“—Cₘ₋ₙ alkyl substituted by —NRR” and “Cₘ₋ₙ alkyl substituted by —OR” similarly refer to an —NRR or —OR group covalently attached to a Cₘ₋ₙ alkylene group and wherein the group is linked to the remainder of the molecule via a carbon atom in the alkylene group.

The term “heterocyclyl”, “heterocyclic” or “heterocycle” includes a non-aromatic saturated or partially saturated monocyclic or fused, bridged, or spiro bicyclic heterocyclic ring system. Monocyclic heterocyclic rings may contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles may contain from 7 to 12-member atoms in the ring. Bicyclic heterocyclic(s) rings may be fused, spiro, or bridged ring systems. The heterocyclyl group may be a 3-12, for example, a 3- to 9- (e.g. a 3- to 7-) membered non-aromatic monocyclic or bicyclic saturated or partially saturated group comprising 1, 2 or 3 heteroatoms independently selected from O, S and N in the ring system (in other words 1, 2 or 3 of the atoms forming the ring system are selected from O, S and N). By partially saturated it is meant that the ring may comprise one or two double bonds. This applies particularly to monocyclic rings with from 5 to 7 members. The double bond will typically be between two carbon atoms but may be between a carbon atom and a nitrogen atom. Bicyclic systems may be spiro-fused, i.e. where the rings are linked to each other through a single carbon atom; vicinally fused, i.e. where the rings are linked to each other through two adjacent carbon or nitrogen atoms; or they may be share a bridgehead, i.e. the rings are linked to each other through two non-adjacent carbon or nitrogen atoms (a bridged ring system). Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 2,5-diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a ring carbon or ring nitrogen atom. For example, the term “piperidino” or “morpholino” refers to a piperidin-1-yl or morpholin-4-yl ring that is linked via the ring nitrogen

The term “aromatic” when applied to a substituent as a whole includes a single ring or polycyclic ring system with 4n+2 electrons in a conjugated π system within the ring or ring system where all atoms contributing to the conjugated π system are in the same plane.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated π system where all atoms contributing to the conjugated π system are in the same plane.

The heteroaryl group can be, for example, a 5- or 6-membered monocyclic ring. The ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not.

Ortho, meta and para substitution are well understood terms in the art. For the absence of doubt, “ortho” substitution is a substitution pattern where adjacent carbons possess a substituent, whether a simple group, for example the fluoro group in the example below, or other portions of the molecule, as indicated by the bond ending in “ [symbol] ”.

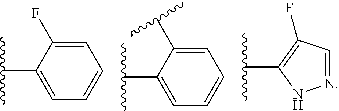

“Meta” substitution is a substitution pattern where two substituents are on carbons one carbon removed from each other, i.e. with a single carbon atom between the substituted carbons. In other words, there is a substituent on the second atom away from the atom with another substituent. For example, the groups below are meta substituted.

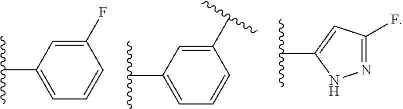

“Para” substitution is a substitution pattern where two substituents are on carbons two carbons removed from each other, i.e. with two carbon atoms between the substituted carbons. In other words, there is a substituent on the third atom away from the atom with another substituent. For example, the groups below are para substituted.

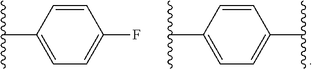

A bond terminating in a “ [symbol] ” or “*” represents that the bond is connected to another atom that is not shown in the structure. A bond terminating inside a cyclic structure and not terminating at an atom of the ring structure represents that the bond may be connected to any of the atoms in the ring structure where allowed by valency.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

The reader's attention is directed to all papers and documents which are filed concurrently with or previous to this specification in connection with this application and which are open to public inspection with this specification, and the contents of all such papers and documents are incorporated herein by reference.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or less than 550. More preferably, the molecular weight is Iess than 525 and, for example, is 500 or less.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%.

The compounds of this invention may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures of compounds of the invention.

Z/E (e.g. cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high pressure liquid chromatography (HPLC) or chiral supercritical fluid chromatography (SFC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Alternatively, when chiral SFC is employed a supercritical fluid, generally CO₂, is used as the mobile phase. The properties of the supercritical fluid may be modified by the inclusion of one or more co-solvents, e.g. an alcohol such as methanol, ethanol or isopropanol, acetonitrile or ethylacetate. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. An enantiomer of a compound may also be prepared using a chiral auxiliary during the synthesis of the compound, in which a suitable chiral intermediate is reacted with an intermediate of the compound followed by one or more diastereoselective transformations. The resulting diastereomers are then separated using conventional methods, such as those described above, followed by removal of the chiral auxiliary to provide the desired enantiomer.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), ³H (also written as “T” for tritium), ¹¹C, ¹³C, ¹⁴C, ¹⁵O, ¹⁷O, ¹⁸O, ¹³N, ¹⁵N, ¹⁸F, ³⁶Cl, ¹²³I, ²⁵I, ³²P, ³⁵S and the like. The radionuclide that is used will depend on the specific application of that radio-labelled derivative. For example, for in vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in-vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. For example, one or more hydrogen atoms in a C₁₋₄-alkyl group may be replaced by deuterium to form a deuterated C₁₋₄-alkyl group, for example CD₃.

Certain compounds of the invention may exist in solvated as well as unsolvated forms such as, for example, hydrated forms. It is to be understood that the invention encompasses all such solvated forms or pharmaceutically acceptable salts thereof.

It is also to be understood that certain compounds of the invention may exhibit polymorphism, and that the invention encompasses all such polymorphic forms of the compounds of the invention.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

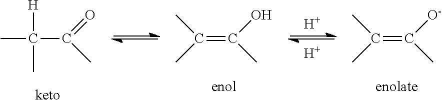

The in vivo effects of a compound of the invention may be exerted in part by one or more metabolites that are formed within the human or animal body after administration of a compound of the invention.

### Compounds

The following paragraphs are applicable to the compounds of the invention.

In some embodiments the compound of formula (I) is of the formula (II), or a pharmaceutically acceptable salt thereof:

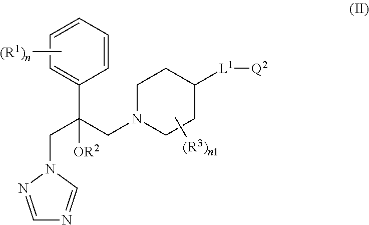

wherein:
   n1 is 0, 1 or 2.

In some embodiments the compound of formula (I) is of the formula (III), or a pharmaceutically acceptable salt thereof:

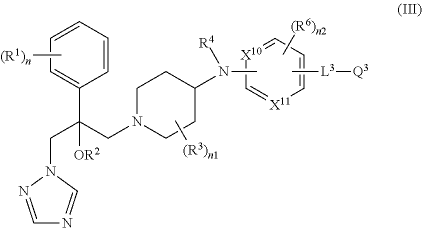

wherein:
   X¹⁰ and X¹¹ are independently selected from N and CH;
   n1 is 0, 1 or 2; and
   n2 is 0, 1 or 2.

In some embodiments the compound of formula (I) is of the formula (IV), or a pharmaceutically acceptable salt thereof:

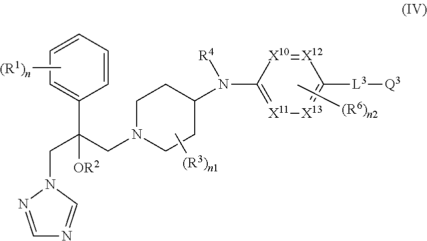

wherein:
   X¹⁰, X¹¹, X¹² and X¹³, are independently selected from CH and N, provided no more than two of X¹⁰, X¹, X¹² and X¹³ are N;
   n1 is 0, 1 or 2; and
   n2 is 0, 1 or 2.

In some embodiments in the compound of formula (IV), X¹⁰, X¹¹, X¹² and X¹³, are independently selected from CH and N, provided no more than one of X¹⁰, X¹¹, X¹² and X¹³ is N; n1 is 0, 1 or 2; and n2 is 0, 1 or 2.

In some embodiments in the compound of formula (IV), one of X¹⁰, X¹¹, X¹² and X¹³ is N and the remaining X¹⁰, X¹¹, X¹² and X¹³ are CH; n1 is 0, 1 or 2; and n2 is 0, 1 or 2.

In some embodiments the compound of formula (I) is of the formula (V), or a pharmaceutically acceptable salt thereof:

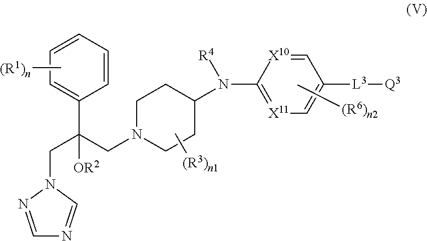

wherein:
   X¹⁰ and X¹¹ are independently selected from N and CH;
   n1 is 0, 1 or 2; and
   n2 is 0, 1 or 2.

In some embodiments the compound of formula (I) is of the formula (VI), or a pharmaceutically acceptable salt thereof:

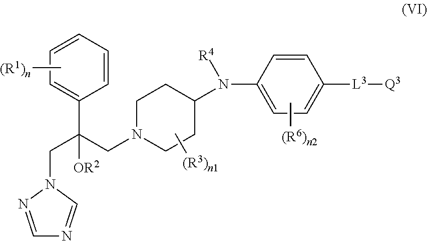

wherein:
   n1 is 0, 1 or 2; and
   n2 is 0, 1 or 2.

In some embodiments the compound of formula (I) is of the formula (VII), or a pharmaceutically acceptable salt thereof:

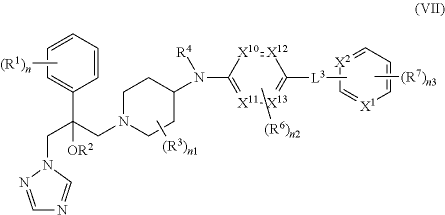

wherein:
   X¹ and X² are independently selected from CH and N;
   X¹⁰, X¹¹, X¹² and X¹³, are independently selected from CH and N, provided no more than two of X¹⁰, X¹¹, X¹² and X¹³ are N;
   n1 is 0, 1 or 2;
   n2 is 0, 1 or 2; and
   n3 is 0, 1 or 2.

In some embodiments the compound of formula (I) is of the formula (VIII), or a pharmaceutically acceptable salt thereof:

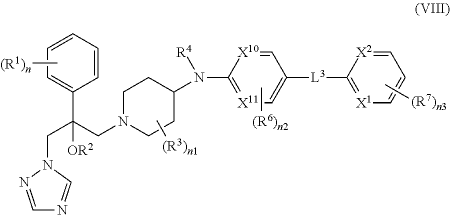

wherein:
   X¹ and X² are independently selected from CH and N;
   X¹⁰ and X¹¹ are independently selected from N and CH;
   n1 is 0, 1 or 2;
   n2 is 0, 1 or 2; and
   n3 is 0, 1 or 2.

In some embodiments the compound of formula (I) is of the formula (IX), or a pharmaceutically acceptable salt thereof:

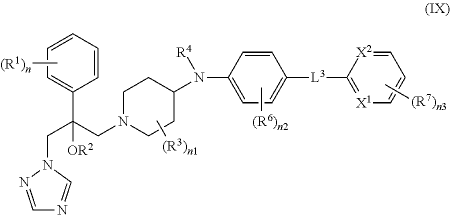

wherein:
   X¹ and X² are independently selected from CH and N;
   n1 is 0, 1 or 2;
   n2 is 0, 1 or 2; and
   n3 is 0, 1 or 2.

In some embodiments the compound of formula (I) is of the formula (X), or a pharmaceutically acceptable salt thereof:

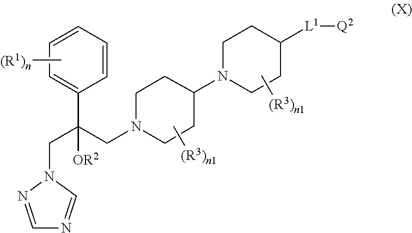

wherein each n1 is independently selected from 0, 1 or 2.

In some embodiments the compound of formula (I) is of the formula (XI), or a pharmaceutically acceptable salt thereof:

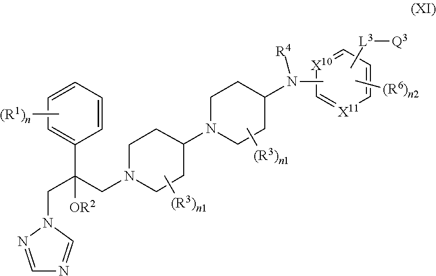

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XII), or a pharmaceutically acceptable salt thereof:

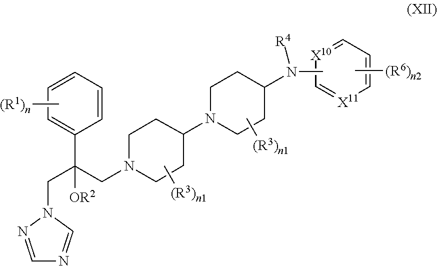

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XIII), or a pharmaceutically acceptable salt thereof:

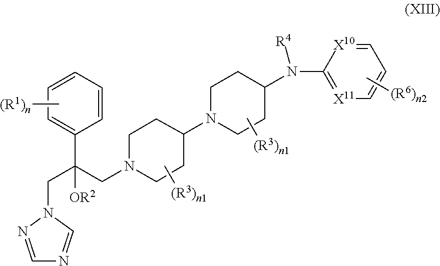

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XIV), or a pharmaceutically acceptable salt thereof:

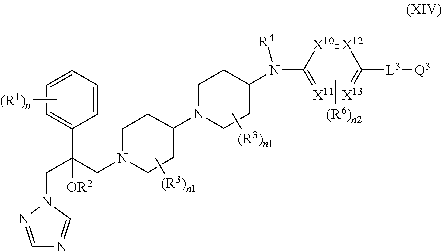

wherein:
   X¹⁰, X¹¹, X¹² and X¹³ are each independently selected from CH and N, provided no more than two of X, X¹¹, X¹² and X¹³ are N;
   each n1 is independently selected from 0, 1 or 2; and
   n2 is 0, 1 or 2.

In some embodiments in the compound of formula (XIV), X¹⁰, X¹¹, X¹² and X¹³ are each independently selected from CH and N, provided no more than one of X¹⁰, X¹¹, X¹² and X¹³ is N.

In some embodiments in the compound of formula (XIV), one of X¹⁰, X¹¹, X¹² and X¹³ is N and the remaining X¹⁰, X¹¹, X¹² and X¹³ are CH.

In some embodiments the compound of formula (I) is of the formula (XV), or a pharmaceutically acceptable salt thereof:

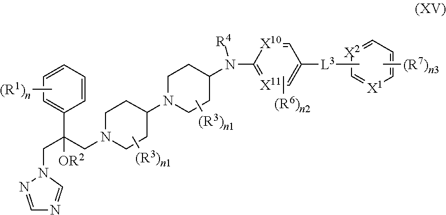

wherein:
   X¹, X², X¹⁰ and X¹¹ are each independently selected from CH and N;
   each n1 is independently selected from 0, 1 or 2;
   n2 is 0, 1 or 2; and
   n3 is 0, 1 or 2.

In some embodiments the compound of formula (I) is of the formula (XVI), or a pharmaceutically acceptable salt thereof:

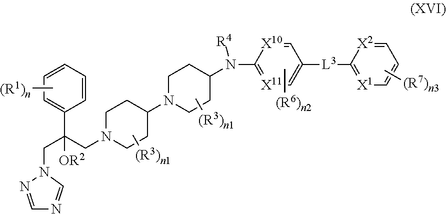

wherein:
   X¹, X², X¹⁰ and X¹¹ are each independently selected from CH and N;
   each n1 is independently selected from 0, 1 or 2;
   n2 is 0, 1 or 2; and
   n3 is 0, 1 or 2.

In some embodiments the compound of formula (I) is of the formula (XVII), or a pharmaceutically acceptable salt thereof:

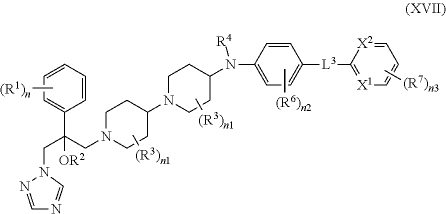

wherein:
   X¹ and X² are each independently selected from CH and N;
   each n1 is independently selected from 0, 1 or 2;
   n2 is 0, 1 or 2; and
   n3 is 0, 1 or 2.

In some embodiments the compound of formula (I) is of the formula (XVIII), or a pharmaceutically acceptable salt thereof:

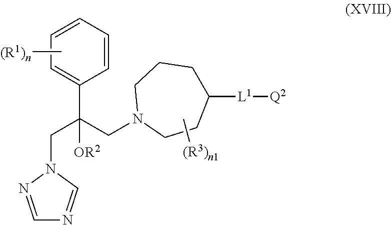

wherein n1 is 0, 1 or 2.

In some embodiments the compound of formula (I) is of the formula (XIX), or a pharmaceutically acceptable salt thereof:

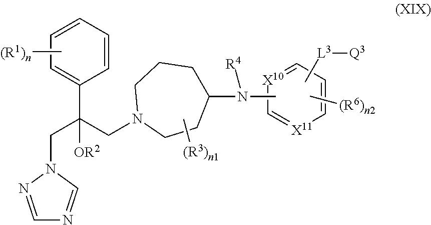

wherein:
   X¹⁰ and X¹¹ are independently selected from CH and N;
   n1 is 0, 1 or 2; and
   n2 is 0, 1 or 2.

In some embodiments the compound of formula (I) is of the formula (XX), or a pharmaceutically acceptable salt thereof:

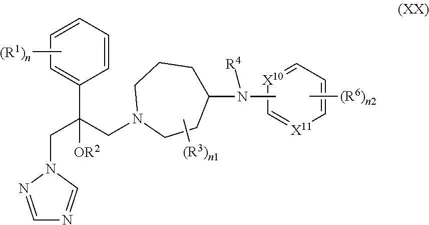

wherein:
   X¹⁰ and X¹¹ are independently selected from CH and N;
   n1 is 0, 1 or 2; and
   n2 is 0, 1 or 2.

In some embodiments the compound of formula (I) is of the formula (XXI), or a pharmaceutically acceptable salt thereof:

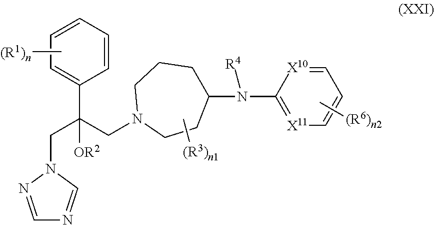

wherein:
   X¹⁰ and X¹¹ are independently selected from CH and N;
   n1 is 0, 1 or 2; and
   n2 is 0, 1 or 2.

In some embodiments the compound of formula (I) is of the formula (XXII), or a pharmaceutically acceptable salt thereof:

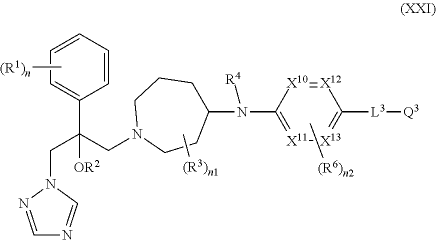

wherein:
   X¹⁰, X¹¹, X¹² and X¹³ are each independently selected from CH and N, provided no more than two of X¹⁰, X¹¹, X¹² and X¹³ are N;
   n1 is 0, 1 or 2; and
   n2 is 0, 1 or 2.

In some embodiments in the compound of formula (XXII), X¹⁰, X¹¹, X¹² and X¹³ are each independently selected from CH and N, provided no more than one of X¹⁰, X¹¹, X¹² and X¹³ is N.

In some embodiments in the compound of formula (XXII), one of X¹⁰, X¹¹, X¹² and X¹³ is N and the remaining X¹⁰, X¹¹, X¹² and X¹³ are CH.

In some embodiments the compound of formula (I) is of the formula (XXIII), or a pharmaceutically acceptable salt thereof:

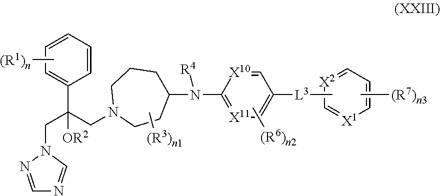

wherein:
   X¹, X², X¹⁰ and X¹¹ are each independently selected from CH and N;
   n1 is 0, 1 or 2;
   n2 is 0, 1 or 2; and
   n3 is 0, 1 or 2.

In some embodiments the compound of formula (I) is of the formula (XXIV), or a pharmaceutically acceptable salt thereof:

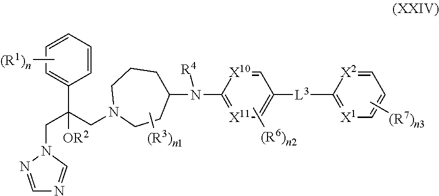

wherein:
   X¹, X², X¹⁰ and X¹¹ are each independently selected from CH and N;
   n1 is 0, 1 or 2;
   n2 is 0, 1 or 2; and
   n3 is 0, 1 or 2.

In some embodiments the compound of formula (I) is of the formula (XXV), or a pharmaceutically acceptable salt thereof:

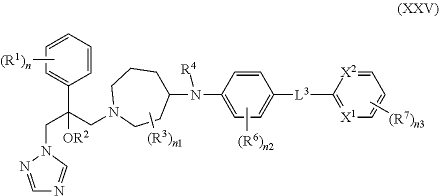

wherein:
   X¹ and X² are each independently selected from CH and N;
   n1 is 0, 1 or 2;
   n2 is 0, 1 or 2; and
   n3 is 0, 1 or 2.

In some embodiments the compound of formula (I) is of the formula (XXVI), or a pharmaceutically acceptable salt thereof:

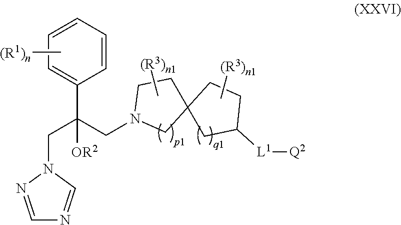

wherein each n1 is independently selected from 0, 1 or 2.

In some embodiments the compound of formula (I) is of the formula (XXVII), or a pharmaceutically acceptable salt thereof:

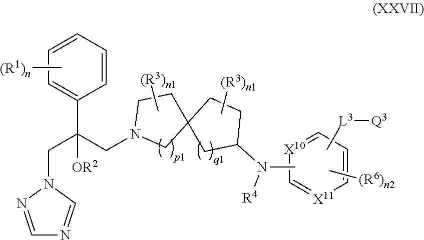

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XXVIII), or a pharmaceutically acceptable salt thereof:

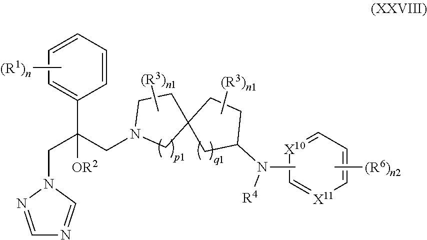

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XXIX), or a pharmaceutically acceptable salt thereof:

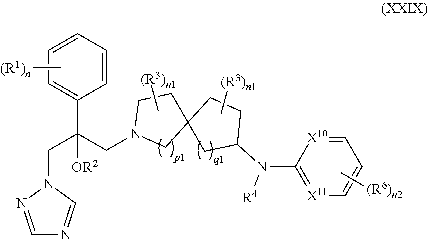

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XXX), or a pharmaceutically acceptable salt thereof:

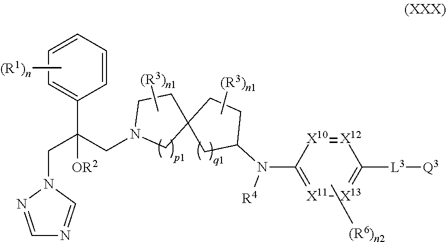

wherein:
   X¹⁰, X¹¹, X¹² and X¹³ are each independently selected from CH and N, provided no more than two of X¹⁰, X¹¹, X¹² and X¹³ are N;
   each n1 is independently selected from 0, 1 or 2; and
   n2 is 0, 1 or 2.

In some embodiments in the compound of formula (XXX), X¹⁰, X¹¹, X¹² and X¹³ are each independently selected from CH and N, provided no more than one of X¹⁰, X¹¹, X¹² and X¹³ is N.

In some embodiments in the compound of formula (XXX), one of X¹⁰, X¹¹, X¹² and X¹³ is N and the remaining X¹⁰, X¹¹, X¹² and X¹³ are CH.

In some embodiments the compound of formula (I) is of the formula (XXXI), or a pharmaceutically acceptable salt thereof:

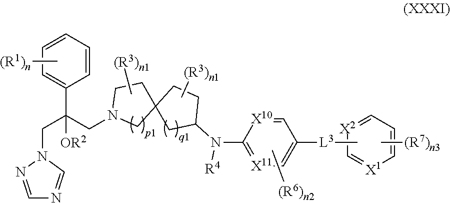

wherein:
   X¹, X², X¹⁰ and X¹¹ are each independently selected from CH and N;
   each n1 is independently selected from 0, 1 or 2;
   n2 is 0, 1 or 2; and
   n3 is 0, 1 or 2.

In some embodiments the compound of formula (I) is of the formula (XXXII), or a pharmaceutically acceptable salt thereof:

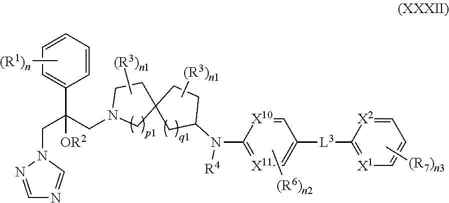

wherein:
   X¹, X², X¹⁰ and X¹¹ are each independently selected from CH and N;
   each n1 is independently selected from 0, 1 or 2;
   n2 is 0, 1 or 2; and
   n3 is 0, 1 or 2.

In some embodiments the compound of formula (I) is of the formula (XXXIII), or a pharmaceutically acceptable salt thereof:

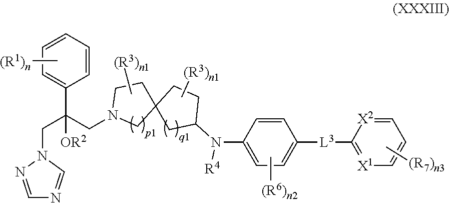

wherein:
   X¹ and X² are each independently selected from CH and N;
   each n1 is independently selected from 0, 1 or 2;
   n2 is 0, 1 or 2; and
   n3 is 0, 1 or 2.

In some embodiments the compound of formula (I) is of the formula (XXXIV), or a pharmaceutically acceptable salt thereof:

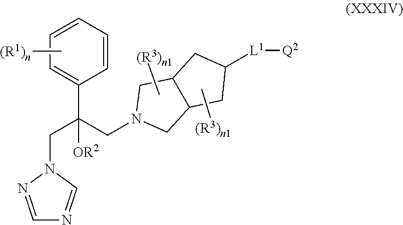

wherein each n1 is independently selected from 0, 1 or 2.

In some embodiments the compound of formula (I) is of the formula (XXXV), or a pharmaceutically acceptable salt thereof:

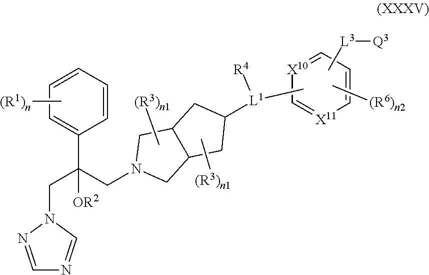

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XXXVI), or a pharmaceutically acceptable salt thereof:

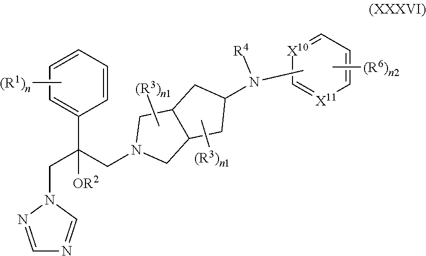

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XXXVII), or a pharmaceutically acceptable salt thereof:

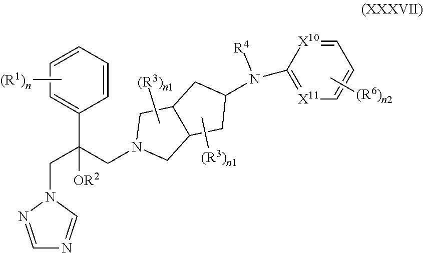

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XXXVIII), or a

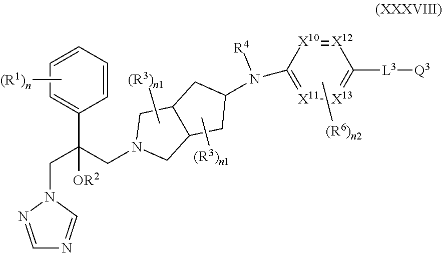

wherein:
   X¹⁰, X¹¹, X¹² and X¹³ are each independently selected from CH and N, provided no more than two of X¹⁰, X¹¹, X¹² and X¹³ are N;
   each n1 is independently selected from 0, 1 or 2; and
   n2 is 0, 1 or 2.

In some embodiments in the compound of formula (XXXVIII), X¹⁰, X¹¹, X¹² and X¹³ are each independently selected from CH and N, provided no more than one of X¹⁰, X¹¹, X¹² and X¹³ is N.

In some embodiments in the compound of formula (XXXVIII), one of X¹⁰, X¹¹, X¹² and X¹³ is N and the remaining X¹⁰, X¹¹, X¹² and X¹³ are CH.

In some embodiments the compound of formula (I) is of the formula (XXXIX), or a pharmaceutically acceptable salt thereof:

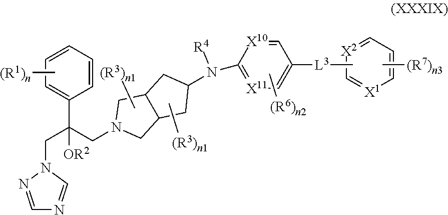

wherein:
   X¹, X², X¹⁰ and X¹¹ are each independently selected from CH and N;
   each n1 is independently selected from 0, 1 or 2;
   n2 is 0, 1 or 2; and
   n3 is 0, 1 or 2.

In some embodiments the compound of formula (I) is of the formula (XL), or a pharmaceutically acceptable salt thereof:

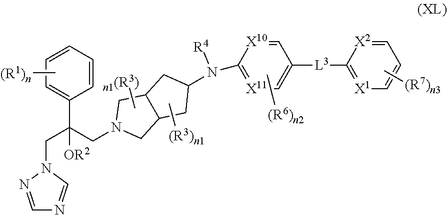

wherein:
   X¹, X², X¹⁰ and X¹¹ are each independently selected from CH and N;
   each n1 is independently selected from 0, 1 or 2;
   n2 is 0, 1 or 2; and
   n3 is 0, 1 or 2.

In some embodiments the compound of formula (I) is of the formula (XLI), or a pharmaceutically acceptable salt thereof:

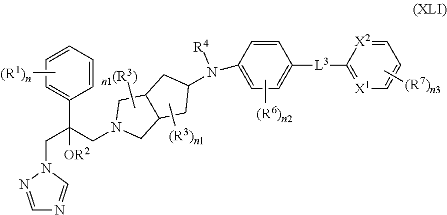

wherein:
   X¹ and X² are each independently selected from CH and N;
   each n1 is independently selected from 0, 1 or 2;
   n2 is 0, 1 or 2; and
   n3 is 0, 1 or 2.

In some embodiments the compound of formula (I) is of the formula (XLII), or a pharmaceutically acceptable salt thereof:

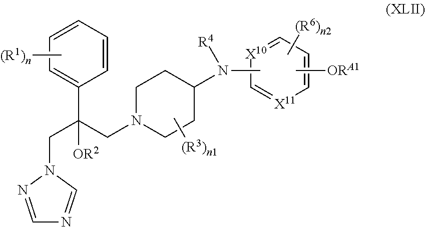

wherein:
   X¹⁰ and X¹¹ are independently selected from N and CH;
   n1 is 0, 1 or 2; and
   n2 is 0, 1 or 2.

In some embodiments the compound of formula (I) is of the formula (XLIII), or a pharmaceutically acceptable salt thereof:

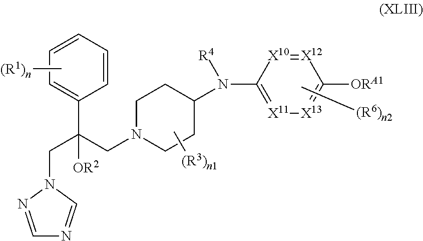

wherein:
   X¹⁰, X¹¹, X¹² and X¹³, are independently selected from CH and N, provided no more than two of X¹⁰, X¹¹, X¹² and X¹³ are N;
   n1 is 0, 1 or 2; and
   n2 is 0, 1 or 2.

In some embodiments in the compound of formula (XLIII), X¹⁰, X¹¹, X¹² and X¹³, are independently selected from CH and N, provided no more than one of X¹⁰, X¹¹, X¹² and X¹³ is N; n1 is 0, 1 or 2; and n2 is 0, 1 or 2.

In some embodiments in the compound of formula (XLIII), one of X¹⁰, X¹¹, X¹² and X¹³ is N and the remaining X¹⁰, X¹¹, X¹² and X¹³ are CH; n1 is 0, 1 or 2; and n2 is 0, 1 or 2.

In some embodiments the compound of formula (I) is of the formula (XLIV), or a pharmaceutically acceptable salt thereof:

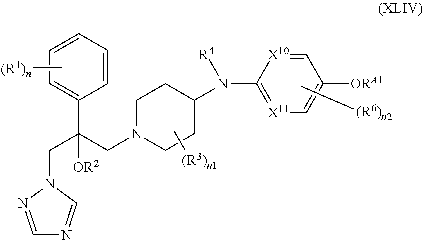

wherein:
   X¹⁰ and X¹¹ are independently selected from N and CH;
   n1 is 0, 1 or 2; and
   n2 is 0, 1 or 2.

In some embodiments the compound of formula (I) is of the formula (XLV), or a pharmaceutically acceptable salt thereof:

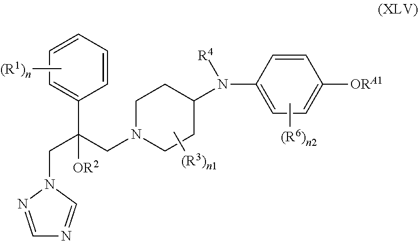

wherein:
   n1 is 0, 1 or 2; and
   n2 is 0, 1 or 2.

In some embodiments the compound of formula (I) is of the formula (XLVI), or a pharmaceutically acceptable salt thereof:

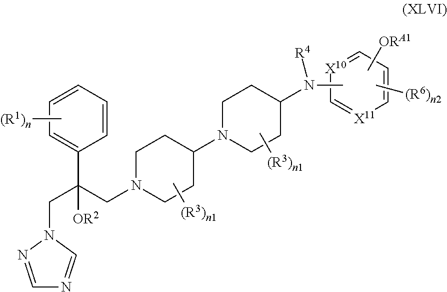

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (XLVII), or a pharmaceutically acceptable salt thereof:

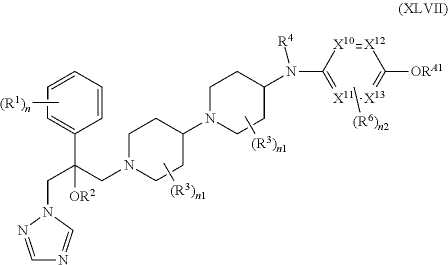

wherein:
   X¹⁰, X¹¹, X¹² and X¹³ are each independently selected from CH and N, provided no more than two of X¹⁰, X¹¹, X¹² and X¹³ are N;
   each n1 is independently selected from 0, 1 or 2; and
   n2 is 0, 1 or 2.

In some embodiments in the compound of formula (XLVII), X¹⁰, X¹¹, X¹² and X¹³ are each independently selected from CH and N, provided no more than one of X¹⁰, X¹¹, X¹² and X¹³ is N.

In some embodiments in the compound of formula (XLVII), one of X¹⁰, X¹¹, X¹² and X¹³ is N and the remaining X¹⁰, X¹¹, X¹² and X¹³ are CH.

In some embodiments the compound of formula (I) is of the formula (XLVIII), or a pharmaceutically acceptable salt thereof:

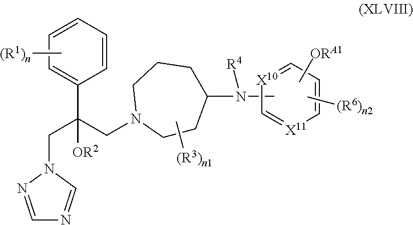

wherein:
   X¹⁰ and X¹¹ are independently selected from CH and N;
   n1 is 0, 1 or 2; and
   n2 is 0, 1 or 2.

In some embodiments the compound of formula (I) is of the formula (XLIX), or a pharmaceutically acceptable salt thereof:

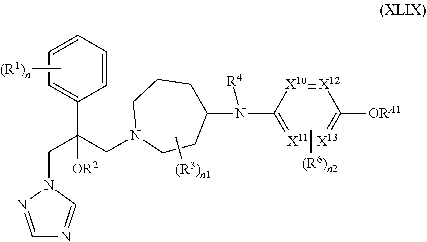

wherein:
   X¹⁰, X¹¹, X¹² and X¹³ are each independently selected from CH and N, provided no more than two of X¹⁰, X¹¹, X¹² and X¹³ are N;
   n1 is 0, 1 or 2; and
   n2 is 0, 1 or 2.

In some embodiments in the compound of formula (XLIX), X¹⁰, X¹¹, X¹² and X¹³ are each independently selected from CH and N, provided no more than one of X¹⁰, X¹¹, X¹² and X¹³ is N.

In some embodiments in the compound of formula (XLIX), one of X¹⁰, X¹¹, X¹² and X¹³ is N and the remaining X¹⁰, X¹¹, X¹² and X¹³ are CH.

In some embodiments the compound of formula (I) is of the formula (L), or a pharmaceutically acceptable salt thereof:

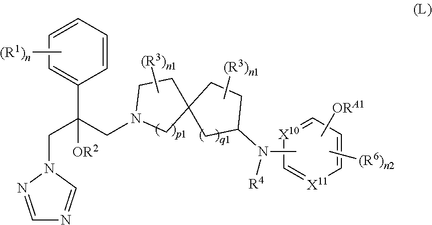

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (LI), or a pharmaceutically acceptable salt thereof:

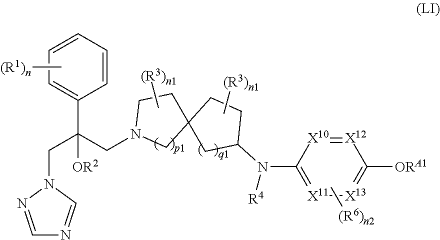

wherein:
   X¹⁰, X¹¹, X¹² and X¹³ are each independently selected from CH and N, provided no more than two of X¹⁰, X¹¹, X¹² and X¹³ are N;
   each n1 is independently selected from 0, 1 or 2; and
   n2 is 0, 1 or 2.

In some embodiments in the compound of formula (LI), X¹⁰, X¹¹, X¹² and X¹³ are each independently selected from CH and N, provided no more than one of X¹⁰, X¹¹, X¹² and X¹³ is N.

In some embodiments in the compound of formula (LI), one of X¹⁰, X¹¹, X¹² and X¹³ is N and the remaining X¹⁰, X¹¹, X¹² and X¹³ are CH.

In some embodiments the compound of formula (I) is of the formula (LII), or a pharmaceutically acceptable salt thereof:

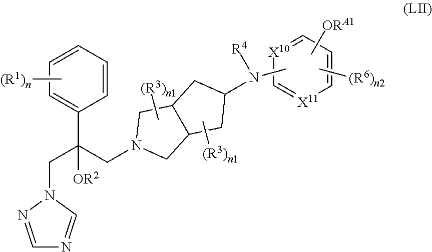

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (LIII), or a pharmaceutically acceptable salt thereof:

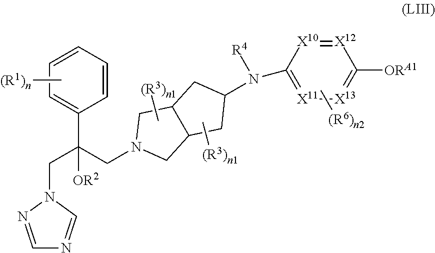

wherein:
   X¹⁰, X¹¹, X¹² and X¹³ are each independently selected from CH and N, provided no more than two of X¹⁰, X¹¹, X¹² and X¹³ are N;
   each n1 is independently selected from 0, 1 or 2; and
   n2 is 0, 1 or 2.

In some embodiments in the compound of formula (LIII), X¹⁰, X¹¹, X¹² and X¹³ are each independently selected from CH and N, provided no more than one of X¹⁰, X¹¹, X¹² and X¹³ is N.

In some embodiments in the compound of formula (LIII), one of X¹⁰, X¹¹, X¹² and X¹³ is N and the remaining X¹⁰, X¹¹, X¹² and X¹³ are CH.

In some embodiments the compound of formula (I) is of the formula (LIV), or a pharmaceutically acceptable salt thereof:

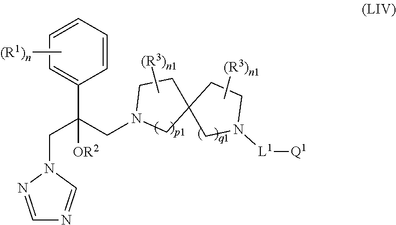

wherein each n1 is independently selected from 0, 1 or 2.

In some embodiments the compound of formula (I) is of the formula (LV), or a pharmaceutically acceptable salt thereof:

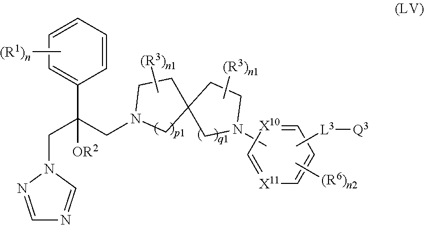

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (LVI), or a pharmaceutically acceptable salt thereof:

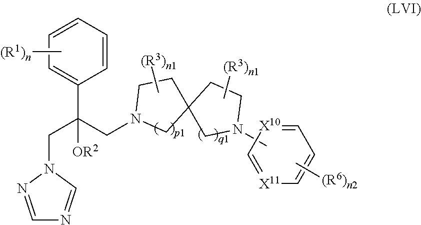

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (LVII), or a pharmaceutically acceptable salt thereof:

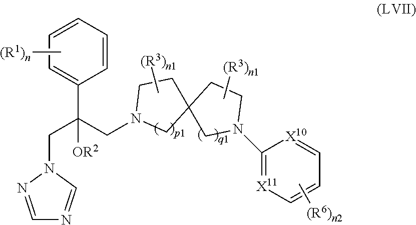

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (LVIII), or a pharmaceutically acceptable salt thereof:

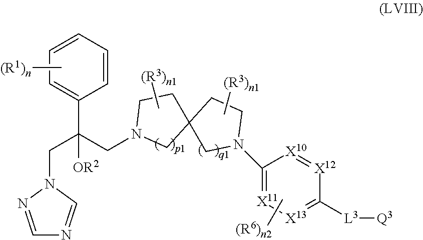

wherein:
   X¹⁰, X¹¹, X¹² and X¹³ are each independently selected from CH and N, provided no more than two of X¹⁰, X¹¹, X¹² and X¹³ are N;
   each n1 is independently selected from 0, 1 or 2; and
   n2 is 0, 1 or 2.

In some embodiments in the compound of formula (LIX), X¹⁰, X¹¹, X¹² and X¹³ are each independently selected from CH and N, provided no more than one of X¹⁰, X¹¹, X¹² and X¹³ is N.

In some embodiments in the compound of formula (LIX), one of X¹⁰, X¹¹, X¹² and X¹³ is N and the remaining X¹⁰, X¹¹, X¹² and X¹³ are CH.

In some embodiments the compound of formula (I) is of the formula (LIX), or a pharmaceutically acceptable salt thereof:

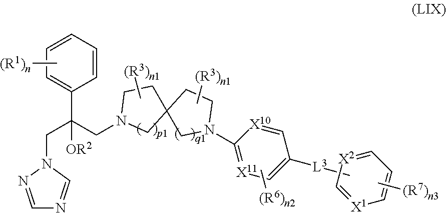

wherein:
   X¹, X², X¹⁰ and X¹¹ are each independently selected from CH and N;
   each n1 is independently selected from 0, 1 or 2;
   n2 is 0, 1 or 2; and
   n3 is 0, 1 or 2.

In some embodiments the compound of formula (I) is of the formula (LX), or a pharmaceutically acceptable salt thereof:

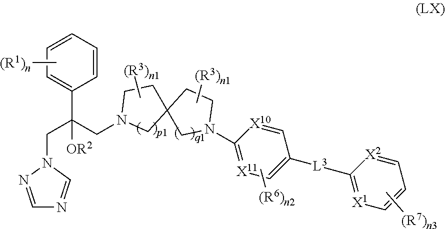

wherein:
   X¹, X², X¹⁰ and X¹¹ are each independently selected from CH and N;
   each n1 is independently selected from 0, 1 or 2;
   n2 is 0, 1 or 2; and
   n3 is 0, 1 or 2.

In some embodiments the compound of formula (I) is of the formula (LXI), or a pharmaceutically acceptable salt thereof:

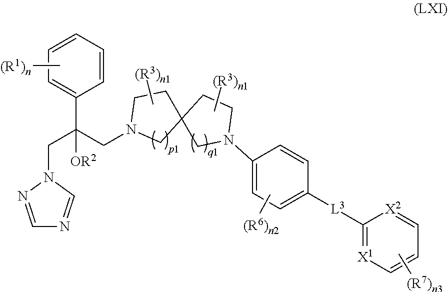

wherein:
   X¹ and X² are each independently selected from CH and N;
   each n1 is independently selected from 0, 1 or 2;
   n2 is 0, 1 or 2; and
   n3 is 0, 1 or 2.

In some embodiments the compound of formula (I) is of the formula (LXII), or a pharmaceutically acceptable salt thereof:

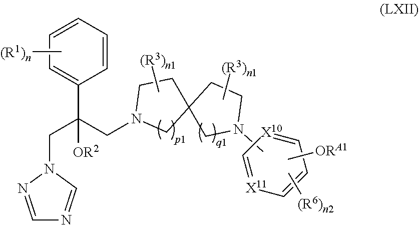

wherein:
   X¹⁰ and X¹¹ are independently selected from CH and N;
   each n1 is independently selected from 0, 1 or 2; and
   n2 is 0, 1 or 2.

In some embodiments the compound of formula (I) is of the formula (LXIII), or a pharmaceutically acceptable salt thereof:

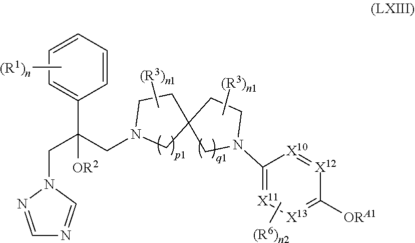

wherein:
   X¹⁰, X¹¹, X¹² and X¹³ are each independently selected from CH and N, provided no more than two of X¹⁰, X¹¹, X¹² and X¹³ are N;
   each n1 is independently selected from 0, 1 or 2; and
   n2 is 0, 1 or 2.

In some embodiments in the compound of formula (LXIII), X¹⁰, X¹¹, X¹² and X¹³ are each independently selected from CH and N, provided no more than one of X¹⁰, X¹¹, X¹² and X¹³ is N.

In some embodiments in the compound of formula (LXIII), one of X¹⁰, X¹¹, X¹² and X¹³ is N and the remaining X¹a, X¹¹, X¹² and X¹³ are CH.

In certain embodiments compounds of the invention include, for example, compounds of formulae (I) to (LXIII), or a pharmaceutically acceptable salt thereof, wherein, unless otherwise stated, each of R¹, R², R³, R⁴, R⁵, R⁶, R⁷, R^{A1}, Q¹, Q² L¹ Q³, L¹, L³, L⁴, L⁵, p¹, p², q¹, q², n, n1, n2 n3 and n4 has any of the meanings defined hereinbefore or in any of paragraphs (1) to (320) hereinafter. These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification:—
1. n is 0.
2. n is 1 or 2, and R¹ is independently selected from halo. It may be that R¹ is independently selected from —F, —Cl and —Br.
3. n is 1 or 2 and R¹ is —F.
4. n is 1 or 2 and R¹ is independently selected from C₁₋₄ alkyl.
5. n is 1 or 2 and R¹ is independently selected from C₁₋₃ alkyl and halo. Thus it may be that n is 1 or 2 and R¹ is independently selected from C₁₋₃ alkyl and F. It may be that n is 1 or 2 and R¹ is independently selected from methyl and F. It may be that n is 1 or 2 and R¹ is independently selected from C₁₋₃ alkyl. It may be that n is 1 or 2 and R¹ is independently selected from methyl and ethyl.
6. n is 1 or 2 and R¹ is methyl.
7. n is 2. It may be that n is 2 and R¹ has any of the values in one of paragraphs 2 to 6.
8. The group

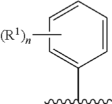

has a structure selected from the group consisting of:

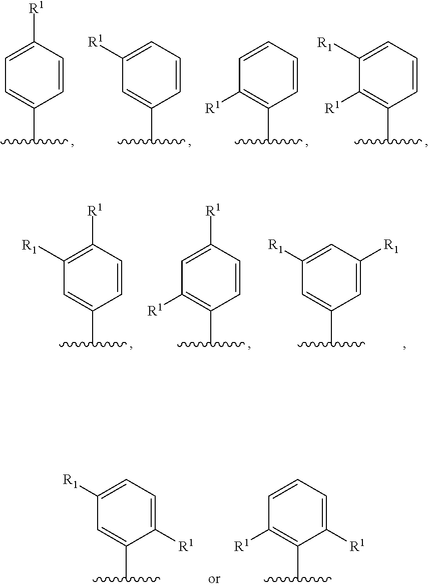

wherein [symbol] indicates the point of attachment to Formula (I). It may be that n is 2 and R¹ has any of the values in one of paragraphs 2 to 6.
9. The group

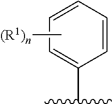

has the structure

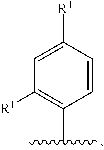

wherein [symbol] indicates the point of attachment to Formula (I). It may be that n is 2 and R¹ has any of the values in one of paragraphs 2 to 6.
10. The group

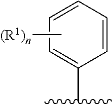

has the structure

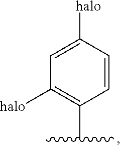

wherein [symbol] indicates the point of attachment to Formula (I).
11. The group

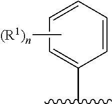

has the structure

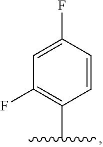 [SMILES: *c1ccc(F)cc1F]

wherein [symbol] indicates the point of attachment to Formula (I).
12. The group

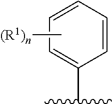

has the structure

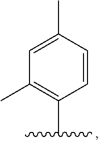 [SMILES: *c1ccc(C)cc1C]

wherein [symbol] indicates the point of attachment to Formula (I).
13. The group

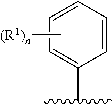

has the structure

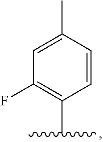 [SMILES: *c1ccc(C)cc1F]

wherein [symbol] indicates the point of attachment to Formula (I).
14. The group

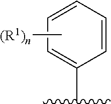

has the structure

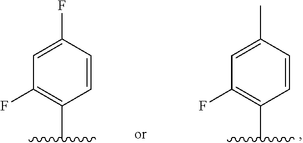

preferably

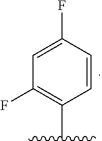 [SMILES: *c1ccc(F)cc1F]

15. R² is H.
16. R² is C₁₋₄ alkyl.
17. R² is C₁₋₃ alkyl. For example, it may be that R² is methyl or ethyl. It may be that R² is methyl.
18. Q¹ is selected from the group consisting of:

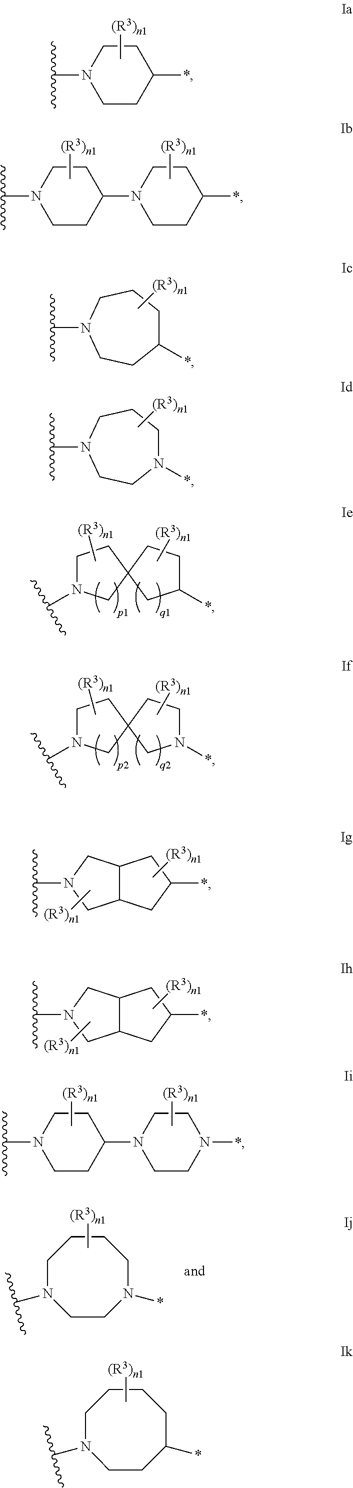

wherein:
   * indicates the point of attachment to L¹;
   p¹, q¹, p² and q² are independently an integer selected from 1 or 2;
   [symbol] indicates the point of attachment to Formula (I); and
   each n1 is independently selected from 0, 1 or 2.
19. Q¹ is selected from the group consisting of:

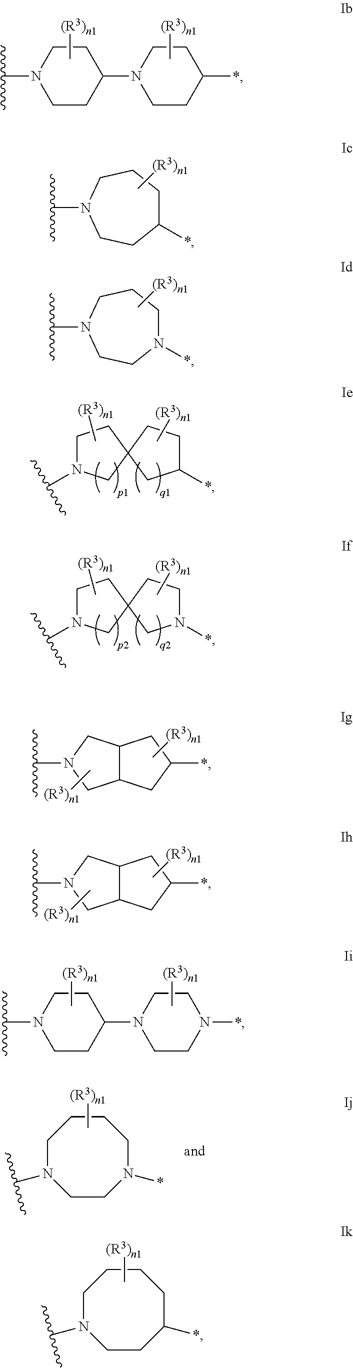

wherein:
   * indicates the point of attachment to L¹;
   p¹, q¹, p² and q² are independently an integer selected from 1 or 2;
   [symbol] indicates the point of attachment to Formula (I); and
   each n1 is independently selected from 0, 1 or 2.
20. Q¹ is selected from the group consisting of:

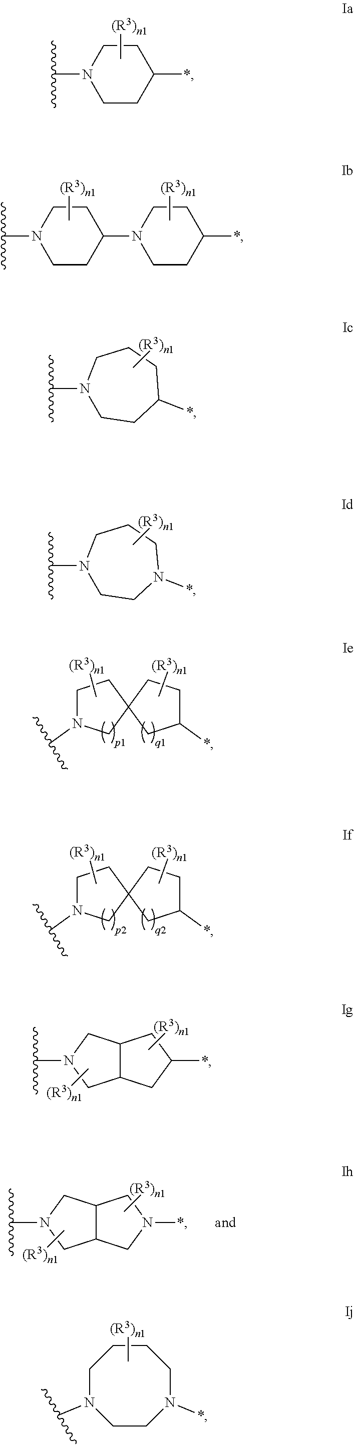

wherein:
   * indicates the point of attachment to L¹;
   p¹, q¹, p² and q² are independently an integer selected from 1 or 2;
   [symbol] indicates the point of attachment to Formula (I); and
   each n1 is independently selected from 0, 1 or 2.
21. Q¹ is selected from the group consisting of:

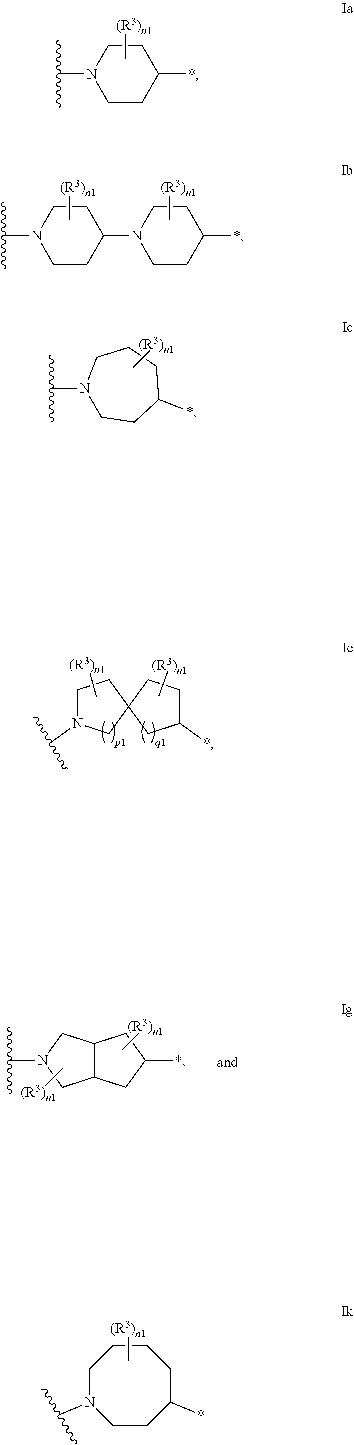

wherein:
   * indicates the point of attachment to L¹;
   p¹ and q¹ are independently an integer selected from 1 or 2;
   [symbol] indicates the point of attachment to Formula (I); and
   each n1 is independently selected from 0, 1 or 2.
22. Q¹ is

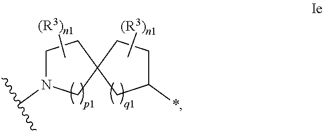

wherein p¹ is 1 and q¹ is 1.
23. Q¹ is

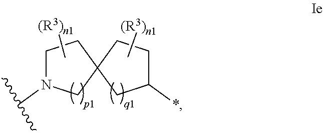

wherein p¹ is 1 and q¹ is 2.
24. Q¹ is

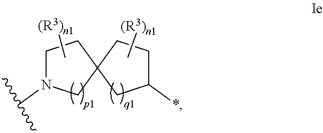

wherein p¹ is 2 and q¹ is 1.
25. Q¹ is

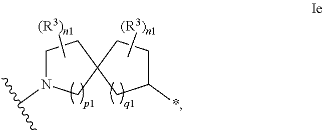

wherein p¹ is 2 and q¹ is 2.
26. Q¹ is

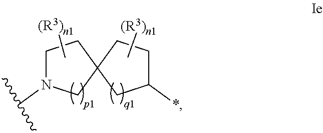

wherein the group Ie is selected from the group consisting of:

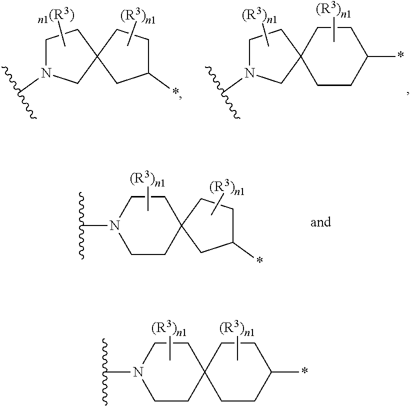

wherein:
   * indicates the point of attachment to L¹;
   [symbol] indicates the point of attachment to Formula (I); and
   each n1 is independently selected from 0, 1 or 2.
27. Q¹ is selected from the group consisting of:

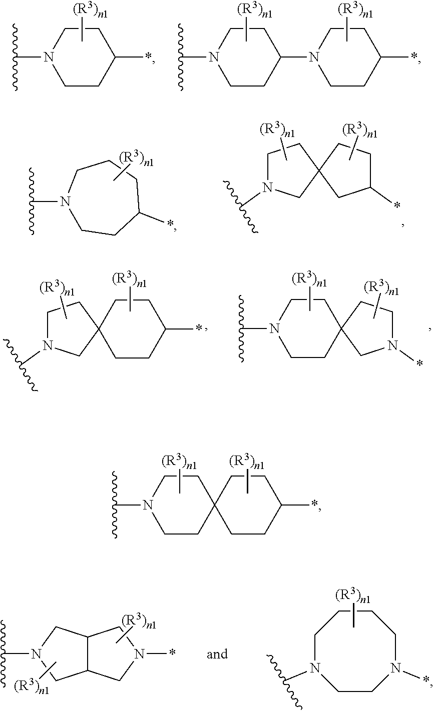

wherein:
   * indicates the point of attachment to L¹;
   [symbol] indicates the point of attachment to Formula (I); and
   each n1 is independently selected from 0, 1 or 2.
28. Q¹ is selected from the group consisting of:

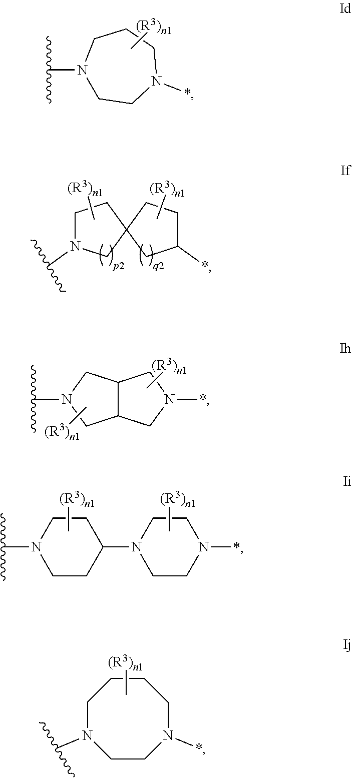

wherein:
   * indicates the point of attachment to L¹;
   p² and q² are independently an integer selected from 1 or 2;
   [symbol] indicates the point of attachment to Formula (I); and
   each n1 is independently selected from 0, 1 or 2.
29. Q¹ is

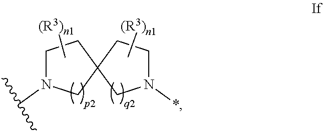

wherein p² is 1 and q² is 1.
30. Q¹ is

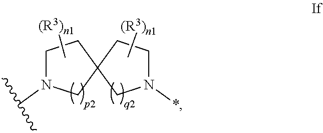

wherein p² is 1 and q² is 2.
31. Q¹ is

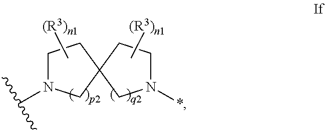

wherein p² is 2 and q² is 1.
32. Q¹ is

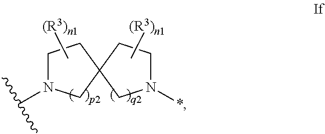

wherein p² is 2 and q² is 2.
33. Q¹ is selected from the group consisting of:

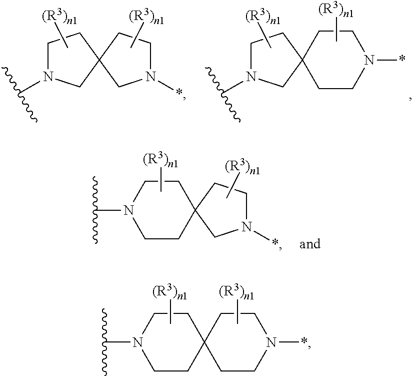

wherein:
   * indicates the point of attachment to L¹;
   [symbol] indicates the point of attachment to Formula (I); and
   each n1 is independently selected from 0, 1 or 2.
34. Q¹ is selected from the group consisting of:

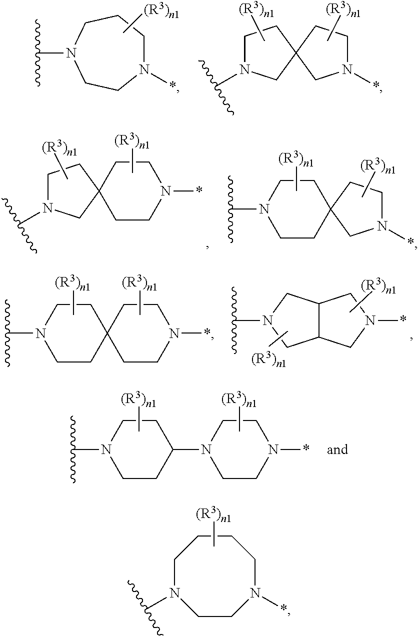

wherein:
   * indicates the point of attachment to L¹;
   [symbol] indicates the point of attachment to Formula (I); and
   each n1 is independently selected from 0, 1 or 2.
35. R³ is absent (i.e. Q¹ is unsubstituted).
36. R³ is C₁₋₃ alkyl at each occurrence. It may be that R³ is methyl. It may be that R³ is ethyl.
37. R³ is ═O at each occurrence.
38. Q¹ is

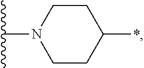

wherein:
   * indicates the point of attachment to L¹; and
   [symbol] indicates the point of attachment to Formula (I).
39. L¹ is a bond.
40. L¹ is a bond and Q¹ is as defined in any one of numbered paragraphs 18 to 34. Thus it may be that L¹ is a bond and Q¹ is as defined in any one of numbered paragraphs 21 to 27. Thus it may be that L¹ is a bond and Q¹ is as defined in any one of numbered paragraphs 28 to 34. Thus, it may be that the group -Q¹-L¹- is selected from the group consisting of:

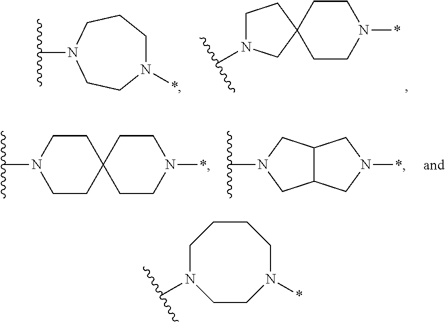

wherein:
   * indicates the point of attachment to Q²; and
   [symbol] indicates the point of attachment to Formula (I).
41. L¹ is —NR⁴—, wherein R⁴ is selected from H and C₁₋₃ alkyl. Thus it may be that L¹ is —NR⁴—, wherein R⁴ is selected from H and C₁₋₃ alkyl and Q¹ is as defined in any one of numbered paragraphs 21 to 27.
42. L¹ is —NH—.
43. L¹ is —N(C₁₋₃ alkyl)-. It may be that L¹ is —N(CH₃)—. It may be that L¹ is —N(Et)-.
44. L¹ is —NH—, and Q¹ is as defined in any one of numbered paragraphs 21 to 27. Thus, it may be that the group -Q¹-L¹- is selected from the group consisting of:

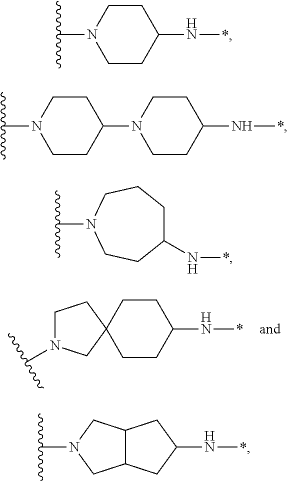

wherein:
   * indicates the point of attachment to Q²; and
   [symbol] indicates the point of attachment to Formula (I). Thus it may be that the group -Q¹-L¹- is

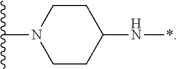

45. L¹ is —CH₂—. Thus it may be that L¹ is —CH₂— and Q¹ is as defined in any one of numbered paragraphs numbered paragraphs 18 to 34. Thus it may be that L¹ is —CH₂— and Q¹ is as defined in any one of numbered paragraphs 21 to 27. Thus it may be that L¹ is —CH₂— and Q¹ is as defined in any one of numbered paragraphs 28 to 34.
46. L¹ is —O—. Thus it may be that L¹ is —O— and Q¹ is as defined in any one of numbered paragraphs 21 to 27.
47. Q² is phenyl.
48. Q² is phenyl optionally substituted with one R⁵ group and optionally one or more R⁶ group. Thus, it may be that Q² is

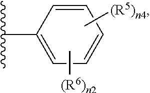

wherein:
   n2 is an integer selected from 0, 1 or 2;
   n4 is an integer selected from 0 or 1; and
   [symbol] indicates the point of attachment to L¹ of Formula (I).
49. Q² is phenyl optionally substituted with one or more R⁶ group, and R⁵ is absent, i.e. n4 is 0. Thus, it may be that Q² is

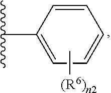

wherein:
   n2 is an integer selected from 0, 1 or 2; and
[symbol] indicates the point of attachment to L¹ of Formula (I). Thus, it may be that Q² is

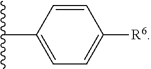

50. R⁵ is Q³-L³-. Thus, it may be that Q² is

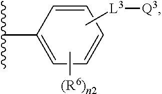

wherein:
   n2 is an integer selected from 0, 1 or 2; and
   [symbol] indicates the point of attachment to L¹ of Formula (I).
51. Q² is

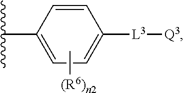

wherein:
   n2 is an integer selected from 0, 1 or 2; and
   [symbol] indicates the point of attachment to L¹ of Formula (I). Thus, it may be that Q² is

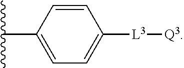

52. Q² is a 5- or 6-membered heteroaryl group containing at least one ring heteroatom, wherein the heteroaryl is optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted. It may be that when Q² is a 5-membered heteroaryl, Q² is bonded to Q¹-L¹- by a ring carbon atom in Q². It may be that when Q² is a 5-membered heteroaryl, Q² is bonded to Q¹-L¹- by a ring nitrogen atom in Q² (where chemically possible).
53. Q² is a 5- or 6-membered heteroaryl group containing at least one ring nitrogen atom, wherein the heteroaryl is optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted. It may be that when Q² is a 5-membered heteroaryl, Q² is bonded to Q¹-L¹- by a ring carbon atom in Q². It may be that when Q² is a 5-membered heteroaryl, Q² is bonded to Q¹-L¹- by a ring nitrogen atom in Q² (where chemically possible).
54. Q² is a 5-membered heteroaryl group containing at least one ring nitrogen atom, wherein the heteroaryl is optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted. It may be that Q² is bonded to Q¹-L¹- by a ring carbon atom in Q². It may be that Q² is bonded to Q¹-L¹- by a ring nitrogen atom in Q² (where chemically possible).
55. Q² is a 5-membered heteroaryl group, wherein said heteroaryl group contains from 1 to 4 ring heteroatoms selected from O, S and N, wherein the heteroaryl is optionally substituted with one R⁵ group and optionally one or more R⁶ group. Thus it may be that Q² is selected from pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl, wherein the heteroaryl is optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted. It may be that Q² is bonded to Q¹-L¹- by a ring carbon atom in Q². It may be that Q² is bonded to Q¹-L¹- by a ring nitrogen atom in Q² (where chemically possible).
56. Q² is a 5-membered heteroaryl group, wherein said heteroaryl group contains 1 ring nitrogen atom and optionally 1 to 3 (e.g. 1 or 2) ring heteroatoms selected from O, S and N, wherein the heteroaryl is optionally substituted with one R⁵ group and optionally one or more R⁶ group. Thus it may be that Q² is selected from pyrrolyl, imidazolyl, furazanyl, oxazolyl, oxadiazolyl, oxatriazolyl, isoxazolyl, thiazolyl, isothiazolyl, pyrazolyl, triazolyl and tetrazolyl, wherein the heteroaryl is optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted. It may be that Q² is bonded to Q¹-L¹- by a ring carbon atom in Q². It may be that Q² is bonded to Q¹-L¹- by a ring nitrogen atom in Q² (where chemically possible).
57. Q² is a 5-membered heteroaryl group, wherein said heteroaryl group contains from 1 to 4 ring nitrogen atoms (e.g. 1 or 2 ring nitrogen atoms), wherein the heteroaryl is optionally substituted with one R⁵ group and optionally one or more R⁶ group. Thus it may be that Q² is selected from pyrrolyl, imidazolyl, pyrazolyl, triazolyl and tetrazolyl, wherein the heteroaryl is optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted. It may be that Q² is bonded to Q¹-L¹- by a ring carbon atom in Q². It may be that Q² is bonded to Q¹-L¹- by a ring nitrogen atom on Q² (where chemically possible).
58. Q² is a 6-membered heteroaryl group containing at least one ring nitrogen atom, wherein the heteroaryl is optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted.
59. Q² is a 6-membered heteroaryl group, wherein said heteroaryl group contains from 1 to 3 ring nitrogen atoms, wherein the heteroaryl is optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted.
60. Q² is a heteroaryl group selected from pyridyl and pyrimidyl, wherein said heteroaryl is optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted.
61. Q² is pyrimidyl optionally substituted with one R⁵ group and optionally one or more R⁶ group. Thus, it may be that Q² is substituted by one or two R⁶ groups. Thus, it may be that Q² is substituted by one R⁶ group. Thus, it may be that Q² is substituted by one R⁵ group. Thus, it may be that Q² is unsubstituted.
62. Q² has the structure:

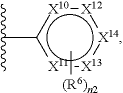

wherein:
X¹⁰, X¹¹, X¹², X¹³ and X¹⁴ are each independently selected from CH and N, wherein at least one and no more than three of X¹⁰, X¹¹, X¹², X¹³ and X¹⁴ is N;
n2 is 0, 1 or 2 (where chemically possible); and
[symbol] indicates the point of attachment to L¹ of Formula (I).
Preferably, no more than two of X¹⁰, X¹¹, X¹², X¹³ and X¹⁴ are N.
63. When Q² is defined as in paragraph 62, one X group can be substituted with one R⁶ group.
64. Q² is selected from:

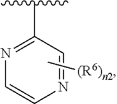

wherein n2 is 0, 1 or 2, and wherein [symbol] indicates the point of attachment to L¹ of Formula (I). Typically, n2 is 0 or 1. For example, n2 is 0. For example, n2 is 1.
65. Q² is

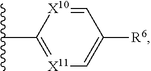

wherein:
   X¹⁰ and X¹¹ are each independently selected from CH and N, wherein at least one of X¹⁰ and X¹¹ is N; and
   [symbol] indicates the point of attachment to L¹ of Formula (I).
66. Q² is selected from the group consisting of:

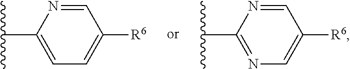

wherein [symbol] indicates the point of attachment to L¹ of Formula (I).
67. In some embodiments, Q² is selected from:

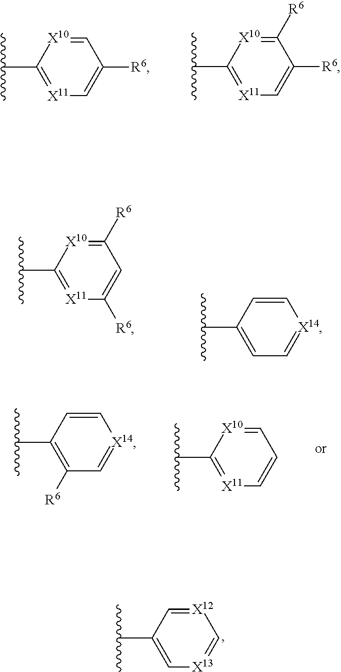

wherein:
   X¹⁰, X¹¹, X¹², X¹³ and X¹⁴ are each independently selected from CH and N; and
   [symbol] indicates the point of attachment to L¹ of Formula (I). Thus it may be that Q² is selected from:

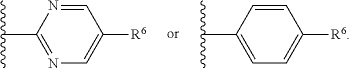

Suitably in this paragraph Q¹ is defined as in paragraph 18.
68. Q² has the structure:

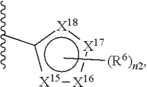

wherein:
   X¹⁵, X¹⁶, X¹⁷ and X¹⁸ are each independently selected from CH and N, wherein at least one of X¹⁵, X¹⁶, X¹⁷ and X¹⁸ is N;
n2 is 0, 1 or 2 (where chemically possible); and
[symbol] indicates the point of attachment to L¹ of Formula (I).
Thus it may be that one to four of X¹⁵, X¹⁶, X¹⁷ and X¹⁸ is N. Thus it may be that one to three of X¹⁵. X¹⁶. X¹⁷ and X¹⁸ is N. Thus it may be that one or two of X¹⁵, X¹⁶, X¹⁷ and X¹⁸ is N.
Thus it may be that one of X¹⁵, X¹⁶, X¹⁷ and X¹⁸ is N. Thus it may be that two of X¹⁵, X¹⁶, X¹⁷ and X¹⁸ are N. Thus it may be that three of X¹⁵, X¹⁶, X¹⁷ and X¹⁸ are N.
69. When Q² is defined as in paragraph 68, one X group can be substituted with one R⁶ group.
70. Q² is selected from:

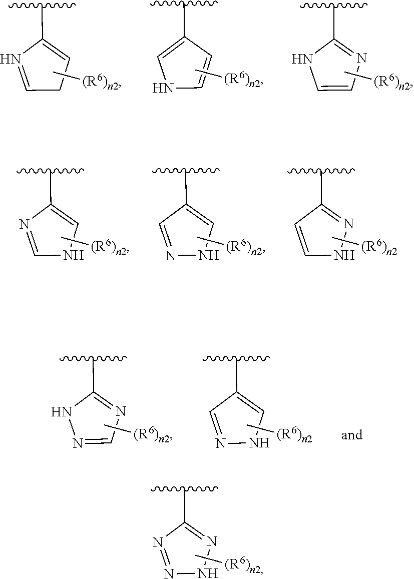

wherein n2 is 0, 1 or 2, and wherein [symbol] indicates the point of attachment to L¹ of Formula (I). Typically, n2 is 0 or 1. For example, n2 is 0. For example, n2 is 1.
Thus it may be that Q² is selected from:

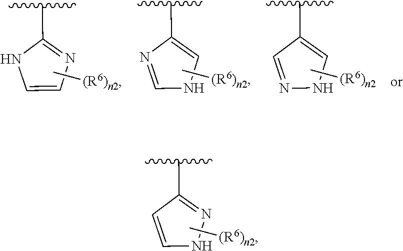

wherein n2 is 0, 1 or 2, and wherein [symbol] indicates the point of attachment to L¹ of Formula (I). Typically, n2 is 0 or 1. For example, n2 is 0. For example, n2 is 1.
71. Q² is

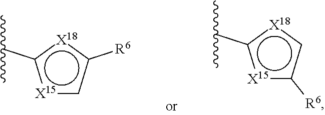

wherein:
   X¹⁵ and X¹⁸ are each independently selected from CH and N, wherein at least one of X¹⁵ and X¹⁸ is N; and
   [symbol] indicates the point of attachment to L¹ of Formula (I).
72. Q² is selected from the group consisting of:

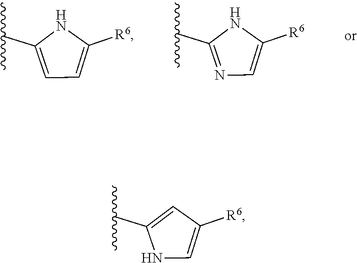

wherein [symbol] indicates the point of attachment to L¹ of Formula (I).
73. R⁶ is absent.
74. R⁶ at each occurrence is independently selected from the group consisting of: halo, ═O, —CN, —NO₂, C₁₋₄ alkyl, C₂₋₄ alkenyl, C₂₋₄ alkynyl, C₁₋₄ haloalkyl, C₁₋₆ cycloalkyl, —OR^{A1}, —S(O)ₓR^{A2} (wherein x is 0, 1, or 2), —NR^{A2}R^{B2}, —C(O)R^{A2}, —OC(O)R^{A2}, —C(O)OR^{A2}, —NR^{B2}C(O)R^{A2}, —NR^{B2}C(O)OR^{A2}, —C(O)NR^{A2}R^{B2}, —NR^{B2}SO₂R^{A2} and —SO₂NRAR^{B2};
   wherein said C₁₋₄alkyl C₂₋₄ alkenyl, C₂₋₄ alkynyl are each is optionally substituted by 1 or 2 substituents independently selected from: halo, —CN, —OR^{A3}, —NR^{A3}R^{B3} and —SO₂R^{A3}; and
   wherein said C₁₋₆ cycloalkyl is optionally substituted by one or more substituents independently selected from halo, ═O, —CN, —NO₂, C₁₋₄ alkyl and C₁₋₄ haloalkyl;
   R^{A1}, R^{A2}, R^{B2}, R^{A3} and R^{B3} are each independently selected from: H, C₁₋₄ alkyl and C₁₋₄ haloalkyl.
75. R⁶ at each occurrence is independently selected from the group consisting of: halo, ═O, —CN, —NO₂, C₁₋₄ alkyl, C₁₋₄ haloalkyl, —OR^{A1}, —S(O)ₓR^{A2} (wherein x is 0, 1, or 2) and —NR^{A2}R^{B2}.
   wherein said C₁₋₄alkyl is optionally substituted by 1 or 2 substituents independently selected from: halo, —CN, —OR^{A3}, —NR^{A3}R^{B3} and —SO₂R^{A3}; and
   R^{A1}, R^{A2}, R^{B2}, R^{A3} and R^{B1} are each independently selected from: H, C₁₋₄ alkyl and C₁₋₄ haloalkyl.
   Thus it may be that R⁶ at each occurrence is independently selected from the group consisting of: halo, —CN, —NO₂, C₁₋₄ alkyl, C₁₋₄ haloalkyl, —OR^{A1} and —NR^{A2}R^{B2}.
76. R⁶ at each occurrence is independently selected from the group consisting of: halo, —CN, C₁₋₄ alkyl and —OR^{A1}.
77. R⁶ is as defined in numbered paragraph 76, wherein when Q² is as defined in any one of numbered paragraphs 47 to 72.
78. R⁶ at each occurrence is independently selected from the group consisting of: halo, ═O, —CN, —NO₂, C₁₋₆ alkyl, C₂₋₆ alkenyl, C₂₋₆ alkynyl, C₁₋₆ haloalkyl, C₁₋₆ cycloalkyl, —ORA, —S(O)ₓR^{A2} (wherein x is 0, 1, or 2), —NR^{A2}R^{B2}, —C(O)R^{A}, —OC(O)R^{A2}, —C(O)OR^{A2}, —NR^{B2}C(O)R^{A2}, —NR^{B2}C(O)OR^{A2}, —C(O)NR^{A2}R^{B2}, —NR^{B2}SO₂R^{A2} and —SO₂NR^{A2}R^{B2};
   wherein said C₁₋₆alkyl C₂₋₆ alkenyl, C₂₋₆ alkynyl are each is optionally substituted by 1 or 2 substituents independently selected from: halo, —CN, —OR^{A3}, —NR^{A3}R^{B3} and —SO₂R^{A3}; and
   wherein said C₁₋₆ cycloalkyl is optionally substituted by one or more substituents independently selected from halo, ═O, —CN, —NO₂, C₁₋₄ alkyl and C₁₋₄ haloalkyl;
   R^{A1}, R^{A2}, R^{B2}, R^{A3} and R^{B3} are each independently selected from: H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl.
79. R^{A1}, R^{A2}, R^{B2}, R^{A3} and R^{B3} are each independently selected from: H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl. It may be that R^{A1} is selected from H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl, and R^{A2}, R^{B2}, R^{A3} and R^{B3} are each independently selected from: H, C₁₋₆ alkyl and C₁₋₆ haloalkyl. It may be that R^{A1} is selected from H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl, and R^{A2}, R^{B2}, R^{A3} and R^{B3} are each independently selected from: H, unsubstituted C₁₋₆ alkyl and C₁₋₆ haloalkyl. Thus, it may be that R^{A1} is C₁₋₆ alkyl optionally substituted by 1 or 2 substituents selected from —OR^{A4}, and R^{A2}, R^{B2}, R^{A3} and R^{B3} are each independently selected from: H, unsubstituted C₁₋₆ alkyl and C₁₋₆ haloalkyl. Thus it may be that R^{A1}, R^{A2}, R^{B2}, R^{A3} and R^{B3} are each independently selected from: H, unsubstituted C₁₋₄ alkyl and C₁₋₄ haloalkyl.
80. R⁶ at each occurrence is independently selected from the group consisting of: halo, ═O, —CN, —NO₂, C₁₋₆ alkyl, C₁₋₆ haloalkyl, —OR^{A1}, —S(O)ₓR^{A2} (wherein x is 0, 1, or 2) and —NR^{A2}R^{B2};
   wherein said C₁₋₆ alkyl is optionally substituted by 1 or 2 substituents independently selected from: halo, —CN, —OR^{A3}, —NR^{A3}R^{B3} and —SO₂R^{A3}; and
   R^{A1}, R^{A2}, R^{B2}, R^{A3} and R^{B3} are each independently selected from: H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl.
   Thus, it may be that R⁶ at each occurrence is independently selected from the group consisting of: halo, —CN, —NO₂, C₁₋₆ alkyl, C₁₋₆ haloalkyl, —OR^{A1} and —NR^{A2}R^{B2}.
81. R⁶ at each occurrence is independently selected from the group consisting of: halo, —CN, C₁₋₆ alkyl and —OR^{A1}.
82. R⁶ is as defined in numbered paragraph 81, wherein when Q² is as defined in any one of numbered paragraphs 45 to 72.
83. R⁶ is halo. For example, R⁶ is —F.
84. R⁶ is —F. Thus, Q² may be selected from

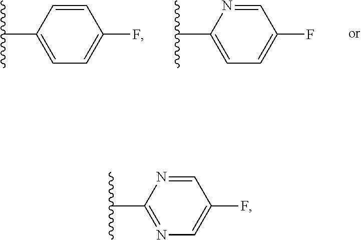

wherein [symbol] indicates the point of attachment to L of Formula (I).
85. R⁶ is C₁₋₄ alkyl optionally substituted by 1 or 2 substituents independently selected from: halo, —CN, —OR^{A3}, —NR^{A3}R^{B3} and —SO₂R^{A3}. It may be that R⁶ is unsubstituted C₁₋₄ alkyl. It may be that R⁶ is methyl.
86. R⁶ is C₁₋₆ alkyl optionally substituted by 1 or 2 substituents independently selected from: halo, —CN, —OR^{A3}, —NR^{A3}R^{B3} and —SO₂R^{A3}. It may be that R⁶ is unsubstituted C₁₋₆ alkyl. It may be that R⁶ is methyl.
87. R⁶ is methyl. Thus, Q² may be selected from

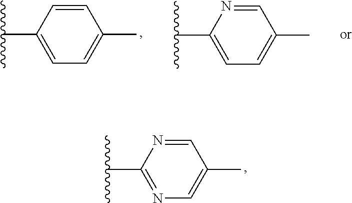

wherein [symbol] indicates the point of attachment to L¹ of Formula (I).
88. R⁶ is —CN.
89. R⁶ is —CN. Thus, Q² may be selected from

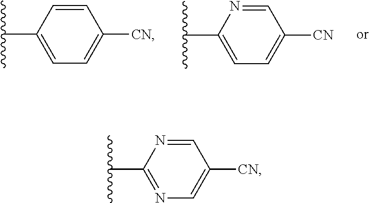 [SMILES: *c1ccc(C#N)cc1 ; *c1ccc(C#N)cn1 ; *c1ncc(C#N)cn1]

wherein [symbol] indicates the point of attachment to L¹ of Formula (I).
90. R⁶ is —OR^{A1}, wherein R^{A1} is selected from: H, C₁₋₄ alkyl and C₁₋₄ haloalkyl. It may be that R^{A1} is C₁₋₄ alkyl. It may be that R^{A1} is methyl. Thus, it may be that R⁶ is —OCH₃.
91. R⁶ is —OCH₃. Thus, Q² may be selected from

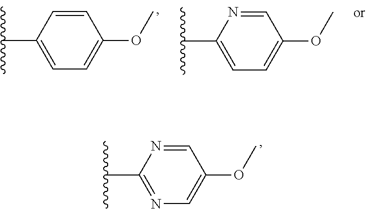

wherein [symbol] indicates the point of attachment to L¹ of Formula (I).
92. R⁶ is —OR^{A1}, wherein R^{A1} is selected from: H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl. It may be that R^{A1} is C₁₋₆ alkyl substituted with —OR^{A4}. It may be that R^{A4} is C₁₋₄ alkyl. It may be that R^{A1} is C₁₋₆ alkyl substituted with —OC₁₋₄ alkyl. It may be that R^{A1} is C₂₋₄ alkyl substituted with —OC₁₋₂ alkyl. It may be that R^{A1} is —(CH₂)₂—OCH₃. Thus, it may be that R⁶ is —O(CH₂)₂—OCH₃.
93. R⁶ is —O(CH₂)₂—OCH₃. Thus, Q² may be selected from

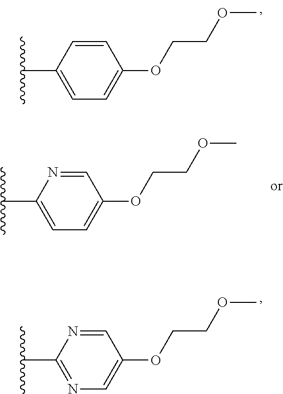 [SMILES: *c1ccc(OCCOC)cc1 ; *c1ccc(OCCOC)cn1 ; *c1ncc(OCCOC)cn1]

wherein [symbol] indicates the point of attachment to L¹ of Formula (I).
94. R⁶ is —OR^{A1}, wherein R^{A1} is selected from: H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl. It may be that R^{A1} is C₁₋₆ alkyl. It may be that R^{A1} is C₃₋₆ alkyl. It may be that R^{A1} is branched C₃₋₆ alkyl. It may be that R^{A1} is —CH₂CH(CH₃)₂ (i.e. isobutyl). Thus, it may be that R⁶ is —OC₃₋₆ alkyl, for example, —OCH₂CH(CH₃)₂ (i.e. iso-butoxy).
95. R⁶ is —OCH₂CH(CH₃)₂. Thus, Q² may be selected from

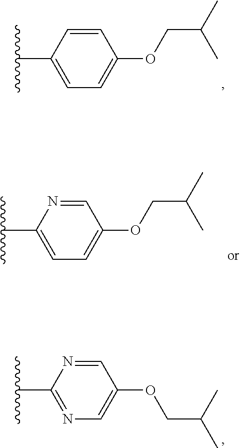 [SMILES: *c1ccc(OCC(C)C)cc1 ; *c1ccc(OCC(C)C)cn1 ; *c1ncc(OCC(C)C)cn1]

wherein [symbol] indicates the point of attachment to L¹ of Formula (I).
96. Q² is

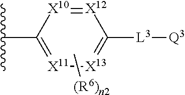

wherein X¹⁰, X¹¹, X¹² and X¹³ are each independently selected from CH and N, provided no more than two of X¹⁰, X¹¹, X¹² and X¹³ are N; n2 is an integer selected from 0, 1 or 2; and
[symbol] indicates the point of attachment to L¹ of Formula (I).
Thus it maybe that Q² is

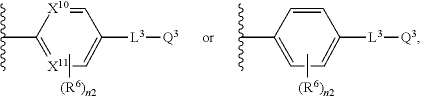

for example it may be that Q² is

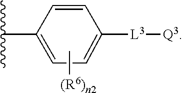

In this paragraph it may be that R⁶ is selected from halo, methyl, —CN or —OCH₃.
97. Q² is

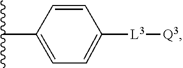

wherein:
   [symbol] indicates the point of attachment to L¹ of Formula (I).
98. Q² is phenyl, pyridyl, or pyrimidyl, and Q² is substituted by X and optionally one or more R⁶, wherein X is selected from Q³-L³-, —O—C₁₋₆ alkyl and —O—C₁₋₆ alkyl-OR^{A4}, and R^{A4} is H or C₁₋₄ alkyl.
99. X is Q³-L³-.
100. X is —O—C₁₋₆ alkyl. It may be that X is —O—C₃₋₆ alkyl. It may be that the C₃₋₆ alkyl is branched. Thus, it may be that X is —OCH₂CH(CH₃)₂ (i.e. iso-butoxy).
101. X is —O—C₁₋₆ alkyl-OR^{A4}, and R^{A4} is H or C₁₋₄ alkyl. It may be that R^{A4} is H, thus X is —O—C₁₋₆ alkyl-OH.
102. X is —O—C₁₋₆ alkyl-OR^{A4}, and R^{A4} is H or C₁₋₄ alkyl. It may be that R^{A4} is C₁₋₄ alkyl. Thus, X is —O—C₁₋₆ alkyl-OC₁₋₄ alkyl. It may be that X is —O—C₂₋₄ alkyl-OC₁₋₂ alkyl. Thus, it may be that X is —O(CH₂)₂—OCH₃.
103. L³ is a bond.
104. L³ is selected from: C₁₋₄ alkylene, -L⁴-O-L⁵-, -L⁴-NR^{A1}-L⁵-, -L⁴-S(O)ₓ-L⁵-, -L⁴-C(═O)-L⁵, -L⁴-NR^{A1}C(═O)-L⁵-, -L⁴-C(═O)NR^{A1}-L⁵-, -L⁴-S(O)₂NR^{A1}-L⁵-, L⁴-NR^{A1}S(O)₂-L⁵, -L⁴-OC(═O)-L⁵ and -L⁴-C(═O)O-L⁵-,
   wherein x is 0, 1 or 2; and
   L⁴ and L⁵ are independently selected from a bond and C₁₋₄ alkylene. Wherein, -L⁴ is the point of attachment to Q² and L⁵- is the point of attachment to Q³ of Formula (I).
105. L⁴ and L⁵ are independently selected from a bond or C₁₋₄ alkylene. It may be that L⁴ and L⁵ are independently selected from a bond or C₁₋₂ alkylene. It may be that L⁴ and L⁵ are independently selected from a bond or —CH₂—. It may be that L⁴ and L⁵ are independently selected from a bond or —CH₂CH₂—.
106. L³ is C₁₋₄ alkylene. It may be that L³ is C₁₋₃ alkylene. It may be that L³ is —CH₂—.
107. L³ is -L⁴-O-L⁵-. It may be that L⁴ and L⁵ are independently selected from a bond or —CH₂—. It may be that L³ is —O—. It may be that L³ is —O—CH₂—. It may be that L³ is —CH₂—O—. It may be that L³ is —CH₂—O—CH₂—.
108. L³ is -L⁴-O-L⁵-. It may be that L⁴ and L⁵ are independently selected from a bond or —CH₂CH₂—. It may be that L³ is —O—. It may be that L³ is —O—CH₂CH₂—. It may be that L³ is —CH₂CH₂—O—. It may be that L³ is —CH₂CH₂—O—CH₂CH₂—.
109. L³ is -L⁴-O-L⁵-. It may be that L⁴ is a bond and L⁵- is C₁₋₃ alkylene. Thus, it may be that L³ is

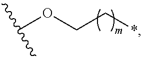

wherein:
   m is 0, 1 or 2;
   [symbol] indicates the point of attachment to Q² in Formula (I); and
   * indicates the point of attachment to Q³ in Formula (I).
110. L³ is -L⁴-NR^{A1}-L⁵-. It may be that L⁴ and L⁵ are independently selected from a bond or —CH₂—. It may be that R^{A1} is H. It may be that L³ is —NH—. It may be that L³ is —NH—CH₂. It may be that L³ is —CH₂—NH—. It may be that L³ is —CH₂—NH—CH₂—.
111. L³ is -L⁴-NR^{A1}-L⁵-. It may be that L⁴ and L⁵ are independently selected from a bond or —CH₂CH₂—. It may be that R^{A1} is H. It may be that L³ is —NH—. It may be that L³ is —NH—CH₂CH₂. It may be that L³ is —CH₂CH₂—NH—. It may be that L³ is —CH₂CH₂—NH—CH₂CH₂—.
112. Q³ is phenyl.
113. Q³ is phenyl substituted with one or more R⁷. Thus, it may be that Q³ is

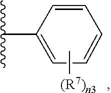

wherein:
   n3 is an integer selected from 1 or 2; and
   [symbol] indicates the point of attachment to L³ of Formula (I).
114. Q³ is selected from:

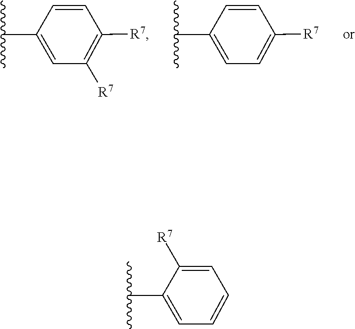

wherein [symbol] indicates the point of attachment to L³ of Formula (I).
115. Q³ is selected from:

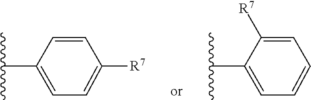

wherein [symbol] indicates the point of attachment to L³ of Formula (I).
116. Q³ is a 5- or 6-membered heteroaryl group containing at least one ring heteroatom selected from O, S and N (e.g. from 1 to 3 heteroatoms, preferably 1 or 2 heteroatoms), wherein the heteroaryl is optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group. It may be that when Q³ is a 5-membered heteroaryl, Q³ is bonded to Q²-L³- by a ring carbon atom in Q³. It may be that when Q³ is a 5-membered heteroaryl, Q³ is bonded to Q²-L³- by a ring nitrogen atom in Q³ (where chemically possible).
117. Q³ is a 5- or 6-membered heteroaryl group containing at least one ring nitrogen atom and optionally one additional heteroatom selected from O, S and N, wherein the heteroaryl is optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group. It may be that when Q³ is a 5-membered heteroaryl, Q³ is bonded to Q²-L³- by a ring carbon atom in Q³. It may be that when Q³ is a 5-membered heteroaryl, Q³ is bonded to Q²-L³- by a ring nitrogen atom in Q³ (where chemically possible).
118. Q³ is a 5-membered heteroaryl group containing at least one ring nitrogen atom, wherein the heteroaryl is optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group. It may be that Q³ is bonded to Q²-L³- by a ring carbon atom in Q³. It may be that Q³ is bonded to Q²-L³- by a ring nitrogen atom in Q³ (where chemically possible).
119. Q³ is a 5-membered heteroaryl group, wherein said heteroaryl group contains from 1 to 4 ring heteroatoms selected from O, S and N, wherein the heteroaryl is optionally substituted with one or more R⁷ group. Thus it may be that Q³ is selected from pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl, wherein the heteroaryl is optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group. It may be that Q³ is bonded to Q²-L³- by a ring carbon atom in Q³. It may be that Q³ is bonded to Q²-L³- by a ring nitrogen atom in Q³ (where chemically possible).
120. Q³ is a 5-membered heteroaryl group, wherein said heteroaryl group contains 1 ring nitrogen atom and optionally 1 to 3 (e.g. 1 or 2) ring heteroatoms selected from O, S and N, wherein the heteroaryl is optionally substituted with one or more R⁷ group. Thus it may be that Q³ is selected from pyrrolyl, imidazolyl, furazanyl, oxazolyl, oxadiazolyl, oxatriazolyl, isoxazolyl, thiazolyl, isothiazolyl, pyrazolyl, triazolyl and tetrazolyl, wherein the heteroaryl is optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group. It may be that Q³ is bonded to Q²-L³- by a ring carbon atom in Q³. It may be that Q³ is bonded to Q²-L³- by a ring nitrogen atom in Q³ (where chemically possible).
121. Q³ is a 5-membered heteroaryl group, wherein said heteroaryl group contains from 1 to 3 ring nitrogen atoms. For example, the heteroaryl group contains 1 or 2 ring nitrogen atoms, wherein the heteroaryl is optionally substituted with one or more R⁷ group. Thus it may be that Q³ is selected from pyrrolyl, imidazolyl, pyrazolyl, triazolyl and tetrazolyl, wherein the heteroaryl is optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group. It may be that Q³ is bonded to Q²-L³- by a ring carbon atom in Q³. It may be that Q³ is bonded to Q²-L³- by a ring nitrogen atom in Q³ (where chemically possible).
122. Q³ is a 6-membered heteroaryl group containing at least one ring nitrogen atom, wherein the heteroaryl is optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group.
123. Q³ is a 6-membered heteroaryl group, wherein said heteroaryl group contains from 1, 2 or 3 ring nitrogen atoms, wherein the heteroaryl is optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group.
124. Q³ is a 6-membered heteroaryl group selected from pyridyl and pyrimidyl, wherein said heteroaryl group contains 1 to 3 ring nitrogen atoms, wherein the heteroaryl is optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group.
125. Q³ is pyrimidyl optionally substituted with one or more R⁷ group. Thus it may be that Q³ is unsubstituted. Thus, it may be that Q³ is substituted by 1 or 2, R⁷ group. Thus, it may be that Q³ is substituted by 1 R⁷ group.
126. Q³ has the structure:

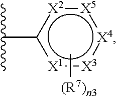

wherein:
   X¹, X², X³, X⁴ and X⁵ are each independently selected from CH and N, wherein at least one and no more than three of X¹, X², X³, X⁴ and X⁵ is N;
   n3 is 0, 1 or 2 (where chemically possible); and
   [symbol] indicates the point of attachment to L³ of Formula (I).
Preferably, no more than two of X¹, X², X³, X⁴ and X⁵ are N.
127. When Q³ is defined as in paragraph 126, one X group can be substituted with one R⁷ group.
128. Q³ is selected from:

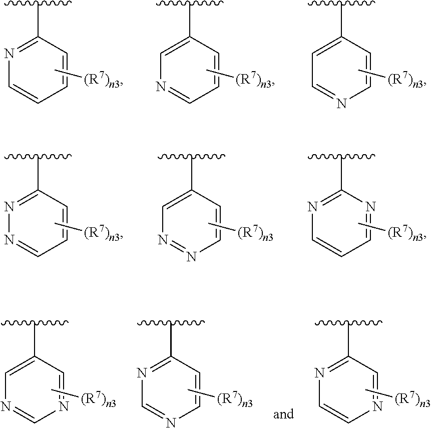

wherein n3 is 0, 1 or 2, and wherein [symbol] indicates the point of attachment to L³ of Formula (I). Typically, n3 is 0 or 1. For example, n3 is 0. For example, n3 is 1.
129. Q³ is

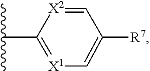

wherein: X¹ and X² are each independently selected from CH and N. Thus it may be that at least one of X¹ and X² is N; and
[symbol] indicates the point of attachment to L³ of Formula (I).
130. Q³ is selected from the group consisting of:

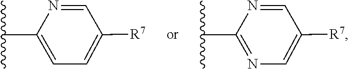

wherein [symbol] indicates the point of attachment to L³ of Formula (I).
131. Q³ is

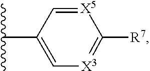

wherein: X³ and X⁵ are each independently selected from CH and N, preferably wherein at least one of X³ and X⁵ is N; and
[symbol] indicates the point of attachment to L³ of Formula (I).
132. Q³ is defined as in paragraph 131, wherein Q¹ is defined in any one of numbered paragraphs 18 to 34, R³ is absent, R² is H, the group

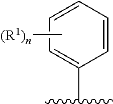

has the structure

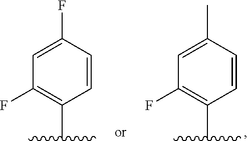

more preferably

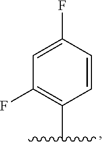 [SMILES: *c1ccc(F)cc1F]

L¹ is —CH₂—, Q² is phenyl substituted by -L³-Q³, L³ is

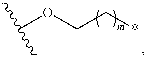

wherein: m is 0, 1 or 2; [symbol] indicates the point of attachment to Q² of Formula (I); and * indicates the point of attachment to Q³, and R⁷ is selected from H, halo and C₁₋₄ alkyl. Preferably, R⁷ is selected from H, —F and methyl.
133. Q³ is selected from the group consisting of:

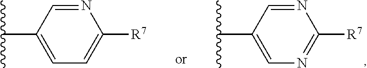

wherein [symbol] indicates the point of attachment to L³ of Formula (I).
134. Q³ is:

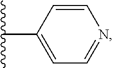 [SMILES: *c1ccncc1]

wherein [symbol] indicates the point of attachment to L³ of Formula (I).
135. Q³ has the structure:

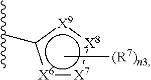

wherein:
   X⁶, X⁷, X⁸ and X⁹ are each independently selected from CH and N, wherein at least one of X⁶, X⁷, X⁸ and X⁹ is N;
n3 is 0, 1 or 2 (where chemically possible); and
[symbol] indicates the point of attachment to L³ of Formula (I).
Thus it may be that one to four of X⁶, X⁷, X⁸ and X⁹ is N. Thus it may be that one to three of X⁶, X⁷, X⁸ and X⁹ is N. Thus it may be that one or two of X⁶, X⁷, X⁸ and X⁹ is N. Thus it may be that one of X⁶, X⁷, X⁸ and X⁹ is N. Thus it may be that two of X⁶, X⁷, X⁸ and X⁹ is N. Thus it may be that three of X⁶, X⁷, X⁸ and X⁹ is N.
136. When Q³ is defined as in paragraph 135, one X group can be substituted with one R⁷ group.
137. Q³ is selected from:

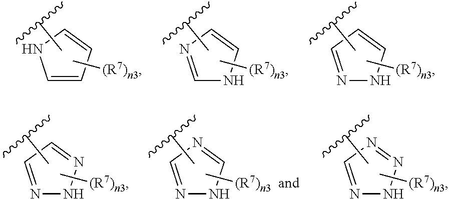

wherein n3 is 0, 1 or 2 (where chemically possible), and wherein [symbol] indicates the point of attachment to L³ of Formula (I). Typically, n3 is 0 or 1. For example, n3 is 0. For example, n3 is 1.
Thus it may be that Q³ is selected from:

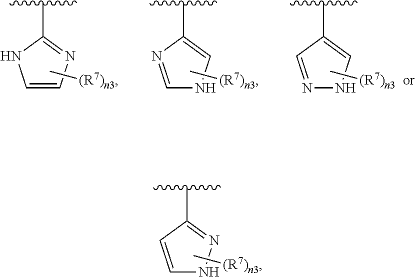

wherein n3 is 0, 1 or 2, and wherein [symbol] indicates the point of attachment to L³ of Formula (I). Typically, n3 is 0 or 1. For example, n3 is 0. For example, n3 is 1.
138. Q³ is

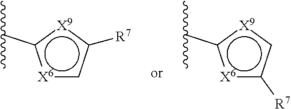

wherein:
   X⁶ and X⁹ are each independently selected from CH and N, wherein at least one of X⁶ and X⁹ is N; and
   [symbol] indicates the point of attachment to L³ of Formula (I).
139. Q³ is selected from the group consisting of:

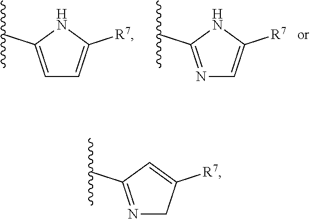

wherein [symbol] indicates the point of attachment to L³ of Formula (I).
140. Q³ is selected from the group consisting of:

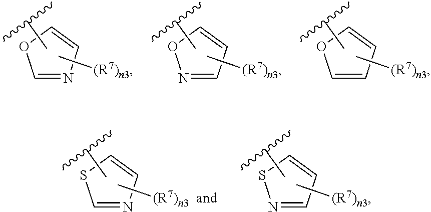

wherein n3 is 0, 1 or 2, and wherein [symbol] indicates the point of attachment to L³ of Formula (I). Typically, n3 is 0 or 1. For example, n3 is 0. For example, n3 is 1.
141. Q³ is selected from the group consisting of:

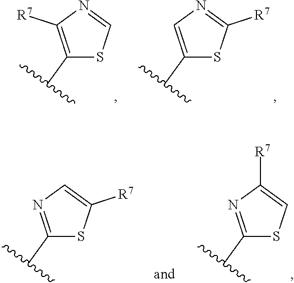

wherein [symbol] indicates the point of attachment to L³ of Formula (I).
142. R⁷ at each occurrence is independently selected from the group consisting of: halo, ═O, —CN, —NO₂, C₁₋₄ alkyl, C₂₋₄ alkenyl, C₂₋₄ alkynyl, C₁₋₄ haloalkyl, C₁₋₆ cycloalkyl, —OR^{A1}, —S(O)ₓR^{A2} (wherein x is 0, 1, or 2), —NR^{A2}R^{B2}, —C(O)R^{A2}, —OC(O)R^{A2}, —C(O)OR^{A2}, —NR^{B2}C(O)R^{A2}, —NR^{B2}C(O)OR^{A2}, —C(O)NR^{A2}R^{B2}, —NR^{B2}SO₂R^{A2} and —SO₂NR^{A2}R^{B2}.
   wherein said C₁₋₄alkyl C₂₋₄ alkenyl, C₂₋₄ alkynyl are each is optionally substituted by 1 or 2 substituents independently selected from: halo, —CN, —OR^{A3}, —NR^{A3}R^{B3} and —SO₂R^{A3}; and
   wherein said C₁₋₆ cycloalkyl is optionally substituted by one or more substituents independently selected from halo, ═O, —CN, —NO₂, C₁₋₄ alkyl and C₁₋₄ haloalkyl;
   R^{A1}, R^{A2}, R^{B2}, R^{A3} and R^{B3} are each independently selected from: H, C₁₋₄ alkyl and C₁₋₄ haloalkyl.
143. R⁷ at each occurrence is independently selected from the group consisting of: halo, ═O, —CN, —NO₂, C₁₋₄ alkyl, C₁₋₄ haloalkyl, —OR^{A1}, —S(O)ₓR^{A2} (wherein x is 0, 1, or 2) and —NR^{A2}R^{B2}.
   wherein said C₁₋₄alkyl is optionally substituted by 1 or 2 substituents independently selected from: halo, —CN, —OR^{A3}, —NR^{A3}R^{B3} and —SO₂R^{A3}; and
   R^{A1}, R^{A2}, R^{B2}, R^{A3} and R^{B3} are each independently selected from: H, C₁₋₄ alkyl and C₁₋₄ haloalkyl.
144. R⁷ at each occurrence is independently selected from the group consisting of: halo, —CN, C₁₋₄ alkyl and —OR^{A1}.
145. R⁷ is halo. For example, R⁷ is —F.
146. R⁷ is C₁₋₄ alkyl optionally substituted by 1 or 2 substituents independently selected from: halo, —CN, —OR^{A3}, —NR^{A3}R^{B3} and —SO₂R^{A3}. It may be that R⁷ is unsubstituted C₁₋₄ alkyl. It may be that R⁷ is methyl.
147. R⁷ is —CN.
148. R⁷ is —OR^{A1}, wherein R^{A1} is selected from: H, C₁₋₄ alkyl and C₁₋₄ haloalkyl. It may be that R^{A1} is C₁₋₄ alkyl. It may be that R^{A1} is methyl. Thus, it may be that R⁷ is —OCH₃.
149. Q³ is a 3- to 9-membered non-aromatic saturated or partially saturated monocyclic heterocyclyl group containing at least one ring heteroatom selected from O, S and N, wherein the heterocyclyl is optionally substituted by one or more R⁸ groups. For example Q³ is a 3- to 9-membered non-aromatic saturated monocyclic heterocyclyl group containing at least one ring heteroatom selected from O, S and N, wherein the heterocyclyl is optionally substituted by one or more R^{B} groups. Thus, it may be that Q³ is unsubstituted. It may be that Q³ is substituted by one R^{B} group.
150. Q³ is a 4- to 7-membered non-aromatic saturated or partially saturated monocyclic heterocyclyl group containing at least one ring heteroatom selected from O, S and N (e.g. 1 or 2 heteroatoms), wherein the heterocyclyl is optionally substituted by one or more R⁸ groups. For example, Q³ is a 4- to 7-membered saturated monocyclic heterocyclyl group containing at least one (e.g. 1 or 2) ring heteroatom selected from O, S and N, wherein the heterocyclyl is optionally substituted by one or more R⁸ groups. Thus, it may be that Q³ is unsubstituted. It may be that Q³ is substituted by one R^{B} group.
151. Q³ is a 4- to 7-membered non-aromatic saturated or partially saturated monocyclic heterocyclyl group containing at least one (e.g. 1 or 2) ring nitrogen atom, wherein the heterocyclyl is optionally substituted by one or more R⁸ groups. For example, Q³ is a 4- to 7-membered non-aromatic saturated or partially saturated monocyclic heterocyclyl group containing at least one ring nitrogen atom, wherein the heterocyclyl is optionally substituted by one or more R¹ groups. Preferably, Q³ is an unsubstituted 4- to 6-membered saturated monocyclic heterocyclyl group containing at least one (e.g. 1 or 2) nitrogen ring atom.
152. It may be that Q³ is a heterocyclyl selected from the group consisting of oxetanyl, tetrahydrofuranyl, tetrahydropyranyl, dioxanyl, azetidinyl, pyrrolidinyl, pyrazolidinyl, imidazolidinyl, piperidinyl, piperazinyl, morpholinyl, thiomorpholinyl, dihydroazetyl, oxetyl, pyrrolinyl, pyrazolinyl, imidazolinyl, tetrahydropyridinyl and dihydropyranyl, wherein the heterocyclyl is optionally substituted by one or more R⁸ groups. For example, Q³ is selected from the group consisting of azetidinyl, pyrrolidinyl, piperidinyl, piperazinyl and morpholinyl, wherein the heterocyclyl is optionally substituted by one or more R⁸ groups. It may be that the heterocyclyl is unsubstituted.
153. Q³ is a 7-membered heterocyclyl group containing at least one ring nitrogen atom, wherein the heterocyclyl is optionally substituted by one or more R⁸ groups. Thus, it may be that Q³ is unsubstituted.
154. Q³ is a 7-membered heterocyclyl group, wherein said heterocyclyl group contains from 1 to 3 heteroatoms selected from O, S and N.
155. Q³ is a 7-membered saturated heterocyclyl group, wherein said heterocyclyl group contains 1 ring nitrogen atom.
156. Q³ is selected from:

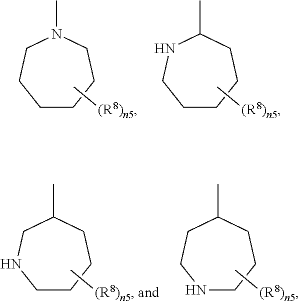

wherein:
   n5 is 0, 1 or 2 (where chemically possible); and
   [symbol] indicates the point of attachment to L³ of Formula (I).
157. Q³ is a 6-membered heterocyclyl group containing at least one ring nitrogen atom, wherein the heterocyclyl is optionally substituted by one or more R⁸ groups. Thus, it may be that Q³ is unsubstituted.
158. Q³ is a 6-membered heterocyclyl group, wherein said heterocyclyl group contains from 1 to 3 heteroatoms selected from O, S and N.
159. Q³ is a 6-membered heterocyclyl group, wherein said heterocyclyl group contains 1 ring nitrogen atom and optionally 1 or 2 ring heteroatoms selected from O, S and N.
160. Q³ is a 6-membered heterocyclyl group, wherein said heterocyclyl group contains 1 ring nitrogen atom and 1 ring heteroatom selected from O, S and N. It may be that said heterocyclyl group contains 1 ring nitrogen atom and 1 ring oxygen atom.
161. Q³ is a 6-membered saturated heterocyclyl group, wherein said heterocyclyl group contains 1 ring nitrogen atom.
162. Q³ is selected from:

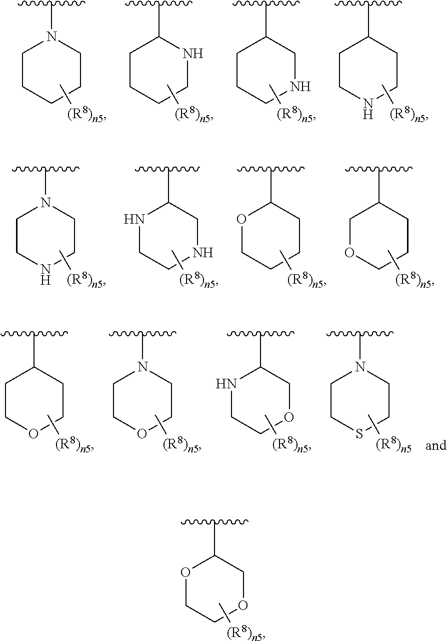

wherein:
   n5 is 0, 1 or 2 (where chemically possible); and
   [symbol] indicates the point of attachment to L³ of Formula (I). Thus it may be that Q³ is selected from:

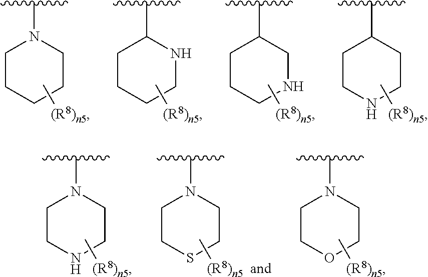

wherein n5 is 0, 1 or 2.
163. Q³ is:

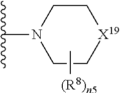

wherein:
   X¹⁹ is selected from NH, S, CH₂ and 0;
   n5 is 0, 1 or 2 (e.g. n5 is 0 or 1); and
   [symbol] indicates the point of attachment to L³ of Formula (I).
Thus it may be that X¹⁹ is selected from N, O and CH₂.
164. Q³ is selected from the group consisting of:

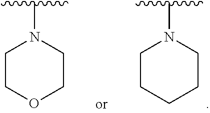 [SMILES: *N1CCOCC1 ; *N1CCCCC1]

165. Q³ is:

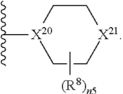

wherein:
   X²⁰ is selected from N and CH;
   X²¹ is selected from NH, S, CH₂ and 0;
   n5 is 0, 1 or 2 (e.g. n5 is 0 or 1); and
   [symbol] indicates the point of attachment to L³ of Formula (I).
Thus, it may be that X²¹ is selected from N, O and CH₂.
166. Q³ is selected from the group consisting of:

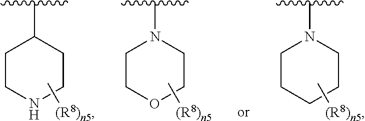

wherein n5 is 0, 1 or 2 (e.g. n5 is 0 or 1); and
   [symbol] indicates the point of attachment to L³ of Formula (I).
Thus, it may be that Q³ is selected from the group consisting of:

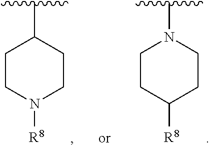

167. Q³ is a 5-membered heterocyclyl group containing at least one ring nitrogen atom, wherein the heterocyclyl is optionally substituted by one or more R⁸ groups. Thus, it may be that Q³ is unsubstituted.
168. Q³ is a 5-membered heterocyclyl group, wherein said heterocyclyl group contains from 1 to 2 heteroatoms selected from O, S and N.
169. Q³ is a 5-membered heterocyclyl group, wherein said heterocyclyl group contains 1 ring nitrogen atom and optionally 1 ring heteroatom selected from O, S and N.
170. Q³ is a 5-membered heterocyclyl group, wherein said heterocyclyl group contains 1 ring nitrogen atom and 1 ring heteroatom selected from O, S and N.
171. Q³ is a 5-membered heterocyclyl group, wherein said heterocyclyl group contains 1 ring nitrogen atom.
172. Q³ is selected from:

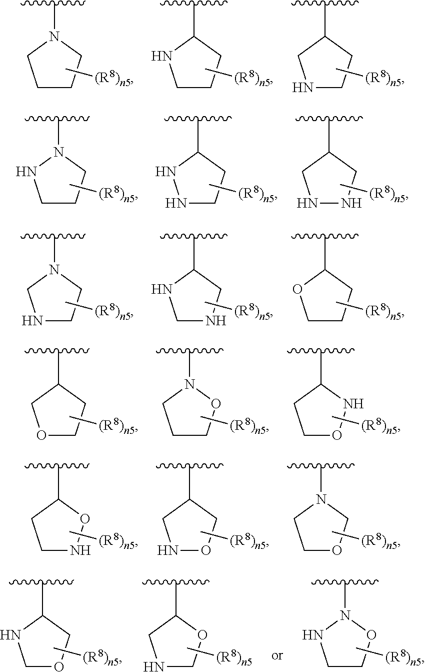

wherein
n5 is 0, 1 or 2 (where chemically possible); and
[symbol] indicates the point of attachment to L³ of Formula (I). Thus it may be that Q³ is

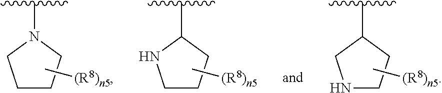

173. Q³ is a 4-membered heterocyclyl group containing one ring heteroatom selected from O and N, wherein the heterocyclyl is optionally substituted by 1 or 2 R⁸ groups.
174. Q³ is an unsubstituted 4-membered heterocyclyl group, wherein said heterocyclyl group contains from 1 heteroatom selected from 0 and N.
175. Q³ is azetidinyl.
176. Q³ is azetidinyl substituted by 1 R⁸ group.
177. Q³ is an oxetanyl optionally substituted by 1 R⁸ group.
178. Q³ is selected from:

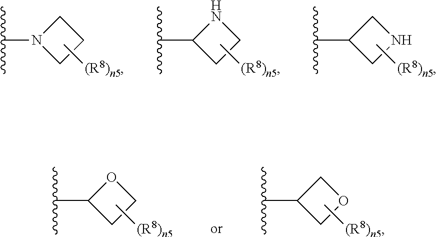

wherein:
n5 is 0, 1 or 2 (where chemically possible); and
[symbol] indicates the point of attachment to L³ of Formula (I). Thus it may be that n5 is 0. Thus it may be that n5 is 1.
179. R⁸ at each occurrence is independently selected from the group consisting of: halo, ═O, —CN, —NO₂, C₁₋₄ alkyl, C₂₋₄ alkenyl, C₂₋₄ alkynyl, C₁₋₄ haloalkyl, C₁₋₆ cycloalkyl, —OR^{A1}, —S(O)ₓR^{A2} (wherein x is 0, 1, or 2), —NR^{A2}R^{B2}, —C(O)R^{A2}, —OC(O)R^{A2}, —C(O)OR^{A2}, —NR^{B2}C(O)R^{A2}, —NR^{B2}C(O)OR^{A2}, —C(O)NR^{A2}R^{B2}, —NR^{B2}SO₂R^{A2} and —SO₂NR^{A2}R^{B2}.
   wherein said C₁₋₄alkyl C₂₋₄ alkenyl, C₂₋₄ alkynyl are each is optionally substituted by 1 or 2 substituents independently selected from: halo, —CN, —OR^{A3}, —NR^{A3}R^{B3} and —SO₂R^{A3}; and
   wherein said C₁₋₃ cycloalkyl is optionally substituted by one or more substituents independently selected from halo, ═O, —CN, —NO₂, C₁₋₄ alkyl and C₁₋₄ haloalkyl;
   R^{A1}, R^{A2}, R^{B2}, R^{A3} and R^{B3} are each independently selected from: H, C₁₋₄ alkyl and C₁₋₄ haloalkyl.
180. R⁸ at each occurrence is independently selected from the group consisting of: halo, ═O, —CN, —NO₂, C₁₋₄ alkyl, C₁₋₄ haloalkyl, —OR^{A1}, —S(O)ₓR^{A2} (wherein x is 0, 1, or 2) and —NR^{A2}R^{B2}.
   wherein said C₁₋₄alkyl is optionally substituted by 1 or 2 substituents independently selected from: halo, —CN, —OR^{A3}, —NR^{A3}R^{B3} and —SO₂R^{A3}; and
   R^{A1}, R^{A2}, R^{B2}, R^{A3} and R^{B3} are each independently selected from: H, C₁₋₄ alkyl and C₁₋₄ haloalkyl.
181. R⁸ at each occurrence is independently selected from the group consisting of: halo, —CN, C₁₋₄ alkyl and —OR^{A1}.
182. R⁸ at each occurrence is independently selected from the group consisting of: halo and C₁₋₃ alkyl.
183. R⁸ is halo. For example, R⁸ is —F.
184. R⁸ is C₁₋₄ alkyl optionally substituted by 1 or 2 substituents independently selected from: halo, —CN, —OR^{A3}, —NR^{A3}R^{B3} and —SO₂R^{A3}. It may be that R⁸ is unsubstituted C₁₋₄alkyl. It may be that R^{e} is methyl.
185. R⁸ is —CN.
186. R⁸ is ═O.
187. R⁸ is —OR^{A1}, wherein R^{A1} is selected from: H, C₁₋₄ alkyl and C₁₋₄ haloalkyl. It may be that R^{A1} is C₁₋₄ alkyl. It may be that R^{A1} is methyl. Thus, it may be that R⁸ is —OCH₃.
188. R⁸ is absent.
189. The group -Q² is selected from the group consisting of:

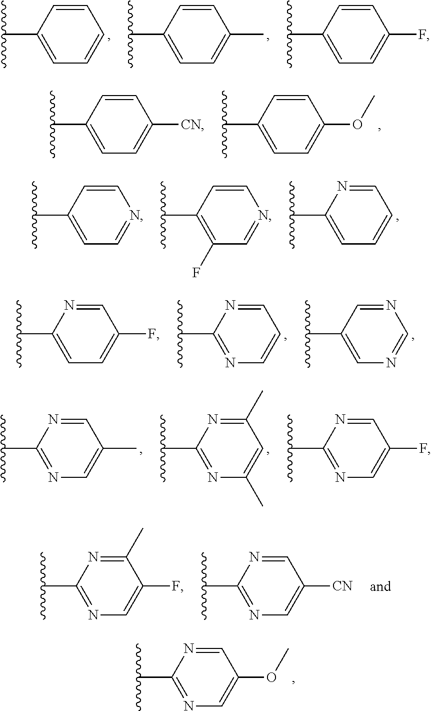

wherein [symbol] indicates the point of attachment to L¹ of Formula (I).
190. The group -Q² is selected from the group consisting of:

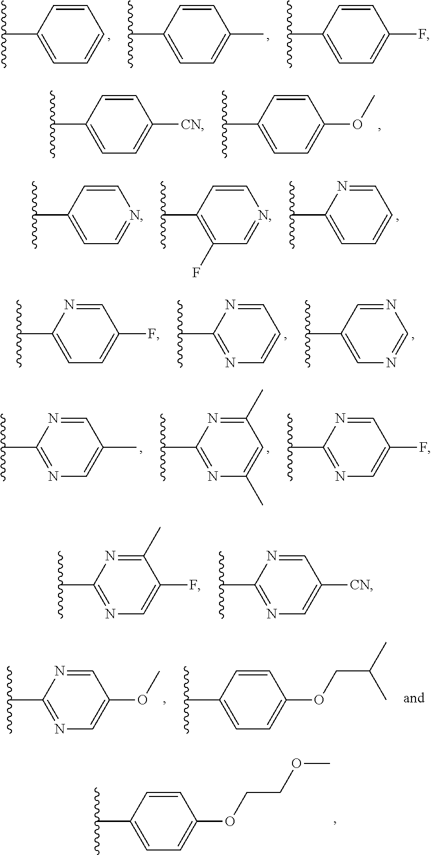

wherein [symbol] indicates the point of attachment to L¹ of Formula (I).
191. The group -Q²-L³-Q³ is:

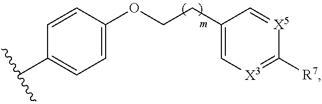

wherein:
   m is 0, 1 or 2;
   X³ and X⁵ are each independently selected from CH and N, optionally wherein at least one of X³ and X⁵ is N; and
   R⁷ is selected from H, halo or C₁₋₄alkyl.
Preferably, it may be that the group -Q²-L³-Q³ is selected from:

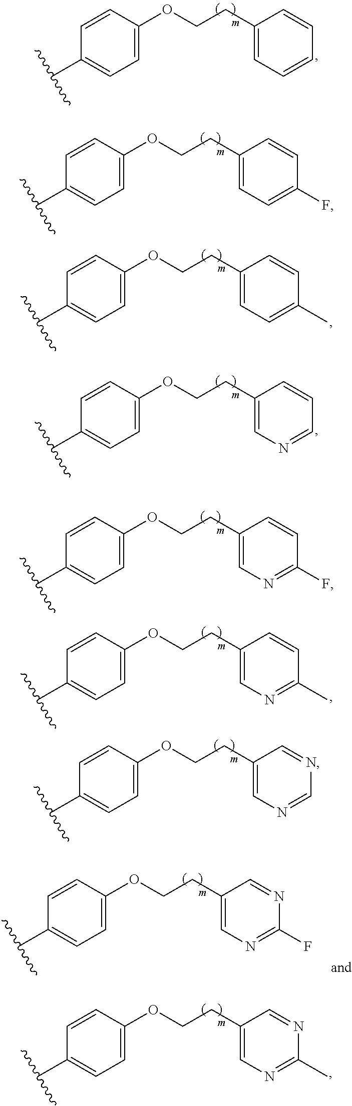

wherein:
   m is 0, 1 or 2; and
   [symbol] indicates the point of attachment to Q² of Formula (I).
In this paragraph it may be the L¹ is —CH₂—. In this paragraph it may be the L¹ is a bond. In this paragraph it may be the L¹ is —NH—.
192. The group -Q²-L³-Q³ is selected from the group consisting of:
wherein:

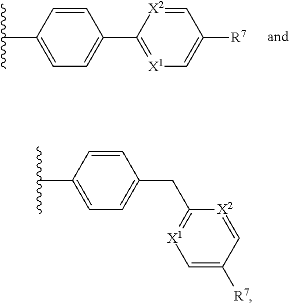

X¹ and X² are each independently selected from CH and N;
[symbol] indicates the point of attachment to L¹ of Formula (I); and
R⁷ is selected from the group consisting of halo, —CN, C₁₋₄ alkyl and —ORA.
Preferably, R⁷ is selected from the group consisting of —F, —CN, -methyl and —OCH₃.
193. The group -Q²-L³-Q³ is selected from the group consisting of:

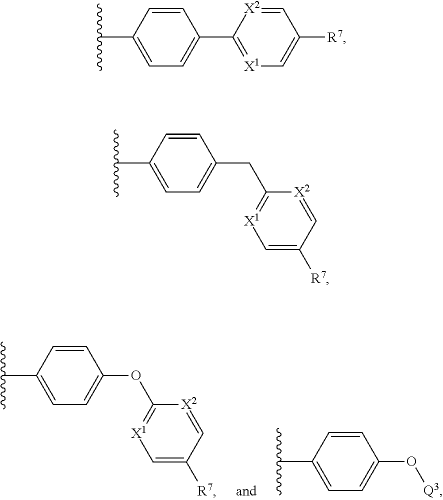

wherein:
   X¹ and X² are each independently selected from CH and N;
   [symbol] indicates the point of attachment to L¹ of Formula (I); and
   R⁷ is selected from the group consisting of halo, —CN, C₁₋₄ alkyl and —OR^{A1}.
Preferably, R⁷ is selected from the group consisting of —F, —CN, -methyl and —OCH₃.
194. The group -Q²-L³-Q³ is selected from the group consisting of:

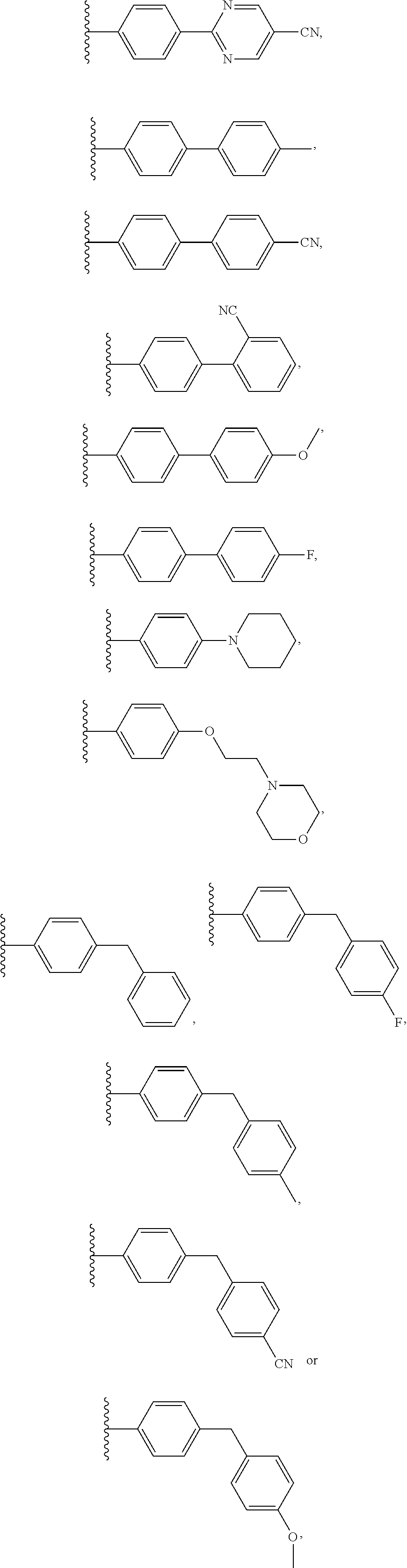

wherein [symbol] indicates the point of attachment to L¹ of Formula (I). Thus it may be that the group -Q²-L³-Q³ is selected from the group consisting of:

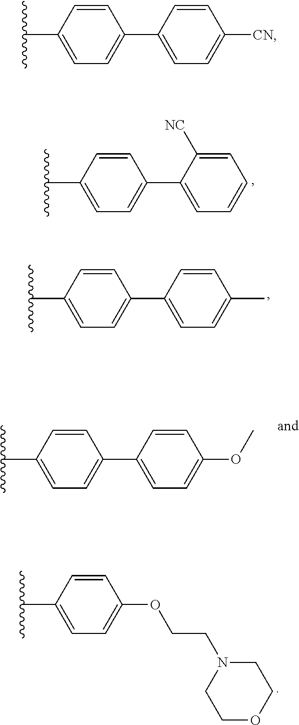 [SMILES: *c1ccc(-c2ccc(C#N)cc2)cc1 ; *c1ccc(-c2ccccc2C#N)cc1 ; *c1ccc(-c2ccc(C)cc2)cc1 ; *c1ccc(-c2ccc(OC)cc2)cc1 ; *c1ccc(OCCN2CCOCC2)cc1]

195. The group -Q²-L³-Q³ is selected from the group consisting of:

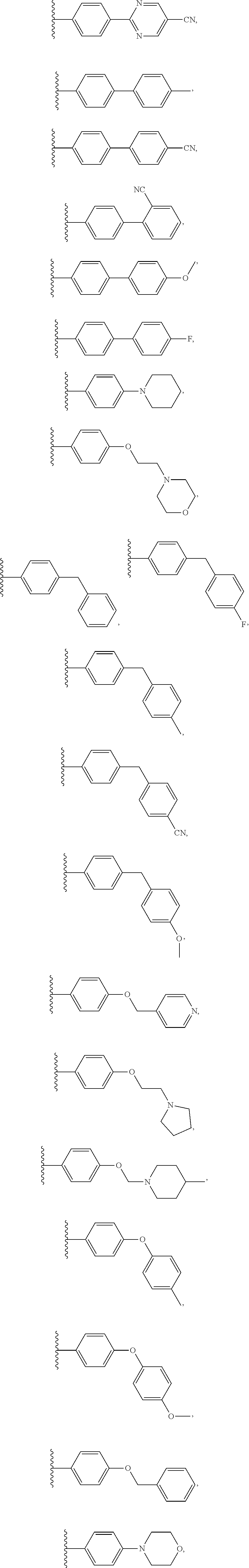

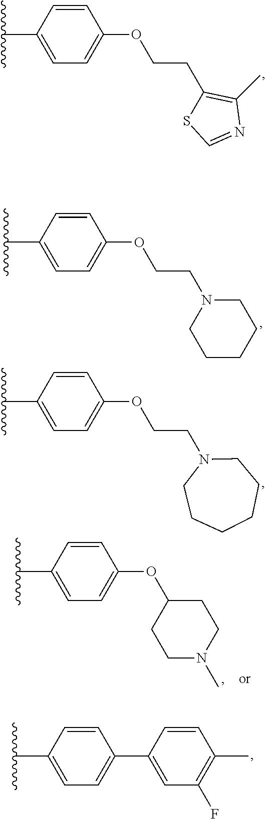

wherein [symbol] indicates the point of attachment to L¹ of Formula (I).
196. The group -Q²-L³-Q³ is selected from the group consisting of:

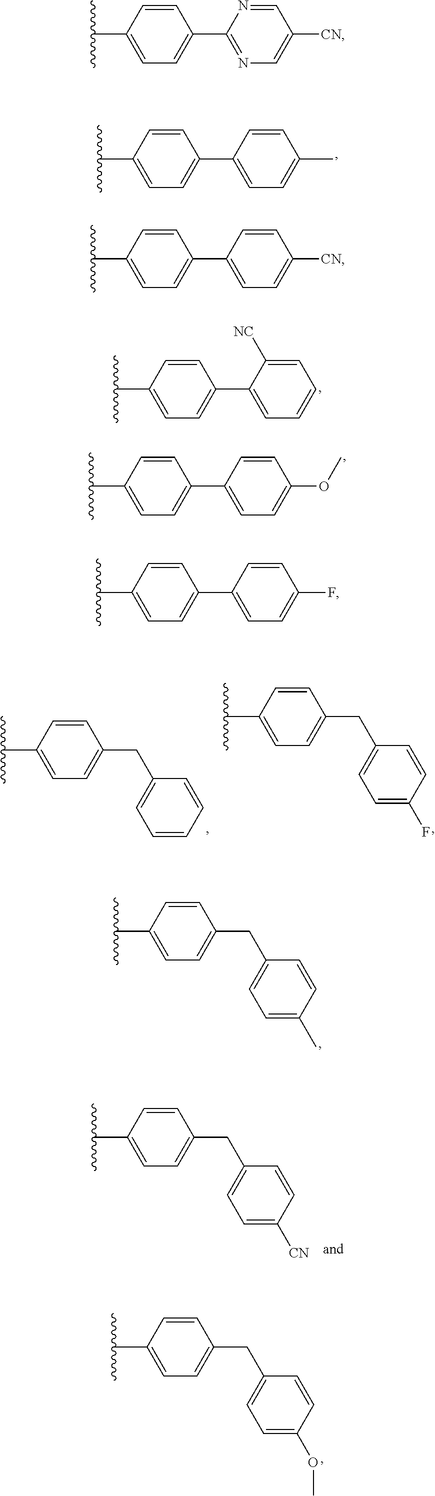

wherein [symbol] indicates the point of attachment to L¹ of Formula (I).
197. The group -Q²-L³-Q³ is selected from the group consisting of:

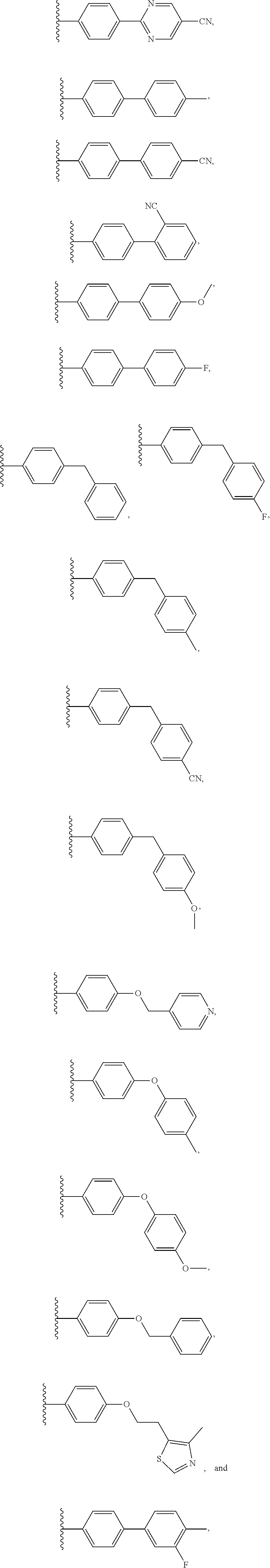

wherein [symbol] indicates the point of attachment to L¹ of Formula (I).
198. The group Q²-L³-Q³ is selected from the group consisting of:

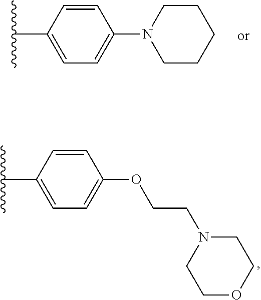 [SMILES: *c1ccc(N2CCCCC2)cc1 ; *c1ccc(OCCN2CCOCC2)cc1]

wherein [symbol] indicates the point of attachment to L¹ of Formula (I).
199. The group Q²-L³-Q³ is selected from the group consisting of:

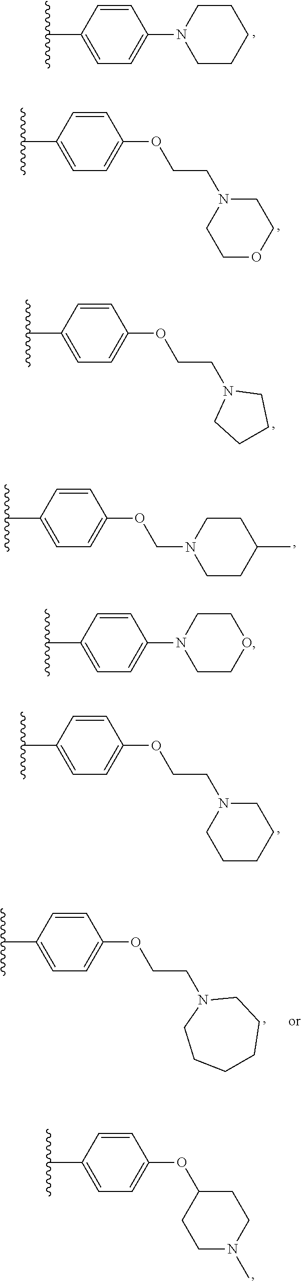

wherein [symbol] indicates the point of attachment to L¹ of Formula (I).
200. The group -L¹-Q²-L³-Q³ is selected from the group consisting of:

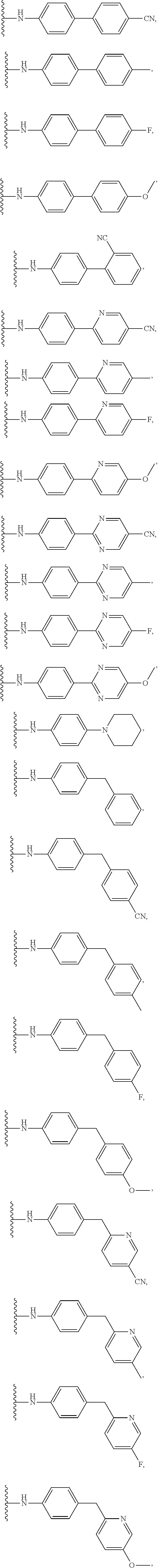

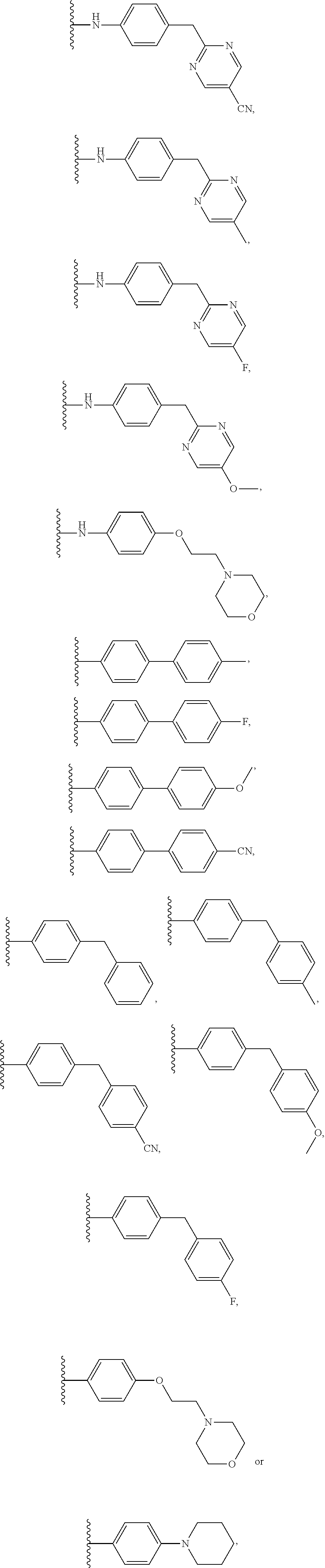

wherein [symbol] indicates the point of attachment to Q¹ of Formula (I).
201. The group -L¹-Q²-L³-Q³ is selected from the group consisting of:

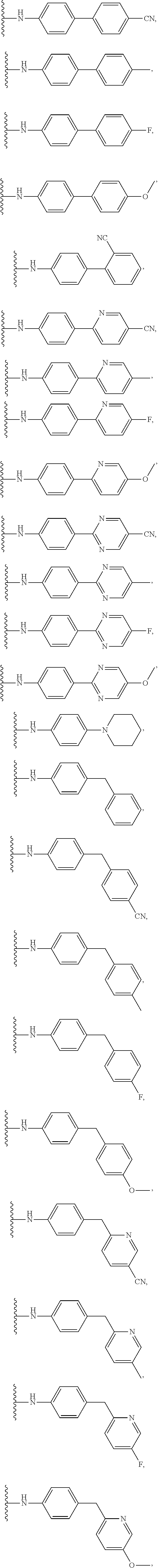

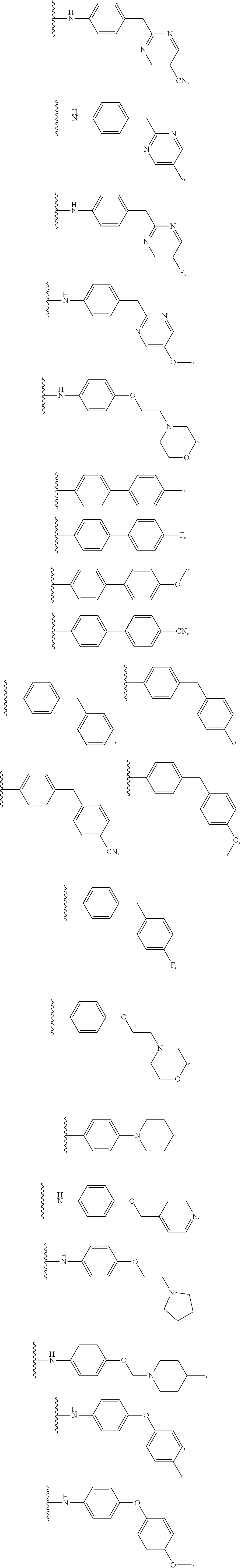

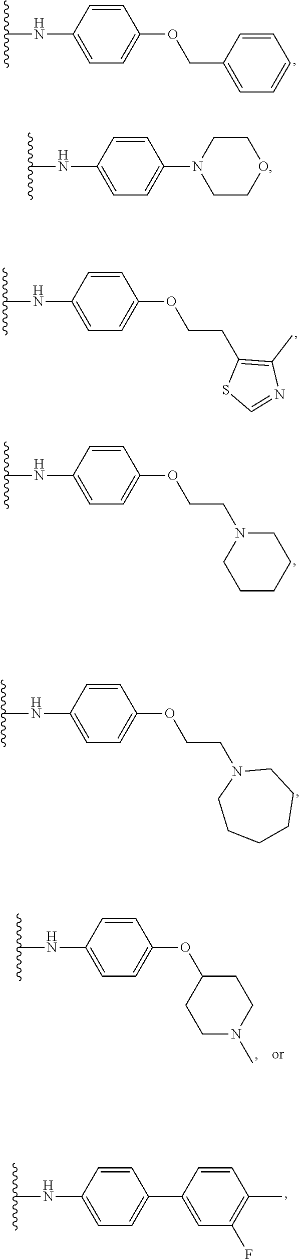

wherein [symbol] indicates the point of attachment to Q¹ of Formula (I).
202. In some embodiments, the group -Q²-L³-Q³ is as defined in numbered paragraph 193, wherein L¹ is —NH₂— and Q¹ is selected from the group consisting of Ia, Ib, Ic, le, Ig and Ik.
203. In some embodiments, the group -Q²-L³-Q³ is as defined in numbered paragraph 195, wherein L¹ is —NH₂— and Q¹ is selected from the group consisting of Ia, Ib, Ic, Ie, Ig and Ik.
204. In some embodiments, the group -Q²-L³-Q³ is as defined in numbered paragraph 193, wherein L¹ is a bond and Q¹ is selected from the group consisting of Id, If, Ih, Ii and Ij.
205. In some embodiments, the group -Q²-L³-Q³ is as defined in numbered paragraph 195, wherein L¹ is a bond and Q¹ is selected from the group consisting of Id, If, Ih, Ii and Ij.
206. In some embodiments, Q¹ is Ia, L¹ is —NH— and Q² is as defined in numbered paragraph 97.
207. In some embodiments, Q¹ is Ia, L¹ is —NH—, Q² is as defined in numbered paragraph 97, wherein L₃ is —CH₂— and Q³ is phenyl.
208. In some embodiments, Q¹ is Ia, L¹ is —NH— and Q² is as defined in numbered paragraph 49.
209. In some embodiments, Q¹ is Ia, L¹ is —NH—, Q² is as defined in numbered paragraph 49, wherein R⁶ is as defined in numbered paragraph 93.
210. In some embodiments, Q¹ is Ia, L¹ is —NH—, Q² is as defined in numbered paragraph 49, wherein R⁶ is as defined in numbered paragraph 95.
211. In some embodiments, Q¹ is Ia, L¹ is —NH—, Q² is as defined in numbered paragraph 97, wherein L₃ is a bond, and Q³ is as defined in one of paragraphs 112 to 178 (e.g. Q³ is as defined in numbered paragraph 114 and R⁷ is as defined in one of numbered paragraphs 144 to 148).
212. In some embodiments, Q¹ is Ia, L¹ is —NH—, Q² is as defined in numbered paragraph 97, wherein L₃ is a bond, and Q³ is as defined in one of paragraphs 156 to 172 (e.g. Q³ is as defined in numbered paragraph 164).
213. In some embodiments, Q¹ is Ia, L¹ is —NH—, Q² is as defined in numbered paragraph 97, wherein L₃ is —CH₂—, and Q³ is as defined in one of paragraphs 112 to 178 (e.g. Q³ is as defined in numbered paragraph 114 and R⁷ is as defined in one of numbered paragraphs 144 to 148).
214. In some embodiments, Q¹ is Ia, L¹ is —NH—, Q² is as defined in numbered paragraph 97, wherein L₃ is a bond, and Q³ is as defined in one of paragraphs 112 to 178 (e.g. Q³ is as defined in numbered paragraph 164).
215. In some embodiments, Q¹ is Ia, L¹ is —NH—, Q² is as defined in numbered paragraph 97, wherein L₃ is

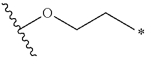 [SMILES: *OCCC]

and Q³ is as defined in one of paragraphs 112 to 178, for example Q³ is as defined in paragraph 164.
216. In some embodiments, Q¹ is Ia, L¹ is —NH—, Q² is as defined in numbered paragraph 97, wherein L₃ is

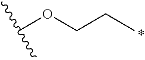 [SMILES: *OCCC]

and Q³ is as defined in numbered paragraphs 156, 164 or 172.
217. In some embodiments, Q¹ is Ia, L¹ is —NH—, Q² is as defined in numbered paragraph 97, wherein L₃ is

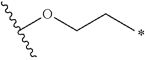 [SMILES: *OCCC]

and Q³ is as defined in numbered paragraph 141, and R⁷ is as defined in any one of paragraphs 142 to 148, for example paragraph 146.
218. In some embodiments, Q¹ is Ia, L¹ is —CH₂—, Q² is phenyl, L₃ is as defined in numbered paragraph 109 and Q³ is as defined in paragraph 112.
219. In some embodiments, Q¹ is Ia, L¹ is —CH₂—, Q² is phenyl, L₃ is as defined in numbered paragraph 109 and Q³ is as defined in paragraph 131.
220. In some embodiments, Q¹ is Ia, L¹ is —CH₂—, Q² is phenyl, L₃ is as defined in numbered paragraph 107 and Q³ is as defined in paragraph 131.
221. In some embodiments, Q¹ is Ia, L¹ is —CH₂—, Q² is phenyl, L₃ is as defined in numbered paragraph 107 and Q³ is as defined in paragraph 166.
222. In some embodiments, Q¹ is Ia, L¹ is —CH₂—, Q² is phenyl, L₃ is as defined in numbered paragraph 109 and Q³ is as defined in paragraph 134.
223. In some embodiments, Q¹ is Ia, L¹ is —CH₂—, Q² is phenyl, L₃ is as defined in numbered paragraph 109 and Q³ is as defined in paragraph 166.
224. In some embodiments, Q¹ is Ib, L¹ is —NH— and Q² is phenyl.
225. In some embodiments, Q¹ is Ib, L¹ is —NH— and Q² is as defined in numbered paragraph 67.
226. In some embodiments, Q¹ is Ib, L¹ is —NH— and Q² is as defined in one of numbered paragraphs 84, 87, 89 and 91.
227. In some embodiments, Q¹ is Ib, L¹ is —NH— and Q² is as defined in numbered paragraph 97.
228. In some embodiments, Q¹ is Ib, L¹ is —NH—, Q² is as defined in numbered paragraph 97, wherein L₃ is —CH₂— and Q³ is phenyl.
229. In some embodiments, Q¹ is Ib, L¹ is —NH—, Q² is as defined in numbered paragraph 97, wherein L₃ is a bond, and Q³ is as defined in one of paragraphs 112 to 178, for example Q³ is as defined in numbered paragraph 114 and R⁷ is as defined in numbered paragraphs 144 to 148.
230. In some embodiments, Q¹ is Ib, L¹ is —NH—, Q² is as defined in numbered paragraph 97, wherein L₃ is —CH₂—, and Q³ is as defined in one of paragraphs 112 to 178, for example Q³ is as defined in numbered paragraph 114 and R⁷ is as defined in numbered paragraphs 144 to 148.
231. In some embodiments, Q¹ is Ib, L¹ is —NH—, Q² is as defined in numbered paragraph 97, wherein L₃ is a bond, and Q³ is as defined in one of paragraphs 112 to 178, for example Q³ is as defined in numbered paragraph 164.
232. In some embodiments, Q¹ is Ib, L¹ is —NH—, Q² is as defined in numbered paragraph 97, wherein L₃ is

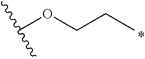 [SMILES: *OCCC]

and Q³ is as defined in one of paragraphs 112 to 178, for example Q³ is as defined in paragraph 164.
233. In some embodiments, Q¹ is Ib, L¹ is —CH₂—, Q² is phenyl, L₃ is as defined in numbered paragraph 109 and Q³ is as defined in paragraph 131.
234. In some embodiments, Q¹ is Ic, L¹ is —NH— and Q² is as defined in numbered paragraph 67.
235. In some embodiments, Q¹ is Ic, L¹ is —NH— and Q² is as defined in one of numbered paragraphs 84, 87, 89 and 91.
236. In some embodiments, Q¹ is Ic, L¹ is —NH— and Q² is as defined in numbered paragraph 97.
237. In some embodiments, Q¹ is Ic, L¹ is —NH—, Q² is as defined in numbered paragraph 97, wherein L₃ is —CH₂— and Q³ is phenyl.
238. In some embodiments, Q¹ is Ic, L¹ is —NH—, Q² is as defined in numbered paragraph 97, wherein L₃ is a bond, and Q³ is as defined in one of paragraphs 112 to 178, for example Q³ is as defined in numbered paragraph 114 and R⁷ is as defined in numbered paragraphs 144 to 148.
239. In some embodiments, Q¹ is Ic, L¹ is —NH—, Q² is as defined in numbered paragraph 97, wherein L₃ is —CH₂—, and Q³ is as defined in one of paragraphs 112 to 178, for example Q³ is as defined in numbered paragraph 114 and R⁷ is as defined in one of numbered paragraphs 144 to 148.
240. In some embodiments, Q¹ is Ic, L¹ is —NH—, Q² is as defined in numbered paragraph 97, wherein L₃ is a bond and Q³ is as defined in one of paragraphs 112 to 178, for example Q³ is as defined in numbered paragraph 164.
241. In some embodiments, Q¹ is Ic, L¹ is —NH—, Q² is as defined in numbered paragraph 97, wherein L₃ is

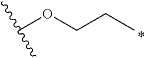 [SMILES: *OCCC]

and Q³ is as defined in one of paragraphs 112 to 178, for example Q³ is as defined in paragraph 164.
242. In some embodiments, Q¹ is Ic, L¹ is —CH₂—, Q² is phenyl, L₃ is as defined in numbered paragraph 109 and Q³ is as defined in paragraph 131.
243. In some embodiments, Q¹ is Id, L¹ is a bond and Q² is as defined in numbered paragraph 67.
244. In some embodiments, Q¹ is Id, L¹ is a bond and Q² is as defined in one of numbered paragraphs 84, 87, 89 and 91.
245. In some embodiments, Q¹ is Id, L¹ is a bond and Q² is as defined in numbered paragraph 97.
246. In some embodiments, Q¹ is Id, L¹ is a bond, Q² is as defined in numbered paragraph 97, wherein L₃ is —CH₂— and Q³ is phenyl.
247. In some embodiments, Q¹ is Id, L¹ is a bond, Q² is as defined in numbered paragraph 97, wherein L₃ is a bond, and Q³ is as defined in one of paragraphs 112 to 178, for example Q³ is as defined in numbered paragraph 114 and R⁷ is as defined in one of numbered paragraphs 144 to 148.
248. In some embodiments, Q¹ is Id, L¹ is a bond, Q² is as defined in numbered paragraph 97, wherein L₃ is —CH₂—, and Q³ is as defined in one of paragraphs 112 to 178, for example Q³ is as defined in numbered paragraph 114 and R⁷ is as defined in one of numbered paragraphs 144 to 148.
249. In some embodiments, Q¹ is Id, L¹ is a bond, Q² is as defined in numbered paragraph 97, wherein L₃ is a bond and Q³ is as defined in one of paragraphs 112 to 178, for example Q³ is as defined in numbered paragraph 164.
250. In some embodiments, Q¹ is Id, L¹ is a bond, Q² is as defined in numbered paragraph 97, wherein L₃ is

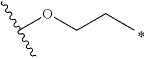 [SMILES: *OCCC]

and Q³ is as defined in one of paragraphs 112 to 178, for example Q³ is as defined in paragraph 164.
251. In some embodiments, Q¹ is Id, L¹ is —CH₂—, Q² is phenyl, L₃ is as defined in numbered paragraph 109 and Q³ is as defined in paragraph 131.
252. In some embodiments, Q¹ is Ie, L¹ is —NH— and Q² is as defined in numbered paragraph 67.
253. In some embodiments, Q¹ is Ie L¹ is —NH— and Q² is as defined in one of numbered paragraphs 84, 87, 89 and 91.
254. In some embodiments, Q¹ is Ie, L¹ is —NH— and Q² is as defined in numbered paragraph 97.
255. In some embodiments, Q¹ is Ie, L¹ is —NH—, Q² is as defined in numbered paragraph 97, wherein L₃ is —CH₂— and Q³ is phenyl.
256. In some embodiments, Q¹ is Ie, L¹ is —NH—, Q² is as defined in numbered paragraph 97, wherein L₃ is a bond, and Q³ is as defined in one of paragraphs 112 to 178, for example Q³ is as defined in numbered paragraph 114 and R⁷ is as defined in one of numbered paragraphs 144 to 148.
257. In some embodiments, Q¹ is Ie, L¹ is —NH—, Q² is as defined in numbered paragraph 97, wherein L₃ is —CH₂—, and Q³ is as defined in one of paragraphs 112 to 178, for example, Q³ is as defined in numbered paragraph 114 and R⁷ is as defined in one of numbered paragraphs 144 to 148.
258. In some embodiments, Q¹ is Ie, L¹ is —NH—, Q² is as defined in numbered paragraph 97, wherein L₃ is a bond and Q³ is as defined in one of paragraphs 112 to 178, for example Q³ is as defined in numbered paragraph 164.
259. In some embodiments, Q¹ is Ie, L¹ is —NH—, Q² is as defined in numbered paragraph 97, wherein L₃ is

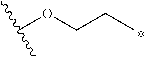 [SMILES: *OCCC]

and Q³ is as defined in one of paragraphs 112 to 178, for example Q³ is as defined in paragraph 164.
260. In some embodiments, Q¹ is Ie, L¹ is —CH₂—, Q² is phenyl, L₃ is as defined in numbered paragraph 109 and Q³ is as defined in paragraph 131.
261. In some embodiments, Q¹ is If, L¹ is a bond and Q² is as defined in numbered paragraph 67.
262. In some embodiments, Q¹ is If, L¹ is a bond and Q² is as defined in one of numbered paragraphs 84, 87, 89 and 91.
263. In some embodiments, Q¹ is If, L¹ is a bond and Q² is as defined in numbered paragraph 97.
264. In some embodiments, Q¹ is If, L¹ is a bond, Q² is as defined in numbered paragraph 97, wherein L₃ is —CH₂— and Q³ is phenyl.
265. In some embodiments, Q¹ is If, L¹ is a bond, Q² is as defined in numbered paragraph 97, wherein L₃ is a bond, and Q³ is as defined in one of paragraphs 112 to 178, for example Q³ is as defined in numbered paragraph 114 and R⁷ is as defined in one of numbered paragraphs 144 to 148.
266. In some embodiments, Q¹ is If, L¹ is a bond, Q² is as defined in numbered paragraph 97, wherein L₃ is —CH₂—, and Q³ is as defined in one of paragraphs 112 to 178, for example Q³ is as defined in numbered paragraph 114 and R⁷ is as defined in one of numbered paragraphs 144 to 148.
267. In some embodiments, Q¹ is If, L¹ is a bond, Q² is as defined in numbered paragraph 97, wherein L₃ is a bond and Q³ is as defined in one of paragraphs 112 to 178, for example Q³ is as defined in numbered paragraph 164.
268. In some embodiments, Q¹ is If, L¹ is a bond, Q² is as defined in numbered paragraph 97, wherein L₃ is

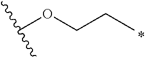 [SMILES: *OCCC]

and Q³ is as defined in one of paragraphs 112 to 178, for example Q³ is as defined in paragraph 164.
269. In some embodiments, Q¹ is If, L¹ is a bond, Q² is as defined in numbered paragraph 97, wherein L₃ is

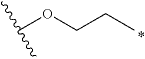 [SMILES: *OCCC]

### and Q³ is

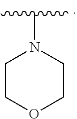 [SMILES: *N1CCOCC1] [0940] 270. In some embodiments, Q¹ is If, L¹ is —CH₂—, Q² is phenyl, L₃ is as defined in numbered paragraph 109 and Q³ is as defined in paragraph 131.
271. In some embodiments, Q¹ is Ig, L¹ is —NH— and Q² is as defined in numbered paragraph 67.
272. In some embodiments, Q¹ is Ig, L¹ is —NH— and Q² is as defined in one of numbered paragraphs 84, 87, 89 and 91.
273. In some embodiments, Q¹ is Ig, L¹ is —NH— and Q² is as defined in numbered paragraph 97.
274. In some embodiments, Q¹ is Ig, L¹ is —NH—, Q² is as defined in numbered paragraph 97, wherein L₃ is —CH₂— and Q³ is phenyl.
275. In some embodiments, Q¹ is Ig, L¹ is —NH—, Q² is as defined in numbered paragraph 97, wherein L₃ is a bond, and Q³ is as defined in one of paragraphs 112 to 178, for example Q³ is as defined in numbered paragraph 114 and R⁷ is as defined in one of numbered paragraphs 144 to 148.
276. In some embodiments, Q¹ is Ig, L¹ is —NH—, Q² is as defined in numbered paragraph 97, wherein L₃ is a bond, and Q³ is as defined in one of paragraphs 112 to 178, for example Q³ is as defined in numbered paragraph 164.
277. In some embodiments, Q¹ is Ig, L¹ is —NH—, Q² is as defined in numbered paragraph 97, wherein L₃ is —CH₂—, and Q³ is as defined in one of paragraphs 112 to 178, for example Q³ is as defined in numbered paragraph 114 and R⁷ is as defined in one of numbered paragraphs 144 to 148.
278. In some embodiments, Q¹ is Ig, L¹ is —NH—, Q² is as defined in numbered paragraph 97, wherein L₃ is a bond and Q³ is as defined in one of paragraphs 112 to 178, for example Q³ is as defined in numbered paragraph 164.
279. In some embodiments, Q¹ is Ig, L¹ is —NH—, Q² is as defined in numbered paragraph 97, wherein L₃ is

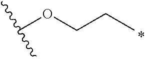 [SMILES: *OCCC]

and Q³ is as defined in one of paragraphs 112 to 178, for example Q³ is as defined in paragraph 164.
280. In some embodiments, Q¹ is Ig, L¹ is —CH₂—, Q² is phenyl, L₃ is as defined in numbered paragraph 109 and Q³ is as defined in paragraph 131.
281. In some embodiments, Q¹ is Ih, L¹ is —CH₂— and Q² is phenyl.
282. In some embodiments, Q¹ is Ih, L¹ is a bond and Q² is as defined in numbered paragraph 67.
283. In some embodiments, Q¹ is Ih, L¹ is a bond and Q² is as defined in one of numbered paragraphs 84, 87, 89 and 91.
284. In some embodiments, Q¹ is Ih, L¹ is a bond and Q² is as defined in numbered paragraph 97.
285. In some embodiments, Q¹ is Ih, L¹ is a bond, Q² is as defined in numbered paragraph 97, wherein L₃ is —CH₂— and Q³ is phenyl.
286. In some embodiments, Q¹ is Ih, L¹ is a bond, Q² is as defined in numbered paragraph 97, wherein L₃ is a bond, and Q³ is as defined in one of paragraphs 112 to 178, for example Q³ is as defined in numbered paragraph 114 and R⁷ is as defined in one of numbered paragraphs 144 to 148.
287. In some embodiments, Q¹ is Ih, L¹ is a bond, Q² is as defined in numbered paragraph 97, wherein L₃ is —CH₂—, Q³ is as defined in one of paragraphs 112 to 178, for example Q³ is as defined in numbered paragraph 114 and R⁷ is as defined in numbered paragraphs 144 to 148.
288. In some embodiments, Q¹ is Ih, L¹ is a bond, Q² is as defined in numbered paragraph 97, wherein L₃ is a bond and Q³ is as defined in one of paragraphs 112 to 178, for example Q³ is as defined in numbered paragraph 164.
289. In some embodiments, Q¹ is Ih, L¹ is a bond, Q² is as defined in numbered paragraph 97, wherein L₃ is

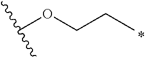 [SMILES: *OCCC]

and Q³ is as defined in one of paragraphs 112 to 178, for example Q³ is as defined in paragraph 164.
290. In some embodiments, Q¹ is Ih, L¹ is —CH₂—, Q² is phenyl, L₃ is as defined in numbered paragraph 109 and Q³ is as defined in paragraph 131.
291. In some embodiments, Q¹ is Ii or Ij, L¹ is —CH₂— and Q² is phenyl.
292. In some embodiments, Q¹ is Ii or Ij, L¹ is a bond and Q² is as defined in numbered paragraph 67.
293. In some embodiments, Q¹ is Ii or Ij, L¹ is a bond and Q² is as defined in one of numbered paragraphs 84, 87, 89 and 91.
294. In some embodiments, Q¹ is Ii or Ij, L¹ is a bond and Q² is as defined in numbered paragraph 97.
295. In some embodiments, Q¹ is Ii or Ij, L¹ is a bond, Q² is as defined in numbered paragraph 97, wherein L₃ is —CH₂— and Q³ is phenyl.
296. In some embodiments, Q¹ is Ii or Ij, L¹ is a bond, Q² is as defined in numbered paragraph 97, wherein L₃ is a bond, and Q³ is as defined in one of paragraphs 112 to 178, for example Q³ is as defined in numbered paragraph 114 and R⁷ is as defined in one of numbered paragraphs 144 to 148.
297. In some embodiments, Q¹ is Ii or Ij, L¹ is a bond, Q² is as defined in numbered paragraph 97, wherein L₃ is —CH₂—, Q³ is as defined in one of paragraphs 112 to 178, for example Q³ is as defined in numbered paragraph 114 and R⁷ is as defined in numbered paragraphs 144 to 148.
298. In some embodiments, Q¹ is Ii or Ij, L¹ is a bond, Q² is as defined in numbered paragraph 97, wherein L₃ is a bond and Q³ is as defined in one of paragraphs 112 to 178, for example Q³ is as defined in numbered paragraph 164.
299. In some embodiments, Q¹ is Ii or Ij, L¹ is a bond, Q² is as defined in numbered paragraph 97, wherein L₃ is

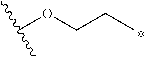 [SMILES: *OCCC]

and Q³ is as defined in one of paragraphs 112 to 178, for example Q³ is as defined in paragraph 164.
300. In some embodiments, Q¹ is Ii or Ij, L¹ is —CH₂—, Q² is phenyl, L₃ is as defined in numbered paragraph 109 and Q³ is as defined in paragraph 131.
301. In some embodiments, Q¹ is Ik, L¹ is —NH— and Q² is as defined in numbered paragraph 67.
302. In some embodiments, Q¹ is Ik, L¹ is —NH— and Q² is as defined in one of numbered paragraphs 84, 87, 89 and 91.
303. In some embodiments, Q¹ is Ik, L¹ is —NH— and Q² is as defined in numbered paragraph 97.
304. In some embodiments, Q¹ is Ik, L¹ is —NH—, Q² is as defined in numbered paragraph 97, wherein L₃ is —CH₂— and Q³ is phenyl.
305. In some embodiments, Q¹ is Ik, L¹ is —NH—, Q² is as defined in numbered paragraph 97, wherein L₃ is a bond, and Q³ is as defined in one of paragraphs 112 to 178, for example Q³ is as defined in numbered paragraph 114 and R⁷ is as defined in one of numbered paragraphs 144 to 148.
306. In some embodiments, Q¹ is Ik, L¹ is —NH—, Q² is as defined in numbered paragraph 97, wherein L₃ is —CH₂—, and Q³ is as defined in one of paragraphs 112 to 178, for example Q³ is as defined in numbered paragraph 114 and R⁷ is as defined in one of numbered paragraphs 144 to 148.
307. In some embodiments, Q¹ is Ik, L¹ is —NH—, Q² is as defined in numbered paragraph 97, wherein L₃ is a bond and Q³ is as defined in one of paragraphs 112 to 178, for example Q³ is as defined in numbered paragraph 164.
308. In some embodiments, Q¹ is Ik, L¹ is —NH—, Q² is as defined in numbered paragraph 97, wherein L₃ is

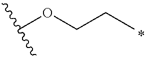 [SMILES: *OCCC]

and Q³ is as defined in one of paragraphs 112 to 178, for example Q³ is as defined in paragraph 164.
309. In some embodiments, Q¹ is Ik, L¹ is —CH₂—, Q² is phenyl, L₃ is as defined in numbered paragraph 109 and Q³ is as defined in paragraph 131.
310. L³ is a bond or is selected from: C₁₋₄ alkylene, -L⁴-O-L⁵-, -L⁴-O-L⁵¹-O-L⁵, -L⁴-NR^{A1}-L⁵-,
   wherein L⁴ and L⁵ are independently selected from a bond and C₁₋₄ alkylene; and
   L⁵¹ is C₁₋₄ alkylene. Wherein, -L⁴ is the point of attachment to Q² and L⁵- is the point of attachment to Q³ of Formula (I).
311. L⁴ and L⁵ are independently selected from a bond or C₁₋₄ alkylene. It may be that L⁴ and L⁵ are independently selected from a bond or C₁₋₂ alkylene. It may be that L⁴ and L⁵ are independently selected from a bond or —CH₂—. It may be that L⁴ and L⁵ are both —CH₂—. It may be that L⁴ is —CH₂— and L⁵ is a bond. It may be that L⁴ is a bond and L⁵ is —CH₂—. It may be that L⁴ is a bond and L⁵ is a bond.
312. L⁵¹ is C₁₋₄ alkylene. It may be that L⁵¹ is C₁₋₂ alkylene. It may be that L⁵¹ is —CH₂—. It may be that L⁵¹ is —CH₂CH₂—.
313. L³ is -L⁴-O-L⁵¹-O-L⁵-. It may be that L⁴ and L⁵ are independently selected from a bond or C₁₋₂ alkylene. It may be that L⁴ and L⁵ are independently selected from a bond or —CH₂—. It may be that L⁴ is a bond and L⁵ is a bond. It may be that L⁵¹ is C₁₋₂ alkylene. It may be that L⁵¹ is —CH₂—. It may be that L⁵¹ is —CH₂CH₂—. Thus, it may be that L³ is selected from:

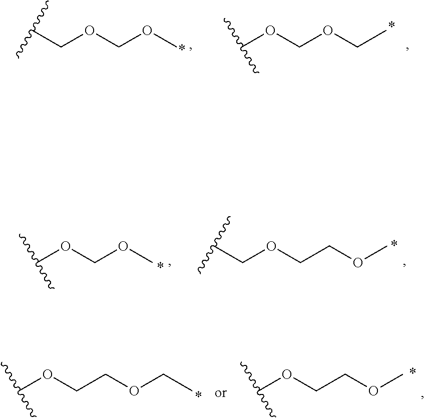

wherein [symbol] indicates the point of attachment to Q² and * indicates the point of attachment to Q³.
Preferably, L³ is

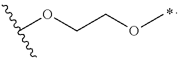 [SMILES: *OCCOC]

314. In some embodiments, L³ is as defined in numbered paragraph 313 and Q³ is as defined in one of paragraphs 112 to 178, for example, Q³ is as defined in numbered paragraph 112.
315. In some embodiments, Q¹ is Ia, L¹ is —NH—, Q² is as defined in numbered paragraph 97, L₃ is

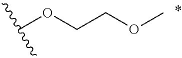 [SMILES: *OCCOC]

wherein [symbol] indicates the point of attachment to Q² and * indicates the point of attachment to Q³, and Q³ is as defined in one of paragraphs 112 to 178, for example Q³ is as defined in paragraph 112.
316. Q³ is selected from: phenyl, a 5- or 6-membered heteroaryl, 3- to 12-membered heterocyclyl and C₃₋₆ cycloalkyl, wherein said phenyl or 5- or 6-membered heteroaryl is optionally substituted by one or more R⁷, and said 3- to 12-membered heterocyclyl or C₃₋₆ cycloalkyl is optionally substituted by one or more R⁸.
317. Q³ is a C₃₋₆ cycloalkyl optionally substituted by one or more R⁸. Thus, it may be that Q³ is selected from:

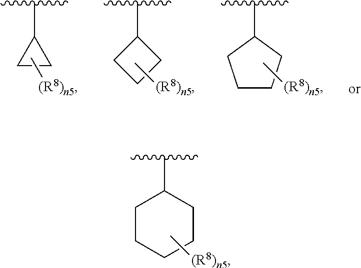

wherein:
   n5 is 0, 1 or 2 (where chemically possible);
   [symbol] indicates the point of attachment to L³ of Formula (I); and
   R⁸ at each occurrence is independently selected from halo, C₁₋₄ alkyl and —OR^{A1}.
318. Q³ is an unsubstituted C₃₋₆ cycloalkyl. It may be that Q³ is cyclopropyl, cyclobutyl, cyclopentyl, or cyclohexyl. It may be that Q³ is unsubstituted cyclopropyl.
319. In some embodiments, L³ is as defined in one of numbered paragraphs 107 to 109 and Q³ is as defined in one of paragraphs 317 or 318. It may be that L³ is —O—CH₂— and Q³ is unsubstituted cyclopropyl. Thus, it may be that the group -Q²-L³-Q³ is selected from:

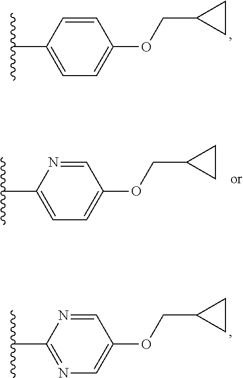 [SMILES: *c1ccc(OCC2CC2)cc1 ; *c1ccc(OCC2CC2)cn1 ; *c1ncc(OCC2CC2)cn1]

wherein [symbol] indicates the point of attachment to L¹ of Formula (I). [symbol]
320. In some embodiments, L³ is as defined in numbered paragraph 313 and Q³ is as defined in one of paragraphs 317 or 318. It may be that L³ is

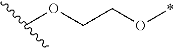 [SMILES: *OCCO*]

and Q³ is unsubstituted cyclopropyl. Thus, it may be that the group -Q²-L³-Q³ is selected from:

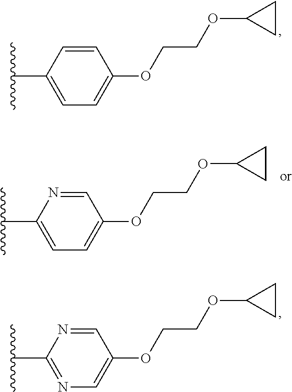 [SMILES: *c1ccc(OCCOC2CC2)cc1 ; *c1ccc(OCCOC2CC2)cn1 ; *c1ncc(OCCOC2CC2)cn1]

wherein [symbol] indicates the point of attachment to L¹ of Formula (I).

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), (XLII), (XLIII), (XLIV), (XLV), (XLVI), (XLVII), (XVIII), (XLIX), (L), (LI), (LII), (LIII), (LIV), (LV), (LVI), (LVII), (LVIII), (LIX), (LX), (LXI), (LXII), or (LXIII), or a pharmaceutically acceptable salt thereof, wherein the group

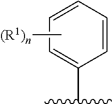

is as defined in any one of numbered paragraphs 1 to 14. Thus, it may be that the group

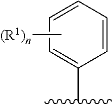

has the structure

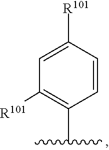

wherein each R¹⁰¹ is independently selected from the group consisting of —F, —Br, —Cl and -Me. Suitably, in these embodiments, the group

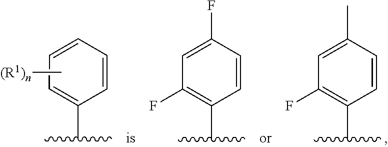

preferably

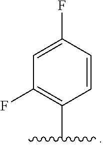 [SMILES: *c1ccc(F)cc1F]

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), (XLII), (XLIII), (XLIV), (XLV), (XLVI), (XLVII), (XVIII), (XLIX), (L), (LI), (LII), (LIII), (LIV), (LV), (LVI), (LVII), (LVIII), (LIX), (LX), (LXI), (LXII), or (LXIII), or a pharmaceutically acceptable salt thereof, wherein R² is as defined in any one of numbered paragraphs 15 to 17. Suitably, in these embodiments R² is H.

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), (XLII), (XLIII), (XLIV), (XLV), (XLVI), (XLVII), (XVIII), (XLIX), (L), (LI), (LII), (LIII), (LIV), (LV), (LVI), (LVII), (LVIII), (LIX), (LX), (LXI), (LXII), or (LXIII), or a pharmaceutically acceptable salt thereof, wherein the group

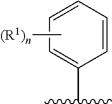

has the structure

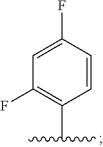 [SMILES: *c1ccc(F)cc1F]

### and R² is H.

In certain embodiments the compound of the invention is a compound of the formula (I), (II), (III), (IV), (V), (VI), (VII), (VIII), (IX), (X), (XI), (XII), (XIII), (XIV), (XV), (XVI), (XVII), (XVIII), (XIX), (XX), (XXI), (XXII), (XXIII), (XXIV), (XXV), (XXVI), (XXVII), (XXVIII), (XXIX), (XXX), (XXXI), (XXXII), (XXXIII), (XXXIV), (XXXV), (XXXVI), (XXXVII), (XXXVIII), (XXXIX), (XL), (XLI), (XLII), (XLIII), (XLIV), (XLV), (XLVI), (XLVII), (XVIII), (XLIX), (L), (LI), (LII), (LIII), (LIV), (LV), (LVI), (LVII), (LVIII), (LIX), (LX), (LXI), (LXII), or (LXIII), or a pharmaceutically acceptable salt thereof, wherein R³ is as defined in any one of numbered paragraphs 34 to 37. Suitably in these embodiments, R³ is absent.

In certain embodiments the compound of the invention is a compound of the formula (I), (II), (X), (XVIII), (XXVI) or (XXXIV), or a pharmaceutically acceptable salt thereof, wherein L¹ is as defined in any one of numbered paragraphs 39 to 44. Suitably, in these embodiments, L¹ is —NH—.

In certain embodiments the compound of the invention is a compound of the formula (LIV), or a pharmaceutically acceptable salt thereof, wherein L¹ is as defined in numbered paragraph 39.

In certain embodiments the compound of the invention is a compound of the formula (I), (II), (X), (XVIII), (XXVI) or (XXXIV), or a pharmaceutically acceptable salt thereof, wherein Q² is as defined in any one of numbered paragraphs 45 to 72. Suitably, in these embodiments L¹ is —NH—, R³ is absent, R² is H and

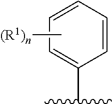

has the structure

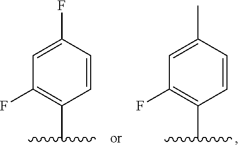

preferably

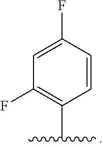 [SMILES: *c1ccc(F)cc1F]

In certain embodiments the compound of the invention is a compound of the formula (LIV), or a pharmaceutically acceptable salt thereof, wherein Q² is as defined in any one of numbered paragraphs 45 to 72. Suitably, in these embodiments L¹ is a bond, R³ is absent, R² is H and

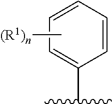

has the structure

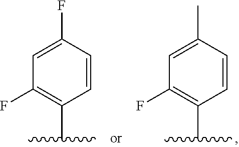

preferably

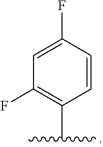 [SMILES: *c1ccc(F)cc1F]

In certain embodiments the compound of the invention is a compound of the formula (I), (II), (X), (XVIII), (XXVI) or (XXXIV), or a pharmaceutically acceptable salt thereof, wherein Q² is as defined in any one of numbered paragraphs 45 to 72. Suitably, in these embodiments R⁵ is absent, and R⁶ at each occurrence is independently selected from the group consisting of: halo, —CN, C₁₋₄ alkyl and —OR^{A1}. Thus, it may be that R⁶ is selected from the group consisting of —F, —Br, —Cl, methyl and —OCH₃. Suitably, in these embodiments L¹ is —NH—, R³ is absent, R² is H and

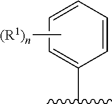

has the structure

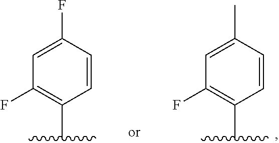

preferably

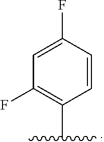 [SMILES: *c1ccc(F)cc1F]

In certain embodiments the compound of the invention is a compound of the formula (X), (XII), (XIII), (XIX), (XX). (XXI), (XXVII), (XXVIII), (XXIX), (XXXV), (XXXVI) or (XXXVII), or a pharmaceutically acceptable salt thereof, wherein R⁶ is as defined in any one of numbered paragraphs 74 to 91. Thus, it may be that R⁶ is selected from the group consisting of —F, —Br, —Cl, methyl and —OCH₃. Suitably, in these embodiments L¹ is —NH—, R³ is absent, R² is H and

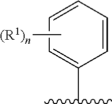

has the structure

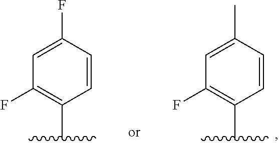

preferably

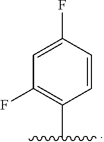 [SMILES: *c1ccc(F)cc1F]

Suitably, in these embodiments Q² is selected from

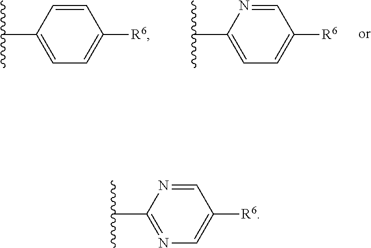

In certain embodiments the compound of the invention is of the formula (LIV), (LV), (LVI), (LVII), (LVIII), (LIX), (LX), (LXI), (LXII), or (LXIII), wherein the group

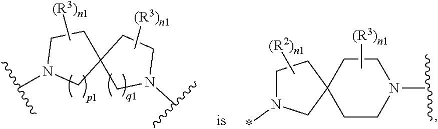

wherein * shows the point of attachment to

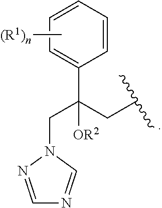

For example, it may be that this group is of the formula

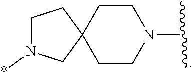

In certain embodiments the compound of the invention is a compound of the formula (XLII), (XLIII), (XLIV), (XLV), (XLVI), (XLVII), (XVIII), (XLIX), (L), (LI), (LII) or (LIII), or a pharmaceutically acceptable salt thereof, wherein R⁶ is —OR^{A1}, and —OR^{A1} is as defined in any one of paragraphs 92 or 94. Thus, it may be that —OR^{A1} is selected from —O(CH₂)₂—OCH₃ or —OCH₂CH(CH₃)₂ (i.e. iso-butoxy). Suitably, in these embodiments L¹ is —NH— (i.e. R⁴ is H), R³ is absent, R² is H, R⁶ is absent and

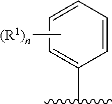

has the structure

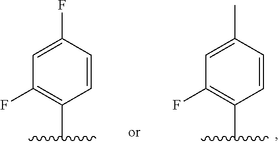

preferably

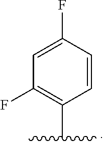 [SMILES: *c1ccc(F)cc1F]

Suitably, in these embodiments Q² is selected from

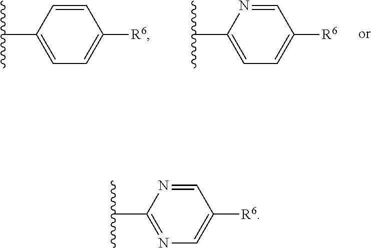

In certain embodiments the compound of the invention is a compound of the formula (LXII) or (LXIII), or a pharmaceutically acceptable salt thereof, wherein —OR^{A1} is as defined in any of paragraphs 92 or 94. Thus, it may be that —OR^{A1} is selected from —O(CH₂)₂—OCH₃ or —OCH₂CH(CH₃)₂ (i.e. iso-butoxy). Suitably, in these embodiments L¹ is a bond, R³ is absent, R² is H, R⁶ is absent and

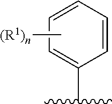

has the structure or

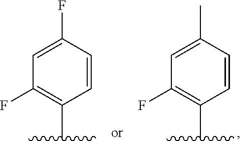

preferably

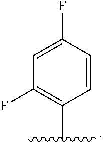 [SMILES: *c1ccc(F)cc1F]

Suitably, in these embodiments Q² is selected from

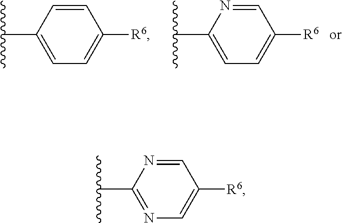

In certain embodiments the compound of the invention is a compound of the formula (III), (IV), (V), (VI), (VII), (VIII), (IX), (XI), (XIV), (XV), (XVI), (XVII), (XIX), (XXII), (XXIII), (XXIV), (XXV), (XXVII), (XXX), (XXXI), (XXXII), (XXXIII), (XXXV), (XXXVIII), (XXXIX), (XL) or (XLI), or a pharmaceutically acceptable salt thereof, wherein L³ is as defined in any one of numbered paragraphs 98 to 111. Thus, it may be that L³ is a bond or —CH₂—. Suitably, in these embodiments L¹ is —NH— (i.e. R⁴ is H), R³ is absent, R² is H and

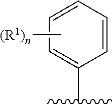

has the structure

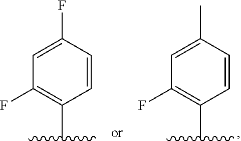

preferably

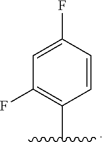 [SMILES: *c1ccc(F)cc1F]

Suitably, in these embodiments, Q² is

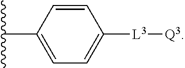

In certain embodiments the compound of the invention is a compound of the formula (LV), (LVIII), (LIX), (LX) or (LXI), or a pharmaceutically acceptable salt thereof, wherein L³ is as defined in any one of numbered paragraphs 98 to 111. Thus, it may be that L³ is a bond or —CH₂—. Suitably, in these embodiments R³ is absent, R² is H and

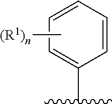

has the structure

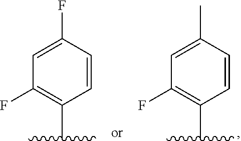

preferably

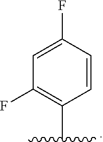 [SMILES: *c1ccc(F)cc1F]

Suitably, in these embodiments, Q² is

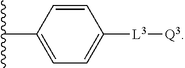

In certain embodiments the compound of the invention is a compound of the formula (III), (IV), (V), (VI), (VII), (VIII), (IX), (XI), (XIV), (XV), (XVI), (XVII), (XIX), (XXII), (XXIII), (XXIV), (XXV), (XXVII), (XXX), (XXXI), (XXXII), (XXXIII), (XXXV), (XXXVIII), (XXXIX), (XL), (XLI), (LV), (LVIII), (LIX), (LX) or (LXI), or a pharmaceutically acceptable salt thereof, wherein L³ is -L⁴-O-L⁵- as defined in any one of numbered paragraphs 107 to 109. Thus, it may be that L³ is —O—C₁₋₄ alkylene. Preferably, it may be that L³ is —O—CH₂— or —O—CH₂CH₂—, wherein the —O is attached to Q² and CH₂— is attached to Q³. Suitably, in these embodiments R⁴ is H (where present), R³ is absent, R² is H and

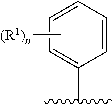

has the structure

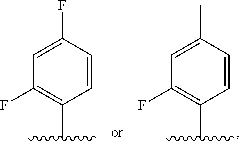

preferably

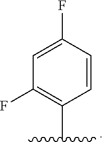 [SMILES: *c1ccc(F)cc1F]

Suitably, in these embodiments, Q² is

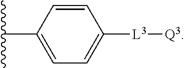

In certain embodiments the compound of the invention is a compound of the formula (III), (IV), (V), (VI), (VII), (VIII), (IX), (XI), (XIV), (XV), (XVI), (XVII), (XIX), (XXII), (XXIII), (XXIV), (XXV), (XXVII), (XXX), (XXXI), (XXXII), (XXXIII), (XXXV), (XXXVIII), (XXXIX), (XL), (XLI), (LV), (LVIII), (LIX), (LX) or (LXI), or a pharmaceutically acceptable salt thereof, wherein L³ is -L⁴-O-L⁵¹-O-L⁵- as defined in numbered paragraph 313. Thus, it may be that L³ is —O—C₁₋₂ alkylene-O—. Preferably, it may be that L³ is

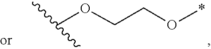 [SMILES: *OCCO*]

wherein [symbol] indicates the point of attachment to Q² and * indicates the point of attachment to Q³. Suitably, in these embodiments R⁴ is H (where present), R³ is absent, R² is H and

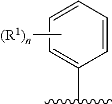

has the structure or

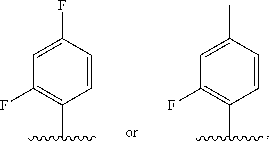

preferably

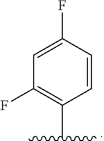 [SMILES: *c1ccc(F)cc1F]

Suitably, in these embodiments, Q² is

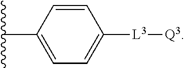

In certain embodiments the compound of the invention is a compound as defined in one of the two paragraphs immediately above, wherein Q³ is as defined in any one of numbered paragraphs 112 to 141, 149 to 178 and 316 to 318.

In certain embodiments the compound of the invention is a compound of the formula (III), (IV), (V), (VI), (XIV), (XIX), (XXII), (XXVII), (XXX), (XXXV) or (XXXVIII), or a pharmaceutically acceptable salt thereof, wherein Q³ is as defined in any one of numbered paragraphs 112 to 141, 149 to 178 and 316 to 318. Suitably, in these embodiments L³ is a bond or —CH₂—, L¹ is —NH— (i.e. R⁴ is H), R³ is absent, R² is H and

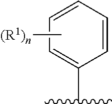

has the structure

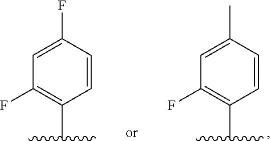

preferably

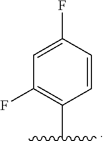 [SMILES: *c1ccc(F)cc1F]

Suitably, in these embodiments, Q² is

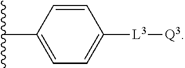

In certain embodiments the compound of the invention is a compound of the formula (LV), or (LVIII), or a pharmaceutically acceptable salt thereof, wherein Q³ is as defined in any one of numbered paragraphs 112 to 141, 149 to 178 and 316 to 318. Suitably, in these embodiments L³ is a bond or —CH₂—, R³ is absent, R² is H and

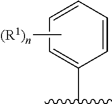

has the structure

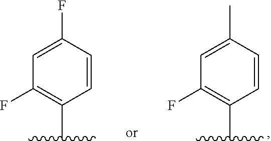

preferably

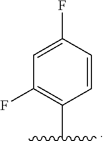 [SMILES: *c1ccc(F)cc1F]

Suitably, in these embodiments, Q² is

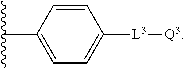

In certain embodiments the compound of the invention is a compound of the formula (VII), (VIII), (IX), (XI), (XV), (XVI), (XVII), (XXIII), (XXIV), (XXV), (XXXI), (XXXII), (XXXIII), (XXXIX), (XL) or (XLI), or a pharmaceutically acceptable salt thereof, wherein R⁷ is as defined in any one of numbered paragraphs 140 to 148. Thus, it may be that R⁷ is selected from the group consisting of —F, —Br, —Cl, methyl and —OCH₃. Suitably, in these embodiments L³ is a bond or —CH₂—, L¹ is —NH— (i.e., R⁴ is H), R³ is absent, R² is H and

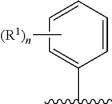

has the structure

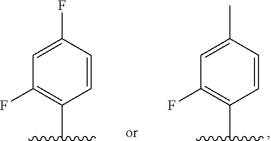

preferably

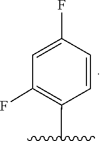 [SMILES: *c1ccc(F)cc1F]

Suitably, in these embodiments, Q² is

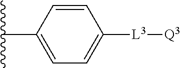

and Q³ is selected from the group consisting of

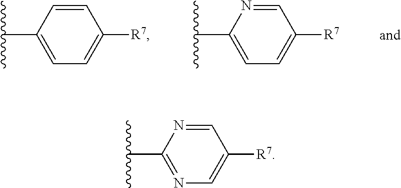

In certain embodiments the compound of the invention is a compound of the formula (LIX), (LX), or (LXI), or a pharmaceutically acceptable salt thereof, wherein R⁷ is as defined in any one of numbered paragraphs 140 to 148. Thus, it may be that R⁷ is selected from the group consisting of —F, —Br, —Cl, methyl and —OCH₃. Suitably, in these embodiments L³ is a bond or —CH₂—, R³ is absent, R² is H and

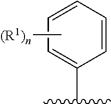

has the structure

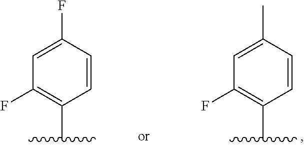

preferably

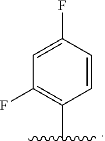 [SMILES: *c1ccc(F)cc1F]

Suitably, in these embodiments, Q² is

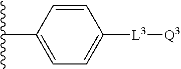

and Q³ is selected from the group consisting of

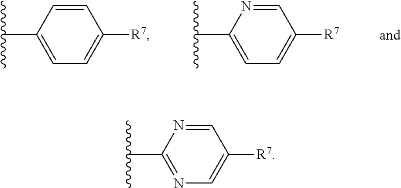

In certain embodiments the compound of the invention is a compound of the formula (IV), (V), (VI), (XIV), (XXII), (XXX) or (XXXVIII), or a pharmaceutically acceptable salt thereof, wherein R¹ is as defined in any one of numbered paragraphs 1 to 14. Suitably, in these embodiments, n1 is preferably 0, R⁴ is preferably H, n2 is preferably 0, L³ is preferably a bond or —CH₂—, and Q³ is as defined in any one of numbered paragraphs 112 to 139.

In certain embodiments the compound of the invention is a compound of the formula (IV), (V), (VI), (XIV), (XXII), (XXX) or (XXXVIII), or a pharmaceutically acceptable salt thereof, wherein Q³ is as defined in any one of numbered paragraphs 153 to 164, preferably, Q³ is

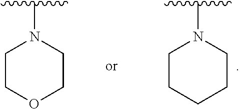 [SMILES: *N1CCOCC1 ; *N1CCCCC1]

Suitably, in these embodiments L³ is a bond or —O—CH₂CH₂—, L¹ is —NH—, R³ is absent, R⁶ is absent, R² is H and

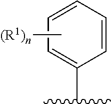

has the structure

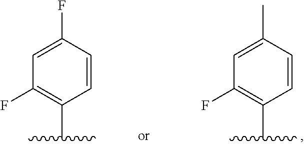

preferably.

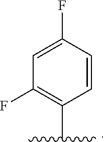 [SMILES: *c1ccc(F)cc1F]

In certain embodiments the compound of the invention is a compound of the formula (II), (X), (XVIII), (XXVI), (XXXIV), or a pharmaceutically acceptable salt thereof, wherein L¹ is —CH₂—, Q² is

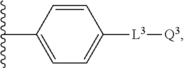

wherein L³ is as defined in numbered paragraph 109, Q³ is as defined in numbered paragraph 131, and R⁷ is selected 5 from the group consisting of halo, —CN, C₁₋₄ alkyl and —OR^{A1}. Preferably, R⁷ is selected from the group consisting of —F, —CH₃ and H.

In certain embodiments the compound of the invention is a compound of the formula (IX), or a pharmaceutically acceptable salt thereof, wherein R⁷ is as defined in any one of numbered paragraphs 146 to 148. Thus, it may be that R⁷ is selected from the group consisting of —CN, -methyl and —OCH₃. Suitably, in these embodiments L³ is a bond, R⁴ is H, R³ is absent, R⁶ is absent, R² is H and

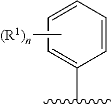

has the structure or

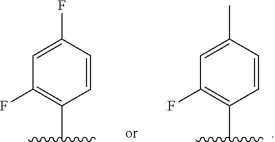

preferably

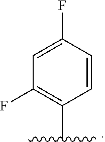 [SMILES: *c1ccc(F)cc1F]

Suitably, in these embodiments, Q³ is selected from the group consisting of

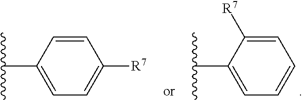

In certain embodiments the compound of the invention is a compound of the formula (I), (II), (X), (XVIII), (XXVI) and (XXXIV), or a pharmaceutically acceptable salt thereof, wherein Q² is -Q²-L³-Q³ and the group -Q²-L³-Q³ is selected from:

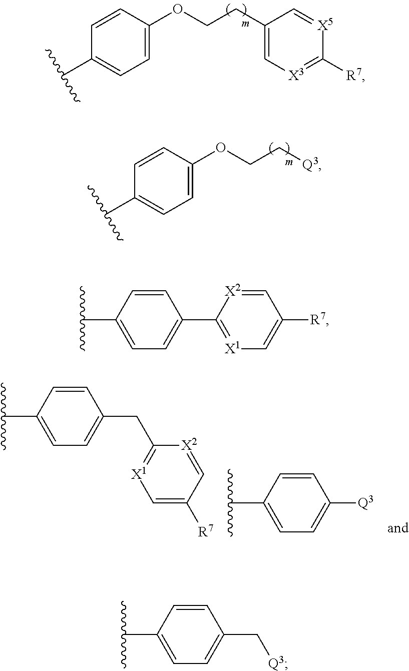

wherein
m is 0, 1 or 2;
X¹ and X² are each independently selected from CH and N (optionally wherein at least one of X¹ and X² is N);
X³ and X⁵ are each independently selected from CH and N (optionally wherein at least one of X³ and X⁵ is N);
Q³ is a 5-membered heteroaryl optionally substituted by one or two R⁷ or a 4- to 7-membered non-aromatic saturated or partially saturated monocyclic heterocyclyl group containing at least one ring heteroatom selected from O, S and N, wherein said 4- to 7-membered heterocyclyl is optionally substituted by one or two R⁸;
R⁷ and R⁸ have any of the values defined herein; and
[symbol] indicates the point of attachment to Q² of Formula (I).

Suitably in this embodiment Q³ is as defined in one of numbered paragraphs 118 to 121, or 135 to 139. Thus it may be that Q³ is a 5-membered heteroaryl containing at least 1 ring nitrogen atom, wherein Q³ is optionally substituted by one or two R¹. Thus it may be that Q³ is selected from pyrrolyl, imidazolyl, pyrazolyl, triazolyl and tetrazolyl, wherein Q³ is optionally substituted with one or two R⁷.

Suitably in this embodiment Q³ is as defined in one of numbered paragraphs 150 to 178

Suitably in this embodiment R⁷ is as defined in any one of numbered paragraphs 140 to 148.

Suitably in this embodiment R⁸ is as defined in any one of numbered paragraphs 179 to 187.

Suitably in this embodiment Q³ is unsubstituted.

Suitably in this embodiment R³ is absent, R⁶ is absent, R² is H and

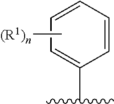

has the structure

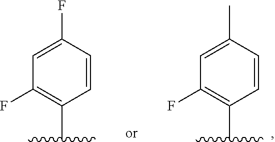

preferably.

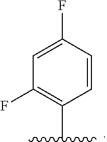 [SMILES: *c1ccc(F)cc1F]

In another embodiment there is provided a compound selected from any one of the Examples herein, or a pharmaceutically acceptable salt thereof.

In another embodiment there is provided a compound selected from Table 1, Table 2, and/or Table 3, or a pharmaceutically acceptable salt or prodrug thereof. In particular there is provided a compound selected from Table 1, Table 2, and/or Table 3, or a pharmaceutically acceptable salt thereof:

TABLE 1

| Example number: | Example structure: |
|---|---|
| 1 | 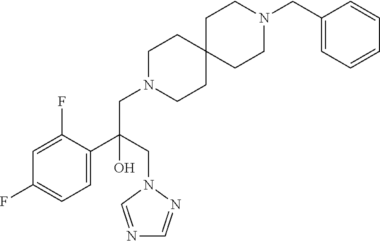 [SMILES: OC(CN1CCC2(CCN(Cc3ccccc3)CC2)CC1)(Cn1cncn1)c1ccc(F)cc1F] |
| 2 | 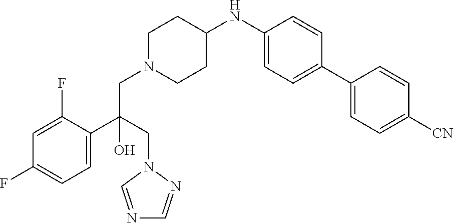 [SMILES: N#Cc1ccc(-c2ccc(NC3CCN(CC(O)(Cn4cncn4)c4ccc(F)cc4F)CC3)cc2)cc1] |
| 3 | 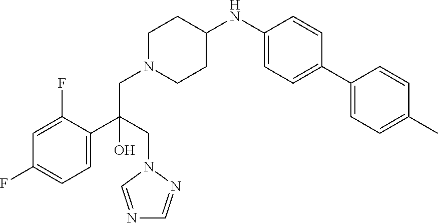 [SMILES: Cc1ccc(-c2ccc(NC3CCN(CC(O)(Cn4cncn4)c4ccc(F)cc4F)CC3)cc2)cc1] |
| 4 | 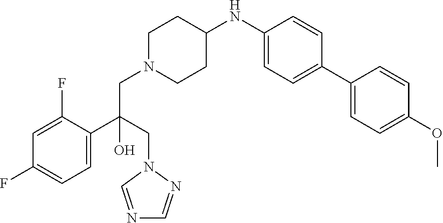 [SMILES: COc1ccc(-c2ccc(NC3CCN(CC(O)(Cn4cncn4)c4ccc(F)cc4F)CC3)cc2)cc1] |
| 5 | 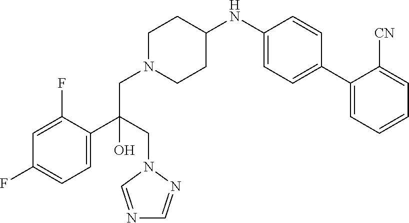 [SMILES: N#Cc1ccccc1-c1ccc(NC2CCN(CC(O)(Cn3cncn3)c3ccc(F)cc3F)CC2)cc1] |
| 6 | 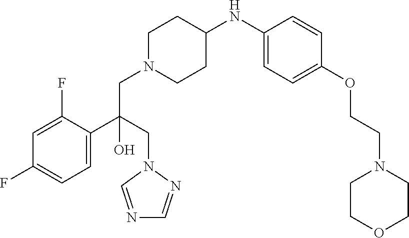 [SMILES: OC(CN1CCC(Nc2ccc(OCCN3CCOCC3)cc2)CC1)(Cn1cncn1)c1ccc(F)cc1F] |
| 7 | 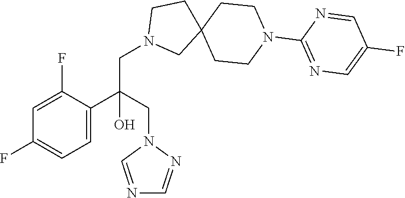 [SMILES: OC(CN1CCC2(CCN(c3ncc(F)cn3)CC2)C1)(Cn1cncn1)c1ccc(F)cc1F] |
| 8 | 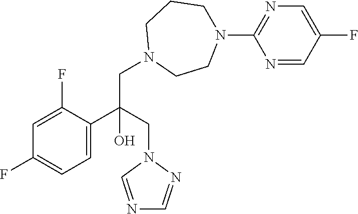 [SMILES: OC(CN1CCCN(c2ncc(F)cn2)CC1)(Cn1cncn1)c1ccc(F)cc1F] |
| 9 | 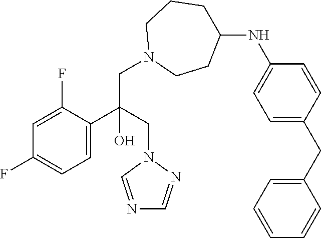 [SMILES: OC(CN1CCCC(Nc2ccc(Cc3ccccc3)cc2)CC1)(Cn1cncn1)c1ccc(F)cc1F] |
| 10 | 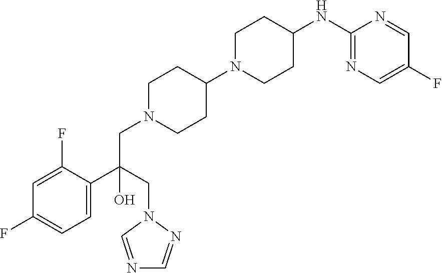 [SMILES: OC(CN1CCC(N2CCC(Nc3ncc(F)cn3)CC2)CC1)(Cn1cncn1)c1ccc(F)cc1F] |
| 11 | 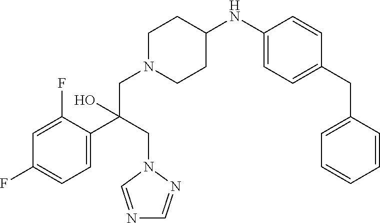 [SMILES: OC(CN1CCC(Nc2ccc(Cc3ccccc3)cc2)CC1)(Cn1cncn1)c1ccc(F)cc1F] |
| 12 | 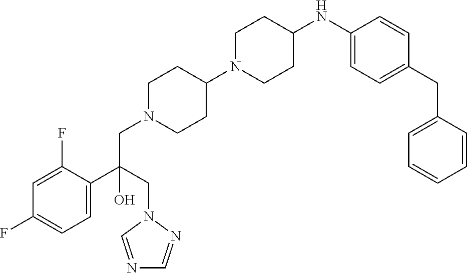 [SMILES: OC(CN1CCC(N2CCC(Nc3ccc(Cc4ccccc4)cc3)CC2)CC1)(Cn1cncn1)c1ccc(F)cc1F] |
| 13 | 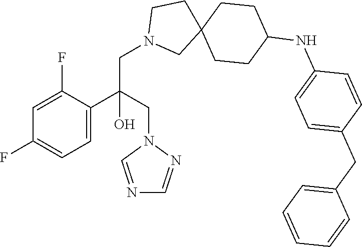 [SMILES: OC(CN1CCC2(CCC(Nc3ccc(Cc4ccccc4)cc3)CC2)C1)(Cn1cncn1)c1ccc(F)cc1F] |
| 14 | 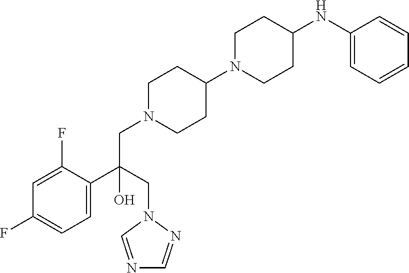 [SMILES: OC(CN1CCC(N2CCC(Nc3ccccc3)CC2)CC1)(Cn1cncn1)c1ccc(F)cc1F] |
| 15 | 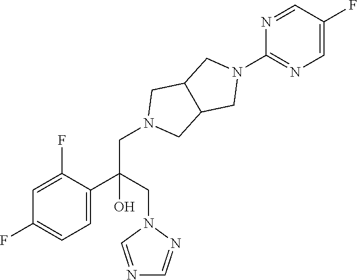 [SMILES: OC(CN1CC2CN(c3ncc(F)cn3)CC2C1)(Cn1cncn1)c1ccc(F)cc1F] |
| 16 | 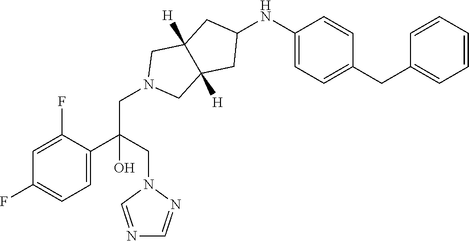 [SMILES: OC(CN1C[C@H]2CC(Nc3ccc(Cc4ccccc4)cc3)C[C@H]2C1)(Cn1cncn1)c1ccc(F)cc1F] |
| 17 | 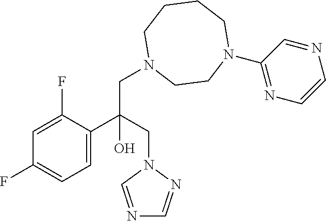 [SMILES: OC(CN1CCCCN(c2cnccn2)CC1)(Cn1cncn1)c1ccc(F)cc1F] |
| 18 | 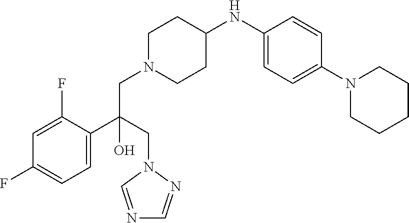 [SMILES: OC(CN1CCC(Nc2ccc(N3CCCCC3)cc2)CC1)(Cn1cncn1)c1ccc(F)cc1F] |
| 19 | 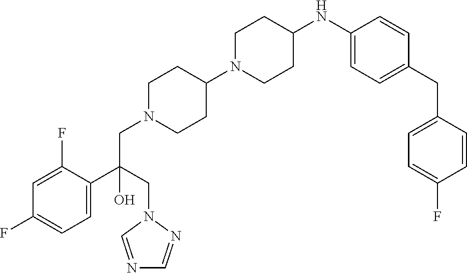 [SMILES: OC(CN1CCC(N2CCC(Nc3ccc(Cc4ccc(F)cc4)cc3)CC2)CC1)(Cn1cncn1)c1ccc(F)cc1F] |
| 20 | 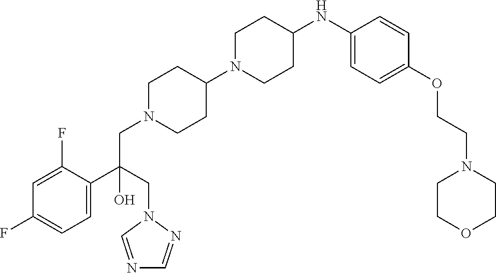 [SMILES: OC(CN1CCC(N2CCC(Nc3ccc(OCCN4CCOCC4)cc3)CC2)CC1)(Cn1cncn1)c1ccc(F)cc1F] |
| 21 | 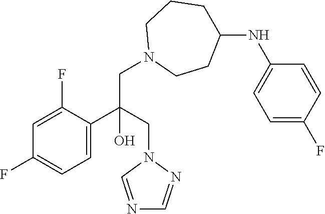 [SMILES: OC(CN1CCCC(Nc2ccc(F)cc2)CC1)(Cn1cncn1)c1ccc(F)cc1F] |
| 22 | 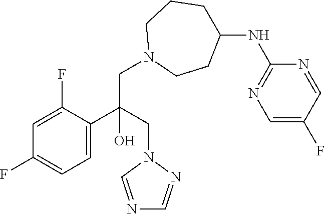 [SMILES: OC(CN1CCCC(Nc2ncc(F)cn2)CC1)(Cn1cncn1)c1ccc(F)cc1F] |
| 23 | 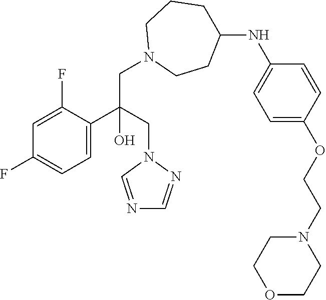 [SMILES: OC(CN1CCCC(Nc2ccc(OCCN3CCOCC3)cc2)CC1)(Cn1cncn1)c1ccc(F)cc1F] |
| 24 | 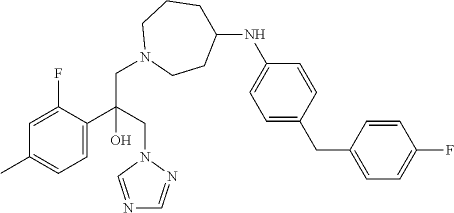 [SMILES: Cc1ccc(C(O)(CN2CCCC(Nc3ccc(Cc4ccc(F)cc4)cc3)CC2)Cn2cncn2)c(F)c1] |
| 25 | 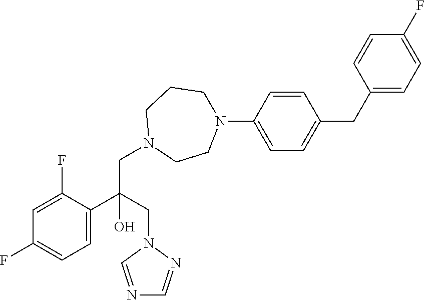 [SMILES: OC(CN1CCCN(c2ccc(Cc3ccc(F)cc3)cc2)CC1)(Cn1cncn1)c1ccc(F)cc1F] |
| 26 | 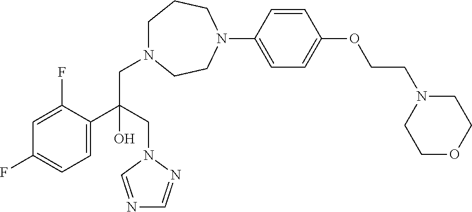 [SMILES: OC(CN1CCCN(c2ccc(OCCN3CCOCC3)cc2)CC1)(Cn1cncn1)c1ccc(F)cc1F] |
| 27 | 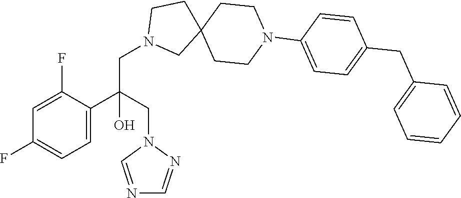 [SMILES: OC(CN1CCC2(CCN(c3ccc(Cc4ccccc4)cc3)CC2)C1)(Cn1cncn1)c1ccc(F)cc1F] |
| 28 | 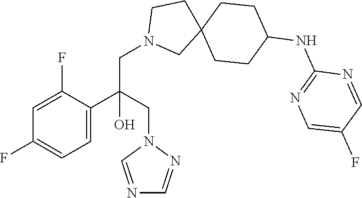 [SMILES: OC(CN1CCC2(CCC(Nc3ncc(F)cn3)CC2)C1)(Cn1cncn1)c1ccc(F)cc1F] |
| 29 | 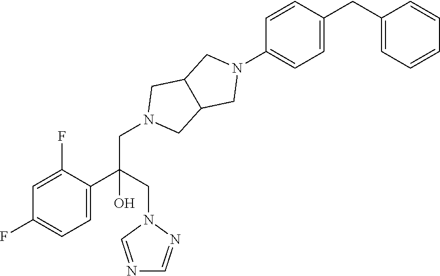 [SMILES: OC(CN1CC2CN(c3ccc(Cc4ccccc4)cc3)CC2C1)(Cn1cncn1)c1ccc(F)cc1F] |
| 30 | 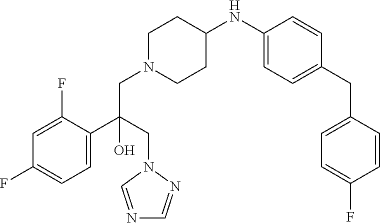 [SMILES: OC(CN1CCC(Nc2ccc(Cc3ccc(F)cc3)cc2)CC1)(Cn1cncn1)c1ccc(F)cc1F] |

TABLE 2

| Example number: | Example structure: |
|---|---|
| 31 | 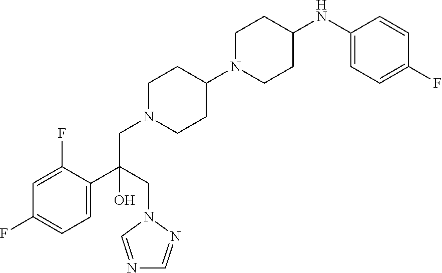 [SMILES: OC(CN1CCC(N2CCC(Nc3ccc(F)cc3)CC2)CC1)(Cn1cncn1)c1ccc(F)cc1F] |
| 32 | 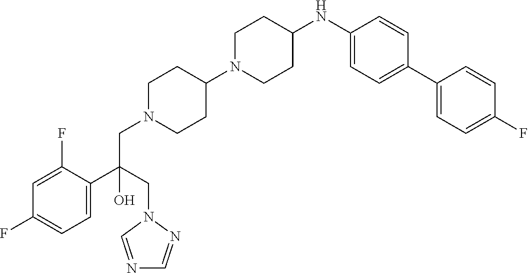 [SMILES: OC(CN1CCC(N2CCC(Nc3ccc(-c4ccc(F)cc4)cc3)CC2)CC1)(Cn1cncn1)c1ccc(F)cc1F] |
| 33 | 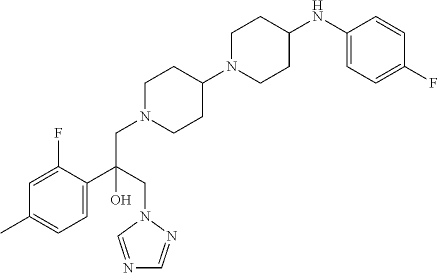 [SMILES: Cc1ccc(C(O)(CN2CCC(N3CCC(Nc4ccc(F)cc4)CC3)CC2)Cn2cncn2)c(F)c1] |
| 34 | 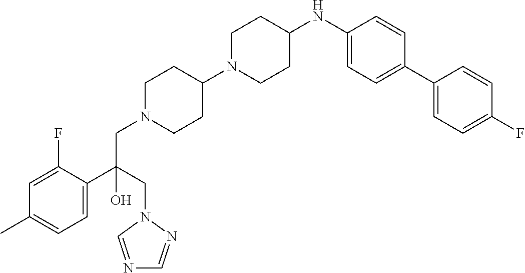 [SMILES: Cc1ccc(C(O)(CN2CCC(N3CCC(Nc4ccc(-c5ccc(F)cc5)cc4)CC3)CC2)Cn2cncn2)c(F)c1] |
| 35 | 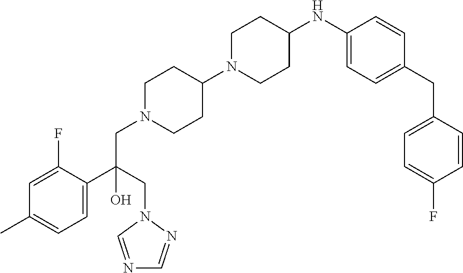 [SMILES: Cc1ccc(C(O)(CN2CCC(N3CCC(Nc4ccc(Cc5ccc(F)cc5)cc4)CC3)CC2)Cn2cncn2)c(F)c1] |
| 36 | 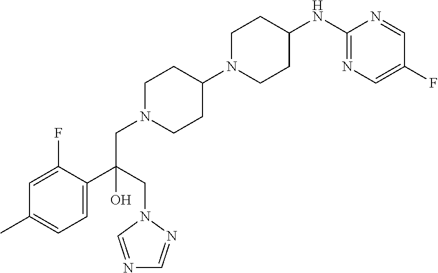 [SMILES: Cc1ccc(C(O)(CN2CCC(N3CCC(Nc4ncc(F)cn4)CC3)CC2)Cn2cncn2)c(F)c1] |
| 37 | 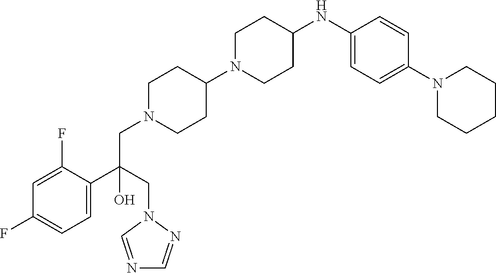 [SMILES: OC(CN1CCC(N2CCC(Nc3ccc(N4CCCCC4)cc3)CC2)CC1)(Cn1cncn1)c1ccc(F)cc1F] |
| 38 | 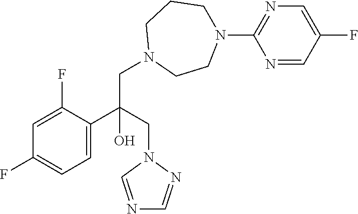 [SMILES: OC(CN1CCCN(c2ncc(F)cn2)CC1)(Cn1cncn1)c1ccc(F)cc1F] |
| 39 | 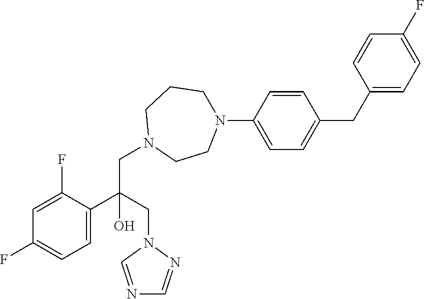 [SMILES: OC(CN1CCCN(c2ccc(Cc3ccc(F)cc3)cc2)CC1)(Cn1cncn1)c1ccc(F)cc1F] |
| 40 | 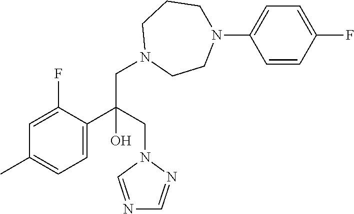 [SMILES: Cc1ccc(C(O)(CN2CCCN(c3ccc(F)cc3)CC2)Cn2cncn2)c(F)c1] |
| 41 | 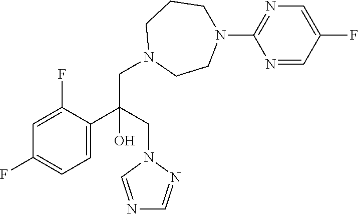 [SMILES: OC(CN1CCCN(c2ncc(F)cn2)CC1)(Cn1cncn1)c1ccc(F)cc1F] |
| 42 | 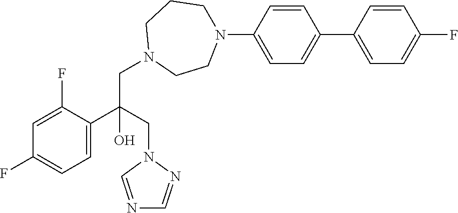 [SMILES: OC(CN1CCCN(c2ccc(-c3ccc(F)cc3)cc2)CC1)(Cn1cncn1)c1ccc(F)cc1F] |
| 43 | 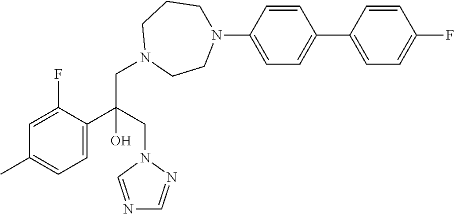 [SMILES: Cc1ccc(C(O)(CN2CCCN(c3ccc(-c4ccc(F)cc4)cc3)CC2)Cn2cncn2)c(F)c1] |
| 44 | 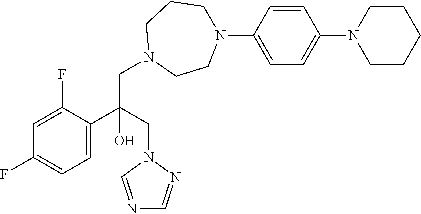 [SMILES: OC(CN1CCCN(c2ccc(N3CCCCC3)cc2)CC1)(Cn1cncn1)c1ccc(F)cc1F] |
| 45 | 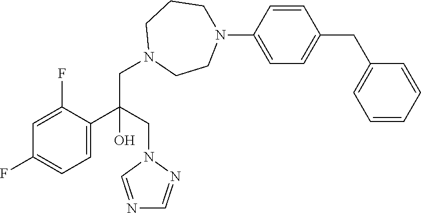 [SMILES: OC(CN1CCCN(c2ccc(Cc3ccccc3)cc2)CC1)(Cn1cncn1)c1ccc(F)cc1F] |
| 46 | 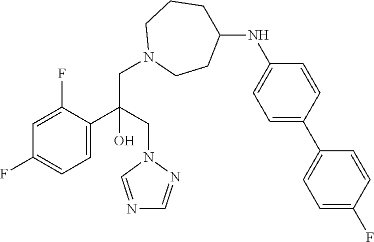 [SMILES: OC(CN1CCCC(Nc2ccc(-c3ccc(F)cc3)cc2)CC1)(Cn1cncn1)c1ccc(F)cc1F] |
| 47 | 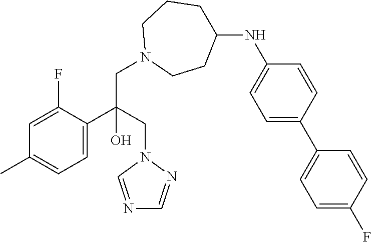 [SMILES: Cc1ccc(C(O)(CN2CCCC(Nc3ccc(-c4ccc(F)cc4)cc3)CC2)Cn2cncn2)c(F)c1] |
| 48 | 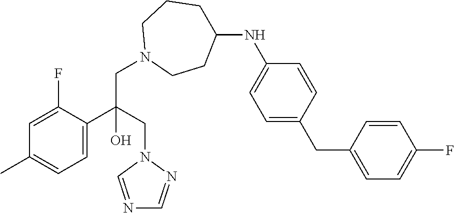 [SMILES: Cc1ccc(C(O)(CN2CCCC(Nc3ccc(Cc4ccc(F)cc4)cc3)CC2)Cn2cncn2)c(F)c1] |
| 49 | 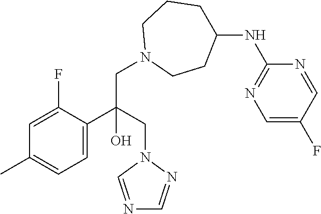 [SMILES: Cc1ccc(C(O)(CN2CCCC(Nc3ncc(F)cn3)CC2)Cn2cncn2)c(F)c1] |
| 50 | 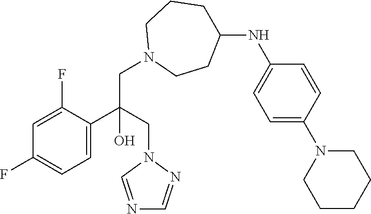 [SMILES: OC(CN1CCCC(Nc2ccc(N3CCCCC3)cc2)CC1)(Cn1cncn1)c1ccc(F)cc1F] |
| 51 | 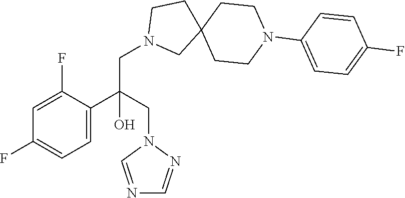 [SMILES: OC(CN1CCC2(CCN(c3ccc(F)cc3)CC2)C1)(Cn1cncn1)c1ccc(F)cc1F] |
| 52 | 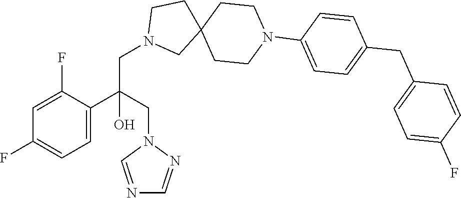 [SMILES: OC(CN1CCC2(CCN(c3ccc(Cc4ccc(F)cc4)cc3)CC2)C1)(Cn1cncn1)c1ccc(F)cc1F] |
| 53 | 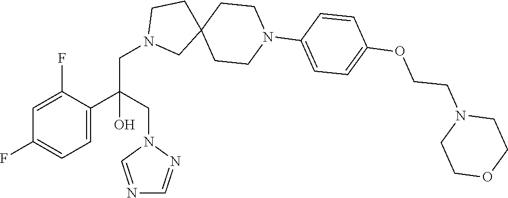 [SMILES: OC(CN1CCC2(CCN(c3ccc(OCCN4CCOCC4)cc3)CC2)C1)(Cn1cncn1)c1ccc(F)cc1F] |
| 54 | 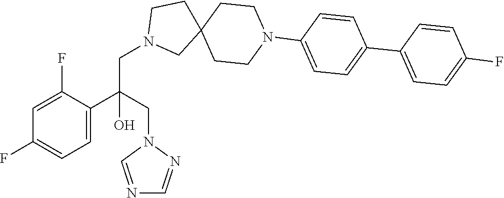 [SMILES: OC(CN1CCC2(CCN(c3ccc(-c4ccc(F)cc4)cc3)CC2)C1)(Cn1cncn1)c1ccc(F)cc1F] |
| 55 | 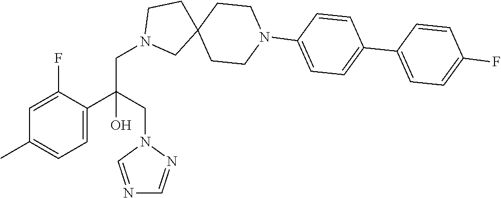 [SMILES: Cc1ccc(C(O)(CN2CCC3(CCN(c4ccc(-c5ccc(F)cc5)cc4)CC3)C2)Cn2cncn2)c(F)c1] |
| 56 | 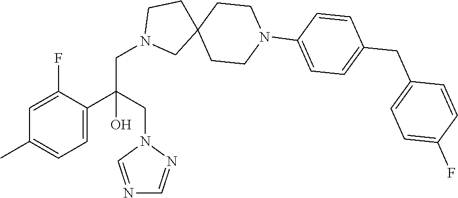 [SMILES: Cc1ccc(C(O)(CN2CCC3(CCN(c4ccc(Cc5ccc(F)cc5)cc4)CC3)C2)Cn2cncn2)c(F)c1] |
| 57 | 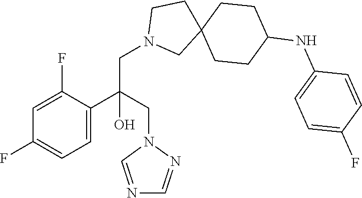 [SMILES: OC(CN1CCC2(CCC(Nc3ccc(F)cc3)CC2)C1)(Cn1cncn1)c1ccc(F)cc1F] |
| 58 | 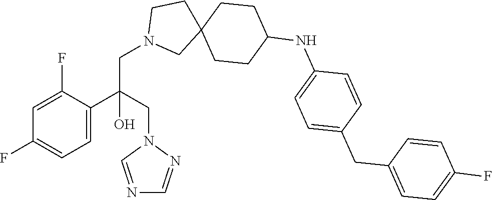 [SMILES: OC(CN1CCC2(CCC(Nc3ccc(Cc4ccc(F)cc4)cc3)CC2)C1)(Cn1cncn1)c1ccc(F)cc1F] |
| 59 | 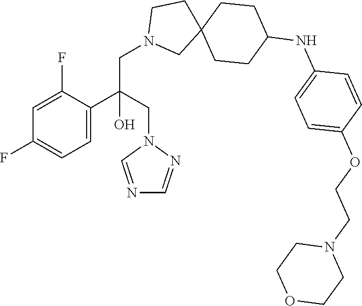 [SMILES: OC(CN1CCC2(CCC(Nc3ccc(OCCN4CCOCC4)cc3)CC2)C1)(Cn1cncn1)c1ccc(F)cc1F] |
| 60 | 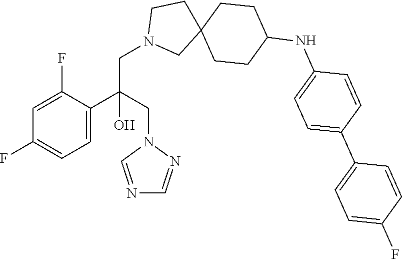 [SMILES: OC(CN1CCC2(CCC(Nc3ccc(-c4ccc(F)cc4)cc3)CC2)C1)(Cn1cncn1)c1ccc(F)cc1F] |
| 61 | 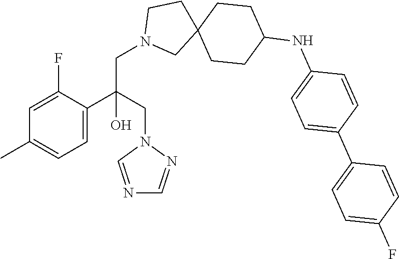 [SMILES: Cc1ccc(C(O)(CN2CCC3(CCC(Nc4ccc(-c5ccc(F)cc5)cc4)CC3)C2)Cn2cncn2)c(F)c1] |
| 62 | 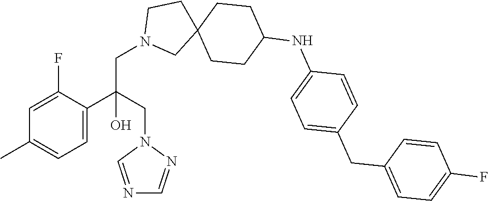 [SMILES: Cc1ccc(C(O)(CN2CCC3(CCC(Nc4ccc(Cc5ccc(F)cc5)cc4)CC3)C2)Cn2cncn2)c(F)c1] |
| 63 | 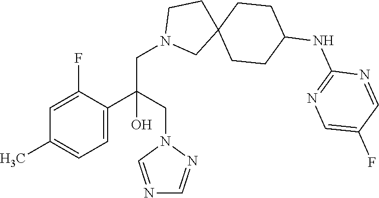 [SMILES: Cc1ccc(C(O)(CN2CCC3(CCC(Nc4ncc(F)cn4)CC3)C2)Cn2cncn2)c(F)c1] |
| 64 | 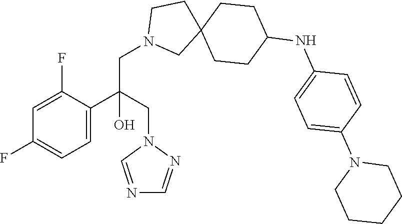 [SMILES: OC(CN1CCC2(CCC(Nc3ccc(N4CCCCC4)cc3)CC2)C1)(Cn1cncn1)c1ccc(F)cc1F] |
| 65 | 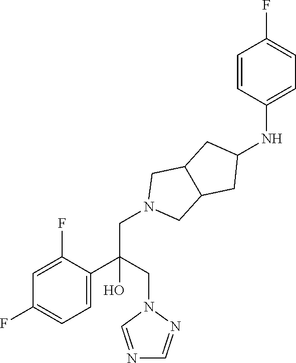 [SMILES: OC(CN1CC2CC(Nc3ccc(F)cc3)CC2C1)(Cn1cncn1)c1ccc(F)cc1F] |
| 66 | 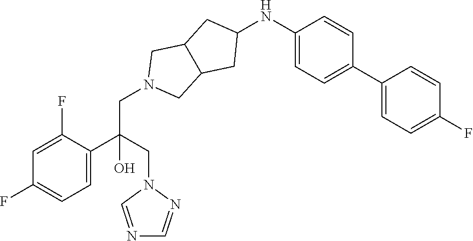 [SMILES: OC(CN1CC2CC(Nc3ccc(-c4ccc(F)cc4)cc3)CC2C1)(Cn1cncn1)c1ccc(F)cc1F] |
| 67 | 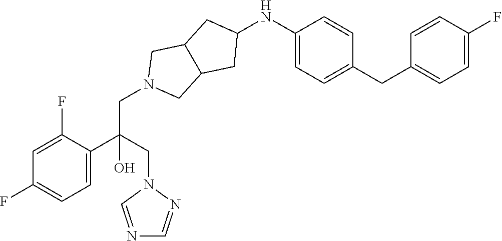 [SMILES: OC(CN1CC2CC(Nc3ccc(Cc4ccc(F)cc4)cc3)CC2C1)(Cn1cncn1)c1ccc(F)cc1F] |
| 68 | 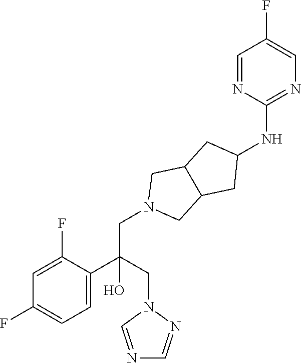 [SMILES: OC(CN1CC2CC(Nc3ncc(F)cn3)CC2C1)(Cn1cncn1)c1ccc(F)cc1F] |
| 69 | 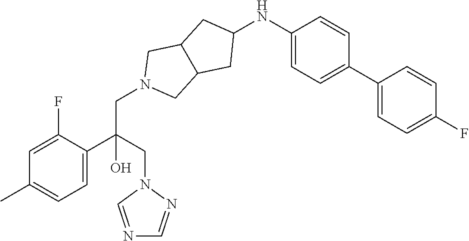 [SMILES: Cc1ccc(C(O)(CN2CC3CC(Nc4ccc(-c5ccc(F)cc5)cc4)CC3C2)Cn2cncn2)c(F)c1] |
| 70 | 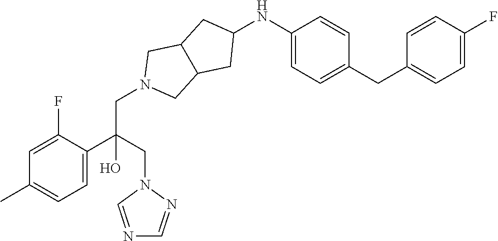 [SMILES: Cc1ccc(C(O)(CN2CC3CC(Nc4ccc(Cc5ccc(F)cc5)cc4)CC3C2)Cn2cncn2)c(F)c1] |
| 71 | 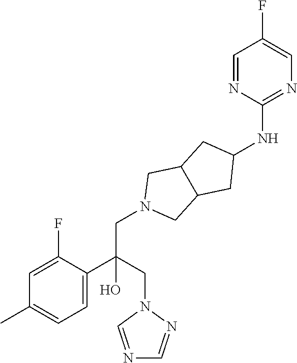 [SMILES: Cc1ccc(C(O)(CN2CC3CC(Nc4ncc(F)cn4)CC3C2)Cn2cncn2)c(F)c1] |
| 72 | 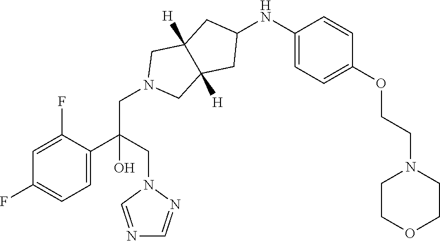 [SMILES: OC(CN1C[C@H]2CC(Nc3ccc(OCCN4CCOCC4)cc3)C[C@H]2C1)(Cn1cncn1)c1ccc(F)cc1F] |
| 73 | 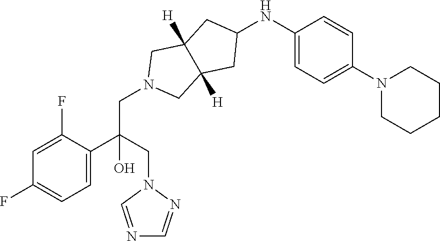 [SMILES: OC(CN1C[C@H]2CC(Nc3ccc(N4CCCCC4)cc3)C[C@H]2C1)(Cn1cncn1)c1ccc(F)cc1F] |
| 74 | 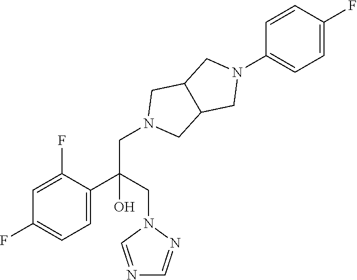 [SMILES: OC(CN1CC2CN(c3ccc(F)cc3)CC2C1)(Cn1cncn1)c1ccc(F)cc1F] |
| 75 | 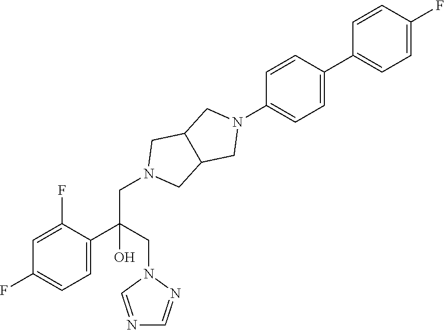 [SMILES: OC(CN1CC2CN(c3ccc(-c4ccc(F)cc4)cc3)CC2C1)(Cn1cncn1)c1ccc(F)cc1F] |
| 76 | 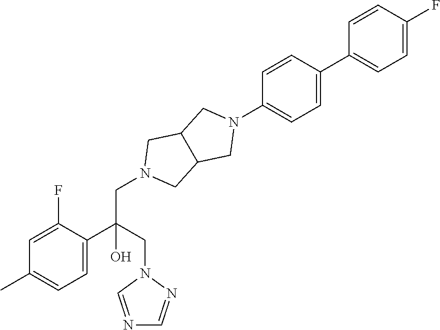 [SMILES: Cc1ccc(C(O)(CN2CC3CN(c4ccc(-c5ccc(F)cc5)cc4)CC3C2)Cn2cncn2)c(F)c1] |
| 77 | 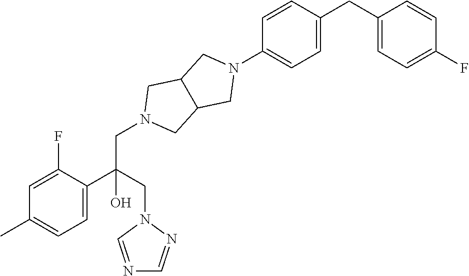 [SMILES: Cc1ccc(C(O)(CN2CC3CN(c4ccc(Cc5ccc(F)cc5)cc4)CC3C2)Cn2cncn2)c(F)c1] |
| 78 | 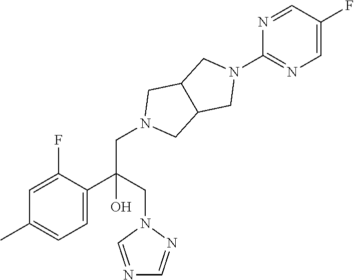 [SMILES: Cc1ccc(C(O)(CN2CC3CN(c4ncc(F)cn4)CC3C2)Cn2cncn2)c(F)c1] |
| 79 | 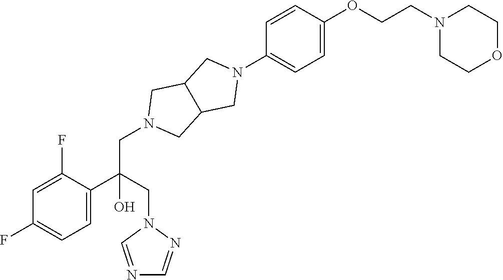 [SMILES: OC(CN1CC2CN(c3ccc(OCCN4CCOCC4)cc3)CC2C1)(Cn1cncn1)c1ccc(F)cc1F] |
| 80 | 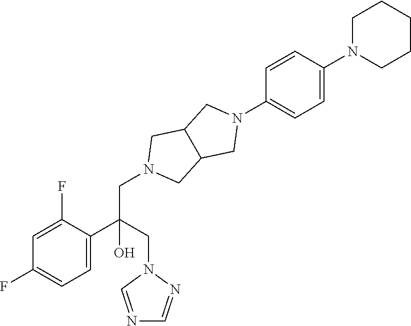 [SMILES: OC(CN1CC2CN(c3ccc(N4CCCCC4)cc3)CC2C1)(Cn1cncn1)c1ccc(F)cc1F] |

TABLE 3

| Example number: | Example structure: |
|---|---|
| 81 | 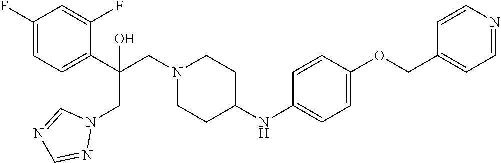 [SMILES: OC(CN1CCC(Nc2ccc(OCc3ccncc3)cc2)CC1)(Cn1cncn1)c1ccc(F)cc1F] |
| 82 | 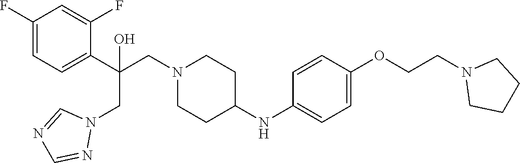 [SMILES: OC(CN1CCC(Nc2ccc(OCCN3CCCC3)cc2)CC1)(Cn1cncn1)c1ccc(F)cc1F] |
| 83 | 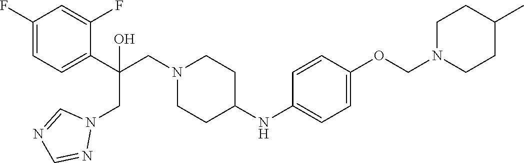 [SMILES: CC1CCN(COc2ccc(NC3CCN(CC(O)(Cn4cncn4)c4ccc(F)cc4F)CC3)cc2)CC1] |
| 84 | 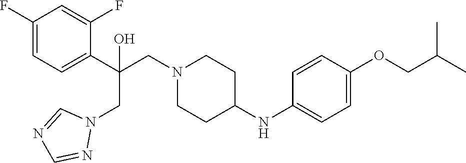 [SMILES: CC(C)COc1ccc(NC2CCN(CC(O)(Cn3cncn3)c3ccc(F)cc3F)CC2)cc1] |
| 85 | 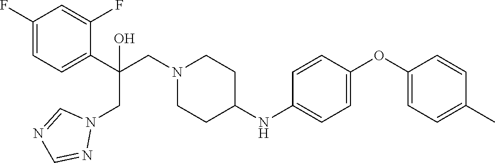 [SMILES: Cc1ccc(Oc2ccc(NC3CCN(CC(O)(Cn4cncn4)c4ccc(F)cc4F)CC3)cc2)cc1] |
| 86 | 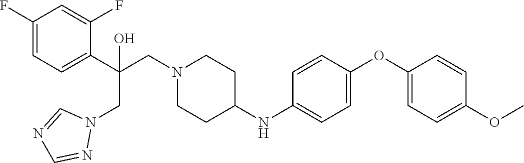 [SMILES: COc1ccc(Oc2ccc(NC3CCN(CC(O)(Cn4cncn4)c4ccc(F)cc4F)CC3)cc2)cc1] |
| 87 | 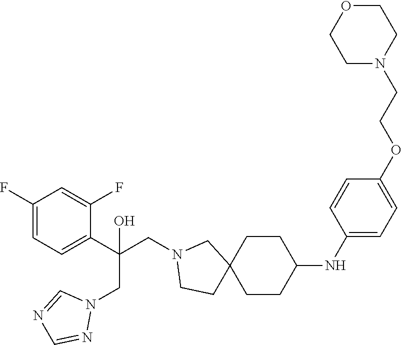 [SMILES: OC(CN1CCC2(CCC(Nc3ccc(OCCN4CCOCC4)cc3)CC2)C1)(Cn1cncn1)c1ccc(F)cc1F] |
| 88 | 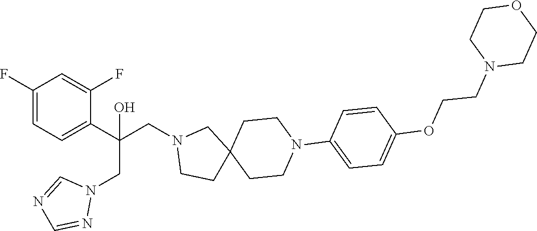 [SMILES: OC(CN1CCC2(CCN(c3ccc(OCCN4CCOCC4)cc3)CC2)C1)(Cn1cncn1)c1ccc(F)cc1F] |
| 89 | 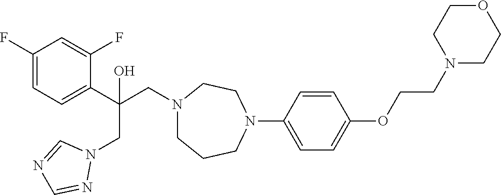 [SMILES: OC(CN1CCCN(c2ccc(OCCN3CCOCC3)cc2)CC1)(Cn1cncn1)c1ccc(F)cc1F] |
| 90 | 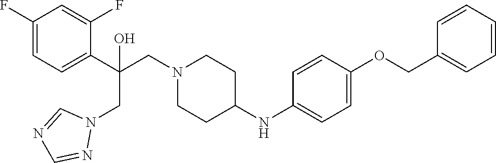 [SMILES: OC(CN1CCC(Nc2ccc(OCc3ccccc3)cc2)CC1)(Cn1cncn1)c1ccc(F)cc1F] |
| 91 | 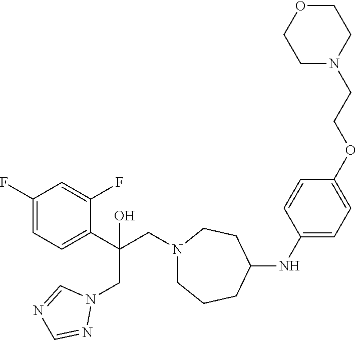 [SMILES: OC(CN1CCCC(Nc2ccc(OCCN3CCOCC3)cc2)CC1)(Cn1cncn1)c1ccc(F)cc1F] |
| 92 | 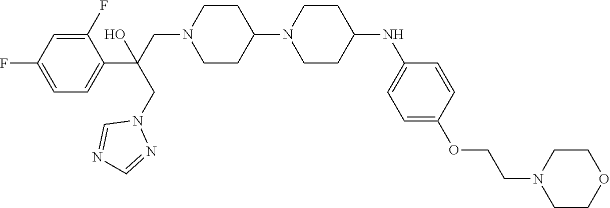 [SMILES: OC(CN1CCC(N2CCC(Nc3ccc(OCCN4CCOCC4)cc3)CC2)CC1)(Cn1cncn1)c1ccc(F)cc1F] |
| 93 | 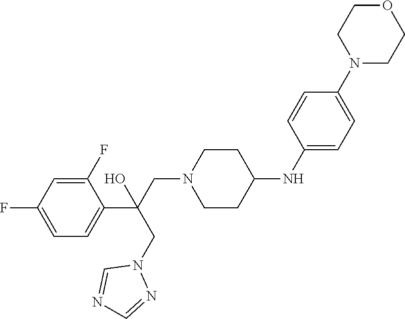 [SMILES: OC(CN1CCC(Nc2ccc(N3CCOCC3)cc2)CC1)(Cn1cncn1)c1ccc(F)cc1F] |
| 94 | 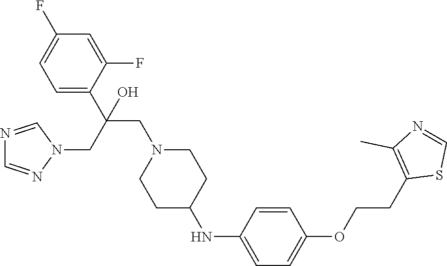 [SMILES: Cc1ncsc1CCOc1ccc(NC2CCN(CC(O)(Cn3cncn3)c3ccc(F)cc3F)CC2)cc1] |
| 95 | 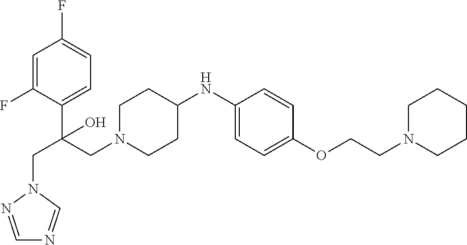 [SMILES: OC(CN1CCC(Nc2ccc(OCCN3CCCCC3)cc2)CC1)(Cn1cncn1)c1ccc(F)cc1F] |
| 96 | 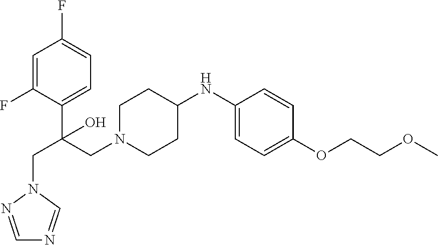 [SMILES: COCCOc1ccc(NC2CCN(CC(O)(Cn3cncn3)c3ccc(F)cc3F)CC2)cc1] |
| 97 | 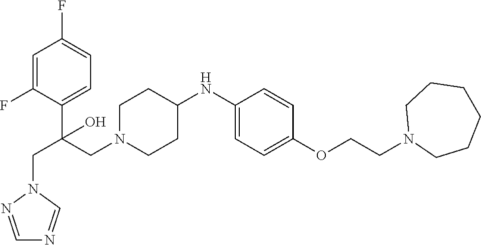 [SMILES: OC(CN1CCC(Nc2ccc(OCCN3CCCCCC3)cc2)CC1)(Cn1cncn1)c1ccc(F)cc1F] |
| 98 | 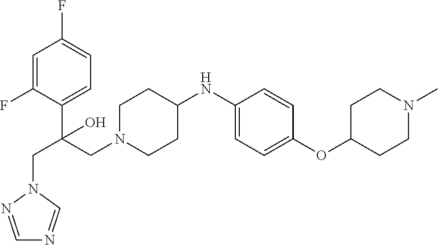 [SMILES: CN1CCC(Oc2ccc(NC3CCN(CC(O)(Cn4cncn4)c4ccc(F)cc4F)CC3)cc2)CC1] |
| 99 | 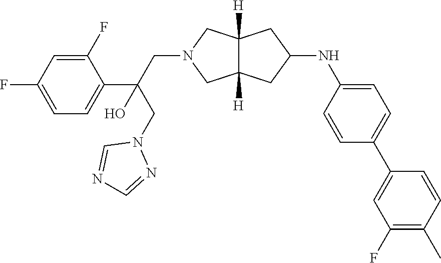 [SMILES: Cc1ccc(-c2ccc(NC3C[C@@H]4CN(CC(O)(Cn5cncn5)c5ccc(F)cc5F)C[C@@H]4C3)cc2)cc1F] |
| 100 | 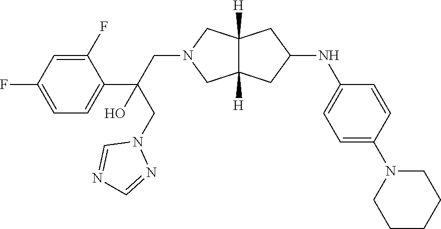 [SMILES: OC(CN1C[C@H]2CC(Nc3ccc(N4CCCCC4)cc3)C[C@H]2C1)(Cn1cncn1)c1ccc(F)cc1F] |
| 101 | 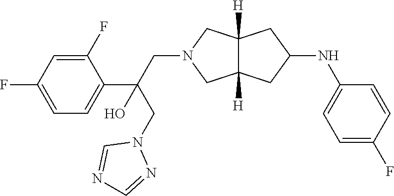 [SMILES: OC(CN1C[C@H]2CC(Nc3ccc(F)cc3)C[C@H]2C1)(Cn1cncn1)c1ccc(F)cc1F] |

In some embodiments the compound of the invention have a lower MIC than fluconazole against a fungal strain, for example one of the fungal strains disclosed herein (e.g. a Candida spp., such as C. albicans, C. glabrata, C. krusei, C. tropicalis, C. parapsilosis or particularly C. auris; or a Aspergillus spp., such as Aspergillus fumigatus) including drug-resistant strains thereof. For example, it may be that the compound of the invention has an MIC that is more than 5, 10, 20, 50, 100, 500, 1000, 5000 or 10000 fold lower than fluconazole against a fungal strain (e.g. a Candida spp., for example Candida albicans or Candida auris) including drug-resistant strains thereof.

The inventors have found that certain compounds of the invention protect against Candida auris in a Galleria mellonella as illustrated in the Examples. This suggests that compounds of the invention exhibit selective toxicity toward fungal cells and may therefore exhibit a wide therapeutic window, with a low risk of undesirable side effects.

### Pharmaceutical Compositions

In accordance with another aspect, the present invention provides a pharmaceutical composition comprising a compound of the invention, or a pharmaceutically acceptable salt thereof, and a pharmaceutically acceptable excipient.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions of the invention may be in a form suitable for oral, buccal, or sublingual use (for example as tablets, lozenges, hard or soft capsules, aqueous or oily suspensions, emulsions, dispersible powders or granules, syrups or elixirs), for topical use (for example as creams, ointments, lotions, foams, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, intraarterial, subcutaneous, intramuscular or intraperitoneal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.1 mg to 0.5 g of active agent (more suitably from 0.5 to 250 mg, for example from 1 to 100 mg, from 1 to 50 mg, from 1 to 25 mg or from 1 to 10 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.1 mg/kg to 100 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general lower doses will be administered when a parenteral route is employed. Thus, for example, for intravenous, subcutaneous, intramuscular or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Suitably the compound of the invention is administered orally, for example in the form of a tablet or capsule dosage form, a powder or a solution or suspension. The daily dose administered orally may be, for example a total daily dose selected from 1 mg to 1000 mg, 5 mg to 1000 mg, 10 mg to 750 mg or 25 mg to 500 mg. Typically, unit dosage forms will contain about 0.5 mg to 0.5 g of a compound of this invention. In a particular embodiment the compound of the invention is administered parenterally, for example by intravenous administration. In another particular embodiment the compound of the invention is administered orally. In another embodiment the compound of the invention is administered topically.

### Therapeutic Uses and Applications

In accordance with another aspect, the present invention provides a compound of the invention, or a pharmaceutically acceptable salt thereof, or a pharmaceutical composition of the invention, for use as a medicament.

Also provided is a compound of the invention, or a pharmaceutically acceptable salt thereof, or a pharmaceutical composition of the invention, for use in the treatment or prevention of a fungal infection in a subject.

Also provided is a method of treating or preventing a fungal infection in a subject, comprising administering to the subject an effective amount of a compound of the invention, or a pharmaceutically acceptable salt thereof, or a pharmaceutical composition of the invention.

Also provided is the use of a compound of the invention, or a pharmaceutically acceptable salt thereof, or a pharmaceutical composition of the invention, for the manufacture of a medicament for the treatment or prevention of a fungal infection in a subject.

In the following sections of the application reference is made to a compound of the invention, or a pharmaceutically acceptable salt thereof, or a composition of the invention for use in the treatment of certain diseases or conditions. It is to be understood that any reference herein to a compound or composition for a particular use is also intended to be a reference to (i) the use of the compound of the invention, or pharmaceutically acceptable salt thereof, or a composition of the invention, for the manufacture of a medicament for the treatment of that disease or condition; and (ii) a method of treating the disease or condition in a subject, the method comprising administering to the subject a therapeutically effective amount of the compound of the invention, or pharmaceutically acceptable salt thereof, or a composition of the invention.

In some embodiments the compound of the invention is for use in the treatment or prevention of a superficial fungal infection. In some embodiments the compound of the invention is for use in the treatment or prevention of a cutaneous fungal infection. In some embodiments the compound of the invention is for use in the treatment or prevention of a subcutaneous fungal infection. In some embodiments the compound of the invention is for use in the treatment or prevention of a skin, nail, hair, scalp, soft tissue (e.g. vagina or vulva) fungal infection, a fungal infection of a bite or wound (e.g. a surgical or trauma wound), or fungal infection of the site of a catheter, intravenous line, intraarterial line or cannula.

In some embodiments the compound of the invention is for use in the treatment or prevention of a systemic fungal infection. It may be that the compound of the invention is for use in the treatment or prevention of a fungal infection of the bloodstream, an internal organ (e.g. the kidney, heart, lungs, liver, spleen, abdomen, central nervous system or brain), mouth, throat, oesophagus or bone.

In some embodiments the compound of the invention is for use in the treatment or prevention of a fungal infection in a subject with a compromised immune system. It may be that the subject is neutropenic, (e.g. a subject with chemotherapy induced neutropenia). It may be that the subject has tuberculosis, typhoid, Dengue fever or a viral infection (e.g. HIV, viral hepatitis, Epstein-Barr virus). It may be that the subject is being treated with an immunosuppressant therapy (e.g. subjects that have undergone transplant therapy (e.g. stem cell transplant or organ transplant)). It may be that the subject has a cancer (e.g. a solid tumour, or a liquid cancer (haematological malignancies such as leukaemia)). It may be that the subject has renal failure or is undergoing haemodialysis. It may be that the subject that has undergone a surgical procedure, particularly major surgery (e.g. abdominal surgery). It may be that the subject is a critically ill subject in intensive care. It maybe that the subject has a catheter (e.g. central venous catheters). It may be that the subject has a CNS medical device (e.g. ventriculostomy drains, shunts, stimulators, prosthetic reconstructive devices, and biopolymer wafers that deliver chemotherapy). It may be that the subject has been or is being treated with antibiotics. It may be that the subject is a pre-term infant, particularly a pre-term infant with low birth weight. In these embodiments the fungal infection may be caused by or associated with, any of the fungal species described herein (e.g. Candida spp. or Aspergillus spp.).

In some embodiments it may be that the fungal infection (e.g. any of the infections described herein) is asymptomatic. In some embodiments it may be that the fungal infection (e.g. any of the infections described herein) is a symptomatic fungal infection. For example a subject with a symptomatic fungal infection may experience one or more of flu-like symptoms, cough, shortness of breath, fever, chills, fatigue, inflammation, pruritis or erythema.

In some embodiments the fungal infection is caused by, or associated with a pathogenic fungal species. For example a fungal species selected from Candida spp. (e.g. C. albicans, C. glabrata, C. tropicalis, C. parapsilosis, C. krusei, C. auris, C. dubliniensis, C. lusitaniae and C. guilliermondii); Aspergillus spp. (e.g. A. fumigatus, A. flavus, A. terreus, A. nidulans, A. niger, or A. versicolor, particularly A. fumigatus or A. flavus), Cryptococcus spp. (e.g. Cryptococcus neoformans or Cryptococcus gattii) and Histoplasma spp. (e.g. Histoplasma. capsulatum).

In some embodiments the fungal infection is caused by or associated with Candida spp. fungus. In some embodiments the fungal infection is caused by or associated with a Candida fungus selected from: C. albicans, C. glabrata, C. tropicalis, C. parapsilosis, C. krusei and C. auris. In some embodiments the fungal infection is caused by or associated with Candida albicans. In some embodiments the fungal infection is caused by or associated with Candida auris.

In some embodiments the fungal infection is caused by or associated with Aspergillus spp. In some embodiments the fungal infection is caused by or associated with Aspergillus fumigatus.

In some embodiments the fungal infection is candidiasis, for example oropharyngeal candidiasis (candidiasis of the mouth, throat or oesophagus), vaginal candidiasis (e.g. vulvovaginal candidiasis) or candida intertrigo. In some embodiments the fungal infection is an invasive candidiasis, for example candidemia (infection of the bloodstream), chronic disseminated candidiasis (hepatosplenic candidiasis), intraabdominal candidiasis, peritoneal candidiasis, candida endocarditis, candidiasis associated with an implanted cardiac device (e.g. pacemakers or defibrillators and prosthetic valves), candida suppurative thrombophlebitis, candida osteomyelitis, candida septic arthritis, an ophthalmic candida infection (e.g. candida endophthalmitis or candida chorioretinitis), central nervous system candidiasis, urinary tract candidiasis (e.g. candiduria, candida cystitis, candida pyelonephritis or candida urinary tract infection associated with fungus balls), or candida infections of the respiratory tract. In some embodiments the compound of the invention is for use in the treatment or prevention of esophageal, oropharyngeal, peritoneal, urinary tract or vulvovaginal candidiasis; a systemic candida infection selected from candidemia, disseminated candidiasis and pneumonia caused by or associated with candida; or cryptococcal meningitis. In some embodiments the compound of the invention is for use in the treatment or prevention of vulvovaginal candidiasis.

In some embodiments the fungal infection is aspergillosis, for example allergic bronchopulmonary aspergillosis, allergic aspergillus sinusitis, aspergilloma, chronic pulmonary aspergillosis, invasive aspergillosis or cutaneous aspergillosis.

In some embodiments a compound of the invention is for use in the treatment or prevention of a fungal lung infection. It may be that the fungal lung infection is fungal asthma, fungal pneumonia, aspergillosis (e.g. invasive pulmonary aspergillosis or chronic pulmonary aspergillosis), coccidioidomycosis (valley fever), histoplasmosis, blastomycosis, paracoccidioidomycosis, cryptococcosis and pulmonary mucormycosis or pulmonary sporotrichosis. It may be that the compound of the invention is for use in the treatment or prevention of a fungal lung infection in a subject with a compromised immune system as described herein, for example subjects with HIV, subjects undergoing cancer chemotherapy, or subjects treated with an immunosuppressant.

In some embodiments a compound of the invention is for use in the treatment or prevention of invasive aspergillosis, fungal meningitis, extrapulmonary coccidioidomycosis, cryptococcosis (e.g. cryptococcal meningitis), Cryptococcus gattii. cryptococcosis, disseminated, and central nervous system (CNS) histoplasmosis, mucormycosis (e.g. rhinocerebral mucormycosis, gastrointestinal mucormycosis or disseminated mucormycosis), sporotrichosis (e.g. disseminated sporotrichosis).

In some embodiments a compound of the invention is for use in the treatment or prevention of cutaneous aspergillosis, cutaneous mucormycosis, ringworm (tinea, e.g. tinea pedis, tinea cruris or tinea capitis) and cutaneous sporotrichosis.

In some embodiments a compound of the invention is for use in the treatment or prevention of a fungal eye infection (e.g. fungal keratitis, exogenous fungal endophthalmitis or fungal endophthalmitis).

In some embodiments a compound of the invention is for use in the treatment or prevention of a fungal nail infection. It may be that the compound of the invention is for use in the treatment or prevention of a fungal nail infection selected from distal or lateral subungual onychomycosis, proximal subungual onychomycosis, superficial onychomycosis, endonyx onychomycosis, dystrophic onychomycosis or yeast onychomycosis. It may be that the fungal nail infection is caused by or associated with a Candida spp. fungus.

In some embodiments a compound of the invention is for use in the treatment of a pathogenic fungus. It may be that the treatment comprises contacting the fungus with a compound of the invention, for example by topically and/or systemically administration of the compound of the invention to a subject. It may be that the pathogenic fungus is one or more of the fungi described herein, for example a Candida spp. or Aspergillus spp. The treatment may reduce the fungal load in subject or eliminate the fungus from the subject. It may be that the compound of the invention has a fungicidal effect on the fungi treated with the compound. In some embodiments the treatment may prevent or inhibit the growth of the fungus. It may be that the compound of the invention has a fungistatic effect on the fungi treated with the compound.

### Antifungal Susceptibility Testing

In some embodiments the fungal infection is tested for susceptibility to treatment with a compound of the invention prior to administering the compound to the subject. The susceptibility of the fungus to treatment may be assessed using well-known methods including broth dilution methods described in the Clinical and Laboratory Standards Institute (CLSI) M27, 4^{th} Edition November 2017, or European Committee on Antimicrobial Susceptibility Testing (EUCAST) methodologies for susceptibility testing of yeasts, v 7.3.1 valid from 15 January, 2017.

Accordingly in some embodiments a compound of the invention is for use in a method of treating a fungal infection in a subject, the method comprising administering to the subject the compound of the invention after determining that the fungal infection is susceptible to treatment with the compound of the invention. Susceptibility to treatment may be assessed using known methods including but not limited to those described herein.

In some embodiments there is provided a method of treating a fungal infection in subject, wherein the fungus causing the infection is susceptible to treatment with a compound of the invention, the method comprising administering an effective amount of said compound of the invention to the subject.

In some embodiments there is provided a method of treating a fungal infection in a subject, the method comprising: (i) obtaining a sample of fungus from the subject; (ii) testing the susceptibility of the fungus to a compound of the invention and (iii) if the fungus is susceptible to said compound, administering an effective amount of said compound to the subject. A sample of the fungus may be obtained by, for example, swabbing the site of fungal infection, by obtaining a sample of tissue infected with the fungus or from a suitable bodily fluid containing the fungus (e.g. blood, serum, plasma, saliva, urine, a mucous or bronchoalveolar lavage).

In the above four embodiments the fungal infection may be any of the fungal infections described herein.

### Resistant Fungi

The widespread use of antifungal agents has led to the emergence of fungal species with acquired resistance to one or more conventional antifungal agents. Additionally, certain fungi are inherently resistant to certain antifungal drugs. For example, Candida glabrata and non-fumigatus Aspergillus are inherently resistant to azole antifungal drugs. Azole antifungal drugs act by act by binding to and inhibiting the intracellular target enzyme ERG11p (Lanosterol 14α-demethylase) involved in the biosynthesis of ergosterol. mechanistically, the azole ring, N4 nitrogen in triazole specifically, binds to the heme iron bearing the protoporphyrin unit at the CYP450DM active site stoichiometrically, and inhibits its binding to the substrates and oxygen.

Antifungal drug resistance can be either inherited vertically or transmitted horizontally to different species. The main antifungal drug resistance mechanisms are one or more of a reduction in the intracellular drug concentration; mutation of the drug target; or modification of the drug metabolic pathways.

In the case of azole antifungal drugs, resistance is primarily attributed to one or more of increased expression of efflux pumps, mutation of the drug target (Erg11 mutation) and/or ERG11 gene upregulation. The presence of biofilms comprising fungi is also a factor in the emergence of antifungal resistance.

### Efflux Pump Activity

The overexpression of efflux pumps in fungi transports antifungal drugs out of the cell effectively reducing the intracellular concentration to a level below which it can influence its target. Eukaryotic fungal cells have two different types of efflux pumps which have been shown to impact drug resistance. ATP binding cassette (ABC) family pumps, which uses ATP as a source of energy to drive the drug efflux, and major facilitator superfamily (MFS) pumps that utilize the proton motive force to expel antifungal drugs. ATP-dependent transporters comprise two transmembrane domains (TMD) and two ATP-hydrolysis catalyzing nucleotide-binding domains (NBD) that are located in cytoplasm. In contrast, MFS transporters usually comprise 12 to 14 TMDs (Castelo-Branco et al., Med. Mycol. 2020 Jan. 17. pii: myz135. doi:10.1093/mmy/myz135. PubMed PMID: 31950176; Parent-Michaud et al., J. Antimicrob. Chemother. 2019 Dec. 31, pii: dkz534. doi: 10.1093/jac/dkz534. PubMed PMID:31891387; and Nishimoto et al., J. Antimicrob. Chemother. 2020 Feb. 1; 75(2):257-270.

The table below illustrates the efflux pumps present in inherently resistant and acquired resistance strains of Candida species:

| | MIC | | |
|---|---|---|---|
| | Fluconazole | Efflux | |
| Fungal Strain | μg/ml | Pump | Pump type |
| C. albicans 3281 | >128 | CDR1 | ABC |
| | | CDR2 | ABC |
| | | MDR1 | MFS |
| | 0.5 | MDR1 | MFS |
| C. parapsilosis 3209 | | MDR2 | MFS |
| C. glabrata 8018 | >128 | CDR1 | ABC |
| | | CDR2/PDH1 | ABC |
| | | SNQ2 | ABC |
| C. krusei 3876 | 32 | ABC1P | ABC |
| C. auris (e.g. | >128 | CDR1 | ABC |
| TDG1912, TDG2506 or 8984) | | MDR1 | MFS |
| | | RDC3 | MFS |
| | | SNQ2 | ABC |
| | | YHD3 | ABC |
| C. tropicalis 8760 | — | CDR1 | ABC |
| | | MDR1 | MFS |

see also Prasad et al., Fungal Genet Biol. 2019 November; 132:103252. doi: 10.1016/j.fgb.2019.103252. Epub 2019 Jul. 11.

### Target Alteration

Target site mutations in the ERG11-encoded lanosterol 14-alpha-demethylase enzyme, are a common cause of azole resistance. Mutations affect the active site of the enzyme meaning that azole compounds no longer bind effectively to the target to inhibit synthesis of essential component of the fungal cell wall (Hou X et al., Antimicrob. Agents Chemother., 2018 Dec. 21; 63(1)). The Examples herein illustrate that compounds of the invention are active against C. auris strains which carry such ERG11 mutations.

### ERG11 Overexpression

Overexpression of Erg11 can also mediate resistance to azoles. This can result from duplication of the gene within Candida species, ether specific to ERG11 or through chromosomal duplication, or through mutations in regulators (e.g. Upc2) affecting the expression of ERG11. Either mechanism can result in reduced efficacy by titrating out the available drug within the cell and allowing the continued function of un-inhibited enzyme (Bhattacharya et al., Sci. Rep. 2019 Mar. 25; 9(1)).

The inventors have found that compounds of the invention are active against a broad panel of Candida strains and that some compounds of the invention are active against Candida strains, including Candida auris, that are inherently resistant to conventional anti-fungal agents such as azoles (e.g. fluconazole). As illustrated in the Examples, compounds of the invention have also been found to be effective against, and multi-drug resistant strains of Candida auris and Candida albicans.

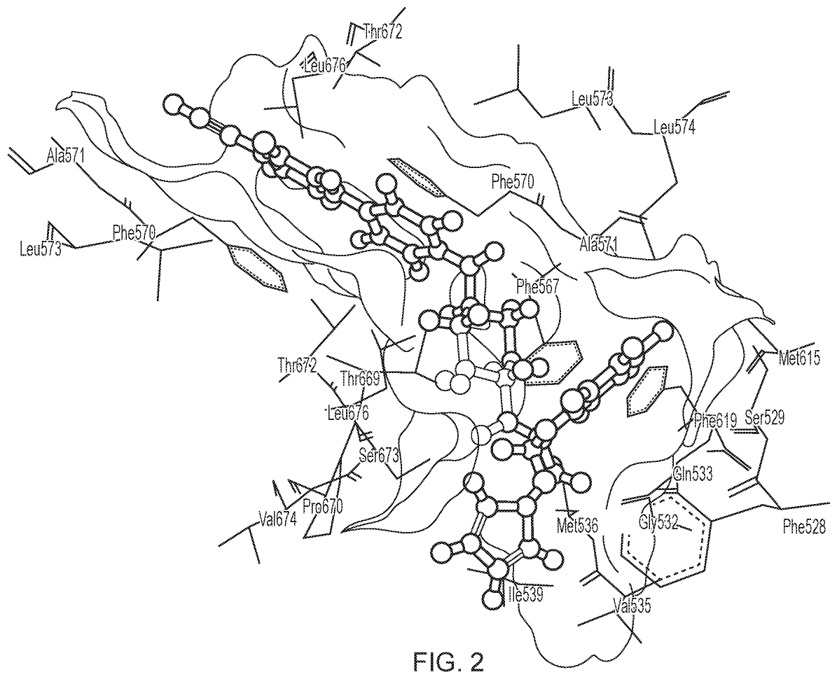

Without wishing to be bound by theory, molecular modelling suggests that compounds of the invention exhibit greater affinity towards the target enzyme ERG11p (Lanosterol 14α-demethylase) through increased hydrophobic and hydrogen bond interactions compared to conventional azole antifungals such as fluconazole, and fit snugly within the binding pocket compared to conventional azole antifungal drugs such as fluconazole (see FIG. 2). Modelling also indicates that compounds of the invention exhibit greater affinity towards efflux pumps commonly found in fungi such as Candida spp. through increased hydrophobic and hydrogen bonding compared to conventional azole antifungal drugs such as fluconazole. The increased affinity reduces the ability of the efflux pumps to expel the compound from the cell. The increased binding affinity to the target enzyme and/or efflux pumps is therefore expected to enhance the antifungal effects of the compounds of the invention.

### Treatment of Drug-Resistant Fungal Infections

Also provided is a compound of the invention for use in the treatment or prevention of a fungal infection in a subject, wherein said fungal infection is caused by or associated with a drug-resistant fungal strain.

Reference to “a drug-resistant fungal strain” refers to fungi that are resistant to at least one antifungal drug that was approved by the FDA or EMA on or before 16 Dec. 2019. It may be that the drug-resistant fungal strain is inherently resistant to one or more existing antifungal drug(s). It may be that the drug-resistant fungal strain is a fungal strain which has acquired resistance to one or more existing antifungal drug(s). It may be that the drug-resistant fungal strain is a multi-drug resistant strain (i.e. a fungal strain that is resistant to treatment with two or more existing antifungal drugs).

It may be that the drug-resistant fungal strain is a drug resistant Candida strain or a drug resistant Aspergillus strain.

It may be that the drug-resistant fungal strain is a drug resistant Candida strain. It may be that the drug-resistant fungal strain is selected from a drug-resistant strain of Candida albicans, Candida glabrata, Candida tropicalis, Candida parapsilosis, Candida krusei and Candida auris. It may be that the drug-resistant fungal strain is selected from Candida glabrata and Candida krusei. It may be that the drug-resistant fungal strain is selected from a drug-resistant strain of Candida albicans. It may be that the drug-resistant fungal strain is selected from a drug-resistant strain of Candida auris.

It may be that the drug-resistant fungal strain is a drug resistant Aspergillus strain. It may be that the drug-resistant fungal strain is a drug resistant Aspergillus fumigatus strain.

In some embodiments the drug-resistant fungal strain is resistant to one or more antifungal drugs selected from: a polyene antifungal drug (e.g. amphotericin B (e.g. amphotericin B deoxycholate, liposomal amphotericin B, amphotericin B lipid complex or amphotericin B colloidal dispersion), candicidin, filipin, hamycin, natamycin, nystatin, or rimocidin); a triazole antifungal drug (also known as azole antifungal drugs), other than a compound of the invention (e.g. albaconazole, efinaconazole, epoxiconazole, fluconazole, isavuconazole, itraconazole, posaconazole, propiconazole, ravuconazole, terconazole or voriconazole); an imidazole antifungal drug (e.g., bifonazole, butoconazole, chlormidazole, clotrimazole, eberconazole, econazole, fenticonazole, flutrimazole, isoconazole, ketoconazole, luliconazole, miconazole, omoconazole, oxiconazole, sertaconazole, sulconazole, or tioconazole); a thiazole antifungal drug (e.g., abafungin); an echinocandin antifungal drug (e.g. anidulafungin, biafungin, caspofungin, or micafungin); an allylamine antifungal drug (e.g. amorolfin, butenafine, naftifine, or terbinafine); amorolfine, ciclopirox olamine, griseofulvin or flucytosine (5-fluorocytosine).

It may be that the drug-resistant fungal strain is a drug-resistant Candida strain that is resistant to one or more of a polyene, triazole or echinocandin antifungal drug. It may be that the drug-resistant fungal strain is a drug-resistant Candida strain that is resistant to a triazole (e.g. fluconazole) and/or or echinocandin antifungal drug. It may be that the drug-resistant fungal strain is a drug-resistant Candida albicans strain that is resistant to one or more of a polyene, triazole or echinocandin antifungal drug. It may be that the drug-resistant fungal strain is a drug-resistant Candida albicans strain that is resistant to a triazole (e.g. fluconazole) and/or or echinocandin antifungal drug. It may be that the drug-resistant fungal strain is a drug-resistant Candida auris strain that is resistant to one or more of a polyene, triazole or echinocandin antifungal drug. It may be that the drug-resistant fungal strain is a drug-resistant Candida auris strain that is resistant to a triazole (e.g. fluconazole) and/or or echinocandin antifungal drug.

The resistance of a fungal strain to an antifungal drug can be determined using well-known methods, including the susceptibility testing described herein.

It may be that the fungal infection caused by or associated with a drug-resistant fungal strain is any one of the fungal infections described herein.

### Prophylaxis

In some embodiments a compound of the invention is for use in the prevention (prophylaxis) of a fungal infection in a subject. Treatment of a subject with a compound of the invention may be used to decolonize, reduce or eradicate pathogenic fungi from the subject thereby reducing the risk of the subject developing a symptomatic fungal infection. Prophylactic treatment with a compound of the invention may be beneficial in subjects at a high risk of developing a fungal infection, for example subjects with a compromised immune system (e.g. subjects being treated with an immunosupressant, subjects that have undergone organ or stem-cell transplant, or subjects with neutropenia (e.g. chemotherapy or radiation therapy induced neutropenia, or subjects with a haematologic malignancy); or subjects in intensive care. A compound of the invention may be for use in the decolonization or eradication of pathogenic fungi in a subject, prior to, during or after the subject undergoing a surgical procedure or receiving an implanted medical device (e.g. a catheter, stent, heart valve, pacemaker, defibrillator, or CNS medical device).

### Biofilms

Certain fungi form, or are associated with, biofilms. Biofilms comprise a community of microorganisms that are attached to a given surface, inert material, or living tissue. The microorganisms comprising the biofilm produce extracellular polymeric substances that forms a structural matrix within which the microorganisms are distributed (Donlan et al., Emerg. Infect. Dis., 2001, 7, 277-81). Fungi present in biofilms can exhibit lower growth rates and can be difficult to treat, because the biofilm matrix can make it difficult for drugs to penetrate access the fungi present in the biofilm. Biofilms can therefore harbour viable fungi and increase the likelihood of an infection recurring. The presence of fungal biofilms is also associated with the development of resistance to antifungal drugs. For example, up-regulation of efflux pumps in biofilms further reduce intracellular concentration of antifungal drugs resulting in an increased mutation rate of fungi present in biofilms. The formation of biofilms on the surface of implanted medical devices is particularly problematic and is a well-known source of fungal infection (Kojic et al., Clin Microbiol Rev. 2004, 17(2), 255-67).

According to another aspect there is provided a compound of the invention for use in the treatment or prevention of a biofilm comprising a pathogenic fungus. In some embodiments the compound of the invention inhibits the formation of a biofilm comprising a pathogenic fungus. In some embodiments the compound of the invention reduces or eliminates fungi from the biofilm. For example, treatment of the biofilm with the compound reduces the fungal load present in a biofilm by 20%, 30%, 40%, 50% 60%, 70%, 80%, 85%, 90%, 95% or 99%. It may be that treatment of the biofilm with the compound substantially eliminates fungi from the biofilm.

In some embodiments, the pathogenic fungi comprising the biofilm may be any of the pathogenic fungi disclosed herein. For example is may be that the pathogenic fungus comprising the biofilm is a Candidia spp. It may be that the pathogenic fungus comprising the biofilm is selected from Candida albicans, Candida glabrata, Candida tropicalis, Candida parapsilosis and Candida auris.

In some embodiments there is provided a compound of the invention for use in preventing or inhibiting the formation of a biofilm comprising a pathogenic fungus on a medical device implanted in a subject.

In another embodiment there is provided a compound of the invention for use in the treatment of a fungal infection associated with a medical device present in a subject.

In some embodiments there is provided a compound of the invention for use in the treatment of a biofilm comprising a pathogenic fungus on a medical device present in a subject.

In some embodiments there is provided a method for preventing or inhibiting the formation of a biofilm comprising a pathogenic fungus on a medical device, the method comprising contacting the medical device with a compound of the invention prior to implanting the device in a subject. It may be that the medical device is immersed in a solution or suspension comprising a compound of the invention prior to implanting the medical device in a subject. The medical device is suitably contacted with the compound of the invention for sufficient time to reduce or eliminate a biofilm from the device, or to reduce or eliminate the fungi present in a biofilm. For example, the medical device may be contacted with a compound of the invention for 1, 2, 3, 4, 5, 6, 7 or 8, 10, 12, or 24 hours prior to implantation in the subject.

Coating or impregnating a medical device with a compound of the invention may be useful to prevent or inhibit the formation of biofilms comprising fungi on the medical device. Accordingly also provided is an implantable medical device coated or impregnated with a compound of the invention.

In the embodiments above relating to medical devices, it may be that the medical device is selected from: catheters (e.g. central venous catheters, dialysis catheters or urinary catheters), joint prostheses (e.g. hip or knee prostheses), arteriovenous prosthetic grafts, arteriovenous fistulas, cardiovascular devices (e.g. pacemakers (including the pulse generator, leads and electrodes), prosthetic heart valves, implanted cardioverter defibrillators or ventricular assist devices), CNS devices (e.g. ventriculostomy drains, shunts, stimulators and biopolymer wafers that deliver chemotherapy).

### Diagnosis of Fungal Infections

The diagnosis of a fungal infection (e.g. a Candida or Aspergillus infection) in a subject may be determined using known methods, for example as described in Arvanitis et al., Clin. Microbiol. Rev. 2014; 27(3):490-526. Suitable diagnostic methods include microscopy and histopathology wherein a biopsy of infected tissue/bodily fluid, staining, culturing and histopathology to identify the infecting organism. β-D-Glucan is a major component of the fungal cell wall and assays to detect β-D-Glucan can be used in the diagnosis of a fungal infection. Candidia albican infections can be diagnosed using the Candida albicans germ tube antibody (CAGTA) assay. Other methods for the diagnosis of a fungal infection include PCR methods, nucleic acid sequence-based amplification (NASBA), matrix-assisted laser desorption ionization (MALDI, including MALDI-TOF MS), fluorescence in situ hybridization (FISH), surface enhanced resonance Raman spectroscopy (SERRS), nuclear magnetic resonance (NMR) spectroscopy.

Diagnosis of C. auris Infections

C. auris infection is difficult to identify and it can easily be misdiagnosed as a different fungal organism when using traditional diagnostic methods. The CDC recommends matrix-assisted laser desorption/ionization time-of-flight (MALDI-TOF), using for example the Bruker Biotyper brand MALDI-TOF using the updated Bruker FDA-approved MALDI Biotyper CA System library (Version Claim 4). Molecular methods based on sequencing the D1-D2 region of the 28s rDNA or the Internal Transcribed Region (ITS) of rDNA can also be used to identify C. auris (CDC, identification of Identification of Candida auris, October 2019 (https://www.cdc.gov/fungal/candida-auris/recommendations.html #identify).

### Combination Therapies

The compounds of the invention may be used alone to provide a therapeutic effect. The compounds of the invention may also be used in combination with one or more additional antifungal agent to provide a combination treatment of, for example a fungal infection. It may be that the additional antifungal agent is one or more of:
(i) a polyene (e.g. amphotericin B (e.g. amphotericin B deoxycholate, liposomal amphotericin B, amphotericin B lipid complex or amphotericin B colloidal dispersion), candicidin, filipin, harnycin, natamycin, nystatin, or rimocidin);
(ii) a triazole, preferably a triazole other than a compound of the invention (e.g. albaconazole, efinaconazole, epoxiconazole, fluconazole, isavuconazole, itraconazole, posaconazole, propiconazole, ravuconazole, terconazole or voriconazole);
(iii) an imidazole (e.g. bifonazole, butoconazole, chlormidazole, clotrimazole, eberconazole, econazole, fenticonazole, flutrimazole, isoconazole, ketoconazole, luliconazole, miconazole, omoconazole, oxiconazole, sertaconazole, sulconazole, or tioconazole);
(iv) a thiazole (e.g. abafungin);
(v) an echinocandin (e.g. anidulafungin, biafungin, caspofungin, or micafungin);
(vi) an allylamine (e.g., amorolfin, butenafine, naftifine, or terbinafine); or
(vii) amorolfine, ciclopirox olamine, griseofulvin or flucytosine (5-fluorocytosine).

The additional antifungal agent may be a single agent or one or more of the additional agents listed herein.

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect “combination” refers to simultaneous administration. In another aspect “combination” refers to separate administration. In a further aspect “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

According to a further aspect of the invention there is provided a compound of the invention as defined hereinbefore and an additional antifungal agent as defined hereinbefore, for use in the conjoint treatment or prevention of a fungal infection.

According to a further aspect of the invention there is provided a pharmaceutical product comprising a compound of the invention as defined hereinbefore and an additional antifungal agent as defined hereinbefore for the conjoint treatment of a fungal infection.

According to a further aspect of the invention there is provided a method of treatment of a subject suffering from a fungal infection comprising administering to the subject a therapeutically effective amount of a compound of the invention, or a pharmaceutically acceptable salt thereof simultaneously, sequentially or separately with an additional antifungal agent as defined hereinbefore.

According to a further aspect of the invention there is provided a compound of the invention, or a pharmaceutically acceptable salt thereof for use simultaneously, sequentially or separately with an additional antifungal agent as defined hereinbefore, in the treatment of a fungal infection.

### Biological Assays

The biological effects of the compounds may be assessed using one of more of the assays described herein in the Examples. For example the efficacy may be assessed by determining the minimum inhibitory concentration of a compound against a target fungus as described in the Examples. Compounds of the invention have been shown to exhibit potent antifungal activity against a panel of fungal species, including resistant strains of C. auris, in the Candida panel MIC described in the Examples.

The efficacy of compounds in-vivo may be assessed using the Galleria mellonella infection model described in the Examples. Compounds of the invention tested in this model exhibited protection against resistant strains of C. auris. The tested compounds were non-toxic in doses up to 50 mg/kg in this model. This suggests that compounds of the invention selectively target fungal cells and are expected to exhibit a wide therapeutic window between anti-fungal efficacy and the onset of undesirable side-effects/toxicity.

### Synthesis

In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃·OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, or sodium hydroxide, or ammonia. Alternatively, an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### General Synthetic Routes

The compounds of the invention may be prepared using analogous methods to those described in the Examples.

Compounds of the formula (I) may be prepared by reacting a compound of the formula (A):

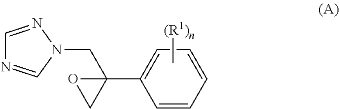

wherein R¹ and n have any of the meanings defined herein, except that any functional group is protected if necessary, with an amine of the formula (B):

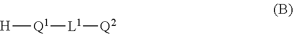

wherein Q¹, L¹ and Q² have any of the meanings defined herein, except that any functional group is protected if necessary;
and thereafter carrying out one or more of the following procedures:
   converting a compound of formula (I) into another compound of formula (I)
   removing any protecting groups
   forming a pharmaceutically acceptable salt.

The reaction is suitable performed in the presence of a suitable base, for example a tertiary organic amine base (e.g. triethylamine). The reaction is suitable performed in a suitable solvent, for example an alcohol such as ethanol. The reaction may be performed at elevated temperature, for example at or close to the reflux temperature of the solvent.

Compounds of the formula (A) may be prepared by, for example, Reaction Scheme 1:

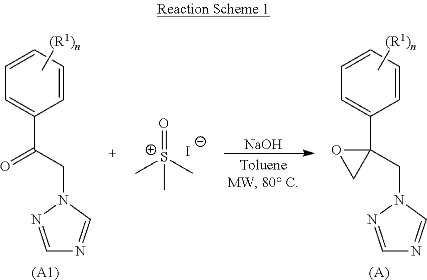

The starting material (A1) is commercially available or can be made using well-known methods.

Amines of the formula (B) are commercially available or can be prepared using well-known methods, for example as illustrated in the following reaction schemes.

When L¹ is —NR⁴— and Q¹ is bonded to L¹ via a ring carbon atom in Q¹ (e.g. Q¹ is Ia, Ib, Ic, le or Ig) the amine of formula (B) may be made according to Reaction Scheme 2 or 3:

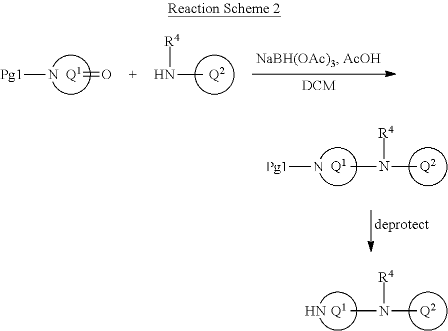

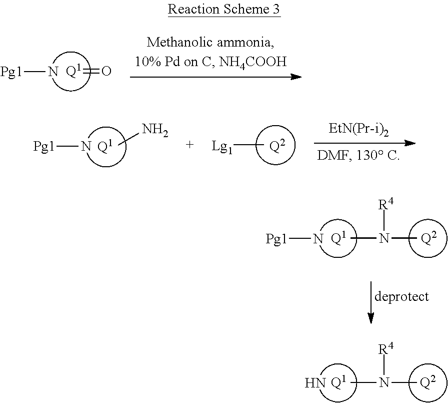

wherein Pg¹ is a suitable amine protecting group (e.g. BOC);
Lg¹ is a suitable leaving group (e.g. halo); and R⁴ has any of the meanings defined herein.

When Q¹ is bonded to L¹ via a ring nitrogen atom in Q¹ (e.g. Q¹ is Id, If, Ih, Ii or Ij) and L¹ is a bond or —CH₂—, the amine of formula (B) ma be prepared using Reaction Scheme 4:

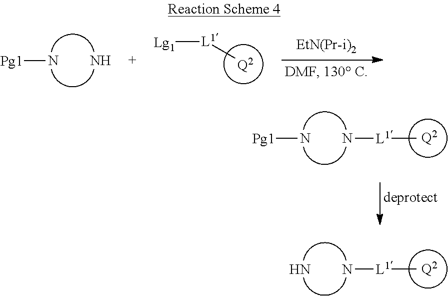

wherein L^{1′} is a bond or —CH₂—; Pg¹ is a suitable amine protecting group (e.g. BOC); and
Lg¹ is a suitable leaving group (e.g. halo).

When Q² is substituted by Q³-L³- and L³ is a bond, the amine of formula (B) may be prepared using the coupling reaction according to Reaction Scheme 5:

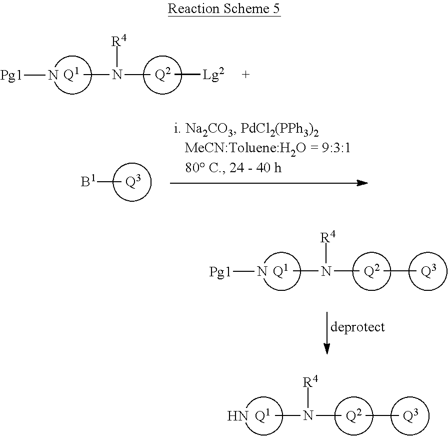

wherein Pg¹ is a suitable amine protecting group (e.g. BOC); Lg¹ is a suitable leaving group (e.g. halo such as Br); and B¹ is boronic acid (—B(OH)₂) or a boronic acid ester.

In Reaction Schemes 2 to 5 the last step the amine protecting group Pg¹ is removed. This can be achieved using well-known methods, for example when the protecting group is a BOC group, by treating the compound with a suitable acid such as HCl.

The synthesis of the compounds of the invention are further illustrated in the Examples herein.

### ABBREVIATIONS

The following abbreviations are used:
DCM: dichloromethane
DIPEA: N,N-diisopropylethylamine
DMSO: dimethylsulfoxide
EtOAc: ethyl acetate
LC-MS: liquid chromatography-mass spectrometry
MIC: minimum inhibitory concentration
MW: microwave
STAB: sodium triacetoxyborohydride
TLC: thin layer chromatography
XPhos: 2-Dicyclohexylphosphino-2′,4′,6′-triisopropylbiphenyl

### Reagents and Conditions

Unless syntheses are given, reagents and starting materials were obtained from commercial sources. All reactions, unless otherwise stated, were carried out under an inert atmosphere of either nitrogen or argon.

### Analytical Methods

LC-MS (Agilent Technologies, UK) analysis was carried out using an Agilent 1290 system with phenomenex Onyx Monolithic C18, LC Column 50×4.6 mm column and gradients of Water (A) and acetonitrile (B) as mobile phases. For 5-minute standard run, the solvent flow was 1.000 ml/min. The solvent gradient at 0.00, 2.00, 2.50, 4.50 and 4.60 minute was A (%)=95.00, 50.00, 10.00, 10.00 and 95.00 and B (%)=5.00, 50.00, 90.00, 90.00 and 5.00 respectively. For 10-minute standard run, the solvent flow was 0.5000 ml/min. The solvent gradient at 0.00, 4.00, 6.00, 6.50, 8.50 and 9.50 minutes was A (%)=90.00, 60.00, 25.00, 10.00, 10.00 and 90.00 and B (%)=10.00, 40.00, 75.00, 90.00, 90.00 and 10.00 respectively.

The compounds were purified using preparative column chromatography suing an Agilent 1290 Infinity II preparative LC-MS (InfinityLab LC/MSD, Agilent technologies, US) with Luna C18 100×21.2 mm column and a gradient of Water (A) and Acetonitrile (B). The solvent that was used was 70:30 acetonitrile (B): water (A)+0.1% formic acid. The solvent flow rate was 20.00 ml/min and the maximum pressure limit was 420 [bar]. The solvent gradient at 0.00, 1.00, 2.50, 4.50, 6.50, 7.00, 8.00, 9.00 and 10.00 minutes was A (%)=90.00, 90.00, 80.00, 55.00, 50.00, 30.00, 15.00, 10.00 and 90.00 and B (%)=10.00, 10.00, 20.00, 45.00, 50.00, 70.00, 85.00, 90.00 and 10.00 respectively.

### NMR

All NMR spectra were obtained using Bruker Ascend 400 MHz and all HRMS analysis were performed on a Thermo Exactive MS System.

### Azole Core Synthesis (Epoxide Intermediate)

### 1-((2-(2,4-difluorophenyl)oxiran-2-yl)methyl)-1H-1,2,4-triazole

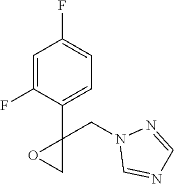 [SMILES: Fc1ccc(C2(Cn3cncn3)CO2)c(F)c1]

Trimethylsulphoxonium iodide (2 equiv.) was added to toluene (0.12 mmol/mL) containing 1-(2,4-difluorophenyl)-2-(1H-1,2,4-triazol-1-yl)ethan-1-one (1 equiv.) and sodium hydroxide 30% (w/w) aqueous solution (10 equiv.). The mixture was heated under microwave (MW) radiation for 50 minutes at 80° C. Then the mixture was diluted with water and extracted with ethyl acetate. The organic layer was combined and washed with saturated brine, dried over anhydrous magnesium sulphate, and concentrated in vacuo; ¹H NMR (400 MHz, CDCl₃) σ: 2.83 (1H, d, J=4.8 Hz), 2.90 (1H, d, J=4.8 Hz), 4.47 (1H, d, J=14.9 Hz), 4.79 (1H, d, J=15.0 Hz), 6.73-6.82 (2H, m), 7.11-7.16 (1H, m), 7.81 (1H, s), 8.03 (1H, s); [M+H]+: 238.1, Rt=2.71 min.

Other epoxide intermediates of the formula:

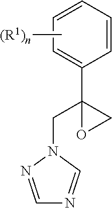

may be prepared using analogous methods from the appropriate 1-((optionally substituted-difluorophenyl)-2-(1H-1,2,4-triazol-1-yl)ethan-1-one starting material.

### Synthesis of BOC-protected amine intermediate

### tert-Butyl-4-((4-bromophenyl)amino)piperidine-1-carboxylate

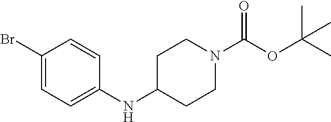 [SMILES: CC(C)(C)OC(=O)N1CCC(Nc2ccc(Br)cc2)CC1]

Bromoaniline (1.2 equiv.) was added into the flask containing N-Boc-4-piperidone (1 equiv.) while stirring in anhydrous DCM (0.75 mol ml-1). Sodium triacetoxyborohydride (STAB) (1.5 equiv.) and glacial acetic acid (1.5 equiv.) were added portionwise and dropwise respectively at 0° C. The reaction mixture was stirred under argon at room temperature overnight. The crude was diluted with 1 N NaOH and extracted with DCM for three times. Combined organic layer was dried over MgSO₄ (anhydrous), filtered, and evaporated in vacuo. Flash column chromatography was carried out with chloroform for further purification; ¹H NMR (400 MHz, methanol-d₄) σ: 1.30 (2H, td, J=19.5 Hz, 4.0 Hz), 1.46 (9H, s), 1.96 (2H, dd, J=13.0 Hz, 2.7 Hz), 2.95 (2H, t, J=13.0 Hz), 3.40 (1H, tt, J=10.2 Hz, 3.7 Hz), 4.00 (2H, dt, J=13.6 Hz, 3.8 Hz), 6.56 (2H, dd, J=8.8 Hz, 3.2 Hz), 7.17 (2H, dd, J=9.0 Hz, 3.1 Hz).

### tert-Butyl-4-((4′-cyano-[1,1′-biphenyl]-4-yl)amino)piperidine-1-carboxylate

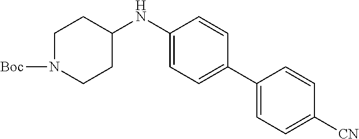 [SMILES: CC(C)(C)OC(=O)N1CCC(Nc2ccc(-c3ccc(C#N)cc3)cc2)CC1]

tert-Butyl-4-((4-bromophenyl)amino)piperidine-1-carboxylate (1 equiv.), 4-cyanophenyl boronic acid (2.0 equiv.), sodium carbonate (3.0 equiv.) and bis(triphenylphosphine)palladium(II) dichloride (0.1 equiv.) were dissolved in the MeCN:Toluene:H₂O 9:3:1 solvent in a round-bottomed flask, and was heated at 80° C. overnight. The flask was cooled down to room temperature and the mixture was filtered through Celite®, diluted with 0.5 M NaOH (aq.) and extracted three times with ethyl acetate. Organic layer was combined, dried over MgSO₄ (anhydrous), filtered, and evaporated under reduced pressure. Gradient flash column chromatography (hexane and ethyl acetate) was used for further purification.

### tert-Butyl-4-((4′-methyl-[1,1′-biphenyl]-4-yl)amino)piperidine-1-carboxylate

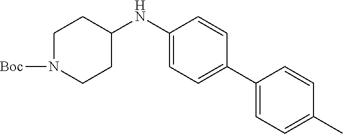 [SMILES: Cc1ccc(-c2ccc(NC3CCN(C(=O)OC(C)(C)C)CC3)cc2)cc1]

tert-Butyl-4-((4-bromophenyl)amino)piperidine-1-carboxylate (1 equiv.), p-tolylboronic acid (3 equiv.), tripotassium phosphate (2 equiv.) and bis(triphenylphosphine)palladium(II) dichloride (0.005 equiv.) was added to the 67.5% DMF (aq.). The mixture was heated under microwave (15 bar, 100° C.) for 40 min until all starting material was consumed. Crude mixture was filtered through Celite®, washed with saturated brine and extracted with ethyl acetate for three times, then organic layer was combined, dried over anhydrous MgSO₄ and evaporated in vacuo. Further purification was carried out using gradient flash column chromatography (hexane and ethyl acetate).

### tert-Butyl-4-((3-fluoropyridin-4-yl)amino)piperidine-1-carboxylate

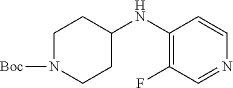 [SMILES: CC(C)(C)OC(=O)N1CCC(Nc2ccncc2F)CC1]

4-Bromo-3-fluoro-pyridine (1.0 equiv.) was added to a round-bottomed flask containing N-Boc-4-amino-piperidine (1 equiv.) dissolving in anhydrous toluene (0.42 mmol ml⁻¹) while stirring, followed by the addition of tris(dibenzylideneacetone)dipalladium(0) (0.08 equiv.), XPhos (0.02 equiv.) and potassium tert-butoxide (2.5 equiv.). The mixture was heated under inert atmosphere at 100° C. overnight until all starting material was consumed. The crude product was filtered through Celite®, evaporated on rotary evaporator, and purified with gradient flash column chromatography using DCM and methanol.

### tert-Butyl-4-((4-(2-morpholinoethoxy)phenyl)amino)piperidine-1-carboxylate

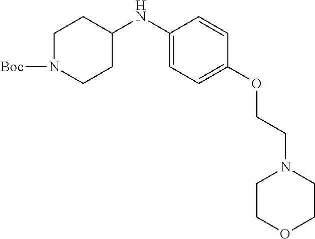 [SMILES: CC(C)(C)OC(=O)N1CCC(Nc2ccc(OCCN3CCOCC3)cc2)CC1]

Following the same procedure as tert-butyl-4-((4-bromophenyl)amino)piperidine-1-carboxylate synthesis using 4-(2-morpholinoethoxy)aniline instead of bromoaniline.

### tert-Butyl-8-(5-fluoropyrimidin-2-yl)-2,8-diazaspiro[4.5]decane-2-carboxylate

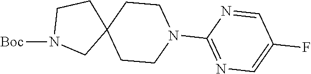 [SMILES: CC(C)(C)OC(=O)N1CCC2(CCN(c3ncc(F)cn3)CC2)C1]

tert-Butyl-2,8-diazaspiro[4.5]decane-2-carboxylate (1.0 equiv.) and 2-chloro-5-fluoropyrimidine (1.275 equiv.) were added to the flask that containing DMF (0.16 mmol ml⁻¹) while stirring, followed by the addition of DIPEA (3.125 equiv.) dropwise. The mixture was heated under argon at 130° C. overnight until no starting material appeared on TLC and LC-MS. The flask was cooled down to room temperature and the mixture was diluted with water then extracted with DCM for three times before dried with MgSO₄ and evaporated in vacuo. Crude product was further purified with gradient flash column chromatography (DCM and ethyl acetate).

### tert-Butyl-4-(5-fluoropyrimidin-2-yl)-1,4-diazepane-1-carboxylate

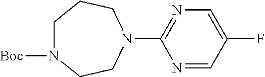 [SMILES: CC(C)(C)OC(=O)N1CCCN(c2ncc(F)cn2)CC1]

The title intermediate was prepared using the same procedure as that used for the preparation of tert-butyl-8-(5-fluoropyrimidin-2-yl)-2,8-diazaspiro[4.5]decane-2-carboxylate using tert-butyl-1,4-diazepane-1-carboxylate instead of tert-butyl 2,8-diazaspiro[4.5]decane-2-carboxylate.

### tert-Butyl-4-((4-benzylphenyl)amino)azepane-1-carboxylate

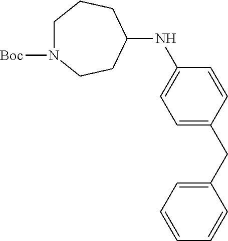 [SMILES: CC(C)(C)OC(=O)N1CCCC(Nc2ccc(Cc3ccccc3)cc2)CC1]

The title intermediate was prepared using the same procedure as tert-butyl-4-((4-bromophenyl)amino)piperidine-1-carboxylate synthesis using 4-benzyl-aniline and N-boc-hexahydro-1H-azepin-4-one instead of bromoaniline and N-Boc-4-piperidone respectively.

### tert-Butyl-4-((5-fluoropyrimidin-2-yl)amino)-[1,4′-bipiperidine]-1′-carboxylate

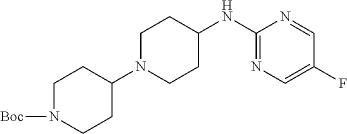 [SMILES: CC(C)(C)OC(=O)N1CCC(N2CCC(Nc3ncc(F)cn3)CC2)CC1]

Methanolic ammonia (2M, 0.48 mmol ml⁻¹) was added to the flask that containing tert-butyl-4-(4-oxopiperidin-1-yl)piperidine-1-carboxylate (1 equiv.), followed by the addition of ammonium formate (4.0 equiv.) and 10% Pd on C (50 mg) while stirring. The mixture was stirred at room temperature overnight until all starting material was consumed. The mixture was filtered through Celite® and solvent was evaporated. The residue was washed with 2M aqueous NaOH and extracted ethyl acetate for three times and dried over anhydrous MgSO₄. The product tert-butyl-4-amino-[1,4′-bipiperidine]-1′-carboxylate was obtained after evaporating solvent in vacuo with about a 100% conversion from starting material and used without further purification.

tert-Butyl-4-amino-[1,4′-bipiperidine]-1′-carboxylate (2.0 equiv.) was added to the flask that containing anhydrous DMSO (0.25 mmol ml⁻¹), followed by the addition of 2-chloro-5-fluoropyrimidine (1.0 equiv.) and potassium carbonate (2.0 equiv.). The mixture was heated at 70° C. for 24 h before cooling down and diluted with saturated brine. Then DCM was used for extraction and MgSO₄ was used for drying of organic layer. After the evaporation under reduced pressure, the crude product was purified with gradient flash column chromatography using DCM, methanol and triethylamine.

### General Experimental Procedure for Boc-Deprotection Reactions

The BOC-protected amine intermediates were de-protected to form amine intermediates used in the synthesis of the compounds of the invention using the following procedure.

4M HCl in 1,4-dioxane (0.2335 mmol ml⁻¹) was added to a flask containing the Boc-protected amine intermediate (1 equiv.) dropwise while stirring. The mixture was stirred at room temperature until all starting material disappeared on TLC and LC-MS. After evaporating down the solvent under reduced pressure, the crude product was used directly in final reaction without further purification assuming 100% conversion.

### General Experimental Procedure for Final Compounds Synthesis

The Examples below were prepared using the following procedure. 1-((2-(2,4-difluorophenyl)oxiran-2-yl)methyl)-1H-1,2,4-triazole (1.0 equiv.) and triethylamine (1.5 equiv.) was added into a solution of the required amine intermediate (1.5 equiv.), or corresponding amine hydrochloride salt in ethanol (0.07 mmol/mL) under stirring. The mixture was heated under stirring at 80° C. overnight until all starting material disappeared. The solvent was removed under reduced pressure and product was purified by flash column chromatography (MeOH in EtOAc, 0-20%).

### Example 1

### 1-(9-Benzyl-3,9-diazaspiro[5.5]undecan-3-yl)-2-(2,4-difluorophenyl)-3-(1H-1,2,4-triazol-1-yl)propan-2-ol

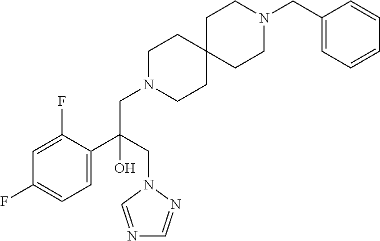 [SMILES: OC(CN1CCC2(CCN(Cc3ccccc3)CC2)CC1)(Cn1cncn1)c1ccc(F)cc1F]

¹H NMR (400 MHz, methanol-d₄) σ: 1.40 (4H, t, J=5.1 Hz), 1.47 (4H, t, J=5.2 Hz), 2.36 (4H, t, J=5.6 Hz), 2.42 (4H, t, J=5.2 Hz), 2.77 (1H, d, J=13.8 Hz), 2.98 (1H, dd, J=13.7 Hz, 1.2 Hz), 3.53 (2H, s), 4.55 (1H, d, J=14.2 Hz), 4.66 (1H, d, J=14.3 Hz), 6.83 (1H, td, J=8.4 Hz, 2.4 Hz), 6.92 (1H, ddd, J=11.6 Hz, 8.9 Hz, 2.5 Hz), 7.23-7.31 (5H, m), 7.47 (1H, td, J=8.9 Hz, 6.8 Hz), 7.74 (1H, s), 8.34 (1H, s). [M+H]⁺: 482.3, Rt=1.78 min.

### Example 2

### 4′-((1-(2-(2,4-difluorophenyl)-2-hydroxy-3-(1H-1,2,4-triazol-1-yl)propyl)piperidin-4-yl)amino)-[1,1′-biphenyl]-4-carbonitrile

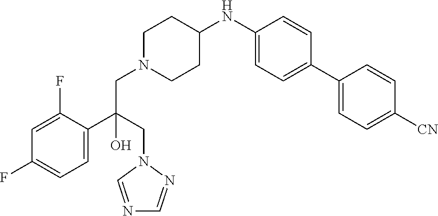 [SMILES: N#Cc1ccc(-c2ccc(NC3CCN(CC(O)(Cn4cncn4)c4ccc(F)cc4F)CC3)cc2)cc1]

¹H NMR (400 MHz, CDCl₃) σ: 1.63-1.77 (2H, m), 2.17 (1H, d, J=12.6 Hz), 2.28 (1H, d, J=14.2 Hz), 2.49 (1H, t, J=9.0 Hz), 2.68-2.78 (2H, m), 2.85-2.90 (1H, m), 3.00 (1H, d, J=13.6 Hz), 3.37 (1H, d, J=13.5 Hz), 3.56 (1H, tt, J=9.8 Hz, 3.5 Hz), 4.78 (1H, d, J=14.2 Hz), 4.83 (1H, d, J=14.1 Hz), 5.57 (1H, br, s), 6.89 (2H, d, J=8.6 Hz), 7.06-7.11 (2H, m), 7.69 (2H, d, J=8.6 Hz), 7.81-7.97 (5H, m), 8.07 (1H, s), 8.43 (1H, s). [M+H]⁺: 515.2, Rt=2.74 min.

### Example 3

### 2-(2,4-Difluorophenyl)-1-(4-((4′-methyl-[1,1′-biphenyl]-4-yl)amino)piperidin-1-yl)-3-(1H-1,2,4-triazol-1-yl)propan-2-ol

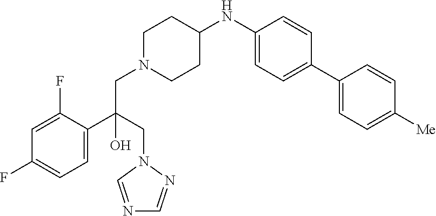 [SMILES: Cc1ccc(-c2ccc(NC3CCN(CC(O)(Cn4cncn4)c4ccc(F)cc4F)CC3)cc2)cc1]

¹H NMR (400 MHz, CDCl₃) σ: 1.30-1.48 (2H, m), 1.90 (1H, d, 11.4 Hz), 2.02 (1H, d, J=12.3 Hz), 2.23 (1H, t, J=10.7 Hz), 2.37 (3H, s), 2.43-2.53 (2H, m), 2.62 (1H, d, J=8.8 Hz), 2.73 (1H, d, J=12.7 Hz), 3.10 (1H, d, J=13.3 Hz), 3.25-3.30 (1H, m), 4.51 (1H, d, J=14.5 Hz), 4.56 (1H, d, J=14.3 Hz), 6.60 (2H, d, J=8.7 Hz), 6.79-6.85 (2H, m), 7.20 (2H, d, J=7.8 Hz), 7.39 (2H, d, J=8.8 Hz), 7.41 (2H, d, J=8.3 Hz), 7.57 (1H, td, J=9.5 Hz, 6.6 Hz), 7.80 (1H, s), 8.18 (1H, s). [M+H]⁺: 504.2, Rt=3.07 min.

### Example 4

### 2-(2,4-difluorophenyl)-1-(4-((4′-methoxy-[1,1′-biphenyl]-4-yl)amino)piperidin-1-yl)-3-(1H-1,2,4-triazol-1-yl)propan-2-ol

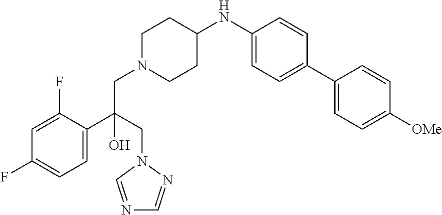 [SMILES: COc1ccc(-c2ccc(NC3CCN(CC(O)(Cn4cncn4)c4ccc(F)cc4F)CC3)cc2)cc1]

¹H NMR (400 MHz, CDCl₃) σ: 1.24-1.34 (1H, m), 1.37-1.47 (1H, m), 1.88 (1H, d, J=13.2 Hz), 2.01 (1H, d, J=12.9 Hz), 2.20 (1H, td, J=11.8 Hz, 2.3 Hz), 2.46 (1H, t, J=10.7 Hz), 2.41 (1H, d, J=13.0 Hz), 2.61 (1H, d J=12.1 Hz), 2.71 (1H, d, J=13.7 Hz), 3.09 (1H, d, J=13.6 Hz), 3.23-3.28 (1H, m), 3.81 (3H, s), 4.52 (2H, d, J=1.8 Hz), 6.59 (2H, d, J=8.6 Hz), 6.78-6.84 (1H, m), 6.92 (2H, d, J=8.8 Hz), 7.18 (1H, dd, J=14.7 Hz, 8.6 Hz), 7.34 (2H, d, J=8.6 Hz), 7.43 (2H, d, J=8.8 Hz), 7.55 (1H, dd, J=14.3 Hz, 8.6 Hz), 7.80 (1H, s), 8.17 (1H, s). [M+H]⁺: 520.2, Rt=2.88 min.

### Example 5

### 4′-((1-(2-(2,4-difluorophenyl)-2-hydroxy-3-(1H-1,2,4-triazol-1-yl)propyl)piperidin-4-yl)amino)-[1,1′-biphenyl]-2-carbonitrile

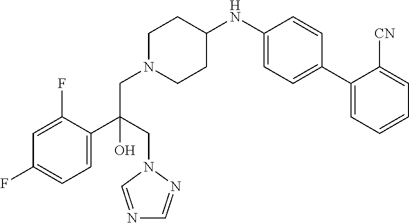 [SMILES: N#Cc1ccccc1-c1ccc(NC2CCN(CC(O)(Cn3cncn3)c3ccc(F)cc3F)CC2)cc1]

¹H NMR (400 MHz, CDCl₃) δ: 0.81 (2H, br S), 1.37 (2H, br S), 1.81 (1H, d, J=12.66), 1.93 (1H, d, J=13.86), 2.13 (1H, t, J=10.75), 2.34 (1H, d, J=12.84), 2.38 (1H, t, J=11.91), 2.53 (1H, d, J=11.19), 2.63 (1H, d, J=13.54), 3.01, (1H, d, J=13.52), 3.21 (1H, m, J=11.55), 6.54 (2H, d, J=8.34), 6.73 (2H, br, s), 7.25 (1H, t, J=7.53), 7.30 (2H, d, J=8.36), 7.37 (1H, d, J=7.84), 7.48 (2H, q, J=7.37), 7.62 (1H, d, J=7.68), 7.72 (1H, s), 8.08 (1H, s). [M+H]⁺: 515.2, Rt=2.78 min.

### Example 6

### 2-(2,4-Difluorophenyl)-1-(4-((4-(2-morpholinoethoxy)phenyl)amino)piperidin-1-yl)-3-(1H-1,2,4-triazol-1-yl)propan-2-ol

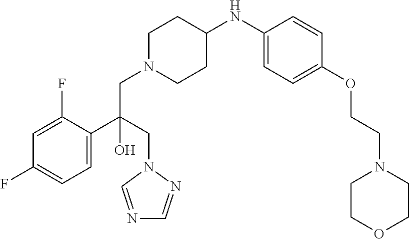 [SMILES: OC(CN1CCC(Nc2ccc(OCCN3CCOCC3)cc2)CC1)(Cn1cncn1)c1ccc(F)cc1F]

¹H NMR (400 MHz, methanol-d₄) σ: 1.26-1.31 (2H, m), 1.34-1.44 (2H, m), 1.74-1.81 (1H, m), 1.85-1.91 (1H, m), 2.23 (1H, td, J=11.5 Hz, 2.5 Hz), 2.40 (1H, td, J=11.5 Hz, 2.5 Hz), 2.58 (4H, dd, J=4.5 Hz, 4.5 Hz), 2.75 (2H, t, J=5.6 Hz), 2.80 (1H, d, J=13.8 Hz), 3.01 (1H, dd, J=13.9 Hz, 1.4 Hz), 3.09 (1H, tt, J=10.3 Hz, 3.7 Hz), 3.71 (4H, dd, J=4.5 Hz, 4.5 Hz), 4.03 (2H, t, J=5.5 Hz), 4.58 (1H, d, J=13.7 Hz), 4.68 (1H, d, J=13.9 Hz), 6.64 (2H, ddd, J=9.9 Hz, 3.5 Hz, 1.9 Hz), 6.76 (2H, ddd, J=10.1 Hz, 3.5 Hz, 2.0 Hz), 6.84 (1H, tdd, J=8.5 Hz, 2.6 Hz, 0.6 Hz), 6.92 (1H, ddd, J=11.7 Hz, 8.9 Hz, 2.6 Hz), 7.48 (1H, td, J=8.9 Hz, 6.7 Hz), 7.75 (1H, s), 8.35 (1H, s). [M+H]⁺: 543.3, Rt=1.68 min.

### Example 7

### 2-(2,4-Difluorophenyl)-1-(8-(5-fluoropyrimidin-2-yl)-2,8-diazaspiro[4.5]decan-2-yl)-3-(1H-1,2,4-triazol-1-yl)propan-2-ol

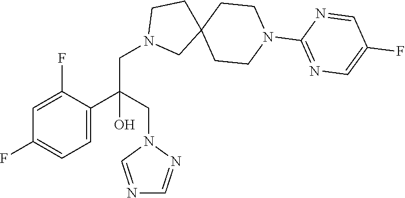 [SMILES: OC(CN1CCC2(CCN(c3ncc(F)cn3)CC2)C1)(Cn1cncn1)c1ccc(F)cc1F]

¹H NMR (400 MHz, methanol-d₄) σ: 1.48 (4H, t, J=5.7 Hz), 1.61 (2H, t, J=7.2 Hz), 2.37 (1H, d, J=9.1 Hz), 2.43 (1H, d, J=9.1 Hz), 2.62 (2H, t, J=7.3 Hz), 2.98 (1H, d, J=13.4 Hz), 3.10 (1H, dd, J=13.5 Hz, 1.8 Hz), 3.58-3.70 (4H, m), 6.86 (1H, tdd, J=8.3 Hz, 2.7 Hz, 0.7 Hz), 6.93 (1H, ddd, J=12.1 Hz, 9.3 Hz, 2.7 Hz), 7.50 (1H, td, J=9.2 Hz, 6.9 Hz), 7.76 (1H, s), 8.22 (2H, d, J=0.6 Hz), 8.35 (1H, s). [M+H]⁺: 474.2, Rt=2.55 min.

### Example 8

### 2-(2,4-Difluorophenyl)-1-(4-(5-fluoropyrimidin-2-yl)-1,4-diazepan-1-yl)-3-(1H-1,2,4-triazol-1-yl)propan-2-ol

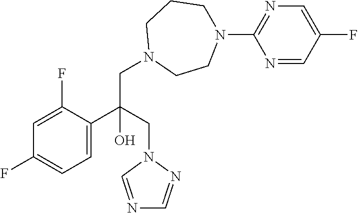 [SMILES: OC(CN1CCCN(c2ncc(F)cn2)CC1)(Cn1cncn1)c1ccc(F)cc1F]

¹H NMR (400 MHz, methanol-d₄) σ: 1.64-1.71 (2H, m), 2.54-2.69 (2H, m), 2.79 (2H, td, J=5.5 Hz, 0.5 Hz), 2.88 (1H, d, J=14.0 Hz), 3.20 (1H, dd, J=14.0 Hz, 1.6 Hz), 3.62-3.76 (4H, m), 4.52 (1H, d, J=14.2 Hz), 4.64 (1H, d, J=14.4 Hz), 6.83 (1H, tdd, J=8.5 Hz, 2.6 Hz, 0.8 Hz), 6.90 (1H, ddd, J=11.7 Hz, 8.9 Hz, 2.5 Hz), 7.42 (1H, td, J=9.0 Hz, 6.7 Hz), 7.75 (1H, s), 8.24 (2H, d, J=0.8 Hz), 8.31 (1H, s). [M+H]⁺: 434.1, Rt=2.29 min.

### Example 9

### 1-(4-((4-Benzylphenyl)amino)azepan-1-yl)-2-(2,4-difluorophenyl)-3-(1H-1,2,4-triazol-1-yl)propan-2-ol

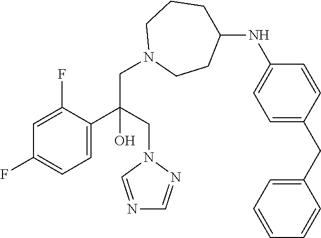 [SMILES: OC(CN1CCCC(Nc2ccc(Cc3ccccc3)cc2)CC1)(Cn1cncn1)c1ccc(F)cc1F]

¹H NMR (400 MHz, methanol-d₄) σ: 1.42-1.66 (4H, m), 1.80-1.91 (2H, m), 2.53-2.73 (4H, m), 2.88 (1H, dd, J=13.9 Hz, 7.0 Hz), 3.23 (1H, ddd, J=14.1 Hz, 5.0 Hz, 1.7 Hz), 3.37-3.47 (1H, m), 3.80 (2H, s), 4.55 (1H, dd, J=14.2 Hz, 6.1 Hz), 4.67 (1H, d, J=14.4 Hz), 6.52 (2H, dt, J=8.5 Hz, 1.9 Hz), 6.83 (1H, dddd, J=16.9 Hz, 5.9 Hz, 2.4 Hz, 0.7 Hz), 6.89-6.95 (3H, m), 7.10-7.15 (3H, m), 7.20-7.24 (2H, m), 7.51 (1H, tdd, J=8.9 Hz, 6.4 Hz, 3.6 Hz), 7.75 (1H, s), 8.34 (1H, d, J=1.7 Hz). [M+H]⁺: 518.2, Rt=3.06 min.

### Example 10

### 2-(2,4-Difluorophenyl)-1-(4-((5-fluoropyrimidin-2-yl)amino)-[1,4′-bipiperidin]-1′-yl)-3-(1H-1,2,4-triazol-1-yl)propan-2-ol

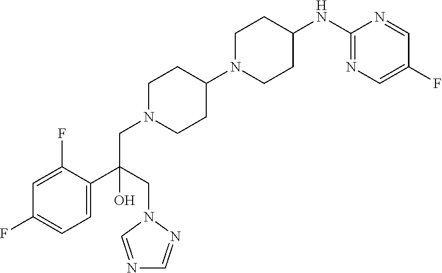 [SMILES: OC(CN1CCC(N2CCC(Nc3ncc(F)cn3)CC2)CC1)(Cn1cncn1)c1ccc(F)cc1F]

¹H NMR (400 MHz, methanol-d₄) σ: 1.45-1.93 (6H, m), 2.09-2.20 (3H, m), 2.35 (1H, td, J=12.0 Hz, 2.3 Hz), 2.55-2.69 (4H, m), 2.79 (1H, d, J=13.9 Hz), 2.90 (1H, m), 3.00 (1H, dd, J=13.9 Hz, 1.3 Hz), 3.14-3.20 (2H, m), 3.81 (1H, tt, 10.3 Hz, 3.8 Hz), 4.60 (1H, d, J=14.7 Hz), 4.7 (1H, d, J=14.6 Hz), 6.86 (1H, dddd, J=8.3 Hz, 8.3 Hz, 2.4 Hz, 0.4 Hz), 6.93 (1H, ddd, J=11.7 Hz, 9.0 Hz, 2.5 Hz), 7.49 (1H, ddd, J=9.0 Hz, 9.0 Hz, 6.6 Hz), 7.76 (1H, s), 8.22 (1H, d, J=0.9 Hz), 8.34 (1H, s). [M+H]⁺: 517.2, Rt=1.90 min.

### Example 11

### 1-(4-((4-benzylphenyl)amino)piperidin-1-yl)-2-(2,4-difluorophenyl)-3-(1H-1,2,4-triazol-1-yl)propan-2-ol

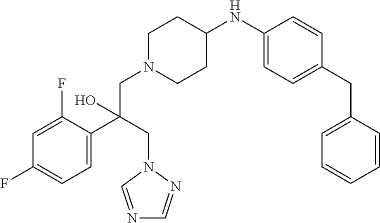 [SMILES: OC(CN1CCC(Nc2ccc(Cc3ccccc3)cc2)CC1)(Cn1cncn1)c1ccc(F)cc1F]

[M+H]⁺: 504.2, Rt=3.21 min.

### Example 12

### 1-(4-((4-benzylphenyl)amino)-[1,4′-bipiperidin]-1′-yl)-2-(2,4-difluorophenyl)-3-(1H-1,2,4-triazol-1-yl)propan-2-ol

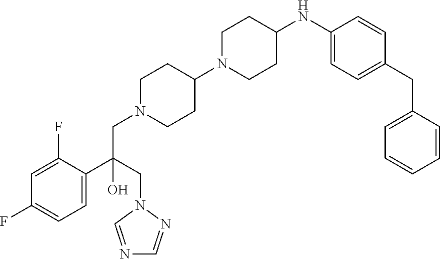 [SMILES: OC(CN1CCC(N2CCC(Nc3ccc(Cc4ccccc4)cc3)CC2)CC1)(Cn1cncn1)c1ccc(F)cc1F]

[M+H]⁺: 587.3, Rt=3.11 min.

### Example 13

### 1-(8-((4-benzylphenyl)amino)-2-azaspiro[4.5]decan-2-yl)-2-(2,4-difluorophenyl)-3-(1H-1,2,4-triazol-1-yl)propan-2-ol

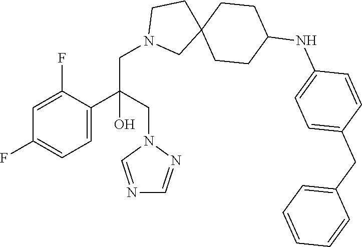 [SMILES: OC(CN1CCC2(CCC(Nc3ccc(Cc4ccccc4)cc3)CC2)C1)(Cn1cncn1)c1ccc(F)cc1F]

[M+H]⁺: 558.3, Rt=3.03 min.

### Example 14

### 2-(2,4-difluorophenyl)-1-(4-(phenylamino)-[1,4′-bipiperidin]-1′-yl)-3-(1H-1,2,4-triazol-1-yl)propan-2-ol

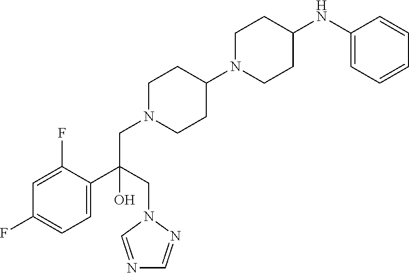 [SMILES: OC(CN1CCC(N2CCC(Nc3ccccc3)CC2)CC1)(Cn1cncn1)c1ccc(F)cc1F]

[M+H]⁺: 497.2, Rt=1.94 min.

### Example 15

### 2-(2,4-difluorophenyl)-1-(5-(5-fluoropyrimidin-2-yl)hexahydropyrrolo[3,4-c]pyrrol-2(1H)-yl)-3-(1H-1,2,4-triazol-1-yl)propan-2-ol

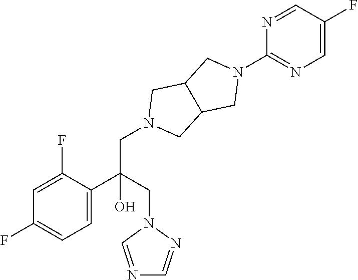 [SMILES: OC(CN1CC2CN(c3ncc(F)cn3)CC2C1)(Cn1cncn1)c1ccc(F)cc1F]

[M+H]⁺: 446.1, Rt=1.80 min.

### Example 16

### 1-((3aR,6aS)-5-((4-benzylphenyl)amino)hexahydrocyclopenta[c]pyrrol-2(1H)-yl)-2-(2,4-difluorophenyl)-3-(1H-1,2,4-triazol-1-yl)propan-2-ol

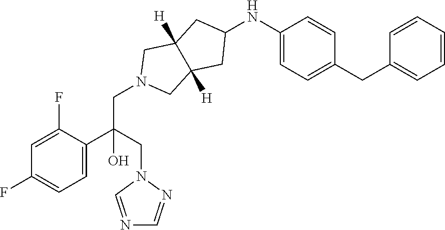 [SMILES: OC(CN1C[C@H]2CC(Nc3ccc(Cc4ccccc4)cc3)C[C@H]2C1)(Cn1cncn1)c1ccc(F)cc1F]

[M+H]⁺: 530.2, Rt=3.20 min.

### Example 17

### 2-(2,4-Difluorophenyl)-1-(4-(pyrazin-2-yl)-1,4-diazocan-1-yl)-3-(1H-1,2,4-triazol-1-yl)propan-2-ol

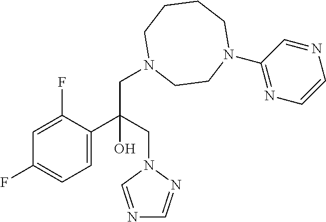 [SMILES: OC(CN1CCCCN(c2cnccn2)CC1)(Cn1cncn1)c1ccc(F)cc1F]

¹H NMR (400 MHz, methanol-d₄) σ: 1.67-1.72 (2H, m), 2.61 (1H, ddd, J=14.1 Hz, 6.1 Hz, 4.2 Hz), 2.69 (1H, ddd, J=14.0 Hz, 6.9 Hz, 5.0 Hz), 2.83 (2H, td, J=6.4 Hz, 1.3 Hz), 2.90 (1H, d, J=14.3 Hz), 3.22 (1H, dd, J=14.1 Hz, 1.5 Hz), 3.58-3.71 (6H, m), 4.52 (1H, d, J=13.3 Hz), 4.63 (1H, d, J=13.0 Hz), 6.81 (1H, tdd, J=8.8 Hz, 2.4 Hz, 0.7 Hz), 6.88 (1H, ddd, J=12.1 Hz, 8.9 Hz, 2.8 Hz), 7.38 (1H, td, J=8.8 Hz, 6.6 Hz), 7.69 (1H, d, J=2.85 Hz), 7.74 (1H, s), 7.97 (1H, d, J=1.2 Hz), 8.03 (1H, dd, J=2.7 Hz. [M+H]⁺: 416.2, Rt=1.99 min.

### Example 18

### 2-(2,4-difluorophenyl)-1-(4-((4-(piperidin-1-yl)phenyl)amino)piperidin-1-yl)-3-(1H-1,2,4-triazol-1-yl)propan-2-ol

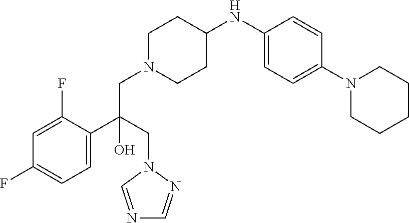 [SMILES: OC(CN1CCC(Nc2ccc(N3CCCCC3)cc2)CC1)(Cn1cncn1)c1ccc(F)cc1F]

[M+H]⁺: 497.2

### Example 19

### 2-(2,4-difluorophenyl)-1-(4-((4-(4-fluorobenzyl)phenyl)amino)-[1,4′-bipiperidin]-1′-yl)-3-(1H-1,2,4-triazol-1-yl)propan-2-ol

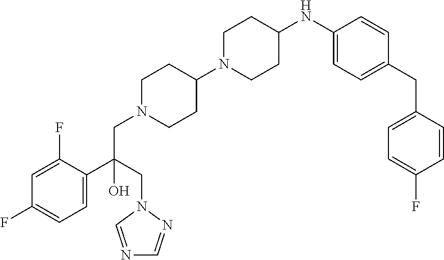 [SMILES: OC(CN1CCC(N2CCC(Nc3ccc(Cc4ccc(F)cc4)cc3)CC2)CC1)(Cn1cncn1)c1ccc(F)cc1F]

[M+H]⁺: 605.7

### Example 20

### 2-(2,4-difluorophenyl)-1-(4-((4-(2-morpholinoethoxy)phenyl)amino)-[1,4′-bipiperidin]-1′-yl)-3-(1H-1,2,4-triazol-1-yl)propan-2-ol

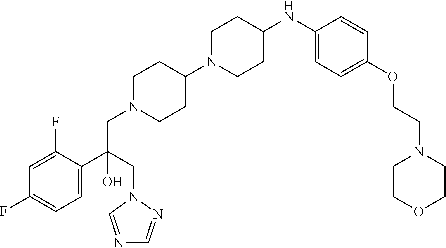 [SMILES: OC(CN1CCC(N2CCC(Nc3ccc(OCCN4CCOCC4)cc3)CC2)CC1)(Cn1cncn1)c1ccc(F)cc1F]

[M+H]⁺: 626.7

### Example 21

### 2-(2,4-difluorophenyl)-1-(4-((4-fluorophenyl)amino)azepan-1-yl)-3-(1H-1,2,4-triazol-1-yl)propan-2-ol

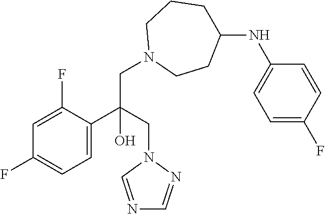 [SMILES: OC(CN1CCCC(Nc2ccc(F)cc2)CC1)(Cn1cncn1)c1ccc(F)cc1F]

[M+H]⁺: 446.5

### Example 22

### 2-(2,4-difluorophenyl)-1-(4-((5-fluoropyrimidin-2-yl)amino)azepan-1-yl)-3-(1H-1,2,4-triazol-1-yl)propan-2-ol

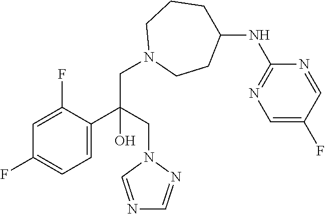 [SMILES: OC(CN1CCCC(Nc2ncc(F)cn2)CC1)(Cn1cncn1)c1ccc(F)cc1F]

[M+H]⁺: 447.7

### Example 23

### 2-(2,4-difluorophenyl)-1-(4-((4-(2-morpholinoethoxy)phenyl)amino)azepan-1-yl)-3-(1H-1,2,4-triazol-1-yl)propan-2-ol

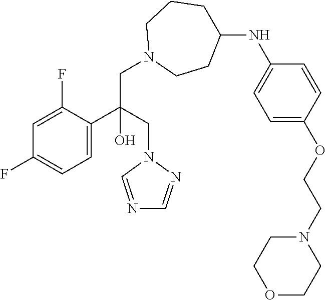 [SMILES: OC(CN1CCCC(Nc2ccc(OCCN3CCOCC3)cc2)CC1)(Cn1cncn1)c1ccc(F)cc1F]

[M+H]⁺: 557.6

### Example 24

### 2-(2-fluoro-4-methylphenyl)-1-(4-((4-(4-fluorobenzyl)phenyl)amino)azepan-1-yl)-3-(1H-1,2,4-triazol-1-yl)propan-2-ol

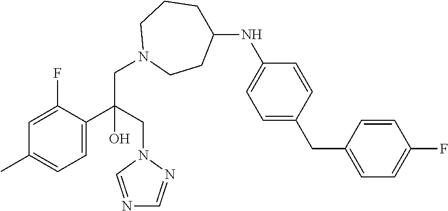 [SMILES: Cc1ccc(C(O)(CN2CCCC(Nc3ccc(Cc4ccc(F)cc4)cc3)CC2)Cn2cncn2)c(F)c1]

[M+H]⁺: 532.4

### Example 25

### 2-(2,4-difluorophenyl)-1-(4-(4-(4-fluorobenzyl)phenyl)-1,4-diazepan-1-yl)-3-(1H-1,2,4-triazol-1-yl)propan-2-ol

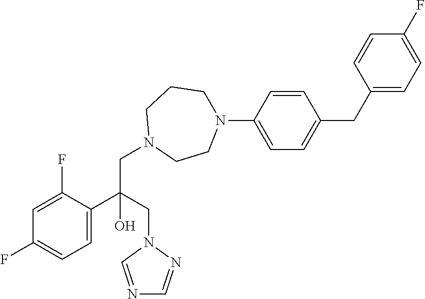 [SMILES: OC(CN1CCCN(c2ccc(Cc3ccc(F)cc3)cc2)CC1)(Cn1cncn1)c1ccc(F)cc1F]

[M+H]⁺: 522.5

### Example 26

### 2-(2,4-difluorophenyl)-1-(4-(4-(2-morpholinoethoxy)phenyl)-1,4-diazepan-1-yl)-3-(1H-1,2,4-triazol-1-yl)propan-2-ol

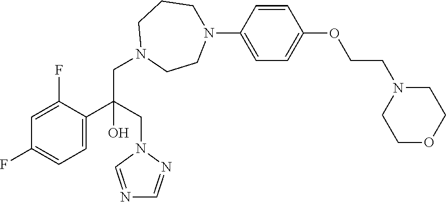 [SMILES: OC(CN1CCCN(c2ccc(OCCN3CCOCC3)cc2)CC1)(Cn1cncn1)c1ccc(F)cc1F]

[M+H]⁺: 543.4

### Example 27

### 1-(8-(4-benzylphenyl)-2,8-diazaspiro[4.5]decan-2-yl)-2-(2,4-difluorophenyl)-3-(1H-1,2,4-triazol-1-yl)propan-2-ol

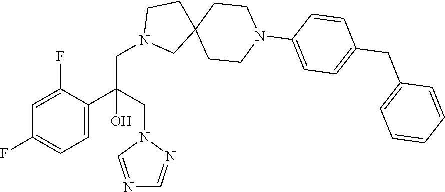 [SMILES: OC(CN1CCC2(CCN(c3ccc(Cc4ccccc4)cc3)CC2)C1)(Cn1cncn1)c1ccc(F)cc1F]

[M+H]⁺: 544.5

### Example 28

### 2-(2,4-difluorophenyl)-1-(8-((5-fluoropyrimidin-2-yl)amino)-2-azaspiro[4.5]decan-2-yl)-3-(1H-1,2,4-triazol-1-yl)propan-2-ol

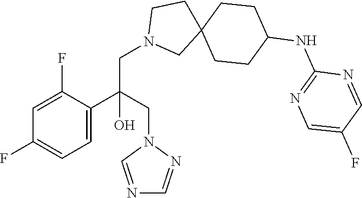 [SMILES: OC(CN1CCC2(CCC(Nc3ncc(F)cn3)CC2)C1)(Cn1cncn1)c1ccc(F)cc1F]

[M+H]⁺: 488.2

### Example 29

### 1-(5-(4-benzylphenyl)hexahydropyrrolo[3,4-c]pyrrol-2(1H)-yl)-2-(2,4-difluorophenyl)-3-(1H-1,2,4-triazol-1-yl)propan-2-ol

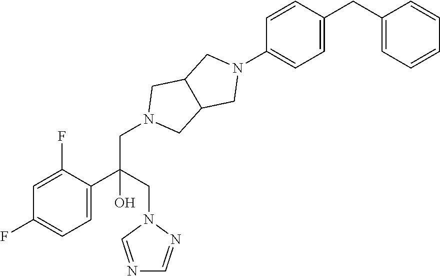 [SMILES: OC(CN1CC2CN(c3ccc(Cc4ccccc4)cc3)CC2C1)(Cn1cncn1)c1ccc(F)cc1F]

[M+H]⁺: 516.5

### Example 30

### 2-(2,4-difluorophenyl)-1-(4-((4-(4-fluorobenzyl)phenyl)amino)piperidin-1-yl)-3-(1H-1,2,4-triazol-1-yl)propan-2-ol

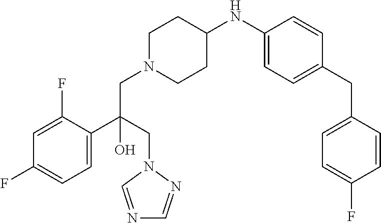 [SMILES: OC(CN1CCC(Nc2ccc(Cc3ccc(F)cc3)cc2)CC1)(Cn1cncn1)c1ccc(F)cc1F]

[M+H]⁺: 522.3.

Compounds numbers 31 to 80 set out in Table 2 may be prepared using analogous methods to those described above for Examples 1 to 30.

### General Procedure for Boc-Protected Intermediate Synthesis

Aniline (1.2 equiv.) was added into the flask containing Boc-protected ketone linker (1 equiv.) while stirring in anhydrous DCM (0.75 mol ml⁻¹). Sodium triacetoxyborohydride (STAB) (1.5 equiv.) and glacial acetic acid (1.5 equiv.) were added portionwise and dropwise respectively at 0° C. The reaction mixture was stirred under argon at room temperature overnight. The crude was diluted with 1 N NaOH and extracted with DCM for three times. Combined organic layer was dried over MgSO₄ (anhydrous), filtered, and evaporated in vacuo. Flash column chromatography was carried out for further purification.

### tert-butyl 4-((4-(pyridin-4-ylmethoxy)phenyl)amino)piperidine-1-carboxylate (Compound Number 81 Intermediate)

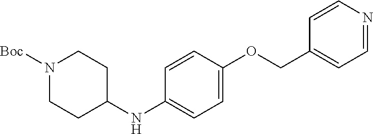 [SMILES: CC(C)(C)OC(=O)N1CCC(Nc2ccc(OCc3ccncc3)cc2)CC1]

N-Boc-4-piperidone and 4-(pyridin-4-ylmethoxy)aniline were used.

[M+H]⁺: 384.2, Rt=2.21 min.

### tert-butyl 4-((4-(2-(pyrrolidin-1-yl)ethoxy)phenyl)amino)piperidine-1-carboxylate (Compound Number 82 Intermediate)

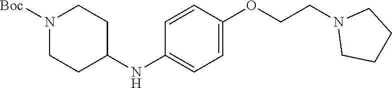 [SMILES: CC(C)(C)OC(=O)N1CCC(Nc2ccc(OCCN3CCCC3)cc2)CC1]

N-Boc-4-piperidone and 4-(2-(pyrrolidin-1-yl)ethoxy)aniline were used.

¹H NMR (400 MHz, CDCl₃) σ: 1.23-1.33 (2H, m), 1.45 (9H, s), 1.96-2.03 (6H, m), 2.89 (2H, t, J=11.9 Hz), 3.15-3.32 (7H, m), 4.04-4.05 (2H, br), 4.29 (2H, t, J=5.1 Hz), 6.56 (2H, dd, J=8.9 Hz, 2.1 Hz), 6.78 (2H, dd, J=8.9 Hz, 2.1 Hz). [M+H]⁺: 390.2, Rt=2.05 min.

### tert-butyl 4-((4-((4-methylpiperidin-1-yl)methoxy)phenyl)amino)piperidine-1-carboxylate (Compound Number 83 Intermediate)

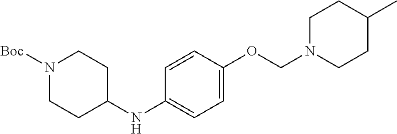 [SMILES: CC1CCN(COc2ccc(NC3CCN(C(=O)OC(C)(C)C)CC3)cc2)CC1]

N-Boc-4-piperidone and 4-((4-methylpiperidin-1-yl)methoxy)aniline were used.

[M+H]⁺:404.2, Rt=2.18 min.

### tert-butyl 4-((4-isobutoxyphenyl)amino)piperidine-1-carboxylate (Compound Number 84 Intermediate)

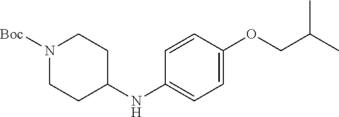 [SMILES: CC(C)COc1ccc(NC2CCN(C(=O)OC(C)(C)C)CC2)cc1]

N-Boc-4-piperidone and 4-isobutoxyaniline were used.

¹H NMR (400 MHz, CDCl₃) σ: 1.00 (6H, d, J=6.7 Hz), 1.28-1.37 (2H, m), 1.45 (9H, s), 2.04-2.07 (3H, m), 2.85 (2H, t, J=12.1 Hz), 3.28-3.35 (1H, m), 3.65 (2H, d, J=6.6 Hz), 3.70-3.74 (1H, m), 4.03-4.05 (2H, br), 6.70 (2H, d, J=8.6 Hz), 6.78 (2H, d, J=8.6 Hz). [M+H]⁺: 349.2, Rt=3.33 min.

### tert-butyl 4-((4-(p-tolyloxy)phenyl)amino)piperidine-1-carboxylate (Compound Number 85 Intermediate)

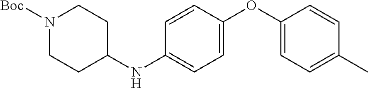 [SMILES: Cc1ccc(Oc2ccc(NC3CCN(C(=O)OC(C)(C)C)CC3)cc2)cc1]

N-Boc-4-piperidone and 4-(p-tolyloxy)aniline were used.

¹H NMR (400 MHz, CDCl₃) σ: 1.28-1.37 (2H, m), 1.46 (9H, s), 1.86-2.07 (3H, m), 2.32 (3H, s), 2.83 (2H, t, J=12.8 Hz), 3.33-3.38 (1H, m), 4.06-4.08 (2H, br), 6.83-6.92 (5H, m), 6.95-7.03 (1H, m), 7.10 (2H, d, J=8.2 Hz). [M+H]⁺: 383.2, Rt=3.79 min.

### tert-butyl 4-((4-(4-methoxyphenoxy)phenyl)amino)piperidine-1-carboxylate (Compound Number 86 Intermediate)

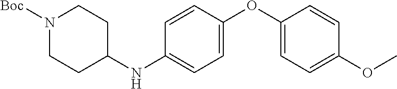 [SMILES: COc1ccc(Oc2ccc(NC3CCN(C(=O)OC(C)(C)C)CC3)cc2)cc1]

N-Boc-4-piperidone and 4-(4-methoxyphenoxy)aniline were used.

¹H NMR (400 MHz, CDCl₃) δ: 1.33-1.38 (2H, m), 1.46 (9H, s), 2.02-2.05 (3H, m), 2.86 (2H, t, J=12.1 Hz), 3.35 (1H, tt, J=10.4 Hz, 3.9 Hz), 3.78 (3H, s), 4.05-4.07 (2H, br), 6.72 (2H, d, J=8.1 Hz), 6.85 (4H, ddd, J=6.6 Hz, 6.3 Hz, 2.5 Hz), 6.89-6.93 (2H, m).

### tert-butyl 8-((4-(2-morpholinoethoxy)phenyl)amino)-2-azaspiro[4.5]decane-2-carboxylate (Compound Number 87 Intermediate)

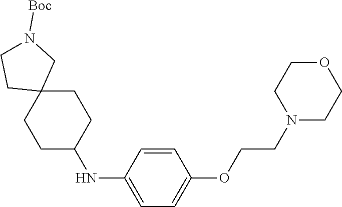 [SMILES: CC(C)(C)OC(=O)N1CCC2(CCC(Nc3ccc(OCCN4CCOCC4)cc3)CC2)C1]

tert-Butyl 8-oxo-2-azaspiro[4.5]decane-2-carboxylate and 4-(2-morpholinoethoxy)aniline were used.

¹H NMR (400 MHz, CDCl₃) σ: 1.14-1.40 (4H, m), 1.45 (9H, s), 1.60-1.73 (4H, m), 1.97-2.03 (2H, m), 2.58 (4H, dd, J=4.5 Hz, 4.5 Hz), 2.77 (2H, d, J=5.7 Hz), 3.07-3.22 (4H, m), 3.31-3.40 (2H, m), 3.73 (4H, t, J=4.7 Hz), 4.04 (2H, t, J=5.7 Hz), 6.54 (2H, d, J=8.9 Hz), 6.76 (2H, d, J=8.9 Hz). [M+H]⁺: 460.3, Rt=2.29 min.

### tert-Butyl 4-((4-(benzyloxy)phenyl)amino)piperidine-1-carboxylate (Compound Number 90 Intermediate)

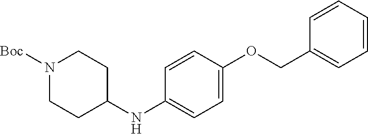 [SMILES: CC(C)(C)OC(=O)N1CCC(Nc2ccc(OCc3ccccc3)cc2)CC1]

N-Boc-4-piperidone and 4-(benzyloxy)aniline were used.

¹H NMR (400 MHz, CDCl₃) σ: 1.39 (9H, s), 1.95 (2H, d, J=15.61 Hz), 2.79 (1H, t, J=24.38 Hz), 3.25 (2H, br), 4.92 (1H, s), 5.22 (2H, s), 6.7 (4H, dd, J=2.31, 6.59 Hz), 7.32 (5H, br).

### tert-butyl 4-((4-(2-morpholinoethoxy)phenyl)amino)azepane-1-carboxylate (Compound Number 91 Intermediate)

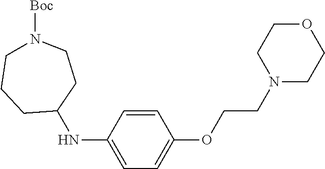 [SMILES: CC(C)(C)OC(=O)N1CCCC(Nc2ccc(OCCN3CCOCC3)cc2)CC1]

tert-butyl 4-oxoazepane-1-carboxylate and 4-(2-morpholinoethoxy)aniline were used.
10 ¹H NMR (400 MHz, CDCl₃) σ: 1.26-1.41 (1H, m), 1.46 (9H, s), 1.51-1.64 (2H, m), 1.80-1.88 (1H, m), 1.95-2.00 (1H, m), 2.09-2.13 (1H, m), 2.58 (4H, dd, J=4.5 Hz, 4.5 Hz), 2.77 (2H, t, J=5.7 Hz), 3.21-3.62 (6H, m), 3.73 (4H, dd, J=4.7 Hz, 4.7 Hz), 4.04 (2H, t, J=5.7 Hz), 6.50 (2H, d, J=8.8 Hz), 6.77 (2H, d, J=8.6 Hz). [M+H]⁺: 420.2, Rt=2.02 min.

### tert-butyl 4-((4-(2-morpholinoethoxy)phenyl)amino)-[1,4′-bipiperidine]-1′-carboxylate (Compound Number 92 Intermediate)

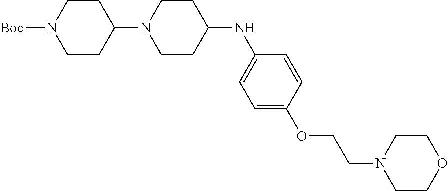 [SMILES: CC(C)(C)OC(=O)N1CCC(N2CCC(Nc3ccc(OCCN4CCOCC4)cc3)CC2)CC1]

tert-butyl 4-oxo-[1,4′-bipiperidine]-1′-carboxylate and 4-(2-morpholinoethoxy)aniline were used.

¹H NMR (400 MHz, CDCl₃) σ: 1.44 (9H, s), 1.60-1.69 (2H, m), 2.02-2.33 (6H, m), 2.57-2.84 (10H, m), 3.06-3.16 (1H, m), 3.59 (2H, dd, J=14.6 Hz, 7.3 Hz), 3.76 (4H, dd, J=4.6 Hz, 4.6 Hz), 4.07 (2H, t, J=5.5 Hz), 4.25-4.28 (2H, br), 6.59 (2H, d, J=8.9 Hz), 6.77 (2H, d, J=8.9 Hz). [M+H]⁺: 489.3, Rt=1.96 min.

### tert-butyl 4-((4-(2-methoxyethoxy)phenyl)amino)piperidine-1-carboxylate (Compound Number 96 Intermediate)

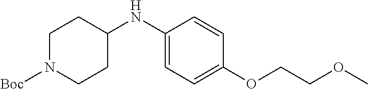 [SMILES: COCCOc1ccc(NC2CCN(C(=O)OC(C)(C)C)CC2)cc1]

N-Boc-4-piperidone and 4-(2-methoxyethoxy)aniline were used.

¹H NMR (400 MHz, CDCl₃) δ: 1.24-1.38 (2H, m), 1.45 (9H, s), 2.02 (2H, d, J=14.8 Hz), 2.84 (2H, t, J=12.2 Hz), 3.32 (1H, tt, J=10.5 Hz, 3.9 Hz), 3.44 (3H, s), 3.46 (1H, s), 3.71 (2H, td, J=4.8 Hz, 3.3 Hz), 4.04-4.06 (4H, m), 6.72 (2H, d, J=8.65 Hz), 6.82 (2H, dd, J=, 9.0 Hz, 2.3 Hz). [M+H]⁺: 351.2, Rt=2.42 min.

### tert-butyl 4-((4-morpholinophenyl)amino)piperidine-1-carboxylate (Compound Number 93 Intermediate)

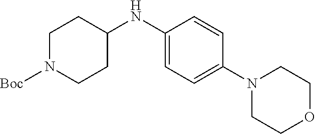 [SMILES: CC(C)(C)OC(=O)N1CCC(Nc2ccc(N3CCOCC3)cc2)CC1]

N-Boc-4-piperidone and 4-morpholinoaniline were used. [M+H]⁺: 362.2, Rt=2.26 min.

### tert-butyl 4-((4-(2-(piperidin-1-yl)ethoxy)phenyl)amino)piperidine-1-carboxylate (Compound Number 95 Intermediate)

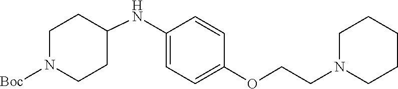 [SMILES: CC(C)(C)OC(=O)N1CCC(Nc2ccc(OCCN3CCCCC3)cc2)CC1]

N-Boc-4-piperidone and 4-(2-(piperidin-1-yl)ethoxy)aniline were used. [M+H]⁺: 404.3, Rt=2.14 min.

### tert-butyl 4-((4-(2-(4-methylthiazol-5-yl)ethoxy)phenyl)amino)piperidine-1-carboxylate (Compound Number 94 Intermediate)

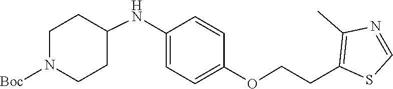 [SMILES: Cc1ncsc1CCOc1ccc(NC2CCN(C(=O)OC(C)(C)C)CC2)cc1]

N-Boc-4-piperidone and 4-(2-(4-methylthiazol-5-yl)ethoxy)aniline were used. [M+H]⁺: 418.2, Rt=2.97 min.

### tert-butyl 4-((4-((1-methylpiperidin-4-yl)oxy)phenyl)amino)piperidine-1-carboxylate (Compound Number 98 Intermediate)

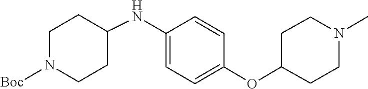 [SMILES: CN1CCC(Oc2ccc(NC3CCN(C(=O)OC(C)(C)C)CC3)cc2)CC1]

N-Boc-4-piperidone and 4-((1-methylpiperidin-4-yl)oxy)aniline were used. [M+H]⁺: 390.2, Rt=2.14 min.

### tert-butyl 4-((4-(2-(azepan-1-yl)ethoxy)phenyl)amino)piperidine-1-carboxylate (Compound Number 97 Intermediate)

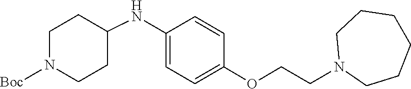 [SMILES: CC(C)(C)OC(=O)N1CCC(Nc2ccc(OCCN3CCCCCC3)cc2)CC1]

N-Boc-4-piperidone and 4-(2-(azepan-1-yl)ethoxy)aniline were used. [M+H]⁺: 418.3, Rt=2.25 min.

### General Procedure for Boc-Protected Intermediate Synthesis

Boc-protected linker (1.0 equiv.) and halo-aromatic building block (1.275 equiv.) were added to the flask that containing DMF (0.16 mmol ml⁻¹) while stirring, followed by the addition of DIPEA (3.125 equiv.) dropwise. The mixture was heated under argon at 125-130° C. overnight until no starting material appeared on TLC and LC-MS. The flask was cooled down to room temperature and the mixture was diluted with water then extracted with DCM for three times before dried with MgSO₄ and evaporated in vacuo. Crude product was further purified with gradient flash column chromatography (DCM and ethyl acetate).

### tert-butyl 8-(4-(2-morpholinoethoxy)phenyl)-2,8-diazaspiro[4.5]decane-2-carboxylate (Compound Number 88 Intermediate)

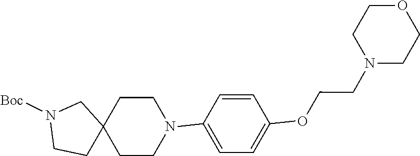 [SMILES: CC(C)(C)OC(=O)N1CCC2(CCN(c3ccc(OCCN4CCOCC4)cc3)CC2)C1]

2,2-dimethyl-1-(2,8-diazaspiro[4.5]decan-2-yl)propan-1-one and 4-(2-(4-bromophenoxy)ethyl)morpholine were used. [M+H]⁺: 446.2, Rt=2.14 min.

### tert-butyl 4-(4-(2-morpholinoethoxy)phenyl)-1,4-diazepane-1-carboxylate (Compound Number 89 Intermediate)

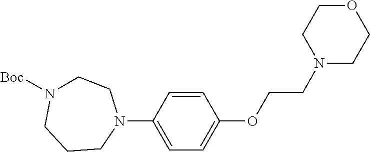 [SMILES: CC(C)(C)OC(=O)N1CCCN(c2ccc(OCCN3CCOCC3)cc2)CC1]

1-(1,4-diazepan-1-yl)-2,2-dimethylpropan-1-one and 4-(2-(4-bromophenoxy)ethyl)morpholine were used. [M+H]⁺: 406.2, Rt=2.54 min.

### General Procedure for Boc-Protected Intermediate Synthesis

Aniline (1.2 equiv.) was added into the flask containing Boc-protected ketone linker (1 equiv.) while stirring in anhydrous DCM (0.75 mol ml⁻¹). Sodium triacetoxyborohydride (STAB) (1.5 equiv.) and glacial acetic acid (1.5 equiv.) were added portionwise and dropwise respectively at 0° C. The reaction mixture was stirred under argon at room temperature overnight. The crude was diluted with 1 N NaOH and extracted with DCM for three times. Combined organic layer was dried over MgSO₄ (anhydrous), filtered, and evaporated in vacuo. Flash column chromatography was carried out for further purification.

### Tert-butyl(3aR,6aS)-5-((3′-fluoro-4′-methyl-[1,1′-biphenyl]-3-yl)amino)hexahydrocyclopenta[c]pyrrole-2(1H)-carboxylate (Compound Number 99 Intermediate)

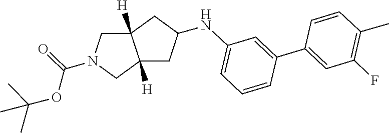 [SMILES: Cc1ccc(-c2cccc(NC3C[C@@H]4CN(C(=O)OC(C)(C)C)C[C@@H]4C3)c2)cc1F]

Tert-butyl (3aR,6aS)-5-oxohexahydrocyclopenta[c]pyrrole-2(1H)-carboxylate and 3-(3-fluoro-4-methylphenyl)aniline were used. [M+H]⁺: 411.2, Rt=4.18 min.

### Tert-butyl (3aR,6aS)-5-((4-(piperidin-1-yl)phenyl)amino)hexahydrocyclopenta[c]pyrrole-2(1H)-carboxylate (Compound Number 100 Intermediate)

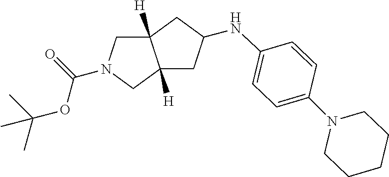 [SMILES: CC(C)(C)OC(=O)N1C[C@H]2CC(Nc3ccc(N4CCCCC4)cc3)C[C@H]2C1]

Tert-butyl (3aR,6aS)-5-oxohexahydrocyclopenta[c]pyrrole-2(1H)-carboxylate and 4-(piperidin-1-yl)aniline were used. [M+H]⁺: 386.2, Rt=3.02 min.

### Tert-butyl (3aR,6aS)-5-((4-fluorophenyl)amino)hexahydrocyclopenta[c]pyrrole-2(1H)-carboxylate (Compound Number 101 Intermediate)

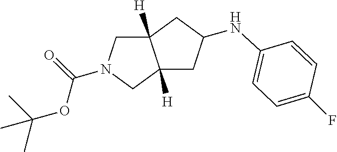 [SMILES: CC(C)(C)OC(=O)N1C[C@H]2CC(Nc3ccc(F)cc3)C[C@H]2C1]

Tert-butyl (3aR,6aS)-5-oxohexahydrocyclopenta[c]pyrrole-2(1H)-carboxylate and 4-fluoroaniline were used. [M+H]⁺: 321.2, Rt=3.50 min.

### General Experimental Procedure for Boc-Deprotection Reactions

4M HCl in 1,4-dioxane (0.2335 mmol ml-1) was added to the flask that contains Boc-protected intermediate (1 equiv.) dropwise while stirring. The mixture was stirred at room temperature until all starting material disappeared on TLC and LC-MS. After evaporating down the solvent under reduced pressure, the crude product was used directly in final reaction without further purification assuming 100% conversion.

### General Experimental Procedure for Final Compounds Synthesis

1-((2-(2,4-difluorophenyl)oxiran-2-yl)methyl)-1H-1,2,4-triazole (1.0 equiv.) (“triazole intermediate”) and triethylamine (1.5 equiv.) was added into the solution of amine (1.5 equiv.) or corresponding amine hydrochloride salt in ethanol (0.07 mmol/mL) under stirring. The mixture was heated under stirring at 80° C. overnight until all starting material disappeared. The solvent was removed under reduced pressure and product was purified by flash column chromatography (MeOH in EtOAc, 0-20% and TEA 0-1%).

### Example 31

### 2-(2,4-difluorophenyl)-1-(4-((4-(pyridin-4-ylmethoxy)phenyl)amino)piperidin-1-yl)-3-(1H-1,2,4-triazol-1-yl)propan-2-ol (Compound Number 81)

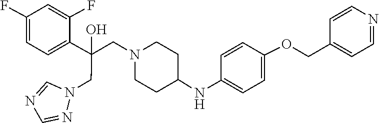 [SMILES: OC(CN1CCC(Nc2ccc(OCc3ccncc3)cc2)CC1)(Cn1cncn1)c1ccc(F)cc1F]

Prepared from triazole intermediate and compound number 81 intermediate.

¹H NMR (400 MHz, methanol-d₄) σ: 1.38-1.42 (2H, m), 1.78 (1H, dddd, J=11.6 Hz, J=11.2 Hz, J=1.8 Hz, J=1.8 Hz), 1.89 (1H, dddd, J=11.6 Hz, J=11.2 Hz, J=1.8 Hz, J=1.8 Hz), 2.25 (1H, td, J=11.6 Hz, 2.5 Hz), 2.42 (1H, td, J=11.6 Hz, 2.5 Hz), 2.55-2.59 (1H, m), 2.74-2.84 (2H, m), 3.02 (1H, dd, J=13.8 Hz, 1.2 Hz), 3.10 (1H, tt, J=10.6 Hz, 4.0 Hz), 4.58 (1H, d, J=14.2 Hz), 4.67 (1H, d, J=14.2 Hz), 5.07 (2H, s), 6.63 (2H, dd, J=10.3 Hz, 2.9 Hz), 6.81 (2H, dd, J=10.3 Hz, 2.9 Hz), 6.84-6.96 (2H, m), 7.45-7.52 (2H, m), 7.75 (1H, s), 8.34 (1H, s), 8.50 (2H, dd, J=4.6 Hz, J=1.5 Hz). [M+H]⁺: 521.2, Rt=1.87 min. HRMS (El, m/z) calculated C₂₈H₃₁F₂N₆O₂⁺: 521.2471, found 521.2467.

### Example 32

### 2-(2,4-difluorophenyl)-1-(4-((4-(2-(pyrrolidin-1-yl)ethoxy)phenyl)amino)piperidin-1-yl)-3-(1H-1,2,4-triazol-1-yl)propan-2-ol (Compound Number 82)

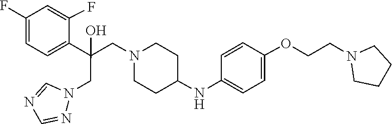 [SMILES: OC(CN1CCC(Nc2ccc(OCCN3CCCC3)cc2)CC1)(Cn1cncn1)c1ccc(F)cc1F]

Prepared from triazole intermediate and compound number 82 intermediate.

¹H NMR (400 MHz, methanol-d₄) σ: 1.29-1.36 (4H, m), 1.38-1.42 (2H, m), 1.78 (1H, dddd, J=11.6 Hz, J=11.2 Hz, J=1.8 Hz, J=1.8 Hz), 1.89 (1H, dddd, J=11.6 Hz, J=11.2 Hz, J=1.8 Hz, J=1.8 Hz), 2.09 (4H, ddd, J=7.1 Hz, 4.0 Hz, 3.1 Hz), 2.25 (1H, td, J=11.6 Hz, 2.5 Hz), 2.42 (1H, td, J=11.6 Hz, 2.5 Hz), 2.55-2.59 (1H, m), 2.75-2.78 (1H, m), 2.82 (1H, d, J=13.8 Hz), 3.02 (1H, dd, J=13.8 Hz, 1.2 Hz), 3.12 (1H, tt, J=10.4 Hz, 4.0 Hz), 3.53 (2H, t, J=5.1 Hz), 4.19 (2H, t, J=5.1 Hz), 4.59 (1H, d, J=14.2 Hz), 4.68 (1H, d, J=14.3 Hz), 6.64 (2H, dd, J=10.3 Hz, 2.9 Hz), 6.83 (2H, dd, J=10.3 Hz, 2.9 Hz), 6.85-6.96 (2H, m), 7.49 (1H, td, J=9.0 Hz, 6.7 Hz), 7.75 (1H, s), 8.35 (1H, s). [M+H]⁺: 527.3, Rt=1.75 min. HRMS (El, m/z): calculated C₂₈H₃₇F₂N₆O₂⁺: 527.2941, found 527.2937.

### Example 33

### 2-(2,4-difluorophenyl)-1-(4-((4-((4-methylpiperidin-1-yl)methoxy)phenyl)amino)piperidin-1-yl)-3-(1H-1,2,4-triazol-1-yl)propan-2-ol (Compound Number 83)

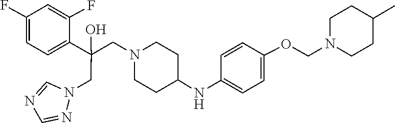 [SMILES: CC1CCN(COc2ccc(NC3CCN(CC(O)(Cn4cncn4)c4ccc(F)cc4F)CC3)cc2)CC1]

Prepared from triazole intermediate and compound number 83 intermediate.

¹H NMR (400 MHz, methanol-d₄) σ: 1.35-1.50 (4H, m), 1.59-1.70 (2H, m), 1.78 (1H, d, J=13.1 Hz), 1.88 (1H, d, J=13.1 Hz), 2.05 (2H, d, J=13.5 Hz), 2.24 (1H, t, J=11.4 Hz), 2.41 (1H, t, J=11.2 Hz), 2.58 (1H, d, J=11.6 Hz), 2.75-2.83 (4H, m), 2.93 (2H, t, J=12.4 Hz), 3.02 (1H, d, J=13.8 Hz), 3.06-3.14 (1H, m), 3.45 (2H, d, J=12.5 Hz), 3.80 (2H, d, J=5.5 Hz), 4.60 (1H, d, J=14.2 Hz), 4.69 (1H, d, J=14.3 Hz), 6.64 (2H, d, J=8.8 Hz), 6.75 (2H, d, J=8.8 Hz), 6.86 (1H, td, J=8.4 Hz, 2.5 Hz), 6.90-6.96 (1H, m), 7.49 (1H, td, J=9.0 Hz, 6.8 Hz), 7.76 (1H, s), 8.36 (1H, s). [M+H]⁺: 541.3, Rt=1.76 min. HRMS (El, m/z) calculated C₂₉H₃₉F₂N₆O₂⁺: 541.3097, found 541.3099.

### Example 34

### 2-(2,4-difluorophenyl)-1-(4-((4-isobutoxyphenyl)amino)piperidin-1-yl)-3-(1H-1,2,4-triazol-1-yl)propan-2-ol (Compound Number 84)

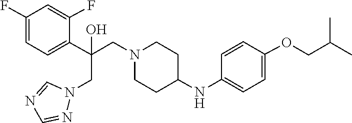 [SMILES: CC(C)COc1ccc(NC2CCN(CC(O)(Cn3cncn3)c3ccc(F)cc3F)CC2)cc1]

Prepared from triazole intermediate and compound number 84 intermediate.

¹H NMR (400 MHz, methanol-d₄) σ: 0.99 (6H, d, J=6.7 Hz), 1.34-1.47 (2H, m), 1.78-1.82 (1H, m), 1.88-1.92 (1H, m), 1.95-2.05 (1H, m), 2.27 (1H, td, J=11.6 Hz, 2.5 Hz), 2.42-2.48 (2H, m), 2.60-2.63 (1H, m), 2.78-2.81 (1H, m), 2.87 (1H, d, J=13.8 Hz), 3.07 (1H, dd, J=13.8 Hz, 1.3 Hz), 3.63 (2H, d, J=6.5 Hz), 4.59 (1H, d, J=14.3 Hz), 4.68 (1H, d, J=14.3 Hz), 6.64 (2H, dd, J=10.3 Hz, 2.9 Hz), 6.73 (2H, dd, J=10.3 Hz, 2.9 Hz), 6.85 (1H, td, J=8.5 Hz, 2.5 Hz), 6.93 (1H, ddd, J=9.2 Hz, 8.8 Hz, 2.5 Hz), 7.49 (1H, td, J=9.0 Hz, 6.6 Hz), 7.76 (1H, s), 8.34 (1H, s). [M+H]⁺: 486.2, Rt=2.80 min. HRMS (El, m/z) calculated C₂₆H₃₄F₂N₅O₂⁺: 486.2675, found 486.2672.

### Example 35

### 2-(2,4-difluorophenyl)-1-(4-((4-(p-tolyloxy)phenyl)amino)piperidin-1-yl)-3-(1H-1,2,4-triazol-1-yl)propan-2-ol (Compound Number 85)

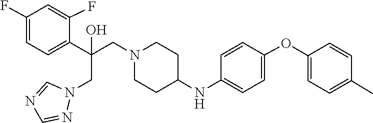 [SMILES: Cc1ccc(Oc2ccc(NC3CCN(CC(O)(Cn4cncn4)c4ccc(F)cc4F)CC3)cc2)cc1]

Prepared from triazole intermediate and compound number 85 intermediate.

¹H NMR (400 MHz, methanol-d₄) σ: 1.31-1.47 (2H, m), 1.80-1.83 (1H, m), 1.91-1.94 (1H, m), 2.22-2.29 (1H, m), 2.26 (3H, s), 2.42 (1H, td, J=11.5 Hz, 2.6 Hz), 2.54-2.60 (1H, m), 2.75-2.78 (1H, m), 2.82 (1H, d, J=13.8 Hz), 3.02 (1H, dd, J=13.8 Hz, 1.5 Hz), 3.15 (1H, tt, J=10.3 Hz, 4.1 Hz), 4.59 (1H, d, J=14.2 Hz), 4.68 (1H, d, J=14.4 Hz), 6.63 (2H, ddd, J=9.0 Hz, 3.4 Hz, 2.3 Hz), 6.73-6.79 (4H, m), 6.84 (1H, tdd, J=8.4 Hz, 2.5 Hz, 0.6 Hz), 6.90-6.96 (1H, m), 7.06 (2H, dd, J=8.7 Hz, 0.6 Hz), 7.49 (1H, td, J=9.0 Hz, 6.7 Hz), 7.75 (1H, s), 8.35 (1H, s). [M+H]⁺: 520.2, Rt=3.15 min. HRMS (El, m/z): calculated for C₂₉H₃₂F₂N₅O₂⁺ 520.2519 found 520.2514.

### Example 36

### 2-(2,4-difluorophenyl)-1-(4-((4-(4-methoxyphenoxy)phenyl)amino)piperidin-1-yl)-3-(1H-1,2,4-triazol-1-yl)propan-2-ol (Compound Number 86)

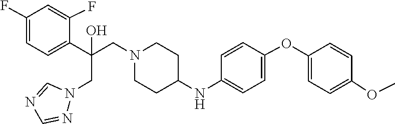 [SMILES: COc1ccc(Oc2ccc(NC3CCN(CC(O)(Cn4cncn4)c4ccc(F)cc4F)CC3)cc2)cc1]

Prepared from triazole intermediate and compound number 86 intermediate.

¹H NMR (400 MHz, methanol-d₄) σ: 1.36-1.47 (2H, m), 1.79-1.82 (1H, m), 1.89-1.94 (1H, m), 2.25 (1H, td, J=11.6 Hz, 2.6 Hz), 2.41 (1H, td, J=11.6 Hz, 2.6 Hz), 2.56-2.59 (1H, m), 2.74-2.78 (1H, m), 2.81 (1H, d, J=13.8 Hz), 3.02 (1H, dd, J=13.8 Hz, 1.5 Hz), 3.14 (1H, tt, J=10.3 Hz, 3.9 Hz), 3.74 (3H, s), 4.63 (1H, d, J=14.2 Hz), 4.68 (1H, d, J=14.4 Hz), 6.62 (2H, ddd, J=9.0 Hz, 3.4 Hz, 2.3 Hz), 6.75 (2H, ddd, J=J=9.0 Hz, 3.4 Hz, 2.3 Hz), 6.80-6.87 (5H, m), 6.89-6.96 (1H, m), 7.49 (1H, td, J=9.0 Hz, 6.6 Hz), 7.75 (1H, s), 8.35 (1H, s). [M+H]⁺: 536.2, Rt=2.97 min. HRMS (El, m/z): calculated for C₂₉H₃₂F₂N₅O₃⁺ 536.2468 found 536.2465.

### Example 37

### 2-(2,4-difluorophenyl)-1-(8-((4-(2-morpholinoethoxy)phenyl)amino)-2-azaspiro[4.5]decan-2-yl)-3-(1H-1,2,4-triazol-1-yl)propan-2-ol (Compound Number 87)

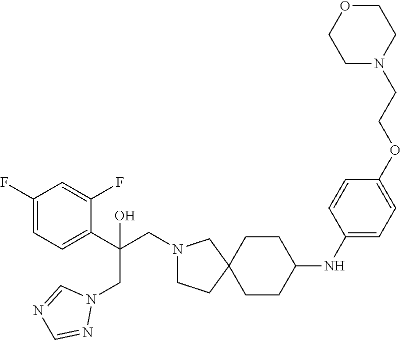 [SMILES: OC(CN1CCC2(CCC(Nc3ccc(OCCN4CCOCC4)cc3)CC2)C1)(Cn1cncn1)c1ccc(F)cc1F]

Prepared from triazole intermediate and compound number 87 intermediate.

¹H NMR (400 MHz, methanol-d₄) σ: 0.99-1.27 (2H, m), 1.31-1.38 (2H, m), 1.50 (td, J=7.3 Hz, 1.4 Hz), 1.56-1.64 (3H, m), 1.82-1.85 (2H, m), 2.26 (1H, d, J=2.1 Hz), 2.39 (1H, dd, J=24.9 Hz, 9.3 Hz), 2.49-2.59 (6H, m), 2.75 (2H, t, J=5.4 Hz), 2.92-3.10 (3H, m), 3.71 (4H, t, J=4.6 Hz), 4.04 (2H, t, J=5.3 Hz), 4.58 (1H, dd, J=14.2 Hz, 1.94 Hz), 4.67 (1H, d, J=14.2 Hz), 6.64 (2H, d, J=8.8 Hz), 6.76 (2H, d, J=8.3 Hz), 6.85 (1H, td, J=8.4 Hz, 2.3 Hz), 6.89-6.96 (1H, m), 7.49 (1H, tdd, J=9.1 Hz, 6.6 Hz, 2.6 Hz), 7.75 (1H, d, J=2.6 Hz), 8.34 (1H, s). [M+H]⁺: 597.3, Rt=1.53 min. HRMS (El, m/z) calculated C₃₂H₄₃F₂N₆O₃⁺: 597.3359 found 597.3360.

### Example 38

### 2-(2,4-difluorophenyl)-1-(8-(4-(2-morpholinoethoxy)phenyl)-2,8-diazaspiro[4.5]decan-2-yl)-3-(1H-1,2,4-triazol-1-yl)propan-2-ol (Compound Number 88)

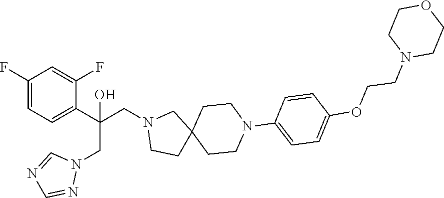 [SMILES: OC(CN1CCC2(CCN(c3ccc(OCCN4CCOCC4)cc3)CC2)C1)(Cn1cncn1)c1ccc(F)cc1F]

Prepared from triazole intermediate and compound number 88 intermediate.

[M+H]⁺: 583.2, Rt=1.30 min.

### Example 39

### 2-(2,4-difluorophenyl)-1-(4-(4-(2-morpholinoethoxy)phenyl)-1,4-diazepan-1-yl)-3-(1H-1,2,4-triazol-1-yl)propan-2-ol (Compound Number 89)

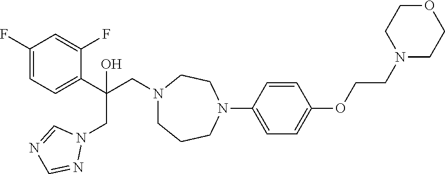 [SMILES: OC(CN1CCCN(c2ccc(OCCN3CCOCC3)cc2)CC1)(Cn1cncn1)c1ccc(F)cc1F]

Prepared from triazole intermediate and compound number 89 intermediate.

¹H NMR (400 MHz, methanol-d₄) σ: 1.61-1.76 (2H, m), 2.50-2.66 (6H, m), 2.76 (2H, t, J=5.5 Hz), 2.79-2.81 (2H, m), 2.86 (1H, d, J=14.0 Hz), 3.19 (1H, dd, J=14.0 Hz, 1.3 Hz), 3.33-3.38 (2H, m), 3.42-3.50 (2H, m), 3.71 (4H, dd, J=4.7 Hz, 4.7 Hz), 4.05 (2H, t, J=5.5 Hz), 4.46 (1H, d, J=14.2 Hz), 4.59 (1H, d, J=14.3 Hz), 6.63 (2H, ddd, J=9.2 Hz, 3.7 Hz, 2.3 Hz), 6.80-6.84 (3H, m), 6.89 (1H, ddd, J=11.6 Hz, 8.9 Hz, 2.5 Hz), 7.37 (1H, td, J=9.0 Hz, 6.7 Hz), 7.72 (1H, s), 8.26 (1H, s). [M+H]⁺: 543.2, Rt=1.83 min. HRMS (El, m/z) calculated C₂₈H₃₇F₂N₆O₃⁺: 543.2890, found 543.2888.

### Example 40

### 2-(2,4-difluorophenyl)-1-(4-((4-(2-morpholinoethoxy)phenyl)amino)azepan-1-yl)-3-(1H-1,2,4-triazol-1-yl)propan-2-ol (Compound Number 91)

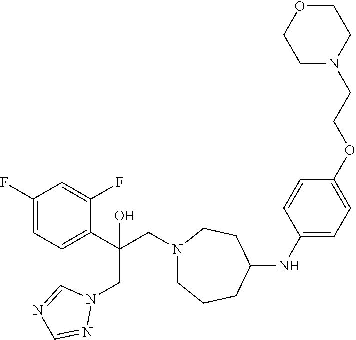 [SMILES: OC(CN1CCCC(Nc2ccc(OCCN3CCOCC3)cc2)CC1)(Cn1cncn1)c1ccc(F)cc1F]

Prepared from triazole intermediate and compound number 91 intermediate.

¹H NMR (400 MHz, methanol-d₄) σ: 1.42-1.66 (4H, m), 1.82-1.89 (2H, m), 2.57-2.71 (8H, m), 2.75 (2H, t, J=5.5 Hz), 2.89 (1H, dd, J=14.0 Hz, 6.7 Hz), 3.23 (1H, ddd, J=14.0 Hz, 5.8 Hz, 1.5 Hz), 3.37 (1H, tt, J=12.4 Hz, 3.9 Hz), 3.71 (4H, dd, J=4.7 Hz, 4.7 Hz), 4.04 (2H, t, J=5.5 Hz), 4.55 (1H, dd, J=14.2 Hz, 6.1 Hz), 4.68 (1H, d, J=14.3 Hz), 6.58 (2H, dd, J=8.9 Hz, 0.8 Hz), 6.77 (2H, ddd, J=8.9 Hz, 3.5 Hz, 1.9 Hz), 6.85 (1H, tdd, J=8.2 Hz, 2.7 Hz, 2.8 Hz), 6.92 (1H, ddd, J=11.6 Hz, 8.9 Hz, 2.4 Hz), 7.52 (1H, tdd, J=9.02 Hz, 6.7 Hz, 2.4 Hz), 7.75 (1H, s), 8.34 (1H, d, J=1.3 Hz). [M+H]⁺: 557.3, Rt=1.64 min.

### Example 41

### 2-(2,4-difluorophenyl)-1-(4-((4-(2-morpholinoethoxy)phenyl)amino)-[1,4′-bipiperidin]-1′-yl)-3-(1H-1,2,4-triazol-1-yl)propan-2-ol (Compound Number 92)

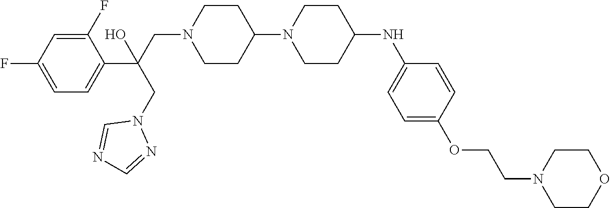 [SMILES: OC(CN1CCC(N2CCC(Nc3ccc(OCCN4CCOCC4)cc3)CC2)CC1)(Cn1cncn1)c1ccc(F)cc1F]

Prepared from triazole intermediate and compound number 92 intermediate.

¹H NMR (400 MHz, methanol-d₄) σ: 1.09-1.5 (2H, m), 1.61-1.81 (4H, m), 1.92-1.95 (1H, m), 2.01-2.04 (1H, m), 2.20-2.27 (3H, m), 2.38 (1H, td, J=11.9 Hz, 2.2 Hz), 2.76-2.83 (6H, m), 2.93 (2H, t, J=5.3 Hz), 3.01 (2H, dd, J=13.5 Hz, 1.5 Hz), 3.07-3.14 (2H, m), 3.48-3.51 (2H, m), 3.76 (4H, dd, J=4.8 Hz, 4.8 Hz), 4.10 (2H, t, J=5.4 Hz), 4.63 (1H, d, J=14.3 Hz), 4.69 (1H, d, J=14.3 Hz), 6.68 (2H, ddd, J=9.0 Hz, 3.4 Hz, 2.3 Hz), 6.81 (2H, ddd, J=9.0 Hz, 3.4 Hz, 2.3 Hz), 6.86 (1H, td, J=8.4 Hz, 2.5 Hz), 6.93 (1H, ddd, J=11.7 Hz, 8.9 Hz, 2.5 Hz), 7.48 (1H, td, J=9.0 Hz, 6.6 Hz), 7.76 (1H, s), 8.35 (1H, s). [M+H]⁺: 626.3, Rt=1.55 min.

### Example 42

### 2-(2,4-difluorophenyl)-1-(4-((4-(2-methoxyethoxy)phenyl)amino)piperidin-1-yl)-3-(1H-1,2,4-triazol-1-yl)propan-2-ol (Compound Number 96)

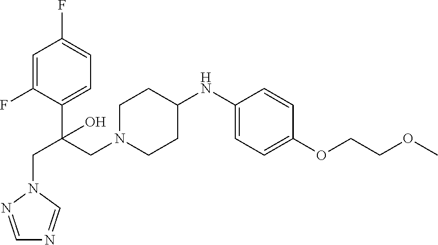 [SMILES: COCCOc1ccc(NC2CCN(CC(O)(Cn3cncn3)c3ccc(F)cc3F)CC2)cc1]

Prepared from triazole intermediate and compound number 96 intermediate. [M+H]⁺: 488.2, Rt=2.15 min.

### Example 43

### 1-(4-((4-(benzyloxy)phenyl)amino)piperidin-1-yl)-2-(2,4-difluorophenyl)-3-(1H-1,2,4-triazol-1-yl)propan-2-ol (Compound Number 90)

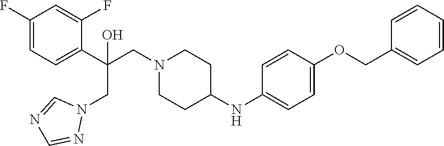 [SMILES: OC(CN1CCC(Nc2ccc(OCc3ccccc3)cc2)CC1)(Cn1cncn1)c1ccc(F)cc1F]

Prepared from triazole intermediate and compound number 90 intermediate.

¹H NMR (400 MHz, methanol-d₄) σ: 1.71 (4H, dd, J=12.65, 44.51 Hz), 2.09 (2H, td, J=2.44, 23.15 Hz), 2.27 (2H, td, J=2.5, 23.07 Hz), 2.43 (1H, d, J=12.87 Hz), 2.67; 2.88 (2H, dd, J=14.32, 82.74 Hz), 4.50 (2H, quin, J=53.21 Hz), 4.74 (1H, s), 4.84 (2H, s), 6.51 (2H, m, J=12.38 Hz), 6.68 (2H, m, J=12.41 Hz), 6.8 (1H, m, J=23.26 Hz), 7.21 (6H, m, J=57.98 Hz), 7.37 (1H, m, J=24.57 Hz), 7.64 (1H, s), 8.23 (1H, s). ¹³C NMR (100 MHz, methanol-d₄) δ: 50.79, 53.52, 54.27, 56.21, 56.26, 62.89, 62.92, 70.48, 73.02, 73.08, 103.25, 103.51, 103.78, 110.51, 110.54, 110.72, 110.75, 115.79, 125.99, 126.03, 126.13, 126.16, 127.18, 127.33, 128.02, 129.31, 129.37, 129.41, 129.47, 137.82, 141.59, 144.69, 149.69, 151.62, 158.02, 158.14, 160.47, 160.59, 161.45, 161.57, 163.91, 164.03.

### Example 44

### 2-(2,4-difluorophenyl)-1-(4-((4-((1-methylpiperidin-4-yl)oxy)phenyl)amino)piperidin-1-yl)-3-(1H-1,2,4-triazol-1-yl)propan-2-ol (Compound Number 98)

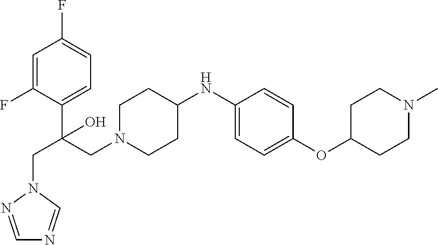 [SMILES: CN1CCC(Oc2ccc(NC3CCN(CC(O)(Cn4cncn4)c4ccc(F)cc4F)CC3)cc2)CC1]

Prepared from triazole intermediate and compound number 98 intermediate. [M+H]⁺: 527.3, Rt=1.74 min.

### Example 45

### 2-(2,4-difluorophenyl)-1-(4-((4-morpholinophenyl)amino)piperidin-1-yl)-3-(1H-1,2,4-triazol-1-yl)propan-2-ol (Compound Number 93)

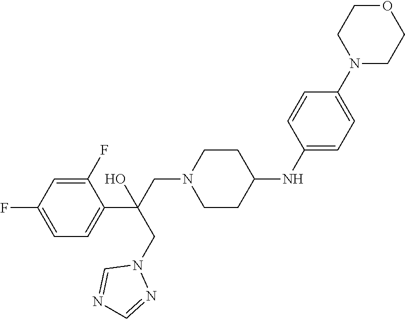 [SMILES: OC(CN1CCC(Nc2ccc(N3CCOCC3)cc2)CC1)(Cn1cncn1)c1ccc(F)cc1F]

Prepared from triazole intermediate and compound number 93 intermediate. [M+H]⁺: 499.2, Rt=1.90 min

### Example 46

### 2-(2,4-difluorophenyl)-1-(4-((4-(2-(piperidin-1-yl)ethoxy)phenyl)amino)piperidin-1-yl)-3-(1H-1,2,4-triazol-1-yl)propan-2-ol (Compound Number 95)

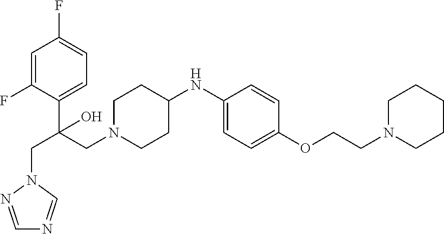 [SMILES: OC(CN1CCC(Nc2ccc(OCCN3CCCCC3)cc2)CC1)(Cn1cncn1)c1ccc(F)cc1F]

Prepared from triazole intermediate and compound number 95 intermediate. [M+H]⁺: 541.3.

### Example 47

### 2-(2,4-difluorophenyl)-1-(4-((4-(2-(4-methylthiazol-5-yl)ethoxy)phenyl)amino)piperidin-1-yl)-3-(1H-1,2,4-triazol-1-yl)propan-2-ol (Compound Number 94)

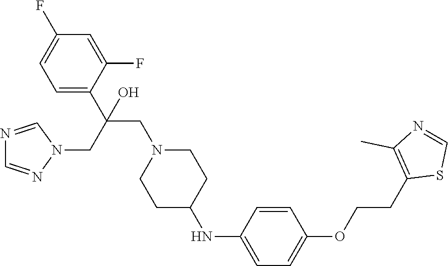 [SMILES: Cc1ncsc1CCOc1ccc(NC2CCN(CC(O)(Cn3cncn3)c3ccc(F)cc3F)CC2)cc1]

Prepared from triazole intermediate and compound number 94 intermediate. [M+H]⁺: 555.2.

### Example 48

2-(2,4-difluorophenyl)-1-((3aR,6aS)-5-((3′-fluoro-4′-methyl-[1,1′-biphenyl]-4-yl)amino)hexahydrocyclopenta[c]pyrrol-2(1H)-yl)-3-(1H-1,2,4-triazol-1-yl)propan-2-ol (Compound Number 99)

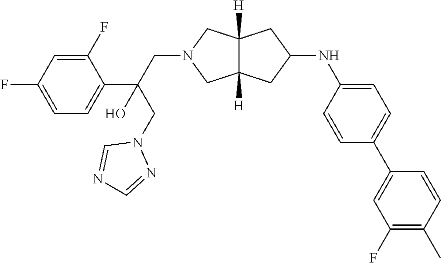 [SMILES: Cc1ccc(-c2ccc(NC3C[C@@H]4CN(CC(O)(Cn5cncn5)c5ccc(F)cc5F)C[C@@H]4C3)cc2)cc1F]

Prepared from triazole intermediate and compound number 99 intermediate.

¹H NMR (400 MHz, DMSO-d₆) δ ppm 0.95-1.10 (m, 2H), 2.08 (s, 3H), 2.09-2.19 (s, 3H), 2.21-2.27 (m, 2H), 2.30-2.40 (m, 3H), 2.81 (d, J=12.84 Hz, 1H), 2.90-2.99 (m, 1H), 4.58 (d, J=2.38 Hz, 2H), 5.60 (d, J=8.07 Hz, 1H), 5.66 (s, 1H), 6.51-6.58 (m, 1H), 6.75 (d, J=7.52 Hz, 1H), 6.81 (s, 1H), 6.95 (td, J=8.67, 2.11 Hz, 1H), 7.05-7.21 (m, 3H), 7.27-7.35 (m, 3H), 7.38-7.47 (m, 1H), 7.75 (s, 1H), 8.29 (s, 1H) [M−H]⁺: 546.2, Rt=3.21 min.

### Example 49

### 2-(2,4-difluorophenyl)-1-((3aR,6aS)-5-((4-(piperidin-1-yl)phenyl)amino)hexahydrocyclopenta[c]pyrrol-2(1H)-yl)-3-(1H-1,2,4-triazol-1-yl)propan-2-ol (Compound Number 100)

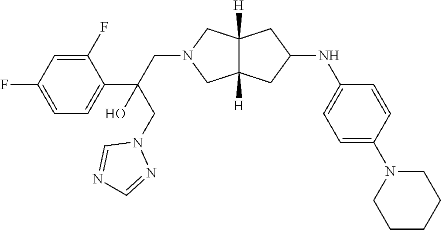 [SMILES: OC(CN1C[C@H]2CC(Nc3ccc(N4CCCCC4)cc3)C[C@H]2C1)(Cn1cncn1)c1ccc(F)cc1F]

Prepared from triazole intermediate and compound number 100 intermediate.

¹H NMR (400 MHz, DMSO-d₆) δ ppm 1.43-1.49 (m, 3H), 1.60 (dt, J=11.00, 5.69 Hz, 9H), 2.04-2.18 (m, 2H), 2.30-2.37 (m, 2H), 2.84-2.88 (m, 4H), 2.93-2.98 (m, 2H), 3.02 (s, 1H), 4.58 (d, J=2.93 Hz, 2H), 4.91 (d, J=8.99 Hz, 1H), 5.64 (s, 1H), 6.45-6.48 (m, 2H), 6.69-6.72 (m, 2H), 6.96 (td, J=8.62, 2.38 Hz, 1H), 7.13-7.18 (m, 1H), 7.42-7.45 (m, 1H), 7.76 (s, 1H), 8.30 (s, 1H)

[M+H]⁺: 523.2, Rt=1.94 min.

### Example 50

### 2-(2,4-difluorophenyl)-1-((3aR,6aS)-5-((4-fluorophenyl)amino)hexahydrocyclopenta[c]pyrrol-2(1H)-yl)-3-(1H-1,2,4-triazol-1-yl)propan-2-ol (Compound Number 101)

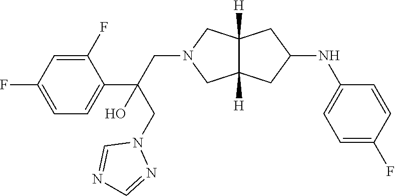 [SMILES: OC(CN1C[C@H]2CC(Nc3ccc(F)cc3)C[C@H]2C1)(Cn1cncn1)c1ccc(F)cc1F]

Prepared from triazole intermediate and compound number 101 intermediate.

¹H NMR (400 MHz, CHLOROFORM-d) δ ppm 1.14 (br. s., 1H) 1.26-1.37 (m, 1H) 2.08-2.28 (m, 4H), 2.46-2.63 (m, 4H), 3.01-3.15 (m, 2H), 3.49-3.58 (m, 1H), 4.56 (s, 2H), 6.61 (dd, J=8.80, 4.40 Hz, 2H), 6.75-6.83 (m, 2H), 6.83-6.91 (m, 2H), 7.46-7.53 (m, 1H), 7.78 (s, 1H), 8.15 (s, 1H) [M+H]⁺: 458.2, Rt=2.57 min.

### Example 51

### 1-(4-((4-(2-(azepan-1-yl)ethoxy)phenyl)amino)piperidin-1-yl)-2-(2,4-difluorophenyl)-3-(1H-1,2,4-triazol-1-yl)propan-2-ol (Compound Number 97)

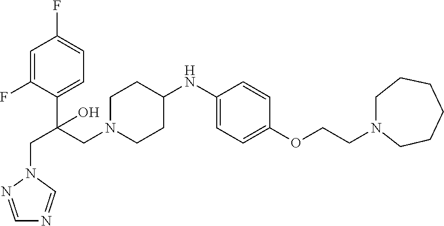 [SMILES: OC(CN1CCC(Nc2ccc(OCCN3CCCCCC3)cc2)CC1)(Cn1cncn1)c1ccc(F)cc1F]

Prepared from triazole intermediate and compound number 97 intermediate.

[M+H]⁺: 555.3.

### Biological Assays

The following assays can be used to measure the effects of the compounds of the present invention.

### Candida Panel Minimum Inhibitory Concentration

The minimum inhibitory concentration of test compounds were determined in a panel of Candida species (see Table 4 below), using the a serial dilution method in 96-well plates using the following protocol.

Starter cultures of fungal strains to be tested were grown in 3 mL of RPMI 2% glucose media incubated at 37° C. with shaking at 200 rpm overnight. The culture media used was 900 mL H₂O; 10.4 g RPMI 1640 (with L-glutamine and a pH indicator but without bicarbonate); 34.5 g MOPS and 18 g glucose. The pH of the media was adjusted to 7.0 with 1 M sodium hydroxide.

Solutions of the compound to be tested were prepared by dissolving the compound in DMSO and subsequently into media to the desired concentrations. 200 μL of compound solution was then added to the wells of the 96-well plate using a serial dilution method row-by row of the plate to give a compound concentration gradient across the plate of from 128 μg/mL to 0.25 μg/mL. The last two rows of each plate were control rows containing the culture media alone and the media plus fungi.

The fungi starter cultures were diluted 1:10 in water in cuvettes. This was then further diluted to an optical density (OD₆₀₀) of 0.01 in media. 100 μL of the fungi was added to each well giving a starting concentration of fungus of 0.5-5×10⁵/mL. One species of fungus was used on each plate. The plates were then held at 37° C. for 24 hours.

A plate reader was used to measure growth in each well at OD_{530 nm} and the MIC was determined for each test compound. The MIC is defined as the lowest concentration giving rise to inhibition of ≥50% of that of the drug-free control well containing the fungus and media.

The MIC (μg/mL) for the compounds tested in the Candida panel are shown in Table 4 below. The compounds exhibited antifungal activity against the Candida strains tested. A number of the compounds where highly active against Candida auris including resistant strains (see, for example the compounds of Examples 2, 3, 4 and 5 (which correspond to compound numbers 2, 3, 4 and 5, respectively)).

TABLE 4
MIC (μg/mL)

| | Common panel | | | | | | Extended panel |
|---|---|---|---|---|---|---|---|
| | | | | C. | C. | | Candida |
| Compound #/ | C. albicans | C. glabrata | C. krusei | tropicalis | parapsilosis | C. auris | auris |
| Code | NCPF3281 | NCPF8018 | NCPF3876 | NCPF8760 | NCPF3209 | TDG1912 | TDG2512 |
| Fluconazole | ≤0.125 | 0.5- | 0.5- | 4- | ≤0.25 | >128 | 16 |
| | | 4 | 8 | 8 | | | |
| Voriconazole | | | | | | >32 | >128 |
| 11 | ≤0.125 | ≤0.125 | ≤0.125 | ≤0.125 | ≤0.125 | 0.125 | ≤0.125 |
| 2 | ≤0.0039 | 0.125 | ≤0.003- | ≤0.003- | ≤0.003 | 0.03 | ≤0.003 |
| | | | 0.125 | 0.25 | | | |
| 5 | 0.002- | 0.125 | 0.002- | 0.008- | 0.004 | 0.03 | ≤0.003 |
| | 0.004 | | 0.125 | 0.25 | | | |
| 3 | ≤0.00098 | 0.125 | 0.004- | 0.015 | 0.004 | 0.008- | ≤0.003 |
| | | | 0.016 | | | 0.06 | |
| 4 | 0.008 | 0.25- | 0.004- | 0.25 | 0.004 | 0.06 | ≤0.003 |
| | | >0.5 | 0.125 | | | | |
| 30 | ≤0.125 | ≤0.125 | ≤0.125 | ≤0.125 | ≤0.125 | ≤0.125 | ≤0.125 |
| 6 | 0.004 | 0.25 | 0.125 | 0.008 | 0.015 | 0.125 | 0.015 |
| 10 | 0.015 | 64 | 32 | 64 | 0.125 | 4 | 0.5 |
| 12 | ≤0.125 | 0.125 | ≤0.125 | ≤0.125 | ≤0.125 | 0.125 | ≤0.125 |
| 14 | ≤0.125 | 16 | 4 | 32 | ≤0.125 | 1 | 0.25 |
| 8 | <0.015 | 0.25 | 0.5 | 1 | 0.015 | 2 | 2 |
| 9 | <0.004 | 0.125 | 0.008 | 0.125 | ≤0.00006 | 0.5 | 0.125 |
| 7 | <0.008 | 0.25 | 0.06 | 0.015 | 0.004 | 0.12 | 0.03 |
| 13 | ≤0.125 | 0.25 | ≤0.125 | ≤0.125 | ≤0.125 | 0.25 | ≤0.125 |
| 16 | ≤0.125 | ≤0.125 | ≤0.125 | 1 | ≤0.125 | 1 | ≤0.125 |
| 15 | 0.12 | 1 | 4 | 8 | 0.125 | 8 | 4 |
| 17 | ≤0.125 | 0.12 | 4 | 4- | ≤0.125 | 4- | |
| | | | | 8 | | 16 | |
| 1 | 0.5- | ≤0.125 | 1- | 0.25- | 4- | ≤0.125 | |
| | 1 | | 2 | 1 | 8 | | |
| 27 | | | | | | | |
| 81 | 0.00781 | 2 | 0.125 | 0.5 | 0.00781 | 0.0313 | 0.00781 |
| 82 | 0.25 | 32 | 8 | >32 | 0.25 | 4- | 1 |
| | | | | | | 16 | |
| 83 | 0.06 | 8 | 4 | 8 | 0.06 | 1- | 0.25 |
| | | | | | | 4 | |
| 84 | 0.00195 | 0.06 | 0.03 | 0.125 | ≤0.00098- | | 0.00195- |
| | | | | | 0.00195 | | 0.00391 |
| 85 | 0.0078 | 1 | 0.0156- | 0.25 | 0.00391- | 0.0625 | 0.00391- |
| | | | 0.25 | | 0.0313 | | 0.0313 |
| 86 | ≤0.0078- | 0.5 | 0.015- | 0.125 | ≤0.00098- | | 0.0078 |
| | 0.0156 | | 0.125 | | 0.0078 | | |
| 87 | 0.313 | 4 | 1 | 2 | 0.313 | 0.5 | 0.0625 |
| 88 | 0.0313 | 4 | 1 | 1 | 0.0313 | 0.5 | 0.0625 |
| 89 | 0.156 | 2 | 0.5 | 2 | 0.0313 | 1 | 0.25 |
| 90 | 0.00391 | 0.0625 | 0.0313 | 0.0625 | 0.00391 | | 0.00391 |
| 91 | 0.01563 | 16 | 2 | 8 | 0.0625 | 4 | 0.125 |
| 92 | 2 | >32 | >32 | >32 | 2- | 32 | 4 |
| | | | | | 8 | | |
| 96 | 0.0078- | 4 | 0.5 | 1 | 0.0156 | 0.125 | 4 |
| | 0.0156 | | | | | | |
| 98 | 0.125 | 8 | 8 | >8 | 0.125 | >8 | 2 |
| 99 | ≤0.03 | 8 | 1 | >32 | ≤0.03 | 16 | 0.5 |
| 100 | ≤0.03 | 2 | 2 | 8 | ≤0.03 | 8 | 0.25 |
| 101 | ≤0.03 | 0.5 | 1 | 4 | ≤0.03 | 1 | 0.125 |

| | Extended panel | | | | | |
|---|---|---|---|---|---|---|
| | Candida | Candida | Candida | | | |
| Compound #/ | auris | auris | auris | C. auris | C. auris | C. auris |
| Code | TDG2506 | TDG1102 | TDG2211 | 8984 | 8971 | 8977 |
| Fluconazole | >128 | 64 | 64 | >128 | 16 | 32 |
| Voriconazole | 2 | >128 | >128 | >128 | >32 | 32 |
| 11 | ≤0.125 | ≤0.125 | ≤0.125 | ≤0.125 | ≤0.125 | ≤0.125 |
| 2 | ≤0.003- | ≤0.003- | 0.25 | | | |
| | 0.15 | 0.5 | | | | |
| 5 | 0.006 | ≤0.003 | 0.006 | | | |
| 3 | 0.006 | ≤0.003 | 0.006 | 0.006 | 0.006 | 0.006 |
| 4 | ≤0.003 | 0.006 | 0.006 | | | |
| 30 | ≤0.125 | ≤0.125 | ≤0.125 | ≤0.125 | ≤0.125 | ≤0.125 |
| 6 | 0.25 | 0.12 | 0.5 | 0.125 | 0.03 | 0.06 |
| 10 | 8 | 4 | 64 | 0.5 | 0.5 | 1 |
| 12 | ≤0.125 | ≤0.125 | ≤0.125 | ≤0.125 | ≤0.125 | ≤0.125 |
| 14 | 2 | 1 | ≤0.125 | 1 | 0.25 | 0.25 |
| 8 | 8 | 1 | 1 | 0.12 | 0.12 | 0.12 |
| 9 | 0.5 | 1 | 0.25 | 0.12 | 0.12 | 0.0001 |
| 7 | 0.5 | 0.25 | 0.125 | 0.12 | 0.03 | 0.002 |
| 13 | ≤0.125 | ≤0.125 | ≤0.125 | ≤0.125 | ≤0.125 | ≤0.125 |
| 16 | 0.5 | 1 | ≤0.125 | 1 | ≤0.125 | ≤0.125 |
| 15 | >128 | 8 | 4 | 32 | 0.5 | 1 |
| 17 | | | | | | |
| 1 | | | | | | |
| 27 | | | | | | |
| 81 | 0.125 | 0.0625 | 0.0313 | 0.125 | 0.0156 | 0.0156 |
| 82 | 4 | 16 | 0.5- | 4 | 2-8 | 4-16 |
| | | | 2 | | | |
| 83 | 0.5- | 4 | 0.25 | 2 | 1 | 1 |
| | 2 | | | | | |
| 84 | 0.03 | 0.0156 | ≤0.00098- | 0.03 | 0.00195- | 0.00391 |
| | | | 0.00195 | | 0.00391 | |
| 85 | 0.0625- | 0.0625- | 0.015- | 0.0625- | 0.00195- | 0.015- |
| | 0.125 | 0.25 | 0.125 | 0.5 | 0.125 | 0.03 |
| 86 | 0.0625 | 0.015- | 0.0078 | 0.0078- | ≤0.00098- | ≤0.00098- |
| | | 0.0625 | | 0.125 | 0.03 | 0.0039 |
| 87 | 0.125 | 0.5 | 0.125 | 0.25 | 0.25 | 0.25 |
| 88 | 0.25 | 0.5 | 0.125 | 0.25 | 0.25 | 0.125 |
| 89 | 4- | 1 | 0.5 | 4- | 0.25 | 0.25 |
| | 8 | | | 8 | | |
| 90 | 0.003 | 0.01563 | 0.003 | 0.003 | 0.003 | 0.00781 |
| 91 | 4 | 2 | 0.25 | 4 | 0.5 | 0.5 |
| 92 | 32 | 32 | 8- | 32 | 16 | 8 |
| | | | 32 | | | |
| 96 | 0.06 | 0.06 | 0.0156- | 0.25 | 0.06 | 0.06 |
| | | | 0.06 | | | |
| 98 | >8 | >8 | >8 | >8 | 8 | 8 |
| 99 | 32 | 16- | 0.125- | 32 | 1- | 1- |
| | | 32 | 16 | | 4 | 4 |
| 100 | 4 | 16 | 2 | 4 | 1 | 2 |
| 101 | 4 | 2 | 0.125- | 4 | 0.25 | 0.25 |
| | | | 2 | | | |

MIC (μg/mL) of Selected Compounds Against Other Key MDR Candida Species

The compounds shown in Table 5 were tested against a further panel of Candida species.

TABLE 5
MIC (μg/mL)

| Compound #/ | C. Glabrata | C. Krusei | C. Tropicalis | C. Parapsilosis |
|---|---|---|---|---|
| Code | NCPF8018 | NCPF3876 | NCPF8760 | NCPF3209 |
| Fluconazole | 0.5-4 | 0.5-8 | 4-8 | ≤0.25 |
| Voriconazole | 1 | 0.5-2 | 4 | ≤0.25 |
| 3 | 0.125 | 0.004-0.016 | 0.015 | 0.004 |
| 6 | 2-4 | 0.125 | 0.008 | 0.015 |
| 12 | 0.25 | ≤0.125 | ≤0.125 | ≤0.125 |
| 9 | 0.125 | 0.008 | 0.5 | ≤0.00006 |
| 7 | 0.25 | 0.06 | 0.015 | 0.004 |
| 90 | 0.0625 | 0.0313 | 0.0625 | 0.00391 |
| 84 | 0.06 | 0.03 | 0.125 | ≤0.00098-0.00195 |
| 86 | 0.5 | 0.015-0.125 | 0.125 | ≤0.00098-0.0078 |

Candida albicans Panel Minimum Inhibitory Concentration

The compounds shown in Table 6 were tested against a further panel of Candida albicans.

TABLE 6
MIC (μg/mL)

| Compound #/ | C. albicans | C. albicans | C. albicans | C. albicans | C. albicans | C. albicans | C. albicans | C. albicans |
|---|---|---|---|---|---|---|---|---|
| Code | NCPF3281 | 3645 | 3990 | 8112 | 3881 | 3880 | 3363 | 3882 |
| Fluconazole | ≤0.125 | ≤0.125 | 8 | 0.125 | 8 | 16 | 8 | 16 |
| Voriconazole | ≤0.007 | ≤0.0078 | 0.25 | ≤0.00781 | 0.25 | 0.25 | 1 | 0.25 |
| 2 | ≤0.0039 | 0.00391 | 0.0156 | 0.00195- | 0.0156 | 0.00781 | 0.00781- | 0.0313 |
| | | | | 0.00781 | | | 0.125 | |
| 11 | ≤0.00098 | 0.00391 | 0.0039- | 0.00195 | 0.0313 | 0.0313 | 0.0156 | 0.0625 |
| | | | 0.0625 | | | | | |
| 12 | 0.002- | 0.00391 | 0.0313 | 0.00391 | 0.0625 | 0.0313 | 0.0625 | 0.0625 |
| | 0.004 | | | | | | | |
| 13 | ≤0.00098 | 0.00781 | 0.0625 | ≤0.00049- | 0.25 | 0.25 | 0.125 | 0.0313- |
| | | | | 0.00391 | | | | 2 |
| 90 | 0.00391 | 0.00391 | 0.0039 | 0.00391 | 0.0078- | 0.00391 | 0.008- | 0.00391 |
| | | | | | 0.0313 | | 0.03 | |
| 6 | ≤0.125 | 0.0156 | 0.25 | 0.0156 | 0.5 | 0.25 | 0.25 | 0.5 |

Activity Against C. auris with Multiple Target Mutations and Efflux Upregulation

The Candida auris strains shown in Table 7 were either taken from the National Collection of Pathogenic Fungi (NCPF) or collected as part of ongoing surveillance studies. The strains were characterised as follows.

Genomic DNA (gDNA) was extracted from yeast using the Wizard Genomic DNA Purification Kit (Promega; UK) according to manufacturer's instructions. In brief; 3 mL of an overnight culture of yeast cultured from a SAB agar plate and grown overnight with shaking in YPD media was centrifuged for 10 minutes at 5000 rpm. Supernatant was removed and the pellet was thoroughly resuspended in 270 μL sorbitol buffer (1M Sorbitol, 100 mM EDTA). 30 μL of lyticase enzyme (300U) was added and solution was incubated at 37° C. for 1 hour, then allowed to cool to room temperature. Sample was pelleted at 13,000 g for 2 minutes and supernatant removed. 300 μL Nuclei Lysis Solution was gently added by pipetting up and down until the pellet was dissolved. Sample was incubated at 37° C. for 30-60 minutes to break open the cells. If the sample was not viscous at this stage, freeze-thaw cycles were performed (1 min in dry ice, 1 min in 37° C. water bath) until sample became viscous. 100 μL Protein Precipitation Solution was added to each tube, vortexed for 20 seconds at maximum speed to precipitate proteins and then samples were sat on ice for 5 minutes. Sample was centrifuged at 13,000 rpm for 3 minutes to pellet the protein, then supernatant was carefully removed into another Eppendorf tube and centrifugation was repeated. The supernatant was transferred into a clean Eppendorf containing 300 μL of isopropanol and gently mixed by inversion until a “cloud” of DNA could be seen. The DNA was pelleted at 13,000 rpm for 2 minutes and the supernatant decanted, draining the tube onto clean absorbent paper. 300 μL 70% Ethanol was added, tube was gently inverted several times to wash the pellet and then centrifuged at 13,000 rpm for 2 minutes to remove salts. The ethanol was removed and the open tube containing the pellet was incubated at 37° C. for 15 minutes to evaporate all traces of ethanol. 50 μL of Rehydration Solution was added followed by 1.5 μL of RNase solution. Sample was vortexed for 1 second, centrifuged briefly (5 seconds) to collect droplets and incubated in the 37° C. heat block for 15 minutes. The DNA was rehydrated at 65° C. for 1 hour. DNA was quantified using a Qubit Fluorometer according the manufacturer's instructions (Invitrogen).

DNA was tagged and multiplexed with the Nextera XT DNA kit (Illumina). Whole-genome sequencing of C. auris isolates was performed by PHE-GSDU (Public Health England Genomic Services and Development Unit) on an Illumina (HiSeq 2500) with paired-end read lengths of 150 bp. A minimum 150 Mb of Q30 quality data were obtained for each isolate. FastQ files were quality trimmed using Trimmomatic. SPAdes 3.11.1 was used to produce draft chromosomal assemblies, and contigs of less than 500 bp were filtered out. Genome analysis of the FASTA files was carried out using SeqBuilder 14 and gene sequences from existing C. auris genomes from NCBI (e.g. PRJNA535510) were analysed using BLAST to compare the presence of specific genes and their corresponding sequences against the respective FASTA files. Variants in genes identified were analysed using Meg Align Pro 14 (DNA star).

The data in Table 7 below shows that the compounds are active against fungal strains with multiple target mutations and efflux upregulation.

TABLE 7

| | | | Mutations | Presence of | MIC (μg/mL) | | | | | |
|---|---|---|---|---|---|---|---|---|---|---|
| | | | in Erg-11 | efflux pumps | Com- | Com- | Com- | Com- | Com- | Com- |
| | | | linked to | associated | pound | pound | pound | pound | pound | pound |
| | MIC (μg/mL) | | azole | with azole | number | number | number | number | number | number |
| | Fluconazole | Voriconazole | resistance | resistance | 11 | 12 | 90 | 13 | 6 | 87 |
| C. auris | 16-128 | >128 | A125V, | CDR1, CDR2, | ≤0.125 | ≤0.125 | ≤0.003 | ≤0.125 | 0.25 | 0.125 |
| 2211 | | | L126F, | MDR1, MDR2, | | | | | | |
| | | | Y132F | SNQ2 | | | | | | |
| C. auris | >128 | 2 | A125V, | CDR1, CDR2, | 0.125 | ≤0.125 | ≤0.003 | ≤0.125 | 0.25 | 0.125 |
| 2506 | | | L126F, | MDR1, MDR2, | | | | | | |
| | | | Y132F | SNQ2 | | | | | | |
| C. auris | >128 | >128 | A125V, | CDR1, CDR2, | 0.125 | ≤0.125 | ≤0.003 | ≤0.003 | 0.125 | 0.25 |
| 8984 | | | L126F, | MDR1, MDR2, | | | | | | |
| | | | Y132F | SNQ2 | | | | | | |
| C. auris | 32 | >32 | None | CDR1, CDR2, | ≤0.125 | ≤0.125 | ≤0.003 | ≤0.125 | 0.03 | 0.25 |
| 8971 | | | identified | MDR1, MDR2, | | | | | | |
| | | | | SNQ2 | | | | | | |
| C. auris | 16 | 32 | None | CDR1, CDR2, | ≤0.125 | ≤0.125 | ≤0.003 | ≤0.125 | 0.06 | 0.25 |
| 8977 | | | identified | MDR1, MDR2, | | | | | | |
| | | | | SNQ2 | | | | | | |

### Aspergillus Panel Minimum Inhibitory Concentration

The compound of Example 2 (which corresponds to compound number 2) was tested against a panel of Aspergillus strains using a method analogous to that described above for the Candida Panel. The results are shown in Table 8:

TABLE 8

| | | Compound |
|---|---|---|
| | Fluconazole | number 2 |
| Aspergillus fumigatus | >64 | 0.25 |
| ATCC 1028 | | |
| Aspergillus fumigatus | >64 | 0.25 |
| ATCC 10894 | | |
| Aspergillus fumigatus | >64 | ≤0.125 |
| ATCC 13073 | | |
| Aspergillus fumigatus | >64 | ≤0.125 |
| ATCC 16424 | | |
| Aspergillus fumigatus | >64 | ≤0.125 |
| ATCC 204305 | | |
| Aspergillus fulavus | >64 | 0.5 |
| ATCC 204304 | | |
| Aspergillus carbonarius | >64 | 0.25 |
| ATCC 8740 | | |

These data show that the compound of Example 2 was highly effective against the Aspergillus strains tested compared to fluconazole.

Galleria mellonella Infection Model

The effects of test compounds in-vivo was assessed using a Galleria mellonella infection model.

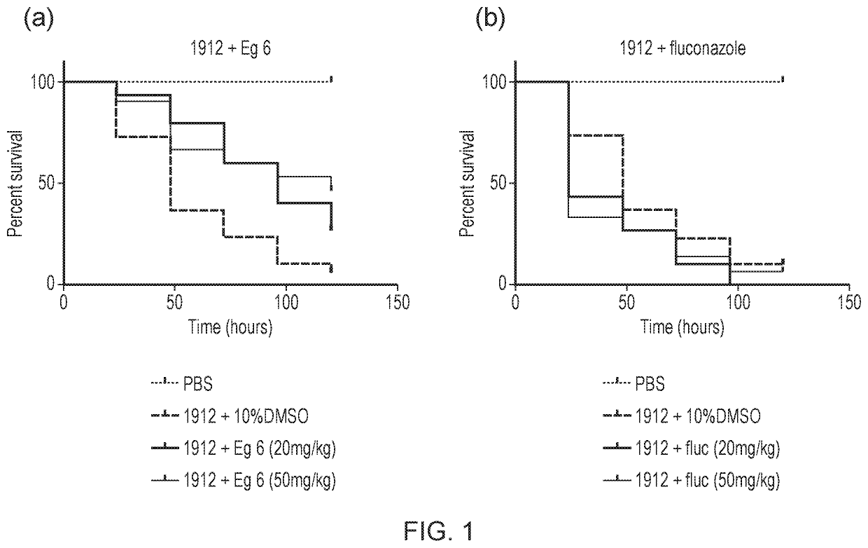

G. mellonella were injected in a proleg with 10 μL of C. auris strain TDG1912 (OD4) at a concentration of 1×10⁶ cfu/10 μL using a Hamilton syringe and left for 30 minutes to recover from injection and to allow infection to begin. The G. mellonella were then injected with 10 μL of the compound of the invention at either 50 mg/kg (10% DMSO) or 20 mg/kg (4% DMSO) or PBS containing 10% DMSO as an infection-only control. 10 larvae were selected for each treatment, and the experiment was repeated 3 times for the test compound and fluconazole. Survival of larvae was monitored for a 5 day period and results are presented as % survival over 120 hours (see FIG. 1).

The compound of Example 6 (which corresponds to compound number 6) showed statistically significant protection against the multi-drug resistant C. auris strain. In contrast no significant effect was observed in the fluconazole treatment arms. The p values are shown in Table 9 below:

TABLE 9

| | | Gehen-Breslaw- |
|---|---|---|
| P values cv 1912 + PBS | Mantel-Cox | Wilcoxon |
| Compound number 6 | 0.0002**** | 0.0002**** |
| (50 mg/kg) | | |
| Compound number 6 | 0.0009**** | 0.0009**** |
| (20 mg/kg) | | |
| Fluconazole (50 mg/kg) | 0.7629 | 0.8499 |
| Fluconazole (20 mg/kg) | 0.2443 | 0.2348 |
| Fluconazole (5 mg/kg) | 0.116 | 0.1366 |

No toxicity to the larvae was observed at a dose of 50 mg/kg of Example 6. This suggests that the compounds are selectively toxic to the fungal cells, indicating that the compounds may have a wide therapeutic window.

### Homology Modelling

Molecular docking studies have been used to investigate the rationale behind the observed activity of compounds of the invention. The compounds show considerably better Chemscore and affinities for both the lanosterol 14α-demethylase enzyme and both ABC and MSF efflux pumps that operate in C. auris. The structures for both enzyme and different efflux pumps were prepared using homology modelling approach. Two closely related genes, annotated within the C. auris genome as encoding ERG11 enzymes (A0A2H4QC40 & A0A2Z4EHX0) were applied to generate the amino acid sequences to build the 3D PDB structure by homology modelling.

Modelling results for binding of some of the compounds Exemplified herein to the target enzymes are shown in Table 10 and Table 11:

TABLE 10

| | carLDM_A0A2H4QC40 | | carLDM_A0A2Z4EHX0 | |
|---|---|---|---|---|
| Ligands | ChemScore | ΔG(kcal/mol) | ChemScore | ΔG(kcal/mol) |
| Fluconazole | 17.09 | −20.40 | 12.74 | −16.87 |
| Voriconazole | 17.80 | −24.26 | 17.42 | −24.39 |
| Compound number 2 | 38.08 | −42.39 | 40.04 | −47.57 |
| Compound number 3 | 43.18 | −49.23 | 40.74 | −50.11 |
| Compound number 4 | 40.42 | −49.27 | 41.41 | −47.33 |
| Compound number 6 | 38.7 | −43.62 | 40.1 | −49.35 |
| Compound number 10 | 39.9 | −43.73 | 37.21 | −40.56 |
| Compound number 11 | 43.39 | −47.08 | 41.39 | −49.15 |
| Compound number 12 | 51.17 | −55.16 | 47.7 | −62.37 |

TABLE 11

| | carLDM_A0A2H4QC40 | | carLDM_A0A2Z4EHX0 | |
|---|---|---|---|---|
| Ligands | ChemScore | ΔG(kcal/mol) | ChemScore | ΔG(kcal/mol) |
| Fluconazole | 16.82 | −18.92 | 14.65 | −17.39 |
| Voriconazole | 20.98 | −25.22 | 20.92 | −24.29 |
| Compound number 87 | 47.05 | −54.47 | 48.02 | −60.27 |
| Compound number 88 | 44.07 | −55.66 | 39.79 | −44.21 |
| Compound number 89 | 39.08 | −42.9 | 36.05 | −44.43 |
| Compound number 91 | 41.53 | −47 | 40.06 | −49.41 |
| Compound number 92 | 43.65 | −54.55 | 43.37 | −56.55 |
| Compound number 100 | 43.56 | −51.55 | 42.96 | −48.45 |
| Compound number 101 | 40 | −41.51 | 40.12 | −43.23 |

Modelling results for binding to efflux pumps commonly found in fungal species such as Candida are shown in Tables 12 to 15:

TABLE 12

| | carCDR1 | | carMDR1 | |
|---|---|---|---|---|
| Ligands | ChemScore | ΔG(kcal/mol) | ChemScore | ΔG(kcal/mol) |
| Fluconazole | 9.15 | −12.50 | 14.12 | −17.54 |
| Voriconazle | 9.12 | −12.75 | 16.28 | −21.35 |
| Compound | 32.07 | −37.23 | 33.38 | −38.82 |
| number 2 | | | | |
| Compound | 31.98 | −36.08 | 37.82 | −43.21 |
| number 3 | | | | |
| Compound | 31.21 | −35.54 | 37.42 | −46.71 |
| number 4 | | | | |
| Compound | 25.21 | −25.99 | 38.01 | −40.74 |
| number 6 | | | | |
| Compound | 20.67 | −22.16 | 31.02 | −35.8 |
| number 10 | | | | |
| Compound | 26.36 | −29.43 | 44.05 | −46.39 |
| number 11 | | | | |
| Compound | 28.04 | −31.04 | 41.78 | −47.94 |
| number 12 | | | | |

TABLE 13

| | carCDR1 | | carMDR1 | |
|---|---|---|---|---|
| Ligands | ChemScore | ΔG(kcal/mol) | ChemScore | ΔG(kcal/mol) |
| Fluconazole | 5.83 | −6.96 | 11.48 | −12.38 |
| Voriconazle | 10.48 | −12.06 | 18.76 | −19.6 |
| Compound | 27.78 | −34.41 | 40.65 | −47.55 |
| number 87 | | | | |
| Compound | 26.4 | −32.27 | 40.55 | −47.96 |
| number 88 | | | | |
| Compound | 24.71 | −27.27 | 40.09 | −42.6 |
| number 89 | | | | |
| Compound | 23.24 | −27.5 | 37.82 | −42.74 |
| number 91 | | | | |
| Compound | 26.62 | −30.87 | 40.41 | −43.99 |
| number 92 | | | | |
| Compound | 21.51 | −31.89 | 43.99 | −48.33 |
| number 100 | | | | |
| Compound | 23.44 | −24.39 | 35.5 | −36.4 |
| number 101 | | | | |

TABLE 14

| | carRDC3 | | carSNQ2 | | carYHD3 | |
|---|---|---|---|---|---|---|
| | | ΔG | | ΔG | | ΔG |
| Ligands | ChemScore | (kcal/mol) | ChemScore | (kcal/mol) | ChemScore | (kcal/mol) |
| Fluconazole | 10.29 | −13.00 | 9.80 | −11.91 | 7.82 | −10.33 |
| Voriconazle | 14.77 | −21.44 | 12.86 | −17.00 | 11.70 | −15.76 |
| Compound | 24.21 | −33.15 | 24.65 | −30.76 | 30.32 | −35.64 |
| number 2 | | | | | | |
| Compound | 27.45 | −36.35 | 27.48 | −35.64 | 30.43 | −35.51 |
| number 3 | | | | | | |
| Compound | 26.94 | −35.04 | 26.99 | −33.93 | 29.35 | −33.37 |
| number 4 | | | | | | |
| Compound | 22.84 | −29.39 | 26.66 | −31.72 | 28.31 | −29.94 |
| number 6 | | | | | | |
| Compound | 17.8 | −26.25 | 24.09 | −28.99 | 25.41 | −26.74 |
| number 10 | | | | | | |
| Compound | 30.17 | −35.34 | 29.53 | −31.6 | 36.72 | −38.09 |
| number 11 | | | | | | |
| Compound | 31.09 | −36.99 | 33.39 | −39.22 | 40.14 | −43.59 |
| number 12 | | | | | | |

TABLE 15

| | carRDC3 | | carSNQ2 | | carYHD3 | |
|---|---|---|---|---|---|---|
| | | ΔG | | ΔG | | ΔG |
| Ligands | ChemScore | (kcal/mol) | ChemScore | (kcal/mol) | ChemScore | (kcal/mol) |
| Fluconazole | 8.3 | −9.97 | 8.92 | −9.77 | 10.95 | −12.2 |
| Voriconazle | 12.58 | −13.71 | 16.57 | −19.12 | 15.99 | −21.76 |
| Compound | 28.76 | −32.63 | 31.16 | −36.59 | 32.74 | −36.12 |
| number 87 | | | | | | |
| Compound | 27.44 | −35.24 | 29.89 | −35.2 | 34.73 | −37.9 |
| number 88 | | | | | | |
| Compound | 27.86 | −28.43 | 28.87 | −30.67 | 31.58 | −34.67 |
| number 89 | | | | | | |
| Compound | 25.95 | −30.28 | 27.01 | −30.96 | 28.84 | −34.85 |
| number 91 | | | | | | |
| Compound | 26.38 | −34.18 | 29.77 | −35.73 | 31.12 | −34.78 |
| number 92 | | | | | | |
| Compound | 26.27 | −32.48 | 30.38 | −36.23 | 33.17 | −35.7 |
| number 100 | | | | | | |
| Compound | 25.38 | −29.09 | 24.46 | −26.55 | 29.04 | −30.57 |
| number 101 | | | | | | |

The data in Tables 10 to 15 shows that the ChemScores are significantly higher and the Gibbs free energy changes (ΔG) for the exemplified compounds are significantly lower than those for fluconazole and voriconazole. This indicates that the compounds have stronger binding affinities to both the target ERG11 enzymes and efflux pumps.

The molecular modelling studies suggest that compounds of the invention show greater affinity towards the target enzyme ERG11p (Lanosterol 14α-demethylase) than fluconazole through increased hydrophobic and hydrogen bond interactions, and fits snugly within the binding pocket. FIG. 2 shows the binding of Example 2.

The molecular modelling data presented in Tables 12 to 15 also suggest that the compounds of the invention show notably greater affinity towards the efflux pumps through increased hydrophobic and hydrogen bond interactions making them less susceptible to efflux.

The invention is further illustrated by the following numbered clauses.

### CLAUSES

1. A compound of the formula (I), or a pharmaceutically acceptable salt thereof:

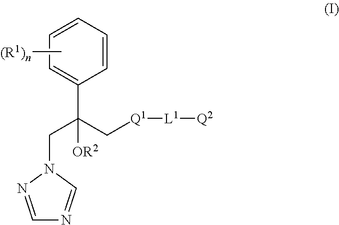

wherein:
R¹ is at each occurrence independently selected from: halo and C₁₋₄ alkyl;
n is 0, 1 or 2;
R² is selected from H and C₁₋₄ alkyl;
Q¹ is selected from:

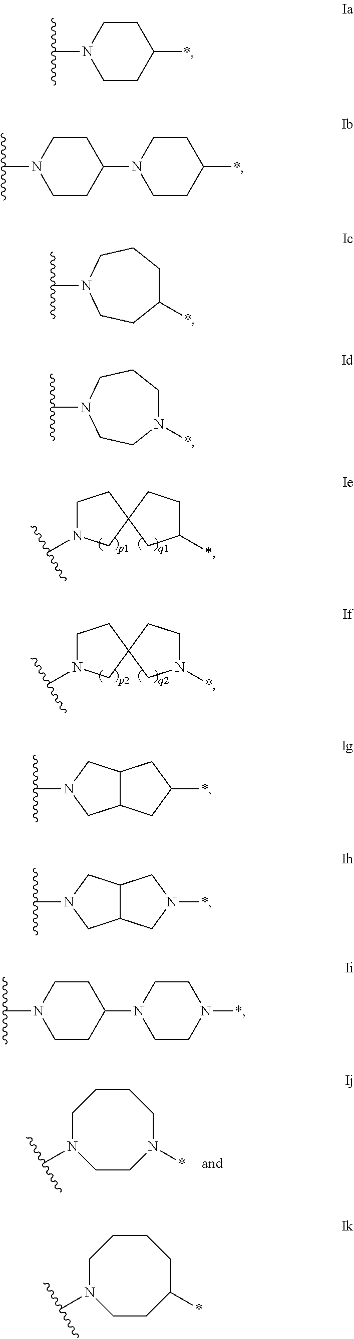

wherein * indicates the point of attachment to L¹,
p¹, q¹, p² and q² are independently an integer selected from 1 or 2,
and wherein Q¹ is optionally substituted by one or more R³;
each R³ is independently selected from: C₁₋₃ alkyl and ═O;
L¹ is a bond or is selected from —CH₂—, —O— and —NR⁴—, provided that L¹ is a bond or —CH₂— when Q¹ is bonded to L¹ via a ring nitrogen in Q¹;
R⁴ is selected from H and C₁₋₃ alkyl;
Q² is selected from phenyl or a 5- or 6-membered heteroaryl, wherein Q² is optionally substituted by one R⁵ and optionally one or more R⁶;
R⁵ is Q³-L³-, wherein
   L³ is a bond or is selected from: C₁₋₄ alkylene, -L⁴-O-L⁵-, -L⁴-NR^{A1}-L⁵-, -L⁴-S(O)ₓ-L⁵-, -L⁴-C(═O)-L⁵-, -L⁴-NR^{A1}C(═O)-L⁵-, -L⁴-C(═O)NR^{A1}-L⁵-, -L⁴-S(O)₂NR^{A1}-L⁵-, -L⁴-NR^{A1}S(O)₂-L⁵-, -L⁴-OC(═O)-L⁵ and -L⁴-C(═O)O-L⁵-,
   wherein x is 0, 1 or 2;
   L⁴ and L⁵ are independently selected from a bond and C₁₋₄ alkylene;
   Q³ is selected from: phenyl, a 5- or 6-membered heteroaryl and 3- to 12-membered heterocyclyl, wherein said phenyl or 5- or 6-membered heteroaryl is optionally substituted by one or more R⁷, and said 3- to 12-membered heterocyclyl is optionally substituted by one or more R⁸;
each R⁶, R⁷ and R⁸ is independently selected from: halo, ═O, —CN, —NO₂, C₁₋₄ alkyl, C₂₋₄ alkenyl, C₂₋₄ alkynyl, C₁₋₄ haloalkyl, C₁₋₆ cycloalkyl, —OR^{A1}, —S(O)ₓR^{A2} (wherein x is 0, 1, or 2), —NR^{A2}R^{B2}, —C(O)R^{A2}, —OC(O)R^{A2}, —C(O)OR^{A2}, —NR^{B2}C(O)R^{A2}, —NR^{B2}C(O)OR^{A2}, —C(O)NR^{A2}R^{B2}, —NR^{B2}SO₂R^{A2} and —SO₂NRIAR^{B2};
   wherein said C₁₋₄alkyl, C₂₋₄ alkenyl, C₂₋₄ alkynyl are each is optionally substituted by 1 or 2 substituents independently selected from: halo, —CN, —OR^{A3}, —NR^{A3}R^{B3} and —SO₂R^{A3}; and
   wherein said C₁₋₆ cycloalkyl is optionally substituted by one or more substituents independently selected from: halo, ═O, —CN, —NO₂, C₁₋₄ alkyl and C₁₋₄ haloalkyl;
   R^{A1}, R^{A2}, R^{B2}, R^{A3} and R^{B3} are each independently selected from: H, C₁₋₄ alkyl and C₁₋₄ haloalkyl;
with the provisos (i) and (ii):
(i) when n is 1 or 2; R¹ is halo;
Q¹ is

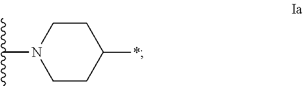

L¹ is —O—, —NH— or —N(CH₃)—; and
Q² is phenyl, pyridyl, or pyrimidyl;
then Q² is substituted by Q³-L³- and optionally one or more R⁶; and
(ii) when n is 1 or 2; R¹ is halo; L¹ is a bond; and
Q¹ is

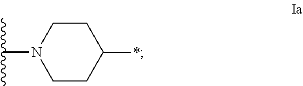

then Q² is phenyl or 6-membered heteroaryl, wherein said Q² is substituted by Q³-L³- and optionally one or more R⁶.
2. The compound of clause 1, wherein R² is H.
3. The compound of clause 1 or clause 2, wherein n is 1 or 2 and R¹ is selected from C₁₋₃ alkyl and halo, for example R¹ is selected from methyl and F.
4. The compound of any one of clauses 1 to 3, wherein the group

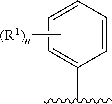

has the structure

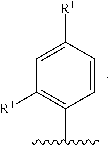

5. The compound of any one or clauses 1 to 4, wherein the group

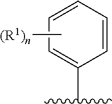

has the structure

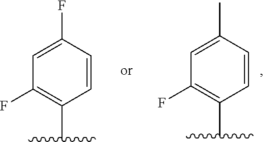

preferably

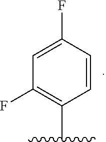 [SMILES: *c1ccc(F)cc1F]

6. The compound of any one of clauses 1 to 5, wherein Q¹ is selected from the group consisting of:

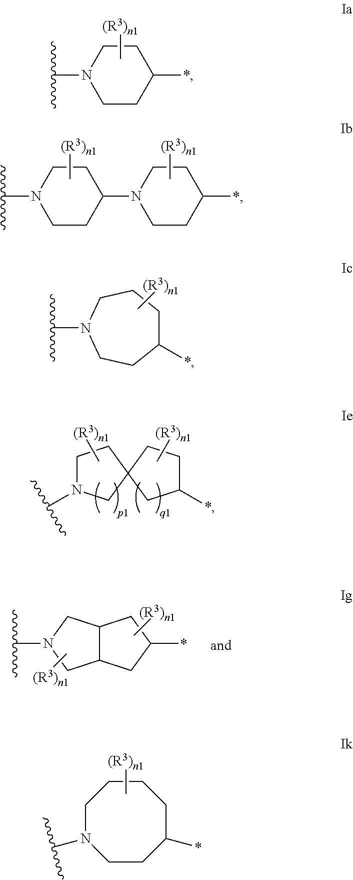

wherein:
   * indicates the point of attachment to L¹;
   p¹ and q¹ are independently an integer selected from 1 or 2;
   [symbol] indicates the point of attachment to Formula (I); and
   each n1 is independently selected from 0, 1 or 2.
7. The compound of any one of clauses 1 to 5, wherein Q¹ is

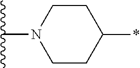 [SMILES: *C1CCN(*)CC1]

wherein * indicates the point of attachment to L¹.
8. The compound of clause 6 or clause 7, wherein L¹ is selected from a bond, —CH₂— and —NH—, for example wherein L¹ is NH.
9. The compound of any one of clauses 1 to 5, wherein Q¹-L¹- is selected from the group consisting of:

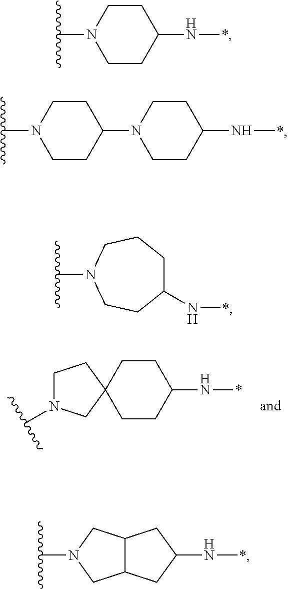

wherein:
   * indicates the point of attachment to Q².
10. The compound of any one of clauses 1 to 5, wherein Q¹-L¹- is

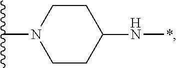

wherein * indicates the point of attachment to Q²
11. The compound of any one of clauses 1 to 5, wherein Q¹ is selected from the group consisting of:

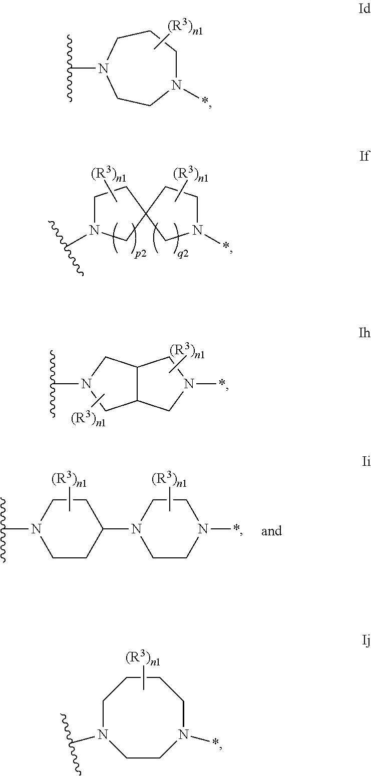

wherein:
   * indicates the point of attachment to L¹;
   p² and q² are independently an integer selected from 1 or 2;
   [symbol] indicates the point of attachment to Formula (I); and
   each n1 is independently selected from 0, 1 or 2.
12. The compound of any one of clauses 1 to 5, wherein Q¹ is selected from the group consisting of:

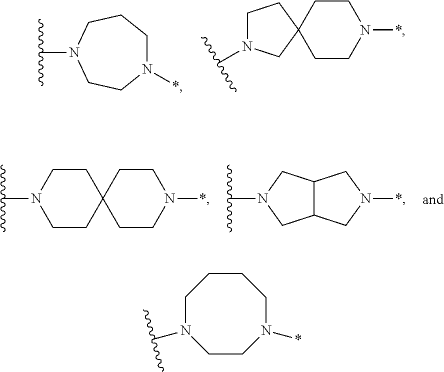

wherein:
   * indicates the point of attachment to Q².
13. The compound of clause 11 or clause 12, wherein L¹ is selected from a bond and —CH₂—, for example wherein L¹ is a bond.
14. The compound of any one of clauses 1 to 13, wherein
   (i) Q² is selected from phenyl, pyridyl or pyrimidyl, wherein said phenyl, pyridyl or pyrimidyl is optionally substituted by one or more (e.g. 1 or 2) R⁶; or
   (ii) Q² is selected from:

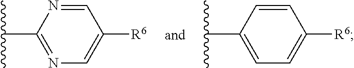

(iii) Q² is a 5-membered heteroaryl containing at least one ring nitrogen atom, wherein said 5-membered heteroaryl is optionally substituted by one or two R⁶; or
(iv) Q² is selected from: pyrrolyl, imidazolyl, pyrazolyl, triazolyl and tetrazolyl, wherein Q² is optionally substituted by one or two R⁶.
15. The compound of any one of clauses 1 to 14, wherein R⁶ is selected from the group consisting of: halo, —CN, —NO₂, C₁₋₄ alkyl, C₁₋₄ haloalkyl, —OR^{A1} and —NR^{A2}R^{B2}, for example wherein R⁶ is selected from the group consisting of: halo, —CN, C₁₋₄ alkyl and —OR^{A1}.
16. The compound of any of clauses 1 to 13, wherein Q² is selected from the group consisting of:

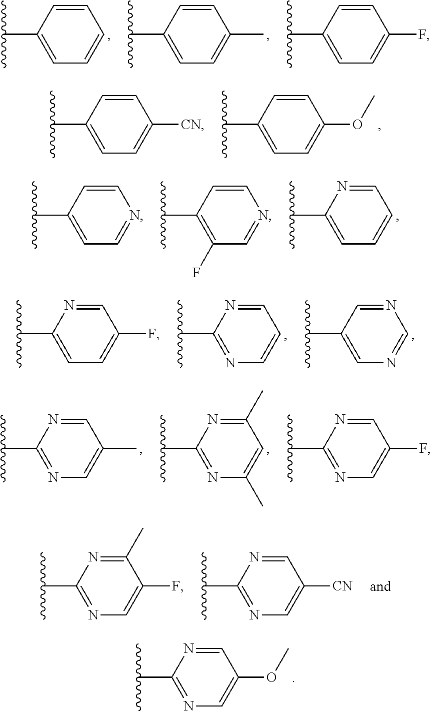

17. The compound of any one of clauses 1 to 13, wherein Q² is:

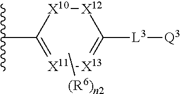

wherein X¹⁰, X¹¹, X¹² and X¹³ are each independently selected from CH and N, provided no more than two of X¹⁰, X¹¹, X¹² and X¹³ are N; n2 is an integer selected from 0, 1 or 2,
for example wherein Q² is:

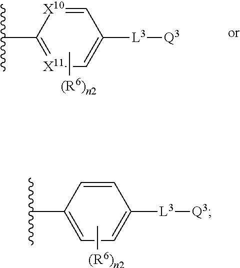

wherein X¹⁰ and X¹¹ are independently selected from CH and N, provided at least one of X¹⁰ and X¹¹ is N;
optionally n2 is 0.
18. The compound of clause 17, wherein:
   (i) L³ is selected from a bond, C₁₋₄ alkylene, —O— and —O—C₁₋₄ alkylene-; or
   (ii) L³ is

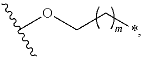

wherein m is 0, 1 or 2;
   [symbol] indicates the point of attachment to Q²; and * indicates the point of attachment to Q³; or
(iii) L³ is selected from a bond, —CH₂—, —O—, —O—CH₂—, —O—CH₂CH₂—; or
(iv) L³ is selected from a bond and —CH₂—.
19. The compound of clause 17 or clause 18, wherein Q³ is Group A, Group B, Group C, Group D or Group E:
   Group A: Q³ is selected from: phenyl, a 5- or 6-membered heteroaryl, wherein said phenyl or 5- or 6-membered heteroaryl is optionally substituted by one or more R⁷; or
   Group B: Q³ is selected from:

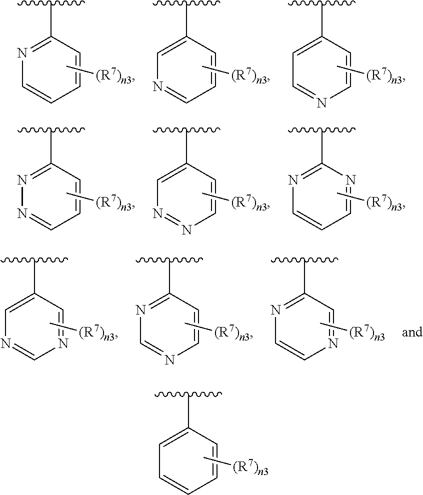

wherein n3 is 0, 1 or 2, and wherein [symbol] indicates the point of attachment to L³ of Formula (I); optionally n3 is 0 or 1; for example, n3 is 0; for example, n3 is 1; or
Group C: Q³ is

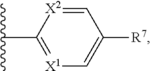

wherein: X¹ and X² are each independently selected from CH and N; optionally wherein at least one of X¹ and X² is N; or
Group D: Q³ is a 5-membered heteroaryl group, wherein said heteroaryl group contains 1 ring nitrogen atom and optionally 1 to 3 (e.g. 1 or 2) ring heteroatoms selected from O, S and N, wherein the heteroaryl is optionally substituted with one or more R⁷ group; or
Group E: Q³ is a 5-membered heteroaryl group selected from pyrrolyl, imidazolyl, pyrazolyl, triazolyl and tetrazolyl, wherein the heteroaryl is optionally substituted with one or more R⁷ group;
optionally wherein in any of Groups A to E, R⁷ is at each occurrence is independently selected from: halo, —CN, C₁₋₄ alkyl and —OR^{A1}.
20. The compound of clause 17 or clause 18, wherein Q³ is Group A or Group B:
   Group A: Q³ is a 4- to 7-membered saturated monocyclic heterocyclyl group containing 1 or 2 ring heteroatoms selected from O, S and N, wherein the heterocyclyl is optionally substituted by one or more R⁸; or
   Group B: Q³ is

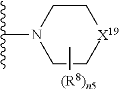

wherein: X¹⁹ is selected from NH, S, CH₂ and 0; and
n5 is 0, 1 or 2 (e.g. n5 is 0 or 1).
optionally wherein in any of Group A and Group B, R⁸ is at each occurrence independently selected from the group consisting of: halo and C₁₋₃ alkyl.
21. The compound of any of clauses 1 to 13, wherein Q² is -Q²-L³-Q³, and the group -Q²-L³-Q³ is selected from:

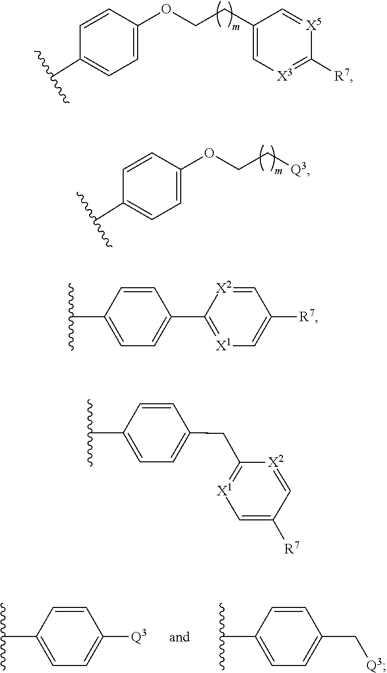

wherein
m is 0, 1 or 2;
X¹ and X² are each independently selected from CH and N (optionally wherein at least one of X¹ and X² is N);
X³ and X⁵ are each independently selected from CH and N (optionally wherein at least one of X³ and X⁵ is N); and
Q³ is a 5-membered heteroaryl optionally substituted by one or two R⁷ or a 4- to 7-membered non-aromatic saturated or partially saturated monocyclic heterocyclyl group containing at least one ring heteroatom selected from O, S and N, wherein said 4- to 7-membered heterocyclyl is optionally substituted by one or two R⁸;
optionally wherein:
(i) -Q²-L³-Q³ is selected from the group consisting of:

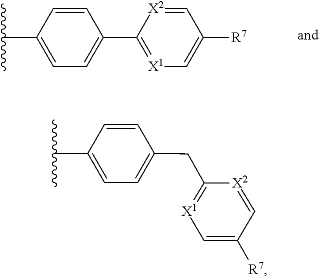

wherein:
   X¹ and X² are each independently selected from CH and N; or
(ii) -Q²-L³-Q³ is selected from the group consisting of:

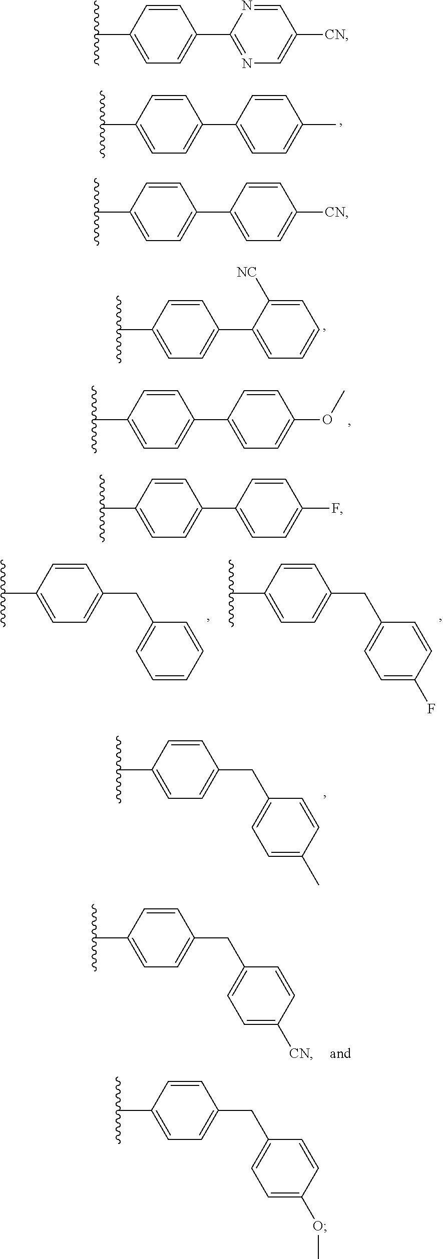

or
(iii) -Q²-L³-Q³ is selected from the group consisting of:

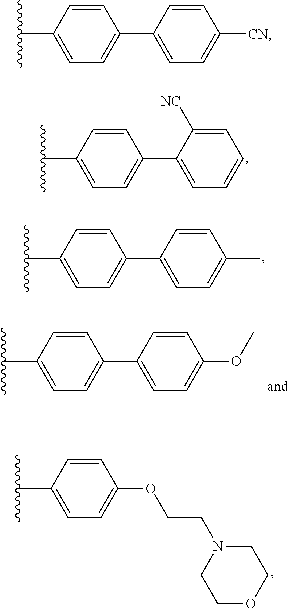

or
(iv) -Q²-L³-Q³ is

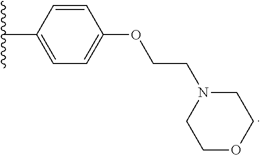 [SMILES: *c1ccc(OCCN2CCOCC2)cc1]

22. A compound selected from Table 1 or Table 2 in the description, or a pharmaceutically acceptable salt thereof.
23. A pharmaceutical composition comprising a compound of any of clauses 1 to 22, or a pharmaceutically acceptable salt thereof, and a pharmaceutically acceptable excipient.
24. A compound of any of clauses 1 to 22, or a pharmaceutically acceptable salt thereof, for use as a medicament.
25. A compound of any of clauses 1 to 22, or a pharmaceutically acceptable salt thereof, for use in the treatment or prevention of a fungal infection in a subject.
26. A method of treating or preventing a fungal infection in a subject, comprising administering to the subject an effective amount of a compound of any of clauses 1 to 22, or a pharmaceutically acceptable salt thereof.
27. The compound for the use of clause 25, or the method of clause 26, wherein:
   (i) the fungal infection is caused by or associated with a fungal species selected from Candida spp., Aspergillus spp., Cryptococcus spp. and Histoplasma spp. (e.g. Histoplasma. capsulatum); or
   (ii) the fungal infection is caused by or associated with a fungal species selected from Candida spp., for example C. albicans, C. glabrata, C. tropicalis, C. parapsilosis, C. krusei and C. auris; or
   (iii) the fungal infection is caused by or associated with C. auris; or
   (iv) the fungal infection is caused by or associated with a drug-resistant fungal strain; or
   (v) the fungal infection is caused by or associated with a drug resistant Candida strain (e.g. a drug-resistant strain of Candida albicans, Candida glabrata, Candida tropicalis, Candida parapsilosis, Candida krusei or Candida auris); or
   (vi) the fungal infection is caused by or associated with a drug resistant strain of Candida auris; and/or
   (vii) the fungal infection is candidiasis.
28. A compound of any of clauses 1 to 22, or a pharmaceutically acceptable salt thereof, for use in the treatment or prevention of a biofilm comprising a pathogenic fungus,
   for example wherein the biofilm comprises a fungal species selected from Candida spp., for example C. albicans, C. glabrata, C. tropicalis, C. parapsilosis, C. krusei and C. auris;
   particularly wherein the biofilm comprises C. auris.
29. The compound for the use or the method of any of clauses 25 to 28, wherein the compound is administered in combination with one or more antifungal agent,
   for example an additional antifungal agent selected from: a polyene, a triazole other than a compound of clause 1, an imidazole a thiazole, an echinocandin, an allylamine or an amorolfine antifungal agent.

1. A compound of the formula (I), or a pharmaceutically acceptable salt thereof: 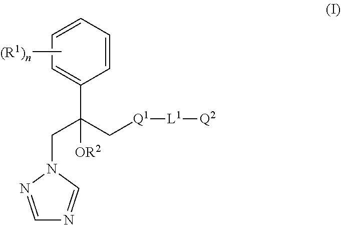
wherein:
R¹ is at each occurrence independently selected from: halo and C₁₋₄ alkyl;
n is 0, 1 or 2;
R² is selected from H and C₁₋₄ alkyl;
Q¹ is selected from: 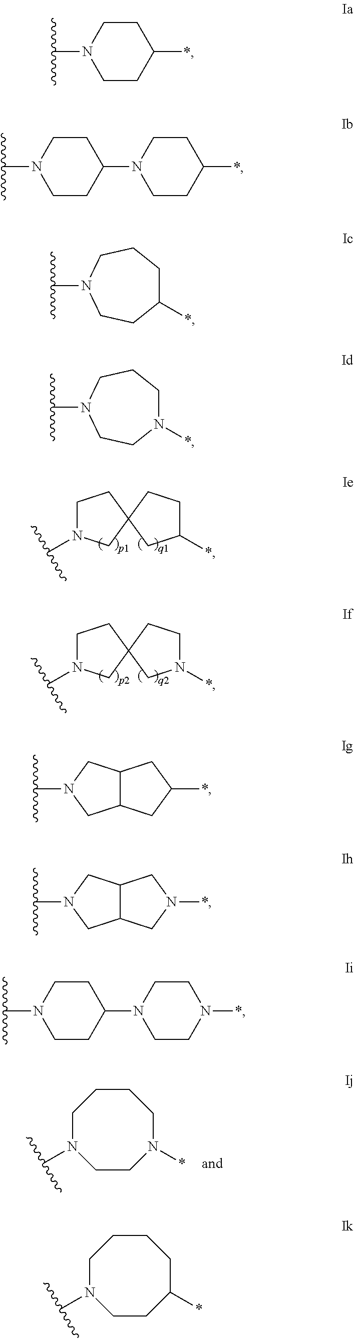
wherein * indicates the point of attachment to L¹,
p¹, q¹, p² and q² are independently an integer selected from 1 or 2,
and wherein Q¹ is optionally substituted by one or more R³;
each R³ is independently selected from: C₁₋₃ alkyl and ═O;
L¹ is a bond or is selected from —CH₂—, —O— and —NR⁴—, provided that L¹ is a bond or —CH₂— when Q¹ is bonded to L¹ via a ring nitrogen in Q¹;
R⁴ is selected from H and C₁₋₃ alkyl;
Q² is selected from phenyl or a 5- or 6-membered heteroaryl, wherein Q² is optionally substituted by one R⁵ and/or optionally one or more R⁶;
R⁵ is Q³-L³-, wherein
L³ is a bond or is selected from: C₁₋₄ alkylene, -L⁴-O-L⁵-, -L⁴-O-L⁵¹-O-L⁵-, -L⁴-NR^{A1}-L⁵-, -L⁴-S(O)ₓ-L⁵-, -L⁴-C(═O)-L⁵-, -L⁴-NR^{A1}C(═O)-L⁵-, -L⁴-C(═O)NR^{A1}-L⁵-, -L⁴-S(O)₂NR^{A1}-L⁵-, -L⁴-NR^{A1}S(O)₂-L⁵-, -L⁴-OC(═O)-L⁵ and -L⁴-C(═O)O-L⁵-,
wherein x is 0, 1 or 2;
L⁴ and L⁵ are independently selected from a bond and C₁₋₄ alkylene;
L⁵¹ is C₁₋₄ alkylene;
Q³ is selected from: phenyl, a 5- or 6-membered heteroaryl, 3- to 12-membered heterocyclyl and C₃₋₆ cycloalkyl, wherein said phenyl or 5- or 6-membered heteroaryl is optionally substituted by one or more R⁷, and said 3- to 12-membered heterocyclyl or C₃₋₆ cycloalkyl is optionally substituted by one or more R⁸;
each R⁶, R⁷ and R⁸ is independently selected from:
halo, ═O, —CN, —NO₂, C₁₋₆ alkyl, C₂₋₆ alkenyl, C₂₋₆ alkynyl, C₁₋₆ haloalkyl, C₁₋₆ cycloalkyl, —OR^{A1}, —S(O)ₓR^{A2} (wherein x is 0, 1, or 2), —NR^{A2}R^{B2}, —C(O)R^{A2}, —OC(O)R^{A2}, —C(O)OR^{A2}, —NR^{B2}C(O)R^{A2}, —NR^{B2}C(O)OR^{A2}, —C(O)NR^{A2}R^{B2}, —NR^{B2}SO₂R^{A2} and SO₂NR^{A2}R^{B2};
wherein said C₁₋₆ alkyl, C₂₋₆ alkenyl, C₂₋₆ alkynyl are each is optionally substituted by 1 or 2 substituents independently selected from: halo, —CN, —OR^{A3}, —NR^{A3}R^{B3} and —SO₂R³; and
wherein said C₁₋₆ cycloalkyl is optionally substituted by one or more substituents independently selected from: halo, ═O, —CN, —NO₂, C₁₋₄ alkyl and C₁₋₄ haloalkyl;
R^{A1}, R^{A2}, R^{B2}, R^{A3} and R^{B3} are each independently selected from: H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl;
with the provisos (i) and (ii):
(i) when n is 1 or 2; R¹ is halo;
Q¹ is 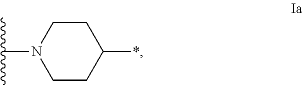
L¹ is —O—, —NH— or —N(CH₃)—; and
Q² is phenyl, pyridyl, or pyrimidyl;
then Q² is substituted by X and optionally one or more R⁶, wherein X is selected from Q³-L³-, —O—C₁₋₆ alkyl and —O—C₁₋₆ alkyl-OR^{A4}, and R^{A4} is H or C₁₋₄ alkyl; and
(ii) when n is 1 or 2; R¹ is halo; L¹ is a bond; and
Q¹ is 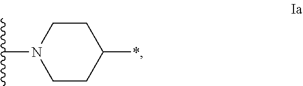
then Q² is phenyl or 6-membered heteroaryl, wherein said Q² is substituted by X and optionally one or more R⁶, wherein X is selected from Q³-L³-, —O—C₁₋₆ alkyl and —O—C₁₋₆ alkyl-OR^{A4}, and R^{A4} is H or C₁₋₄ alkyl.

2. The compound of claim 1, wherein R² is H.

3. The compound of claim 1, wherein n is 1 or 2 and R¹ is selected from C₁₋₃ alkyl and halo, for example R¹ is selected from methyl and F.

4. The compound of claim 1, wherein the group 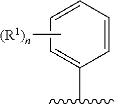
has the structure

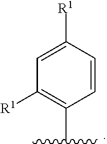

5. The compound of claim 1, wherein the group 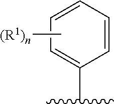
has the structure

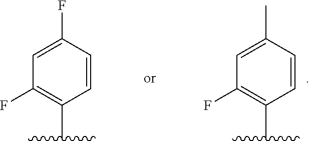

6. The compound of claim 1, wherein Q¹ is selected from the group consisting of: 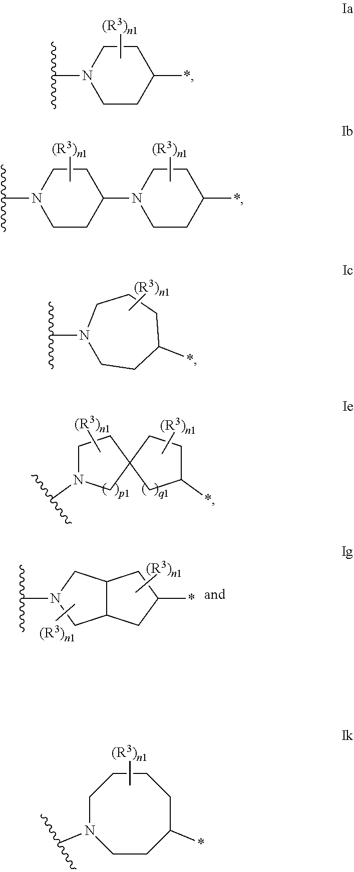
wherein:
* indicates the point of attachment to L¹;
p¹ and q¹ are independently an integer selected from 1 or 2; [symbol]
indicates the point of attachment to Formula (I); and
each n1 is independently selected from 0, 1 or 2.

7. The compound of any one of claim 1, wherein Q¹ is 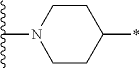 [SMILES: *C1CCN(*)CC1]
wherein * indicates the point of attachment to L¹.

8. The compound of claim 6, wherein L¹ is selected from a bond, —CH₂— and —NH—.

9. The compound of claim 1, wherein Q¹-L¹- is selected from the group consisting of: 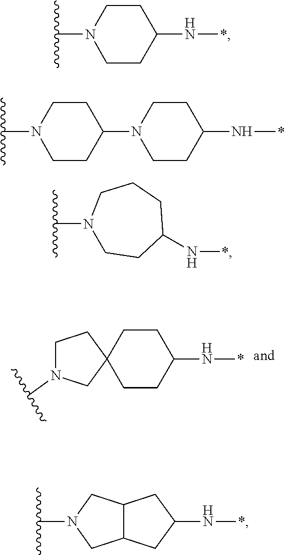
wherein:
* indicates the point of attachment to Q².

10. The compound claim 1, wherein -Q¹-L¹- is 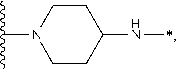
wherein * indicates the point of attachment to Q².

11. The compound of claim 1, wherein Q¹ is selected from the group consisting of: 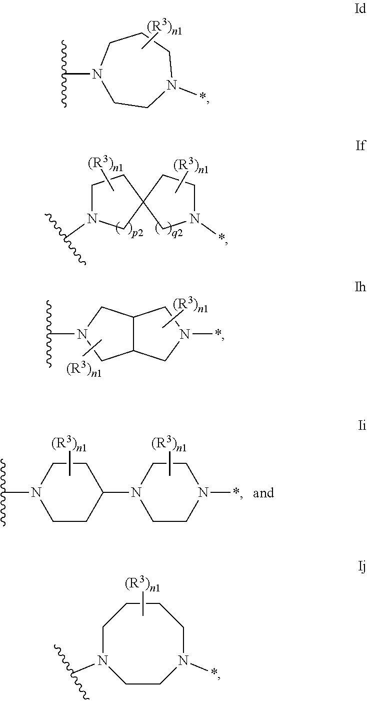
wherein:
* indicates the point of attachment to L¹;
p² and q² are independently an integer selected from 1 or 2; [symbol]
indicates the point of attachment to Formula (I); and
each n1 is independently selected from 0, 1 or 2.

12. The compound of claim 1, wherein Q¹ is selected from the group consisting of: 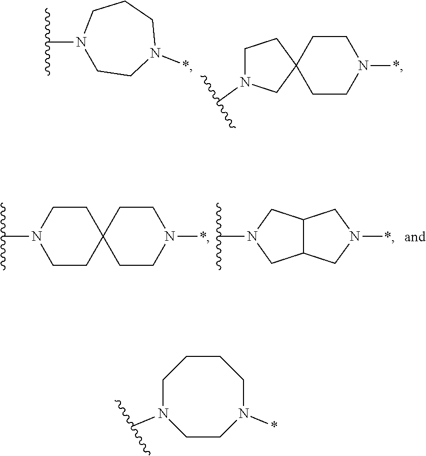
wherein:
* indicates the point of attachment to Q².

13. The compound of claim 11, wherein L¹ is selected from a bond and —CH₂—.

14. The compound of claim 1, wherein
(i) Q² is selected from phenyl, pyridyl or pyrimidyl, wherein said phenyl, pyridyl or pyrimidyl is optionally substituted by one or more R⁶; or
(ii) Q² is selected from: 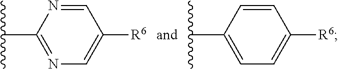
or
(iii) Q² is a 5-membered heteroaryl containing at least one ring nitrogen atom, wherein said 5-membered heteroaryl is optionally substituted by one or two R⁶; or
(iv) Q² is selected from: pyrrolyl, imidazolyl, pyrazolyl, triazolyl and tetrazolyl, wherein Q² is optionally substituted by one or two R⁶.

15. The compound of claim 1, wherein R⁶ is selected from the group consisting of: halo, —CN, —NO₂, C₁₋₆ alkyl, C₁₋₆ haloalkyl, —OR^{A1} and —NR^{A2}R^{B2}, for example wherein R⁶ is selected from the group consisting of: halo, —CN, C₁₋₄ alkyl and —OR^{A1}.

16. The compound of claim 15, wherein R⁶ is —OR^{A1}, and R^{A1} is selected from the group consisting of: H, C₁₋₆ alkyl and C₁₋₆ haloalkyl, wherein said C₁₋₆ alkyl is optionally substituted by 1 or 2 substituents selected from —OR^{A4}, and R^{A4} is H or C₁₋₄ alkyl.

17. The compound of claim 16, wherein R^{A1} is:
(i) C₁₋₆ alkyl substituted with —OR^{A4}, for example —(CH₂)₂—OCH₃; or
(ii) C₁₋₆ alkyl; or
(iii) C₃₋₆ alkyl.

18. The compound of claim 1, wherein Q² is selected from the group consisting of:

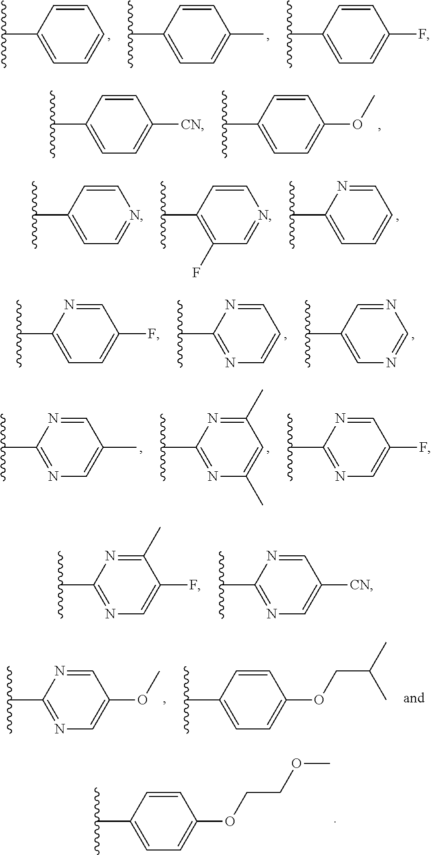

19. The compound of claim 1, wherein Q² is: 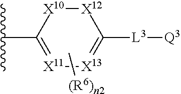
wherein X¹⁰, X¹¹, X¹² and X¹³ are each independently selected from CH and N, provided no more than two of X¹⁰, X¹¹, X¹² and X¹³ are N; n2 is an integer selected from 0, 1 or 2.

20. The compound of claim 19, wherein:
(i) L³ is selected from a bond, C₁₋₄ alkylene, —O— and —O—C₁₋₄ alkylene-; or
(ii) L³ is 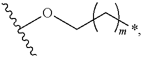
wherein m is 0, 1 or 2; [symbol]
indicates the point of attachment to Q²; and * indicates the point of attachment to Q³; or
(iii) L³ is selected from a bond, —CH₂—, —O—, —O—CH₂—, —O—CH₂CH₂—; or
(iv) L³ is selected from a bond and —CH₂—; or
(v) L³ is

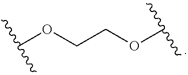 [SMILES: *OCCO*]

21. The compound of claim 19, wherein Q³ is Group A, Group B, Group C, Group D or Group E:
Group A: Q³ is selected from: phenyl, a 5- or 6-membered heteroaryl, wherein said phenyl or 5- or 6-membered heteroaryl is optionally substituted by one or more R⁷; or
Group B: Q³ is selected from: 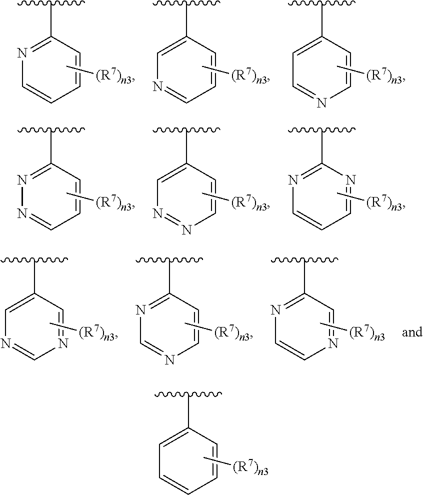
wherein n3 is 0, 1 or 2, and wherein [symbol] indicates the point of attachment to L³ of Formula (I); optionally n3 is 0 or 1;
Group C: Q³ is 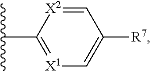
wherein: X¹ and X² are each independently selected from CH and N; optionally wherein at least one of X¹ and X² is N; or
Group D: Q³ is a 5-membered heteroaryl group, wherein said heteroaryl group contains 1 ring nitrogen atom and optionally 1 to 3 ring heteroatoms selected from O, S and N, wherein the heteroaryl is optionally substituted with one or more R⁷ group; or
Group E: Q³ is a 5-membered heteroaryl group selected from pyrrolyl, imidazolyl, pyrazolyl, triazolyl and tetrazolyl, wherein the heteroaryl is optionally substituted with one or more R⁷ group;
optionally wherein in any of Groups A to E, R⁷ is at each occurrence is independently selected from: halo, —CN, C₁₋₄ alkyl and —OR^{A1}.

22. The compound of claim 19, wherein Q³ is Group A, Group B or Group C:
Group A: Q³ is a 4- to 7-membered saturated monocyclic heterocyclyl group containing 1 or 2 ring heteroatoms selected from O, S and N, wherein the heterocyclyl is optionally substituted by one or more R⁸; or
Group B: Q³ is 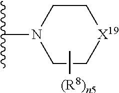
wherein: X¹⁹ is selected from NH, S, CH₂ and 0; and
n5 is 0, 1 or 2; or
Group C: Q³ is 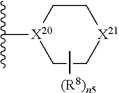
wherein: X²⁰ is selected from N and CH;
X²¹ is selected from NH, S, CH₂ and O; and
n5 is 0, 1 or 2,
optionally wherein in any of Group A, Group B and Group C, R⁸ is at each occurrence independently selected from the group consisting of: halo and C₁₋₃ alkyl.

23. The compound of claim 1, wherein Q² is -Q²-L³-Q³, and the group -Q²-L³-Q³ is selected from: 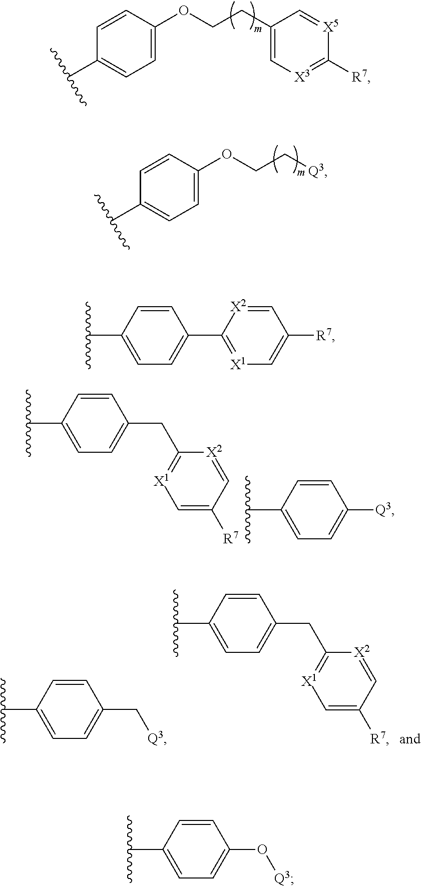
wherein
m is 0, 1 or 2;
X¹ and X² are each independently selected from CH and N;
X³ and X⁵ are each independently selected from CH and N; and
Q³ is a 5-membered heteroaryl optionally substituted by one or two R⁷ or a 4- to 7-membered non-aromatic saturated or partially saturated monocyclic heterocyclyl group containing at least one ring heteroatom selected from O, S and N, wherein said 4- to 7-membered heterocyclyl is optionally substituted by one or two R⁸.

24. A compound selected from Table 1, Table 2 and/or Table 3 in the description, or a pharmaceutically acceptable salt thereof.

25. A pharmaceutical composition comprising a compound of claim 1, or a pharmaceutically acceptable salt thereof, and a pharmaceutically acceptable excipient.

26. (canceled)

27. (canceled)

28. A method of treating or preventing a fungal infection in a subject, comprising administering to the subject an effective amount of a compound of claim 1, or a pharmaceutically acceptable salt thereof.

29. The method of claim 28, wherein:
(i) the fungal infection is caused by or associated with a fungal species selected from Candida spp., Aspergillus spp., Cryptococcus spp. and Histoplasma spp.; or
(ii) the fungal infection is caused by or associated with a fungal species selected from Candida spp.; or
(iii) the fungal infection is caused by or associated with C. auris; or
(iv) the fungal infection is caused by or associated with a drug-resistant fungal strain; or
(v) the fungal infection is caused by or associated with a drug resistant Candida strain; or
(vi) the fungal infection is caused by or associated with a drug resistant strain of Candida auris; and/or
(vii) the fungal infection is candidiasis.

30. (canceled)

31. The method of claim 28, wherein the compound is administered in combination with one or more antifungal agent.

32. The compound of claim 1, wherein Q² is selected from the group consisting of:

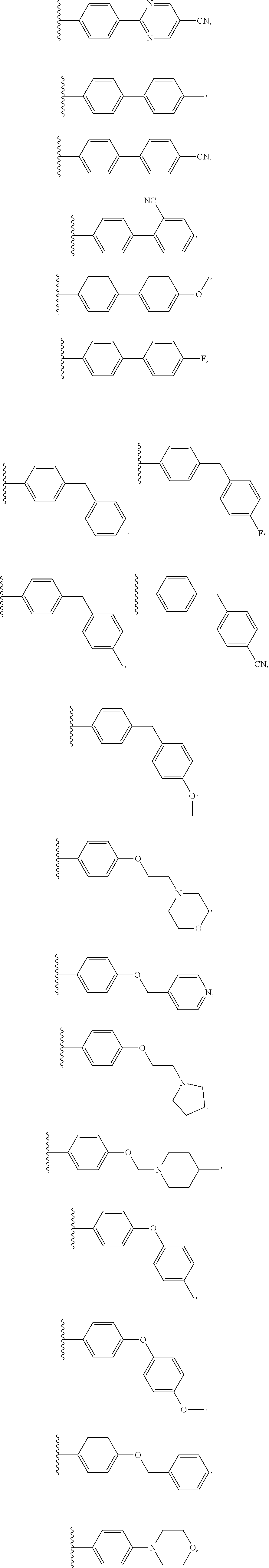

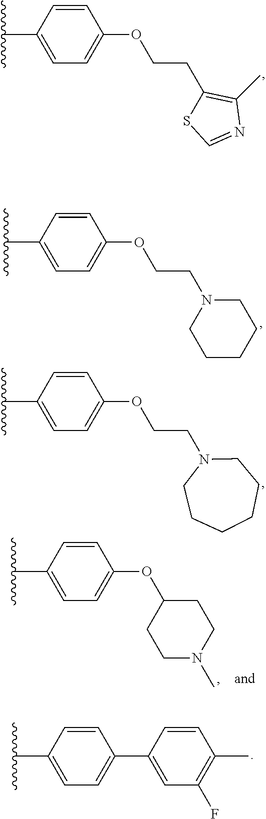

33. The compound of claim 1, wherein Q² is: 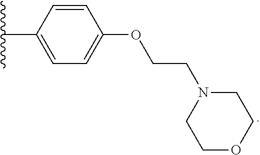 [SMILES: *c1ccc(OCCN2CCOCC2)cc1]
